(19) United States
(12) Patent Application Publication
(10) Pub. No.: US 2011/0189692 A1
(43) Pub. Date: Aug. 4, 2011

(54) ASSAY FOR PATHOGENIC CONFORMERS

(75) Inventors: David Peretz, El Cerrito, CA (US); Xuemei Wang, Castro Valley, CA (US); Carol Man Gao, San Ramon, CA (US); Alice Yam, Fremont, CA (US); Anthony Lau, San Francisco, CA (US); Ping Wu, Danville, CA (US)

(73) Assignee: Novartis AG

(21) Appl. No.: 12/990,480

(22) PCT Filed: Apr. 29, 2009
(86) PCT No.: PCT/US09/42185

§ 371 Date: Mar. 10, 2011

Related U.S. Application Data
(60) Provisional application No. 61/049,396, filed on Apr. 30, 2008.

(51) Int. Cl. G01N 33/68

(52) U.S. Cl. 435/7.1; 436/501

(57) ABSTRACT

The invention provides methods for detecting the presence of a non-prion pathogenic conformer in a sample by contacting the sample suspected of containing a non-prion pathogenic conformer with a pathogenic conformer-specific binding reagent under conditions that allow the binding of the reagent to the pathogenic conformer, if present; and detecting the presence the pathogenic conformer, if any, in the sample by its binding to the reagent; where the pathogenic conformer-specific binding reagent is typically derived from a prion protein fragment and interacts preferentially with a pathogenic prion protein. Methods for diagnosis of conformational diseases are also provided.

### CROSS-REFERENCE TO RELATED APPLICATIONS

This application claims the benefit of U.S. Provisional Application No. 61/049,396, filed Apr. 30, 2008, the contents of which are hereby incorporated by reference in their entirety.

### BACKGROUND

Protein conformational diseases include a variety of clinically unrelated diseases, such as transmissible spongiform encephalopathies, Alzheimer's disease, ALS, and diabetes, that arise from an aberrant conformational transition of a normal protein into a pathogenic conformer. This transition, in turn, can lead to self-association of the pathogenic conformer with consequent tissue deposition and is hypothesized to lead to damage of the surrounding tissue. These diseases share similarities in clinical presentations, typically a rapid progression from diagnosis to death following varying lengths of incubation.

Detection of the pathogenic conformers of conformational disease proteins in living subjects and samples obtained from living subjects has proven difficult. The current techniques for confirming the presence of pathogenic conformers in living patients are crude and invasive. For example, histopathological examination would require biopsies that are risky to the subject. Histopathology is inherently prone to sampling error as lesions and amyloid deposits can be missed depending on the area where the biopsy is taken. Thus, definitive diagnosis and palliative treatments for these conditions before death of the subject remains a substantially unmet challenge.

Deposition of amyloid-beta protein (Aβ) aggregates, mainly Aβ 1-40 (Aβ40) and 1-42 (Aβ42), has been exhaustively linked to Alzheimer's disease (AD) and is considered the gold-standard marker for the disease. However, the only definitive test for AD is immunohistochemical staining of Aβ plaques from post-mortem brain samples. Currently, there are no FDA-approved ante-mortem diagnostic tests for AD. Plasma or CSF samples could be used for ante-mortem tests. Some ante-mortem AD tests have focused on the cerebrospinal fluid (CSF) and attempt to quantitate soluble Aβ42. However, this biomarker only serves as an indirect measurement of AD.

A test that can specifically detect aggregated Aβ directly from the CSF or other body fluids such as plasma would have a great advantage. Early detection of aggregated Aβ will allow faster and more efficient diagnosis and evaluation of potential therapies for Alzheimer's disease.

Tests that can detect the pathogenic conformer of the other conformational disease proteins are also desired, as they would also allow faster and more efficient diagnosis and evaluation of potential therapies for these conformational diseases.

### BRIEF SUMMARY OF PREFERRED EMBODIMENTS

The present invention relates, in part, to pathogenic conformer-specific binding reagents which interact preferentially with both a pathogenic prion protein and other non-prion pathogenic conformers, In some embodiments, the PCSB reagent is derived from a prion protein fragment, such as PrP19-30 (SEQ ID NO: 242), PrP23-30 (SEQ ID NO: 243), PrP100-111 (SEQ ID NO: 244), PrP101-110 (SEQ ID NO: 245), PrP154-165 (SEQ ID NO: 246) and PrP226-237 (SEQ ID NO: 247). In other embodiments, the pathogenic conformer-specific binding reagent has amino acid sequence of: SEQ ID NO: 242, SEQ ID NO: 243, SEQ ID NO: 244, SEQ ID NO: 245, SEQ ID NO: 246 and SEQ ID NO: 247.

In other embodiments, the PCSB reagent is a peptoid analog of a prion protein fragment. In some embodiments the peptoid analog has one of the following structures:

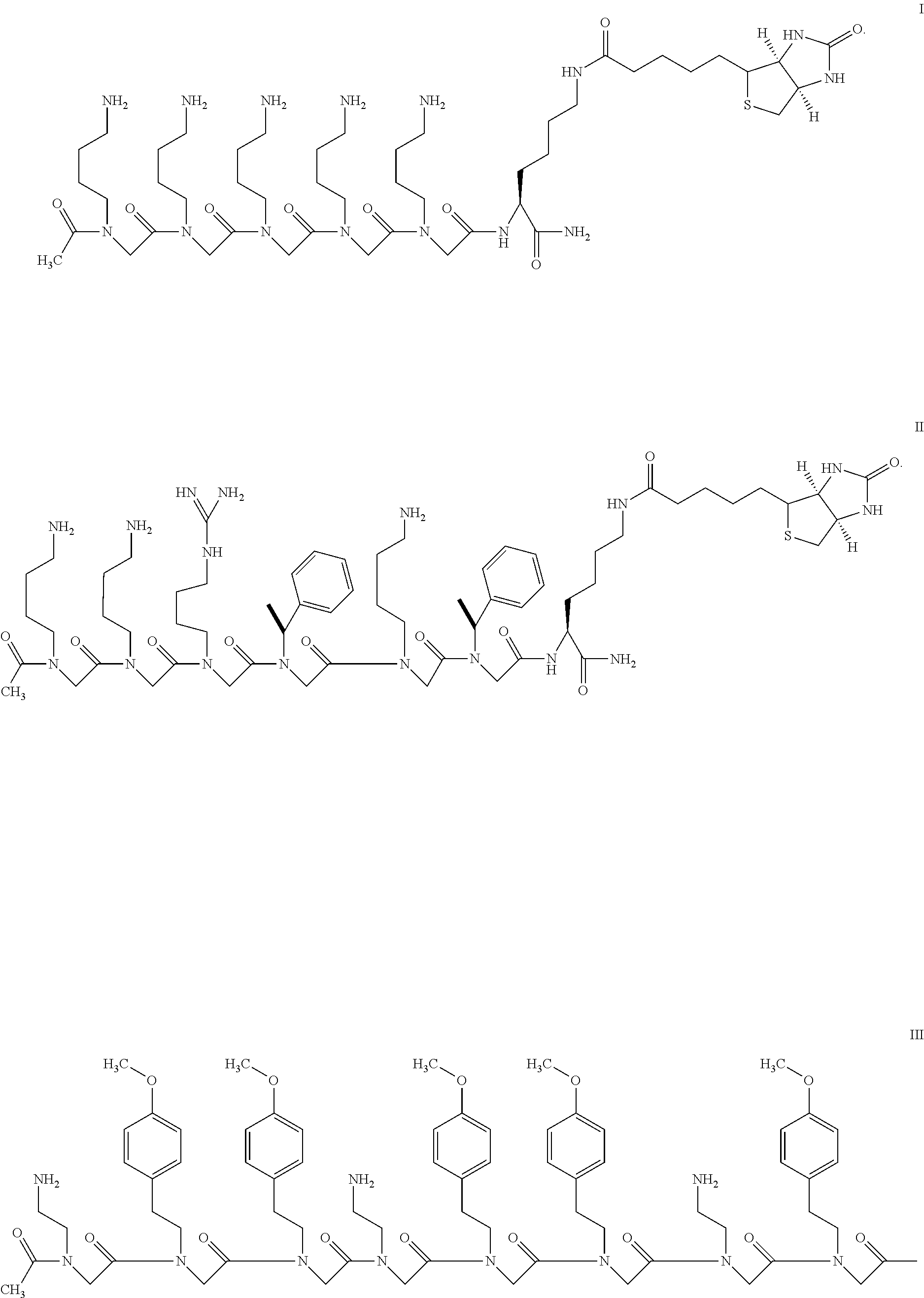

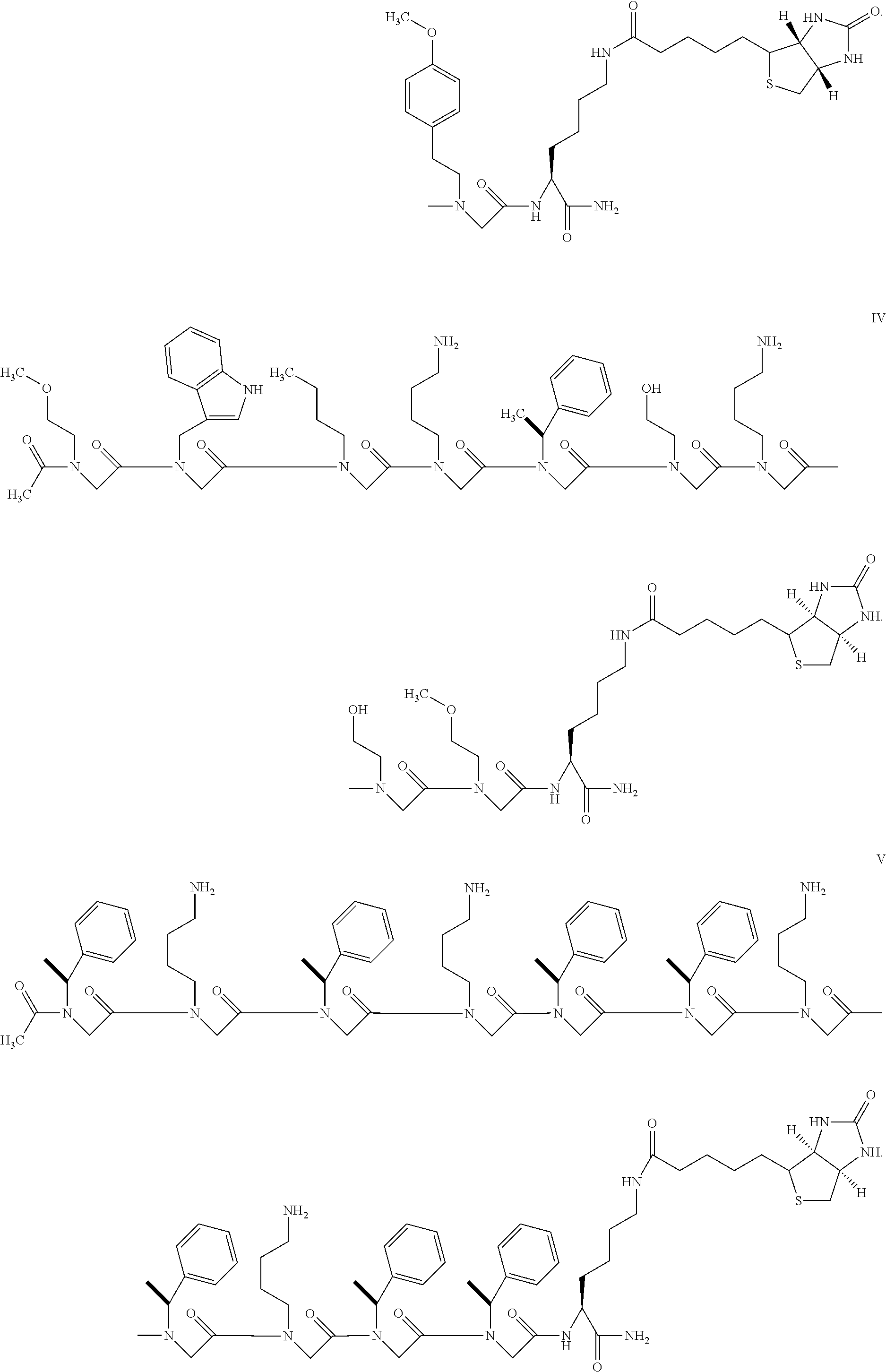

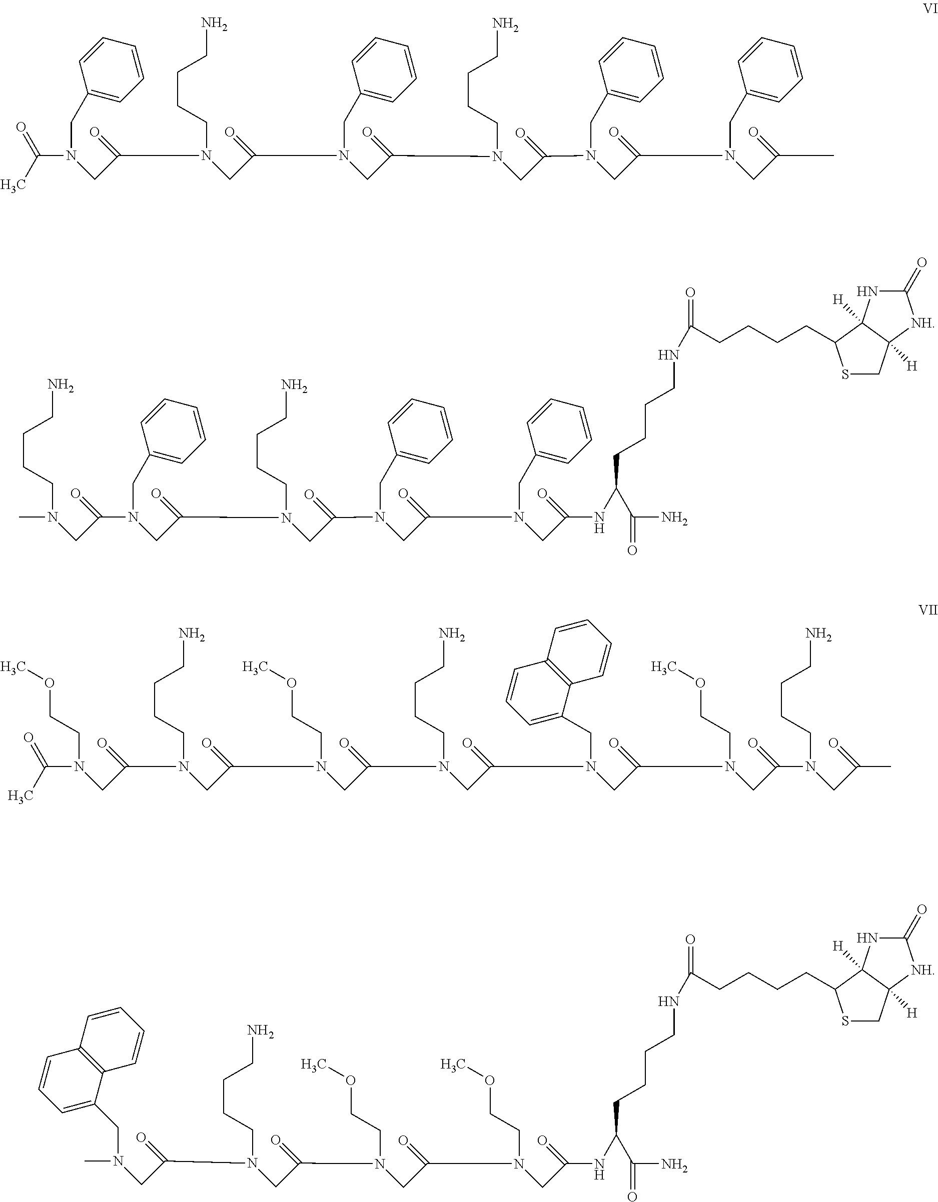

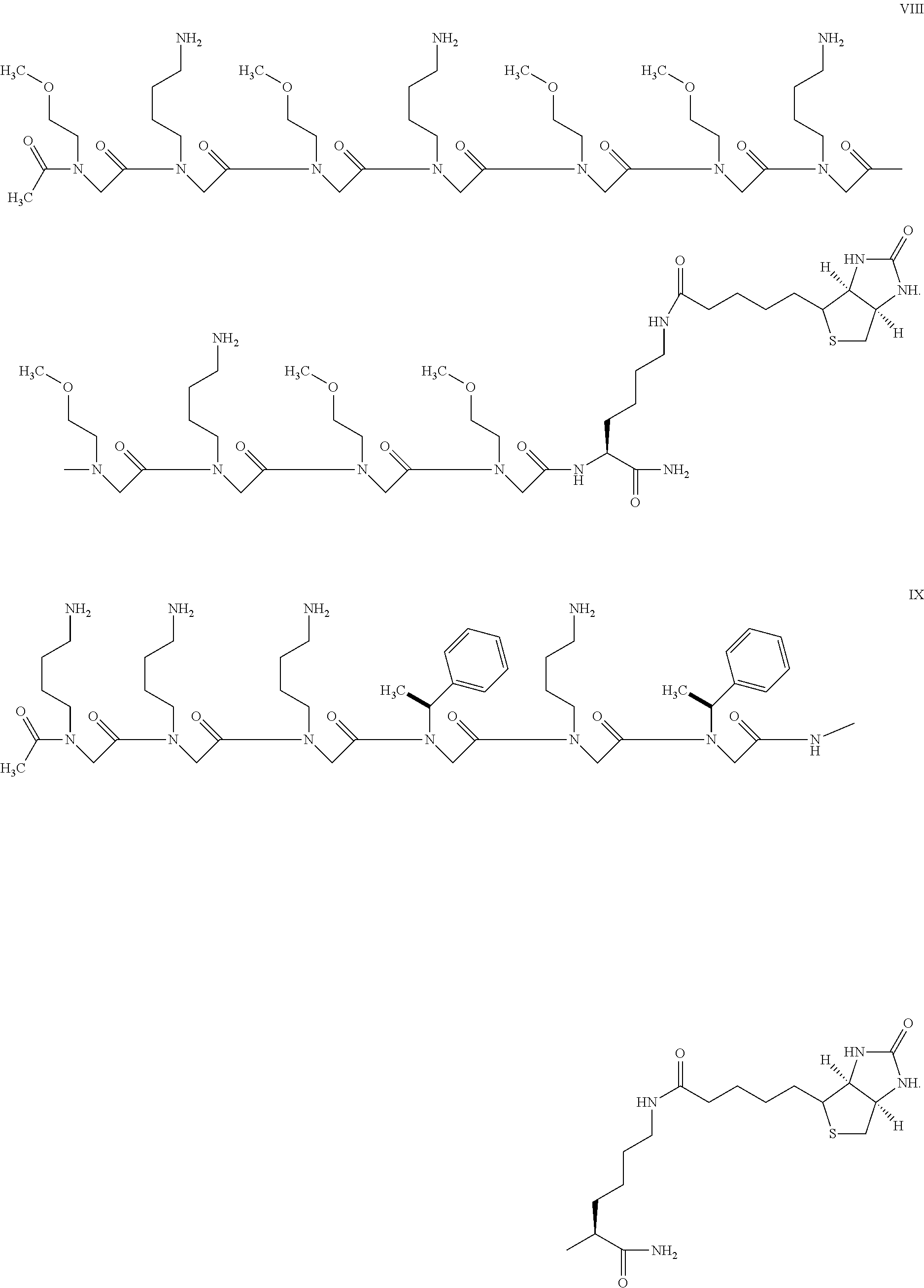

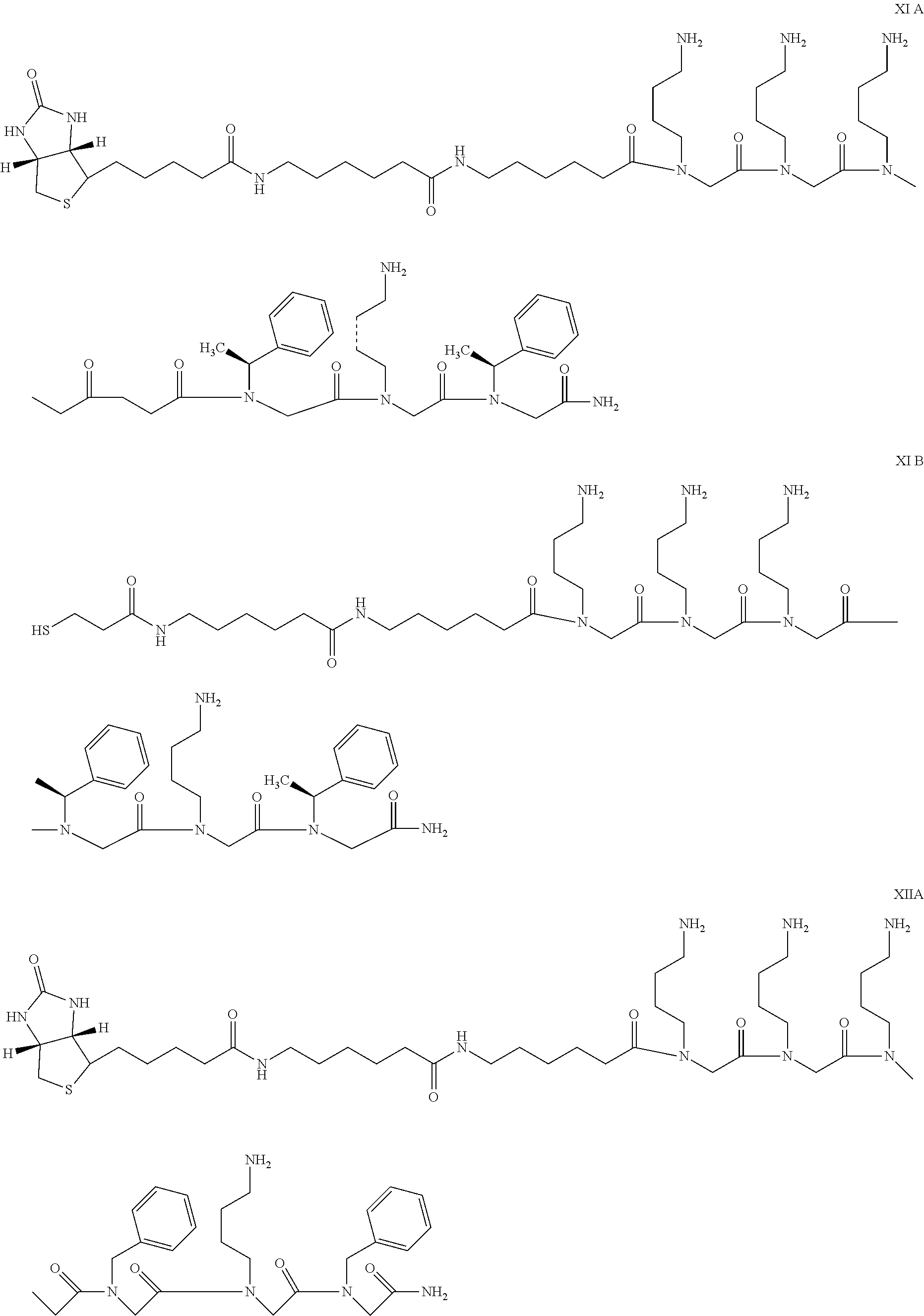

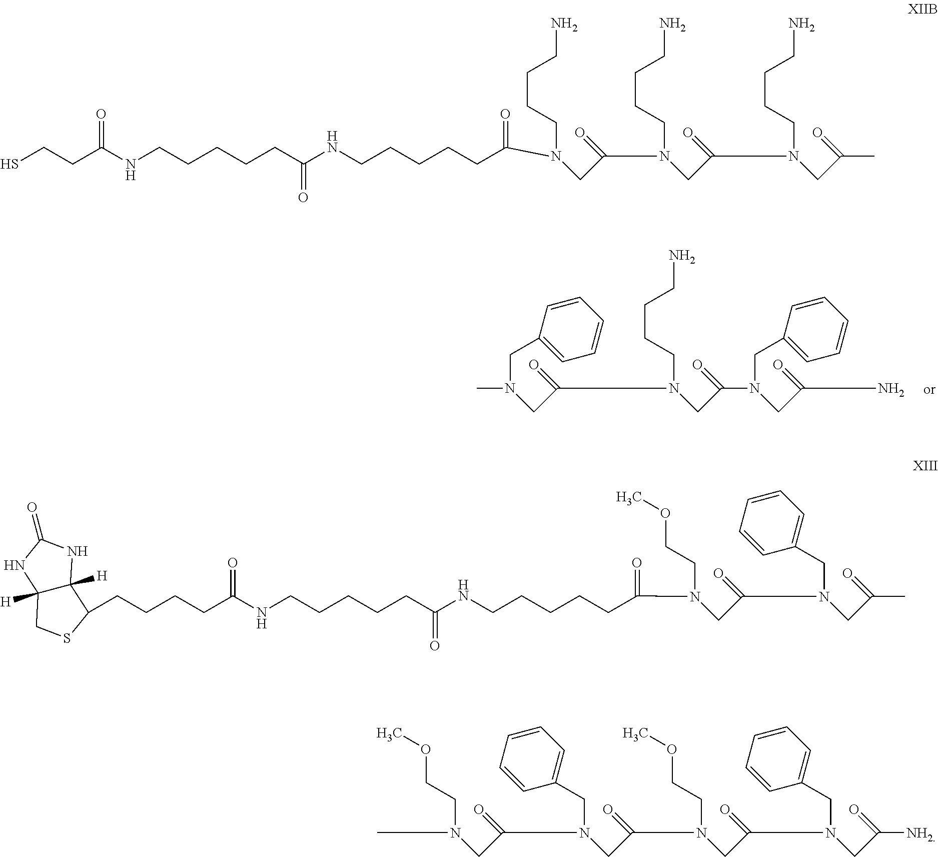

In preferred embodiments, the pathogenic conformer-specific binding reagent has a net charge of at least positive three at physiological pH or least positive four at physiological pH.

In one aspect, methods for detecting the presence of a non-prion pathogenic conformer are provided. The detection method includes the steps of contacting a sample suspected of containing the non-prion pathogenic conformer with a pathogenic conformer-specific binding reagent under conditions that allow binding of the reagent to the non-prion pathogenic conformer, if present, to form a complex; and detecting the non-prion pathogenic conformer, if any, in the sample by its binding to the pathogenic conformer-specific binding reagent; wherein the pathogenic conformer-specific binding reagent is derived from a prion protein fragment and interacts preferentially with a pathogenic prion protein.

The non-prion pathogenic conformer detected by the reagent may be a conformer associated with an amyloid disease, such as a systemic amyloidosis, tauopathy, or synucleinopathy. For example, the non-prion pathogenic conformer may be one associated with Alzheimer's disease, ALS, immunoglobulin-related diseases, serum amyloid A-related diseases, or diabetes type II.

In preferred embodiments, the non-prion pathogenic conformer detected by the PCSB reagent is an Alzheimer's disease conformer, such as an amyloid-β or tau protein. In such cases, the preferred pathogenic conformer-specific binding reagent is derived from prion fragments PrP19-30 (SEQ ID NO: 242), PrP23-30 (SEQ ID NO: 243), PrP100-111 (SEQ ID NO: 244), PrP101-110 (SEQ ID NO: 245), PrP154-165 (SEQ ID NO: 246), PrP226-237 (SEQ ID NO: 247), SEQ ID NO: 14, SEQ ID NO: 50, or SEQ ID NO: 68 and includes

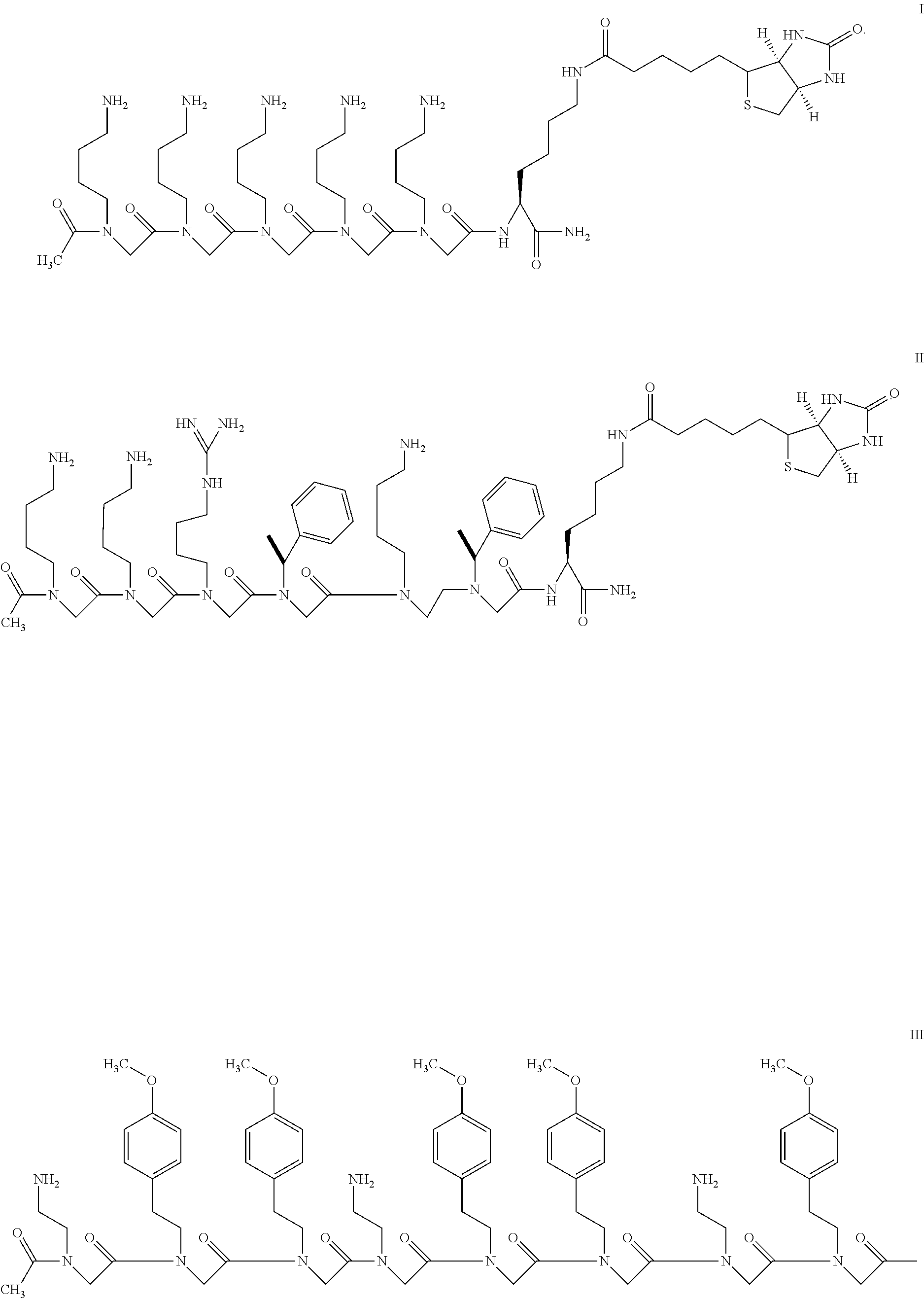

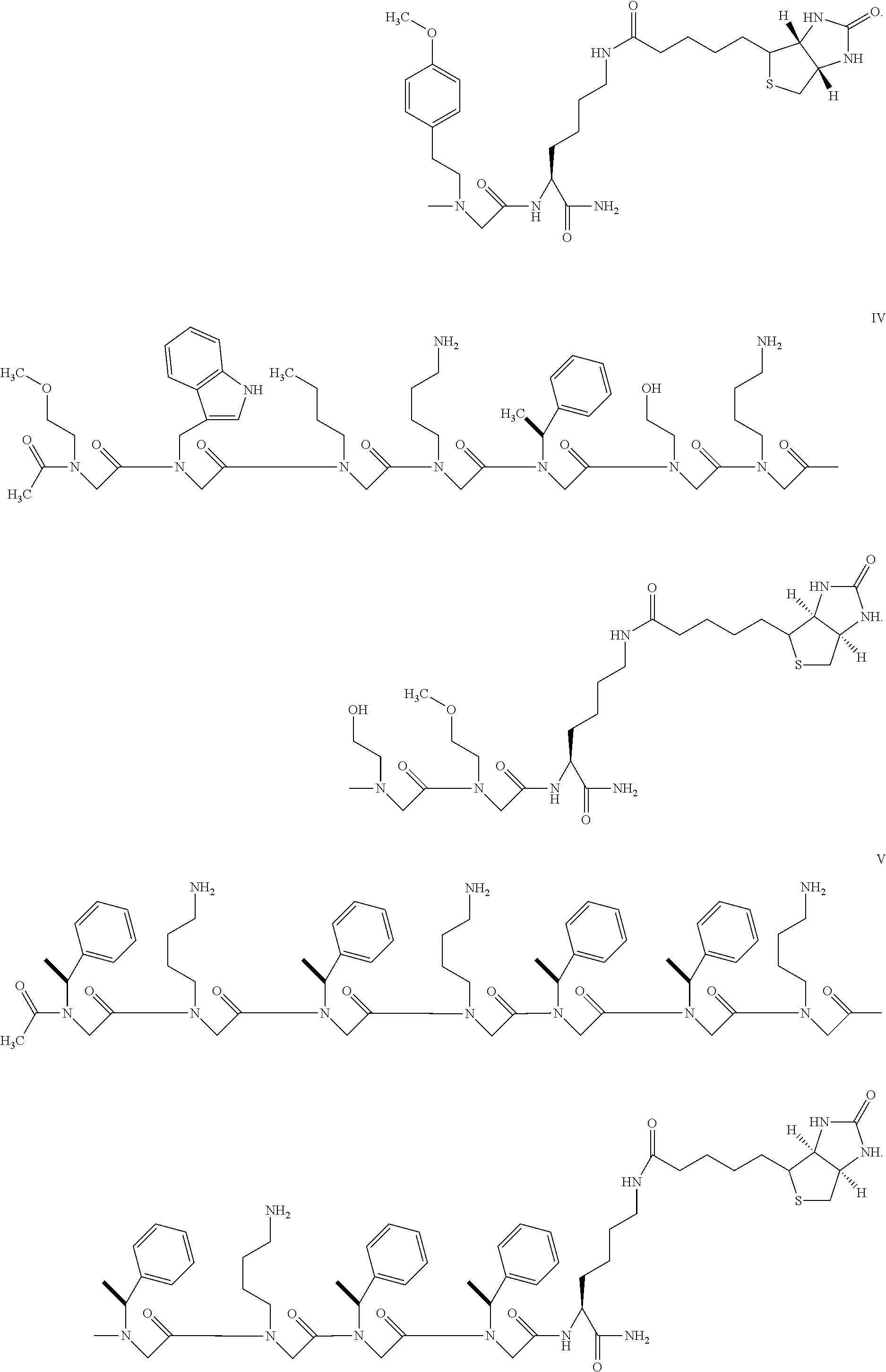

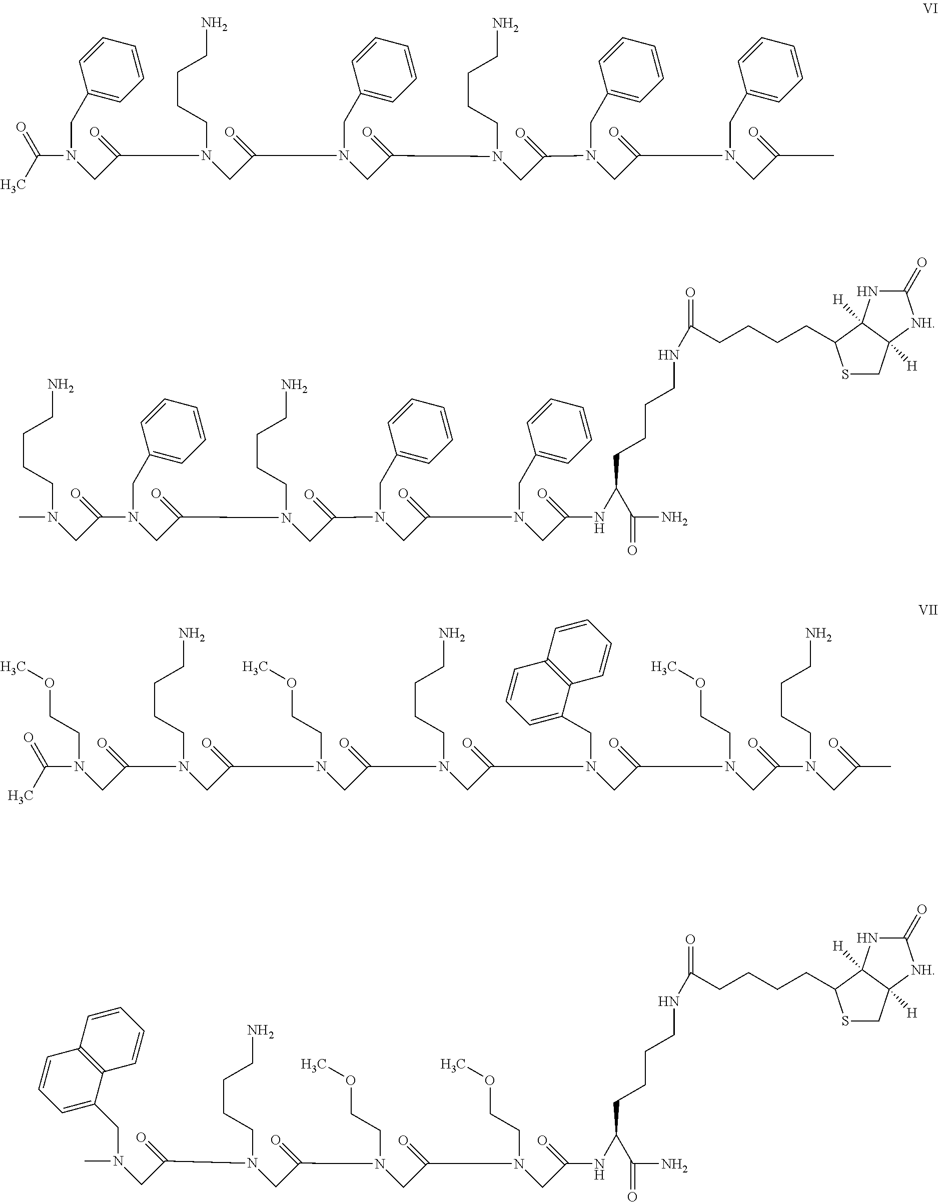

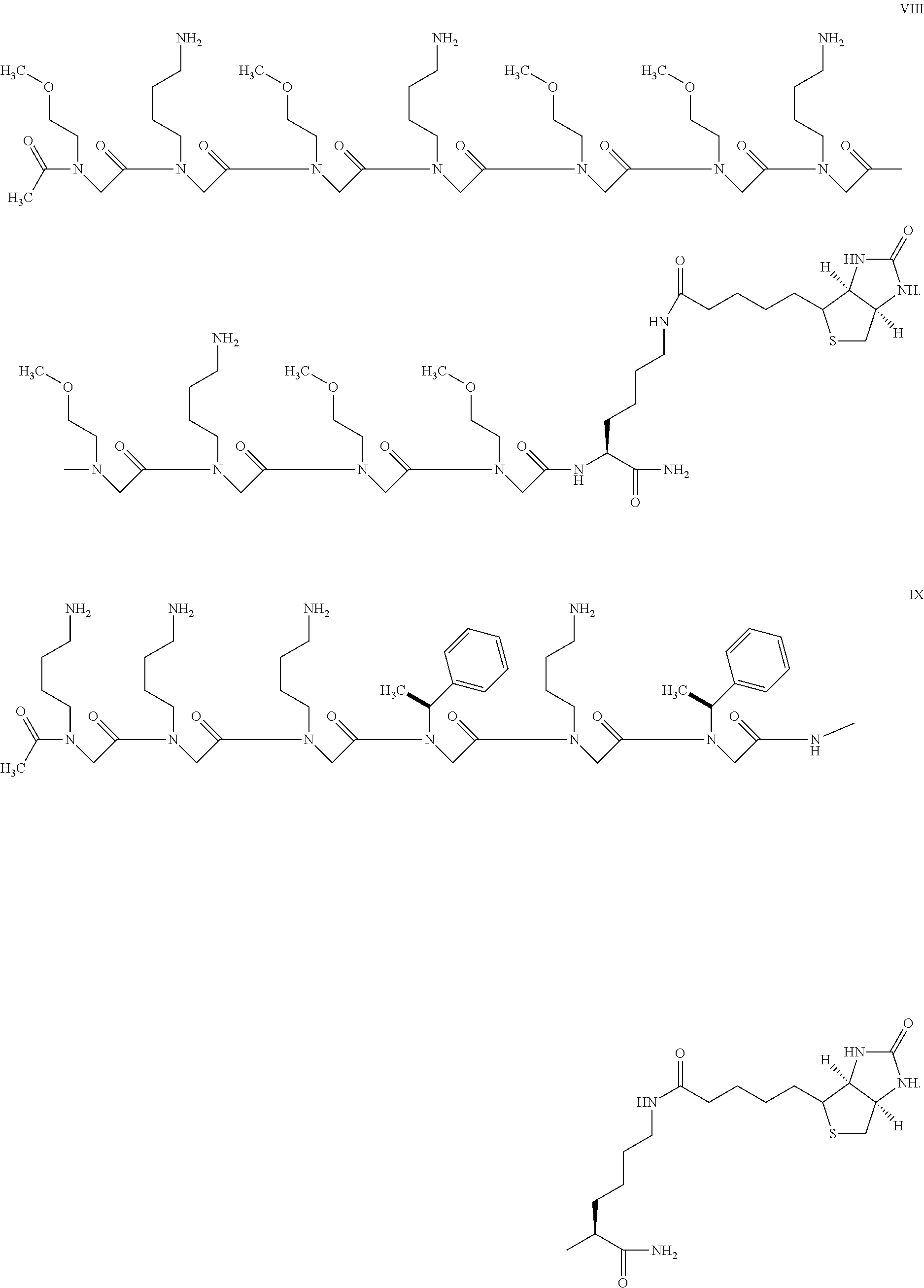

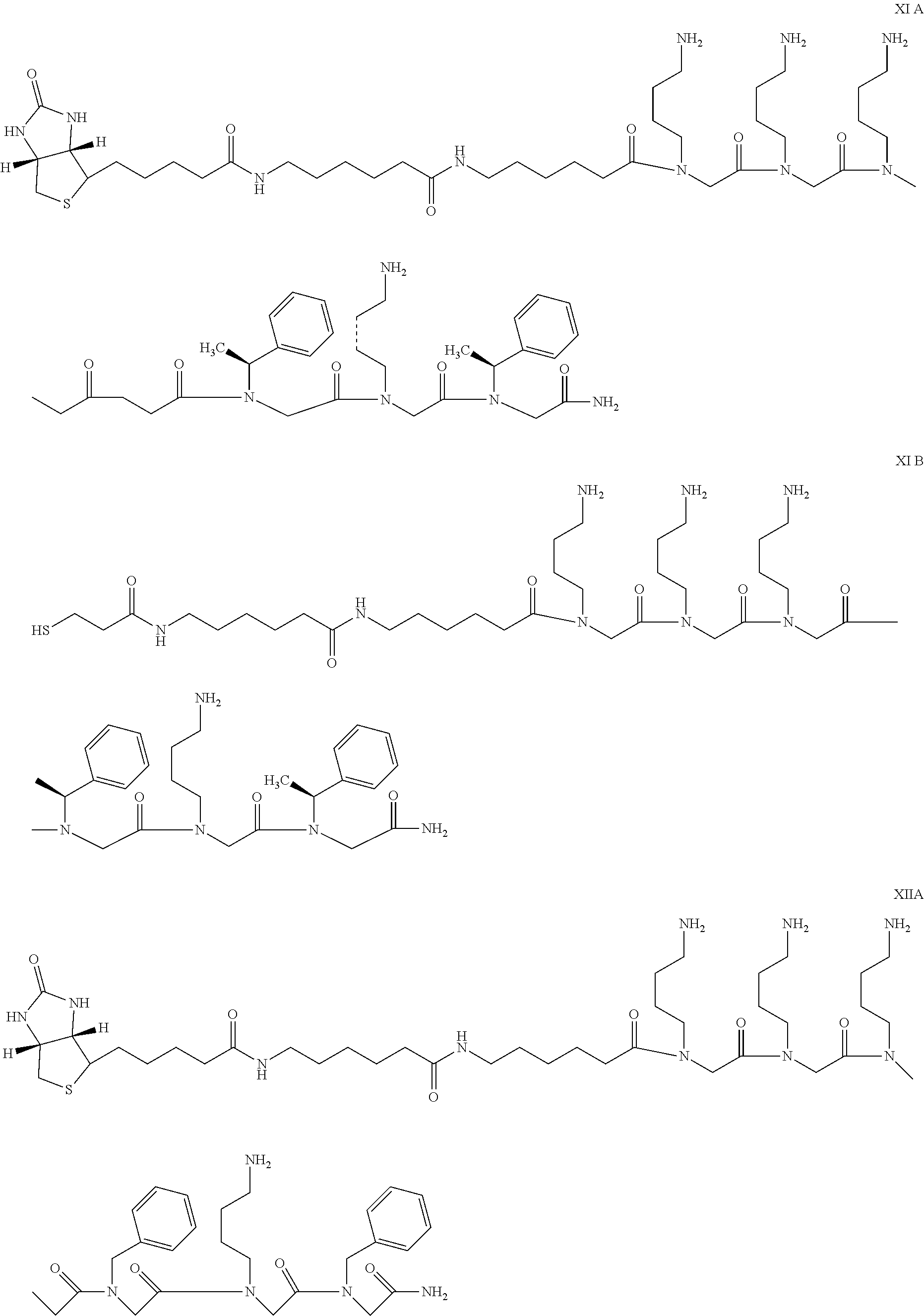

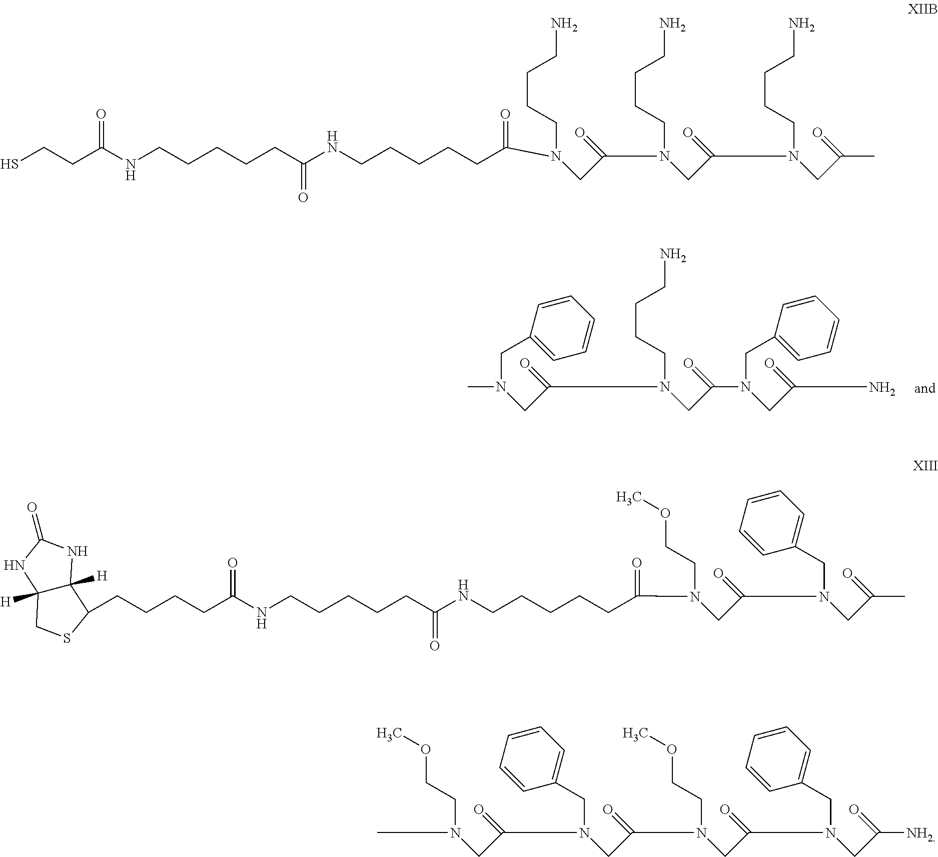

The samples to be tested may be organs, whole blood, blood fractions, blood components, plasma, platelets, serum, cerebrospinal fluid (CSF), brain tissue, nervous system tissue, muscle tissue, bone marrow, urine, tears, non-nervous system tissue, biopsies or necropsies In preferred embodiments, the sample is plasma or cerebrospinal fluid.

In methods of this invention, the pathogenic conformer-specific binding reagent is typically detectably labeled, for example, with biotin. The reagent is typically attached to a solid support, such as nitrocellulose, polystyrene latex, polyvinyl fluoride, diazotized paper, nylon membranes, activated beads, and magnetically responsive beads.

In another aspect, other methods for detecting the presence of a non-prion pathogenic conformer are provided. The methods may include the steps of contacting a sample suspected of containing the non-prion pathogenic conformer with a pathogenic conformer-specific binding reagent under conditions that allow the binding of the reagent to the non-prion pathogenic conformer, if present, to form a complex; and contacting the complex with a conformational disease protein-specific binding reagent under conditions that allow binding; and detecting the presence of the non-prion pathogenic conformer, if any, in the sample by its binding to the conformational disease protein-specific binding reagent; wherein the pathogenic conformer-specific binding reagent is derived from a prion protein fragment and interacts preferentially with a pathogenic prion protein. The conformational disease protein-specific binding reagent can be a labeled antibody. When the non-prion pathogenic conformer is an Aβ protein the conformational disease protein-specific binding reagent is an anti-Aβ antibody.

In one embodiment, the method further includes removing unbound sample materials after forming the complex.

Other methods for detecting the presence of a non-prion pathogenic conformer have at least the steps of contacting a sample suspected of containing the non-prion pathogenic conformer with a pathogenic conformer-specific binding reagent under conditions that allow the binding of the reagent to the non-prion pathogenic conformer, if present, to form a first complex; removing unbound sample materials; dissociating the non-prion pathogenic conformer from the first complex thereby providing dissociated non-prion pathogenic conformer; contacting the dissociated non-prion pathogenic conformer with a conformational disease protein-specific binding reagent under conditions that allow binding to form a second complex; and detecting the presence of the non-prion pathogenic conformer, if any, in the sample by detecting the formation of the second complex; wherein the pathogenic conformer-specific binding reagent is derived from a prion protein fragment and interacts preferentially with a pathogenic prion protein.

The formation of the second complex can be detected using a detectably labeled second conformational disease protein-specific binding reagent.

In some embodiments, the pathogenic conformer-specific binding reagent and/or conformational disease protein-specific binding reagent are coupled to solid supports.

The non-prion pathogenic conformer can be dissociated from the first complex (with the PCSB reagent) by exposing the complex to guanidine thiocyanate, exposing the complex to sodium hydroxide, or exposing the complex to high pH or low pH and in some cases, neutralizing the high pH or the low pH after the dissociating.

In embodiments where the non-prion pathogenic conformer is Aβ, the protein is preferably dissociated from the complex by exposure to a high pH condition, such as sodium hydroxide, preferably at about 0.1N NaOH at about 80° C.

Another method for detecting the presence of a non-prion pathogenic conformer has at least the steps of contacting a sample suspected of containing the non-prion pathogenic conformer with a first pathogenic conformer-specific binding reagent under conditions that allow binding of the first reagent to the non-prion pathogenic conformer, if present, to form a first complex; and contacting the sample suspected of containing the non-prion pathogenic conformer with a second pathogenic conformer-specific binding reagent under conditions that allow binding of the second reagent to the non-prion pathogenic conformer in the first complex, wherein the second reagent comprises a detectable label; and detecting the non-prion pathogenic conformer, if any, in a sample by its binding to the second reagent; wherein the first and second pathogenic conformer-specific binding reagents are derived from a prion protein fragment and interact preferentially with a pathogenic prion protein.

Yet another a method for detecting the presence of a non-prion pathogenic conformer has at least the steps of (a) contacting a sample suspected of containing the non-prion pathogenic conformer with a conformational disease protein-specific binding reagent under conditions that allow binding of the CDPSB reagent to the non-prion pathogenic conformer, if present, to form a complex; (b) removing unbound sample materials; (c) contacting the complex with a pathogenic conformer-specific binding reagent under conditions that allow the binding of the pathogenic conformer-specific binding reagent to the non-prion pathogenic conformer, wherein the pathogenic conformer-specific binding reagent comprises a detectable label; and detecting the non-prion pathogenic conformer, if any, in the sample by its binding to the pathogenic conformer-specific binding reagent; wherein the pathogenic conformer-specific binding reagent is derived from a prion protein fragment and interacts preferentially with a pathogenic prion protein.

Still yet another method for detecting the presence of a non-prion pathogenic conformer has at least the steps of providing a solid support comprising a pathogenic conformer-specific binding reagent; combining the solid support with a detectably labeled ligand, wherein the pathogenic conformer-specific binding reagent's binding affinity to the detectably labeled ligand is weaker than the reagent's binding affinity to the non-prion pathogenic conformer; combining a sample with the solid support under conditions which allow the non-prion pathogenic conformer, when present in the sample, to bind to the reagent and replace the ligand; and detecting complexes formed between the reagent and the non-prion pathogenic conformer from the sample; wherein the pathogenic conformer-specific binding reagent is derived from a prion protein fragment and interacts preferentially with a pathogenic prion protein.

A method for discriminating between a non-prion pathogenic conformer and a non-prion non-pathogenic conformer is also provided. The method has at least the steps of contacting a sample suspected of containing the non-prion pathogenic conformer with a pathogenic conformer-specific binding reagent under conditions that allow binding of the reagent to the non-prion pathogenic conformer, if present, to form a complex; and discriminating between the non-prion pathogenic conformer and the non-prion non-pathogenic conformer by binding of the pathogenic conformer to the reagent; wherein the pathogenic conformer-specific binding reagent is derived from a prion protein fragment and interacts preferentially with a pathogenic prion protein.

A method for diagnosing a non-prion conformational disease is also provided. The method has at least the steps of: contacting a sample suspected of containing a non-prion pathogenic conformer with a pathogenic conformer-specific binding reagent under conditions that allow binding of the reagent to the non-prion pathogenic conformer, if present, to form a complex; detecting the non-prion pathogenic conformer, if any, in the sample by its binding to the reagent; and diagnosing a conformational disease if the non-prion pathogenic conformer is detected; wherein the pathogenic conformer-specific binding reagent is derived from a prion protein fragment and interacts preferentially with a pathogenic prion protein.

In another aspect, the invention provides a method for detecting the presence of a non-prion pathogenic conformer having at least the steps of: contacting a sample suspected of containing the non-prion pathogenic conformer with a pathogenic conformer-specific binding reagent under conditions that allow binding of the reagent to the non-prion pathogenic conformer, if present, to form a complex; and detecting the non-prion pathogenic conformer, if any, in the sample by its binding to the pathogenic conformer-specific binding reagent; wherein the pathogenic conformer-specific binding reagent comprises a peptoid region comprising SEQ ID NO: 229, 230, 231, 232, 233, 234, 235, 236, 237, 238, 239, 240, or 241.

In still yet another aspect, the invention provides a method for detecting the presence of a non-prion pathogenic conformer having at least the steps of: contacting a sample suspected of containing the non-prion pathogenic conformer with a pathogenic conformer-specific binding reagent under conditions that allow binding of the reagent to the non-prion pathogenic conformer, if present, to form a complex; and detecting the non-prion pathogenic conformer, if any, in the sample by its binding to the pathogenic conformer-specific binding reagent; wherein the pathogenic conformer-specific binding reagent is selected from:

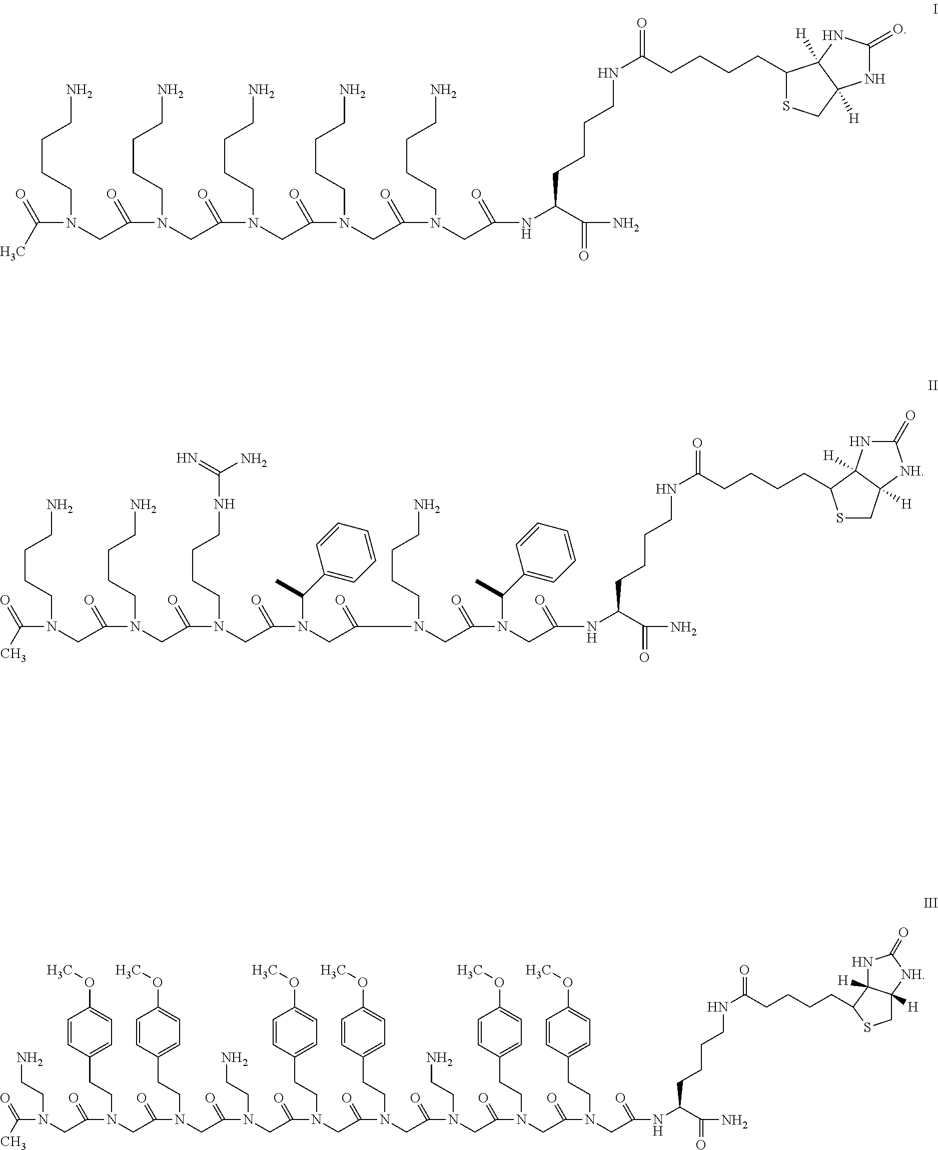

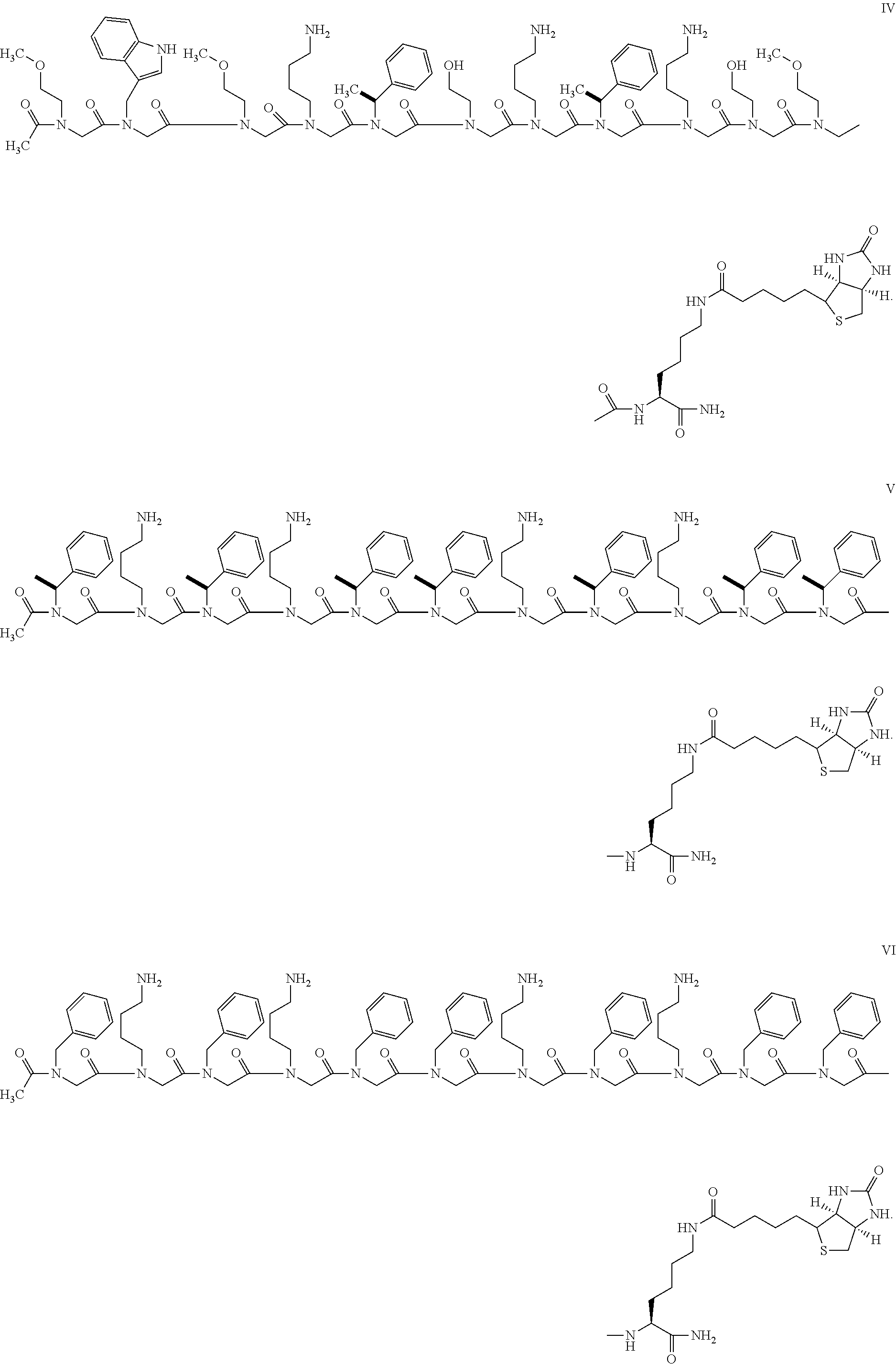

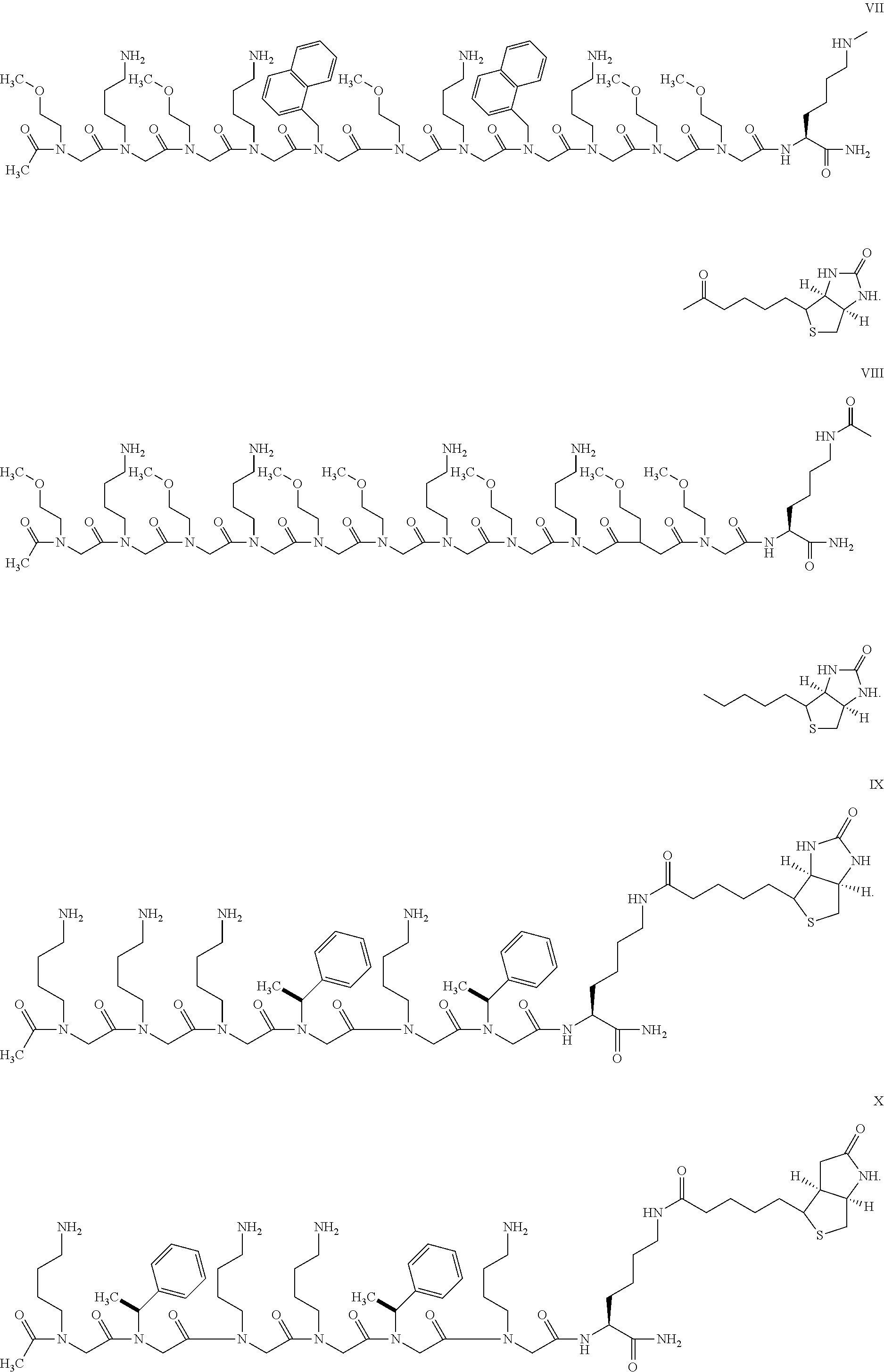

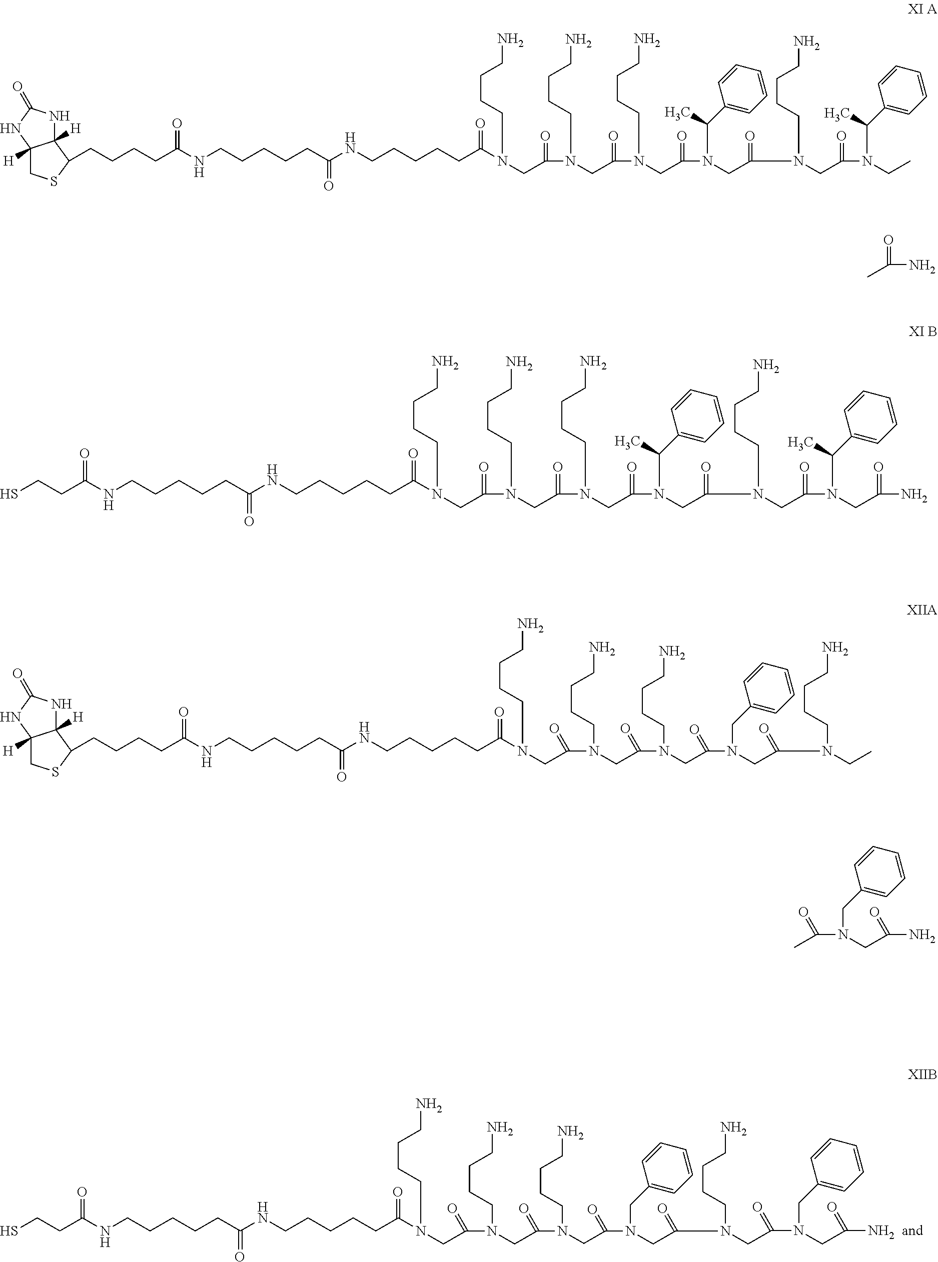

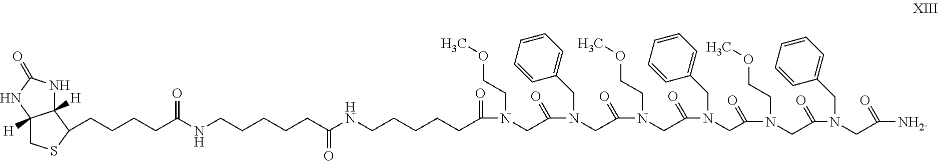

### BRIEF DESCRIPTION OF THE DRAWINGS

FIGS. 1A and 1B demonstrate the accumulation of misfolded Aβ40 and Aβ42 in a panel of Alzheimer's samples but not normal samples and that an Aβ ELISA recognizes these misfolded Aβ peptides only after denaturation. 10% (w/v) brain homogenate (BH) from normal or Alzheimer's diseased individuals were treated with either water (native, white bars) or 5.4M GdnSCN (denatured, black bars) for 30 minutes at room temperature before dilution into TBST (50 mM Tris, pH 7.5; 150 mM NaCl; 0.05% Tween-20) such that 100 mL of 10% BH was applied per 100 μL sample to each ELISA well. ELISA capture plates were coated with (1A) 11A50-B10 antibody (specific for the C-terminus of Aβ40) or (1B) 12F4 antibody (specific for the C-terminus of Aβ42) in individual wells at 2.5 μg/mL. After incubation for 1 hour at 37° C., the plates were washed 4 times with TBST and bound Aβ peptide was detected with 0.2 μg/ml of Alkaline Phosphatase (AP)-conjugated 4G8 detection antibody (recognizing residues 17-24 of Aβ) diluted in TBST+0.1% bovine serum albumin. After 1 hour at 37° C., the plates were again washed 4 times with TBST. LumiphosPlus was the chemiluminescent substrate. Patient identification numbers for normal (320, 326, 327, 328) and Alzheimer's patients (remaining numbered samples) are as indicated. A buffer control (bkgd) was used to determine background signal of the ELISA.

FIG. 2 demonstrates that misfolded Aβ42 is in an insoluble aggregated form that can be pelleted with centrifugation and that denaturation of the aggregates results in solubility and detection with the described ELISA. 100 nl of 10% BH was treated with either water (native, white bars) or 5.4M GdnSCN (denatured, gray bars) for 30 minutes at room temperature before dilution into 100 μl TBST. One set of samples was applied directly to the sandwich ELISA (total samples). Another set was centrifuged at 135,520 g for 1.5 hr at 4° C. Pellet fractions were denatured in 6M GdnSCN for 30 min at room temperature and diluted into 100 μl TBST. Then both supernatant and pellet fractions were applied to the ELISA. Aβ42 peptide was captured via 12F4 antibodies and detected with 4G8-AP detection antibody as previously described.

FIG. 3 demonstrates that PSR1 binding (i.e. pulldown) of Aβ42 aggregates in plasma can be attributed to peptoid XIIb rather than the bead. 75 mL of 10% BH from normal or Alzheimer's diseased patients was spiked into 100 μL of 80% plasma in TBSTT (50 mM Tris, pH 7.5; 150 mM NaCl; 1% Tween-20; 1% Triton-X100). BH-spiked solutions were incubated with either PSR1 or negative (GLUT) beads for 1 hour at 37° C., and then washed 5 times with TBST. The captured material was denatured with 6M GdnSCN for 30 min at room temperature and diluted into TBST before application onto 12F4 (recognizing the C terminus of Aβ42) capture plates. Captured material was detected with 4G8-AP as previously described. Patient identification numbers for normal (320, 326, 327, 328) and Alzheimer's patients (remaining numbered samples) are as indicated. A buffer control (bkgd) was used to determine background signal of the ELISA.

FIGS. 4A and 4B show endogenous soluble Aβ40 and Aβ42 levels in normal human plasma as detected by ELISA. Increasing concentrations of pooled human plasma were diluted into TBST buffer and applied to 11A50-B10 (4A) or 12F4 (4B) plates to capture Aβ40 and Aβ42, respectively. Captured peptides were detected with 4G8-AP detection antibody as previously described.

FIG. 5 (A-F) shows that PSR1 binds Aβ40 and Aβ42 aggregates but does not recognize Aβ aggregates that have been solubilized by denaturant. FIG. 5A is a standard curve of Aβ42, prepared by applying denatured synthetic Aβ42 to 12F4-coated ELISA plates. FIG. 5B shows detection of Aβ42 from increasing amounts of native or denatured Alzheimer's 10% BH (patient #291). The BH was treated with water (native, white triangles) or 5.4M GdnSCN (denatured, gray circles) for 30 minutes at room temperature before dilution into 100 μl TBST and being applied to 12F4 (specific for the C-terminus of Aβ42) capture plates to assess the levels of Aβ in the BH. FIG. 5C shows the amount of Aβ42 captured from Alzheimer's 10% BH (patient #291) treated with either water (native, white circles and triangles) or 5.4M GdnSCN (denatured, gray circles and triangles) for 30 minutes at room temperature before dilution into 100 μl 80% human plasma in TBSTT buffer and incubated with either PSR1 (triangles) or negative (GLUT, circles) beads for 1 hour at 37° C. Following the pulldown, beads were washed 5 times in TBST and bound material was eluted with 6M GdnSCN for 30 min at room temperature. Samples were diluted into TBST and applied to 12F4 capture plates. Captured material was detected with 4G8-AP as described previously. FIG. 5D is a standard curve of Aβ40, prepared by denaturing various concentrations of synthetic Aβ40 and applying to an 11A50-B10-coated (specific for the C-terminus of Aβ40) ELISA capture plate. FIG. 5E shows the amount of Aβ40 detected in Alzheimer's 10% BH (patient #291) treated with water (native, white triangles) or 5.4M GdnSCN (denatured, gray circles) for 30 minutes at room temperature before dilution into 100 TBST. Samples were directly applied to 11A50-B10 capture plates to assess the levels of Aβ42 in the BH. FIG. 5F shows the amount of Aβ40 captured from Alzheimer's 10% BH (patient #291) treated with either water (native, white circles and triangles) or 5.4M GdnSCN (denatured, gray circles and triangles) for 30 minutes at room temperature before dilution into 100 μl 80% human plasma in TBSTT buffer and incubated with either PSR1 or negative (GLUT) beads for 1 hour at 37° C. Following the pulldown, beads were washed 5 times in TBST and bound material was eluted with 6M GdnSCN for 30 min at room temperature. Samples were diluted into TBST and applied to 11A50-B10 capture plates. Captured material was detected with 4G8-AP as described previously.

FIGS. 6A and 6B compare the capture profile of various pathogenic conformer-specific binding reagents (six different peptides and PSR1) for PrP^{Sc} in vCJD samples with the capture profile for Aβ42 aggregates in AD samples containing either buffer or 50% plasma, and thus demonstrate that PSR1 and prion-derived peptides do bind PrP^{Sc} and Aβ aggregates in buffer and plasma. Biotinylated peptides were coated onto M280-streptavidin beads prior to incubation with sample. PSR1 peptoid was coupled to Dynal M270-carboxylic acid beads. For the vCJD experiments, 100 mL of 10% vCJD BH was diluted into 100 μL TBSTT (8A, top panel) or 50% plasma in TBSTT (8B, top panel) and incubated with the indicated pathogenic conformer-specific binding reagents for 1 hour at 37° C. The beads were washed 6 times in TBST and eluted with 0.1M NaOH for 10 minutes at room temperature. The elution was neutralized with 0.3M NaH₂PO₄ for 5 minutes at room temperature before being applied to 3F4-coated capture plates (2.5 μg/mL 3F4 antibody, recognizing residues 109-112 of the human PrP sequence). The material was captured for 1 hour at 37° C., washed 6 times in TBST, and detected with AP-conjugated POM2 antibody (recognizing the prion octarepeat sequence) in 0.01×CaseinBlocker in TBST. After a 1 hour incubation at 37° C., the plate was again washed and detected with LumiphosPlus substrate. For the AD experiments, 50 mL of 10% AD BH was similarly spiked into TBSTT (8A, lower panel) or 50% plasma in TBSTT (8B, lower panel). The samples were similarly pulled down with the conformer specific-binding reagents, eluted with 6M GdnSCN for 30 minutes at room temperature, and diluted with TBST. The eluates were applied to 12F4 capture plates and detected by horse radish peroxidase (HRP)-conjugated 4G8. Data for vCJD has been previously published as Lau, A. L, et al. Proc Natl Acad Sci USA. 104(28): 11551-11556 (2007).

FIG. 7 shows the capture profile of the same panel of prion-derived peptides and PSR1 for aggregated Aβ42 from AD BH spiked into 50% CSF in TBSTT. Pulldowns and Aβ42-specific sandwich ELISA detection were performed as described above.

FIG. 8 depicts NaOH concentration titration and temperature screening for optimization of denaturation of Aβ42 in AD brain homogenate vs. Normal Brain Homogenate (NBH).

FIG. 9, panels A-F, depict exemplary peptoid substitutions that may be made to prepare any of the PCSB reagents described herein. The peptoids are circled in each panel and are shown in an exemplary reagent as described herein (QWNKPSKPKTNG, SEQ ID NO: 250), in which a proline residue (residue 8) is replaced with an N-substituted glycine (peptoid) residue. Panel A shows a peptide reagent in which a proline residue is substituted with the peptoid residue: N—(S)-(1-phenylethyl)glycine; panel B shows a peptide reagent in which a proline residue is substituted with the peptoid residue: N-(4-hydroxyphenyl)glycine; panel C shows a peptide reagent in which a proline residue is substituted with the peptoid residue: N-(cyclopropylmethyl)glycine; panel D shows a peptide reagent in which a proline residue is substituted with the peptoid residue: N-(isopropyl)glycine; panel E shows a peptide reagent in which a proline residue is substituted with the peptoid residue: N-(3,5-dimethoxybenzyl)glycine; and panel F shows a peptide reagent in which a proline residue is substituted with the peptoid residue: N-butylglycine.

FIG. 10 depicts the structures of exemplary PEG-linked pathogenic conformer-specific binding reagents as described herein.

FIG. 11 shows that PSR1 and PrP23-30 capture of Aβ is superior to capture by β-sheet blockers. AL30 is the Aβ20-16 reverse sequence (uses D-amino acids instead of L-amino acids) and has the following structure: biotin-AHX-D(FFVLK)-CONH2 (SEQ ID NO: 252). AL32 is Aβ20-16 and has the following structure: biotin-AHX-FFVLK-CONH2 (SEQ ID NO: 253). AL33 is Aβ16-20-NmeL and has the following structure: biotin-AHX-KLVFF-NmeL-CONH2 (SEQ ID NO: 254). NmeL is N-methylated lysine, a “standard” amino acid modification available from most custom peptide synthesis companies. AL34 is Aβ(16-20-NmeL)₂ and has the following structure: biotin-AHX-KLVFF-NmeL-AHX-KLVFF-NmeL-CONH2 (SEQ ID NO: 255).

FIG. 12 shows that significant levels of total tau are detectable in both normal and Alzheimer's Disease brains. Normal brain (patient ID #320, 326, 327, 328) or AD brain (patient ID # 325, 334, 325, 264-1, 230-1, 218-2, 221-1, 201-2, 184-1, 177-1, and 291) homogenates were either untreated (native, white bars) or treated with 3M GnSCN (black bars). Total tau was quantitated using the BioSource Tau Immunoassay Kit.

FIG. 13 depicts the amount of tau which specifically binds to PSR1 beads as opposed to the control glutathione beads. Normal brain (patient ID #320, 326, 327, 328) or AD brain (patient ID # 325, 334, 325, 264-1, 230-1, 218-2, 221-1, 201-2, 184-1, 177-1, and 291) homogenates diluted into buffer and either incubated with M270-glutathione (white bars) or PSR1 (black bars).

FIG. 14 shows that PSR1 binds tau aggregates but does not recognize tau aggregates which have been solubilized by denaturant. Normal brains (patient ID #320, 326) or AD brains (patient ID # 334, 230) were either treated with water (N) or 5M GdnSCN (D), diluted in 25% plasma in TBSTT, and then incubated with either M270 glutathione control beads (white bar) or PSR1 (black bars). Following pulldown, the beads were washed with TBSTT and incubated with GdnSCN. Captured tau was quantitated using the BioSource Tau Immunoassay Kit.

FIGS. 15A and B depict data used to calculate the LOD for a sandwich ELISA for monomeric soluble Aβ and PSR1 Pulldown for aggregated Aβ. In FIG. 15A, varying amounts of synthetic soluble Aβ (pg/mL) are detected by sandwich ELISA. In FIG. 15B, varying amounts of 10% AD brain homogenate spiked into 200 ul of pooled normal human CSF are subject to PSR1 pulldown and detected by sandwich ELISA. Filled circles represent Aβ40. Open circles represent Aβ42.

FIG. 16 compares the total amount of Aβ42 aggregates in AD BH (square) with the Aβ42 aggregates bound by PSR1 (triangle).

FIG. 17 depicts the effect of increasing concentrations of plasma on binding of monomeric Aβ42 (triangles) and aggregated Aβ42 (circles).

FIG. 18 compares signal for NBH (normal brain homogenate, open bar) and AD (brain homogenate from Alzheimer's disease patient, filled bar) for various dissociation conditions.

FIG. 19 depicts data used to calculate the LOD for ELISA and PSR1 bead pulldown of Total Tau (A&B), P-Tau231 (C&D) and P-Tau181 (E&F). FIG. 19A shows a Tau ELISA standard curve. FIG. 19B shows Tau Pulldown in AD BH spiked CSF (200 ul assay). FIG. 19C shows a P-Tau231 ELISA standard curve. FIG. 19D shows P-Tau231 Pulldown in AD BH spiked CSF (70 uL CSF). FIG. 19E shows a P-Tau181 ELISA standard curve. FIG. 19F shows P-Tau181 Pulldown in AD BH spiked CSF (70 uL CSF).

### BRIEF DESCRIPTION OF TABLES

Table 1 lists exemplary conformational diseases and the associated conformational disease proteins.

Table 2 lists additional conformational disease proteins and related conformational diseases.

Table 3 lists exemplary peptide sequences used to make PCSB reagents.

Table 4 lists exemplary peptoid regions suitable for making PCSB reagents.

Table 5 provides a key to the abbreviations used in Table 4.

Table 6 provides the relevant structures of each of the sequences listed in Table 4.

Table 7 quantitates the pulldown efficiency of PSR1.

Table 8 quantitates tau levels measured in Example 10.

Table 9 quantitates tau levels measured in Example 11.

Table 10 quantitates the binding of Aβ40 and 42 from the CSF of individuals without Alzheimer's disease to PSR1 bead.

Table 11 quantitates the binding of the PSR1 to monomeric and aggregated Aβ in the presence of increasing concentrations of plasma.

### BRIEF DESCRIPTION OF SEQUENCE LISTING

SEQ ID NO:s 1 to 11 provide the amino acid sequence of prion proteins from different species: human (SEQ ID NO:1), mouse (SEQ ID NO:2), human (SEQ ID NO:3), Syrian hamster (hamster) (SEQ ID NO:4), bovine (SEQ ID NO:5), sheep (SEQ ID NO:6), mouse(SEQ ID NO:7), elk (SEQ ID NO:8), fallow deer (fallow) (SEQ ID NO:9), mule deer (mule) (SEQ ID NO:10), and white tailed deer (white) (SEQ ID NO:11).

SEQ ID NO:s 12 to 228 provide the amino acid sequence of exemplary peptide sequences used to make PCSB reagents.

SEQ ID NO:s 229 to 241 provide the modified amino acid sequences of exemplary peptoid regions used to make PCSB reagents.

SEQ ID NO:s 242 to 249 provide the amino acid sequences of the exemplary prion protein fragments used to make PCSB reagents.

SEQ ID NO: 250 provides the amino acid sequence of an exemplary peptide sequence used to make PCSB reagents.

SEQ ID NO: 251 provides the amino acid sequence residues 19 to 30 of the human prion protein as indicated in SEQ ID NO: 1.

SEQ ID NO:s 252 to 255 provide the amino acid sequences of the β-sheet breakers AL30, AL32, AL33, and AL34.

SEQ ID NO:s 256 to 261 provide the amino acid sequences of the modified prion protein fragments tested in Example 3.

### DETAILED DESCRIPTION OF THE INVENTION

This invention relates to the surprising and unexpected discovery that PCSB reagents which interact preferentially with pathogenic conformers of the prion protein also interact preferentially with pathogenic conformers of other conformational diseases such as Alzheimer's disease, diabetes, systemic amyloidoses, etc. These PCSB reagents are typically derived from prion protein fragments.

The discovery that these PCSB reagents also interact preferentially with non-prion pathogenic conformers allows the development of detection assays, diagnostic assays and purification or isolation methods utilizing these PCSB reagents for conformational diseases and conformational disease proteins beyond prions and prion-related diseases.

While not wishing to be held to any theory, it is believed that the ability of these PCSB reagents to preferentially bind and detect non-prion pathogenic conformers is due to the existence of a structural motif common to certain pathogenic conformers. Lau, A. L, et al. Proc Natl Acad Sci USA. 104(28): 11551-11556 (2007).), which is hereby incorporated by reference as if it were contained herein, suggest that the interaction between PCSB reagents derived from prion protein fragments and PrP^{Sc} is largely dependent on positive charge. The interaction does not appear to be affected by scrambling the sequence, but the properties of individual amino acids beyond their positive charge also appears to play some role in the interaction. These studies suggest that these PCSB reagents bind a structural motif rather than a linear sequence domain of PrP^{Sc} that is associated with disease.

Many pathogenic conformers which form amyloids share similar physical properties. For example, PrP^{Sc}, the pathogenic conformer of the prion protein, exhibits the following characteristics: increased β-sheet content (˜3% in PrP^{C} to >40% in PrP^{Sc}) and PrP^{Sc} fibers are composed of β-sheets that are oriented perpendicularly along the fiber axis. Applicants believe that binding to one of the structural motifs common to all amyloid-forming proteins is the mechanism by which reagents that interact preferentially with pathogenic prion proteins also interact preferentially with pathogenic conformers of non-prion proteins.

These PCSB reagents need not be part of a larger structure or other type of scaffold molecule in order to exhibit this preferential interaction with the pathogenic conformer. While not wanting to be held to any particular theory, it appears that these PCSB reagents spontaneously take on a conformation that allows binding to the pathogenic conformer but not the non-pathogenic conformer. It will be apparent to one of ordinary skill in the art that, while the exemplified PCSB reagents provide a starting point (in terms of size or sequence characteristics, for example) for PCSB reagents useful in methods of this invention that many modifications can be made to produce PCSB reagents with more desirable attributes (e.g, higher affinity, greater stability, greater solubility, less protease sensitivity, greater specificity, easier to synthesize, etc.).

In general, the PCSB reagents described herein are able to interact preferentially with the pathogenic conformers. Thus, these reagents allow for ready detection of the presence of pathogenic conformers for example by ordering, aggregating or otherwise inducing the disease-forming proteins to a state that can then be detected and, hence, diagnosis of pathogenic conformers in virtually any sample, biological or non-biological, including living or dead brain, spinal cord, cerebrospinal fluid, or other nervous system tissue as well as blood and spleen. The PCSB reagents are useful in a wide range of isolation, purification, detection, diagnostic and therapeutic applications.

The PCSB reagents used in methods of this invention are described in further detail in WO05/016137 and WO07/030,804 which are hereby incorporated by reference.

The practice of the present invention will employ, unless otherwise indicated, conventional methods of chemistry, biochemistry, molecular biology, immunology and pharmacology, within the skill of the art. Such techniques are explained fully in the literature. See, e.g., Remington's Pharmaceutical Sciences, 18th Edition (Easton, Pa.: Mack Publishing Company, 1990); Methods In Enzymology (S. Colowick and N. Kaplan, eds., Academic Press, Inc.); and Handbook of Experimental Immunology, Vols. I-IV (D. M. Weir and C. C. Blackwell, eds., 1986, Blackwell Scientific Publications); Sambrook, et al., Molecular Cloning: A Laboratory Manual (2nd Edition, 1989); Handbook of Surface and Colloidal Chemistry (Birdi, K. S. ed., CRC Press, 1997); Short Protocols in Molecular Biology, 4th ed. (Ausubel et al. eds., 1999, John Wiley & Sons); Molecular Biology Techniques: An Intensive Laboratory Course, (Ream et al., eds., 1998, Academic Press); PCR (Introduction to Biotechniques Series), 2nd ed. (Newton & Graham eds., 1997, Springer Verlag); Peters and Dalrymple, Fields Virology (2d ed), Fields et al. (eds.), B. N. Raven Press, New York, N.Y.

It is understood that the reagents and methods of this invention are not limited to particular formulations or process parameters as such may, of course, vary. It is also to be understood that the terminology used herein is for the purpose of describing particular embodiments of the invention only, and is not intended to be limiting.

All publications, patents and patent applications cited herein are hereby incorporated by reference in their entirety.

### I. DEFINITIONS

In order to facilitate an understanding of the invention, selected terms used in the application will be discussed below.

As used herein, the term “pathogenic” may mean that the protein or conformer actually causes the disease or it may simply mean that the protein or conformer is associated with the disease and therefore is present when the disease is present. Thus, a pathogenic protein or conformer as used in connection with this disclosure is not necessarily a protein that is the specific causative agent of a disease and therefore may or may not be infectious. The term “pathogenic conformer” is used more specifically to refer to the conformation of the protein associated with disease and/or the beta-sheet-rich conformation. A “pathogenic conformer” is any conformation of the protein associated with disease, regardless of whether that conformation is a misfolded conformer, a misfolded conformer in aggregated form, or a mixture of the two. The terms “non-pathogenic” and “cellular” when used with respect to conformational disease proteins or conformers are used interchangeably to refer to the normal conformer of the protein whose presence is not associated with sickness. A pathogenic conformer associated with a particular disease, for example, Alzheimer's disease, may be described as a “pathogenic Alzheimer's disease conformer”.

The term “pathogenic conformer-specific binding reagent” or “PCSB reagent” refers to any type of reagent, including but not limited to peptides and peptoids, which interacts preferentially with a pathogenic conformer as opposed to the non-pathogenic conformer due to increased affinity or specificity. A preferential interaction does not necessarily require interaction between specific amino acid residues and/or motifs of each peptide. For example, in certain embodiments, the pathogenic conformer-specific binding reagents described herein interact preferentially with pathogenic conformers but, nonetheless, may also be capable of binding non-pathogenic conformers at a weak, yet detectable, level (e.g., 10% or less of the binding shown to the polypeptide of interest). Typically, weak binding, or background binding, is readily discernible from the preferential interaction with the pathogenic conformer of interest, e.g., by use of appropriate controls. In general, pathogenic conformer-specific binding reagents used in methods of the invention bind pathogenic conformers in the presence of excess of non-pathogenic forms.

A pathogenic conformer-specific binding reagent is said to “interact” with another peptide or protein if it binds specifically, non-specifically or in some combination of specific and non-specific binding. A reagent is said to “interact preferentially” with a pathogenic conformer if it bind with greater affinity and/or greater specificity to the pathogenic conformer than to non-pathogenic conformer. The terms “interact preferentially,” “preferentially interact,” “bind selectively,” “selectively bind,” and “selectively capture” are use interchangeably herein. It is to be understood that a preferential interaction does not necessarily require interaction between specific amino acid residues and/or motifs of each peptide. For example, in certain embodiments, the PCSB reagents described herein interact preferentially with pathogenic conformers but, nonetheless, may be capable of binding non-pathogenic conformers at a weak, yet detectable, level (e.g., 10% or less of the binding shown to the polypeptide of interest). Typically, weak binding, or background binding, is readily discernible from the preferential interaction with the compound or polypeptide of interest, e.g., by use of appropriate controls. In general, reagents described herein bind pathogenic conformers in the presence of more than 100-fold excess of non-pathogenic conformers.

The PCSB reagents utilized in the methods described herein are derived from a prion protein fragment and interact preferentially with the pathogenic form of the prion protein. The term “derived from a prion protein fragment” as used herein refers to reagents having a chemical structure based on that of a prion protein fragment. Such reagents can be peptide fragments having the native prion protein sequence, peptide fragments having a native prion protein sequence with conservative amino acid substitutions, or a peptoid analog of a peptide fragment of a prion protein.

The term “derived from a prion protein fragment and interacts preferentially with a pathogenic prion protein” as used herein refers to a reagent having the previously defined features in combination. By way of an example, such a reagent will have a chemical structure based on that of a prion protein fragment as defined above and also binds with greater affinity and/or greater specificity to a pathogenic prion protein than to a non-pathogenic prion protein.

The term “non-prion pathogenic conformer” as used herein refers to a pathogenic conformer of a conformational disease protein other than one associated with a prion disease as defined herein.

“Conformational disease protein” refers to the pathogenic and non-pathogenic conformers of a protein associated with a conformational disease where the structure of the protein has changed (e.g., misfolded) such that it results in an abnormal conformation such as unwanted fibril or amyloid polymerization in the context of a β-pleated sheet. Examples of conformational disease proteins include, without limitation, Alzheimer's disease proteins, such as Aβ and tau; prion proteins such as PrP^{Sc} and PrP^{C}, and the diabetes protein amylin. A non-limiting list of diseases with associated proteins that assume two or more different conformations is shown below.

TABLE 1

| | Conformational Disease |
|---|---|
| Disease | Protein(s) |
| Prion diseases | PrP^{Sc} |
| (e.g., Creutzfeldt-Jakob disease, scrapie, | |
| bovine spongiform encephalopathy) | |
| Alzheimer's Disease | Aβ peptides |
| | non-Aβ component |
| ALS | SOD1, tau |
| Pick's disease | Pick body (tau) |
| Parkinson's disease | Lewy body (tau, |
| | alpha-synuclein) |
| Diabetes Type II | Amylin |
| Multiple myeloma - plasma cell dyscrasias | IgG light chain |
| | IgG heavy chain |
| Familial amyloidotic polyneuropathy | Transthyretin |
| Medullary carcinoma of thyroid | Procalcitonin |
| Chronic Renal failure | beta2-microglobulin |
| Congestive heart failure | atrial natriuretic factor |
| senile cardiac and systemic amyloidosis | Transthyretin |
| Chronic inflammation | Serum amyloid A |
| Atherosclerosis | ApoA1 |
| Familial amyloidosis | Gelsolin |
| All tauopathies, including argyrophilic grain | Tau |
| dementia, corticobasal degeneration, | |
| dementia pugilistica, Hallervorden-Spatz | |
| disease, myotonic dystrophy, etc. | |
| Synucleinopathies, including Gaucher's | Alpha-synuclein |
| disease, multisystem atrophy, Lewy body | |
| dementia, etc. | |
| Corneal dystrophy, gelatinous drop-like | Possibly lactoferrin |
| Aortic amyloidosis in the elderly | Medin |
| Cutaneous amyloidosis | Keratin |
| Heriditary cerebral hemorrhage (Icelandic) | Cystatin C |

A “conformational disease protein” as used herein is not limited to polypeptides having the exact sequence as those described herein. It is readily apparent that the terms encompass conformational disease proteins from any of the identified or unidentified species or diseases (e.g., Alzheimer's, Parkinson's, etc.). One of ordinary skill in the art in view of the teachings of the present disclosure and the art can determine regions corresponding to the sequences shown in the Figures in any other prion proteins, using for example, sequence comparison programs (e.g., BLAST and others described herein) or identification and alignment of structural features or motifs.

“Conformational disease protein-specific binding reagent” or “CDPSB reagent” refers to any type of reagent which interacts preferentially with a specific conformational disease protein as opposed to other conformational disease proteins and other types of the proteins. Preferably, conformational disease protein-specific binding reagents bind to both pathogenic and non-pathogenic conformers of a conformational disease protein. However, in many instances the conformational disease protein-specific binding reagent may only bind to a soluble form of a conformational disease protein and therefore cannot bind the aggregated/misfolded pathogenic conformer. In that case, it may be necessary to denature the insoluble pathogenic conformer in order for it to be detected. Typically, such reagents are monoclonal or polyclonal antibodies.

The terms “prion”, “prion protein”, “PrP protein” and “PrP” are used interchangeably herein to refer to both the pathogenic conformer (variously referred to as scrapie protein, pathogenic protein form, pathogenic isoform, pathogenic prion and PrP^{Sc}) and the non-pathogenic conformer (variously referred to as cellular protein form, cellular isoform, non-pathogenic isoform, non-pathogenic prion protein, and PrP^{C}), as well as the denatured form and various recombinant forms of the prion protein which may not have either the pathogenic conformation or the normal cellular conformation. The pathogenic conformer is associated with disease state (spongiform encephalopathies) in humans and animals. The non-pathogenic conformer is normally present in animal cells and may, under appropriate conditions, be converted to the pathogenic PrP^{Sc} conformation. Prions are naturally produced in a wide variety of mammalian species, including human, sheep, cattle, and mice. A representative amino acid sequence of a human prion protein is set forth as SEQ ID NO:1. A representative amino acid sequence of a mouse prion protein is set forth as SEQ ID NO:2. Other representative sequences are provided as SEQ ID NO:s 3 to 11. Fragments of the prion proteins are designated by a SEQ ID NO: corresponding to the actual sequence or by indicating the amino acid position of the first and last amino acids of the fragment. Unless otherwise indicated, fragments referred to by indicating the first and last amino acids of the fragment are based on the sequence of the human prion protein as indicated in SEQ ID NO: 1. For example, the term “PrP₁₉₋₃₀” refers to a peptide having a sequence of LGLCKKRPKPGG (SEQ ID NO: 251).

The term “Alzheimer's disease (AD) protein” or “AD protein” are used interchangeably herein to refer to both the pathogenic conformer (variously referred to as pathogenic protein form, pathogenic isoform, pathogenic Alzheimer's disease protein, and Alzheimer's disease conformer) and the non-pathogenic conformer (variously referred to as normal cellular form, non-pathogenic isoform, non-pathogenic Alzheimer's disease protein), as well as the denatured form and various recombinant forms of the Alzheimer's disease protein which may not have either the pathogenic conformation or the normal cellular conformation. Exemplary Alzheimer's disease proteins include Aβ and the tau protein.

The terms “amyloid-beta,” “amyloid-β,” “Abeta”, “Aβ” “Aβ42”, “Aβ40,” and “Aβ40/42” as used herein all refer to amyloid-β peptides, which are 39 to 43 amino acid long fragments formed by cleavage of the amyloid precursor protein (APP). The term Aβ is used to refer generally to the amyloid-β peptides in any form. The term “Aβ42” refers to a fragment corresponding to amino acids 1 to 42 of APP. The term “Aβ40” refers to a fragment corresponding to amino acids 1 to 40 of APP. The term Aβ40/42 is used to refer to both the Aβ40 and Aβ42 isoforms.

The term “diabetes protein” is used herein to refer to both the pathogenic conformer (variously referred to as pathogenic protein form, pathogenic isoform, pathogenic diabetes disease protein) and the non-pathogenic conformer (variously referred to as normal cellular form, non-pathogenic isoform, non-pathogenic diabetes disease protein), as well as the denatured form and various recombinant forms of the diabetes disease protein which may not have either the pathogenic conformation or the normal cellular conformation. An exemplary Type II diabetes protein is amylin, which is also known as Islet Amyloid Polypeptide (IAPP).

A “fragment” as used herein refers to a peptide consisting of only a part of the intact full-length protein and structure as found in nature. For instance, a fragment can include a C-terminal deletion and/or an N-terminal deletion of a protein. Typically, the fragment retains one, some or all of the functions of the full-length polypeptide sequence from which it is derived. Typically, a fragment will comprise at least 5 consecutive amino acid residues of the native protein; preferably, at least about 8 consecutive amino acid residues; more preferably, at least about 10, 11, 12, 13, 14, 15, 16, 17, 18, 19, 20, 21, 22, 23, 24, 25, 26, 27, 28, 29, or 30 consecutive amino acid residues of the native protein.

By “isolated” is meant, when referring to a polynucleotide or a polypeptide, that the indicated molecule is separate and discrete from the whole organism with which the molecule is found in nature or, when the polynucleotide or polypeptide is not found in nature, is sufficiently free of other biological macromolecules so that the polynucleotide or polypeptide can be used for its intended purpose.

“Peptoid” is used generally to refer to a peptide mimic that contains at least one, preferably two or more, amino acid substitutes, preferably N-substituted glycines. Peptoids are described in, inter alia, U.S. Pat. No. 5,811,387. As used herein, a “peptoid reagent” is a molecule having an amino-terminal region, a carboxy-terminal region, and at least one “peptoid region” between the amino-terminal region and the carboxy-terminal region. The amino-terminal region refers to a region on the amino-terminal side of the reagent that typically does not contain any N-substituted glycines. The amino-terminal region can be H, alkyl, substituted alkyl, acyl, an amino protecting group, an amino acid, a peptide, or the like. The carboxy-terminal region refers to a region on the carboxy-terminal end of the peptoid that does not contain any N-substituted glycines. The carboxy-terminal region can include H, alkyl, alkoxy, amino, alkylamino, dialkylamino, a carboxy protecting group, an amino acid, a peptide, or the like.

The “peptoid region” is the region starting with and including the N-substituted glycine closest to the amino-terminus and ending with and including the N-substituted glycine closest to the carboxy-terminus. The peptoid region generally refers to a portion of a reagent in which at least three of the amino acids therein are replaced by N-substituted glycines.

“Physiologically relevant pH” refers to a pH of about 5.5 to about 8.5; or about 6.0 to about 8.0; or usually about 6.5 to about 7.5.

“Aliphatic” refers to a straight-chained or branched hydrocarbon moiety. Aliphatic groups can include heteroatoms and carbonyl moieties.

“Alkyl,” whether used alone or as part of another group, refers to an aliphatic hydrocarbon chain and includes, but is not limited to, straight and branched chains containing from 1 to 6, 1 to 5, 1 to 4, or 1 to 3 carbon atoms, unless explicitly specified otherwise. For example, methyl, ethyl, propyl, isopropyl, butyl, isobutyl, tert-butyl, etc. are encompassed by the term “alkyl.”

“Alkenyl” is intended to denote alkyl groups that contain at least one double bond, e.g., 2 to 7, 2 to 6, 2 to 5, or 2 to 4 carbon atoms, including, for example but not limited to, vinyl, allyl, 2-methyl-allyl, 4-but-3-enyl, 4-hex-5-enyl, 3-methyl-but-2-enyl and the like.

“Alkynyl” is intended to denote alkyl groups that have at least one triple carbon-carbon bond, e.g., 2 to 7, 2 to 6, 2 to 5, or 2 to 4 carbon atoms. Example alkynyl groups include ethynyl, propynyl, and the like.

“Alkoxy,” whether used alone or as part of another group, has its normal meaning of a group of formula —O-alkyl, e.g., methoxy, where alkyl is as defined herein.

“Halo” or “halogen,” when used alone or as part of another group, has its normal meaning of Group VII elements, e.g., F, Cl, Br and I.

“Aryl,” when used alone or as part of another group, means an aromatic hydrocarbon system, e.g., of 6 to 20, 6 to 14, or 6 to 10 ring carbon atoms, e.g., of 1, 2 or 3 rings, for example, phenyl, benzyl, naphthyl, naphthalene, anthracene, phenanthrenyl, anthracenyl, pyrenyl and the like. Also included in the definition of aryl are aromatic systems containing one or more fused non-aromatic carbocyclyl or heterocyclyl rings, for example, 1,2,3,4-tetrahydronaphthalene and indan. The aryl group containing an fused non-aromatic ring can be attached through the aromatic portion or the non-aromatic portion.

“Aryl-alkyl” or “aralkyl” means a group of formula -alkyl-aryl, wherein aryl and alkyl have the definitions herein.

“Aryloxy,” has its normal meaning of a group of formula —O-aryl, e.g., hydroxyphenyl, where aryl is as defined herein.

“Aralkoxy,” has its normal meaning of a group of formula —O-alkyl-aryl, e.g., methoxyphenyl, where alkoxy and aryl are as defined herein.

“Cycloalkyl,” whether used alone or as part of another group, has its normal meaning of a cyclic alkyl, alkenyl, or alkynyl group, e.g., a mono, bi-, tri-cyclic, fused, bridged or spiro saturated hydrocarbon moiety, e.g., of 3-10 carbon atoms, e.g., cyclopropyl. The term “cycloalkyl-aryl” is intended to denote a group of formula -aryl-cycloalkyl where aryl and cycloalkyl are as defined herein. “Cycloalkyalkyl” is intended to denote a group of formula -alkyl-cycloalkyl, for example, a cyclopropylmethyl or cyclohexylmethyl group, where alkyl and cycloalkyl are as defined herein.

As used herein, “heteroaryl” groups refer to an aromatic heterocycle having at least one heteroatom ring member such as sulfur, oxygen, or nitrogen. Heteroaryl groups include monocyclic and polycyclic (e.g., having 2, 3 or 4 fused rings) systems. Examples of heteroaryl groups include without limitation, pyridyl, pyrimidinyl, pyrazinyl, pyridazinyl, triazinyl, furyl (furanyl), quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 3 to about 7, or 5 to 6 ring-forming atoms. In some embodiments, the heteroaryl group has 1 to about 4, 1 to about 3, or 1 to 2 heteroatoms.

As used herein, “heterocycloalkyl” refers to non-aromatic heterocycles including cyclized alkyl, alkenyl, and alkynyl groups where one or more of the ring-forming carbon atoms is replaced by a heteroatom such as an O, N, or S atom. Example “heterocycloalkyl” groups include morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, and the like. Also included in the definition of heterocycloalkyl are moieties that have one or more aromatic rings fused (i.e., having a bond in common with) to the nonaromatic heterocyclic ring, for example, phthalimidyl, naphthalimidyl, and benzo derivatives of heterocycles such as indolene and isoindolene groups. In some embodiments, the heterocycloalkyl group has from 1 to about 20 carbon atoms, and in further embodiments from about 3 to about 20 carbon atoms. In some embodiments, the heterocycloalkyl group contains 3 to about 14, 3 to about 7, or 5 to 6 ring-forming atoms. In some embodiments, the heterocycloalkyl group has 1 to about 4, 1 to about 3, or 1 to 2 heteroatoms. In some embodiments, the heterocycloalkyl group contains 0 to 3 double bonds. In some embodiments, the heterocycloalkyl group contains 0 to 2 double or triple bonds.

“Heteroarylalkyl” refers to a group of formula -alkyl-heteroaryl, where alkyl and heteroaryl are as defined herein.

“Acyl” refers to a group of formula —C(O)-alkyl. In some embodiments, the acyl group has from 1 to 10, 1 to 8, 1 to 6, or 1 to 4 carbon atoms.

“Aminoacyl” refers to a group of formula —C(O)-alkyl-amino, where alkyl is as defined herein.

“Alkylamino” refers to a group of formula —NH-alkyl, where alkyl is as defined herein.

“Dialkylamino” refers to group of formula —N(alkyl)₂, where alkyl is as defined herein.

“Haloalkyl” refers to an alkyl group substituted by one or more halogens, where alkyl and halogen are as defined herein.

“Alkoxyalkyl” refers to a group of formula -alkyl-alkoxy, where alkyl and alkoxy are as defined herein.

“Carboxyalkyl” refers to a group of formula -alkyl-COOH, where alkyl is as defined herein.

“Carbamyl” refers to a group of formula —C(O)NH₂.

“Carbamylalkyl” refers to a group of formula -alkyl-C(O)NH₂, where alkyl is as defined herein.

“Guanidinoalkyl” refers to a group of formula -alkyl-NHC(═NH)NH₂, where alkyl is as defined herein.

“Thiol” refers to a group of formula —SH.

“Alkylthiol” refers to a group of formula —S-alkyl, where alkyl is as defined herein.

“Alkylthioalkyl” refers to a group of formula -alkly-S-alkyl, where alkyl is as defined herein.

“Imidazolylalkyl” refers to a group of formula -alkyl-imidazolyl, where alkyl is as defined herein.

“Piperidylalkyl” refers to a group of formula -alkyl-piperidinyl, where alkyl is as defined herein.

“Naphthylalkyl” means a group of formula -alkyl-naphthyl, e.g., (8′-napthyl)methyl, where naphthyl has its normal meaning and alkyl is as defined herein.

“Indolylalkyl” means a group of formula -alkyl-indole, e.g., 3′-indolylethyl, and 3′-indolylmethyl, where indole has its normal meaning and alkyl is as defined herein.

“N-containing heterocyclyl” is meant to refer to any heteroaryl or heterocycloalkyl group containing at least one ring-forming N atom. Example N-containing heterocyclyl groups include pyridinyl, imidazolyl, piperidinyl, piperazinyl, pyrrolyl, indolyl, and the like.

“N-containing heterocyclylalkyl” is meant to refer to alkyl substituted by N-containing heterocyclylalkyl.

“Amino” and “primary amino” refer to NH₂. “Secondary amino” refers to NHR and “tertiary amino” refers to NR₂, where R is any suitable substituent.

“Ammonium” is meant to refer to the group —N(R)³⁺ where R can be any appropriate moiety such as alkyl, cycloalkyl, aryl, cycloalkylalkyl, arylalkyl, etc.

“Amino acid” refers to any of the twenty naturally occurring and genetically encoded α-amino acids or protected derivatives thereof. Protected derivatives of amino acids can contain one or more protecting groups on the amino moiety, carboxy moiety, or side chain moiety. Examples of amino-protecting groups include formyl, trityl, phthalimido, trichloroacetyl, chloroacetyl, bromoacetyl, iodoacetyl, and urethane-type blocking groups such as benzyloxycarbonyl, 4-phenylbenzyloxycarbonyl, 2-methylbenzyloxycarbonyl, 4-methoxybenzyloxycarbonyl, 4-fluorobenzyloxycarbonyl, 4-chlorobenzyloxycarbonyl, 3-chlorobenzyloxycarbonyl, 2-chlorobenzyloxycarbonyl, 2,4-dichlorobenzyloxycarbonyl, 4-bromobenzyloxycarbonyl, 3-bromobenzyloxycarbonyl, 4-nitrobenzyloxycarbonyl, 4-cyanobenzyloxycarbonyl, t-butoxycarbonyl, 2-(4-xenyl)-isopropoxycarbonyl, 1,1-diphenyleth-1-yloxycarbonyl, 1,1-diphenylprop-1-yloxycarbonyl, 2-phenylprop-2-yloxycarbonyl, 2-(p-toluoyl)-prop-2-yloxycarbonyl, cyclopentanyloxy-carbonyl, 1-methylcyclopentanyloxycarbonyl, cyclohexanyloxycarbonyl, 1-methylcyclohexanyloxycarbonyl, 2-methylcyclohexanyloxycarbonyl, 2-(4-toluoylsulfonyl)-ethoxycarbonyl, 2-(methylsulfonyl)ethoxycarbonyl, 2-(triphenylphosphino)-ethoxycarbonyl, fluorenylmethoxycarbonyl (“FMOC”), 2-(trimethylsilyl)ethoxycarbonyl, allyloxycarbonyl, 1-(trimethylsilylmethyl)prop-1-enyloxycarbonyl, 5-benzisoxalylmethoxycarbonyl, 4-acetoxybenzyloxycarbonyl, 2,2,2-trichloroethoxycarbonyl, 2-ethynyl-2-propoxycarbonyl, cyclopropylmethoxycarbonyl, 4-(decycloxy)benzyloxycarbonyl, isobornyloxycarbonyl, 1-piperidyloxycarbonlyl and the like; benzoylmethylsulfonyl group, 2-nitrophenylsulfenyl, diphenylphosphine oxide and like amino-protecting groups. Examples of carboxy-protecting groups include methyl, p-nitrobenzyl, p-methylbenzyl, p-methoxybenzyl, 3,4-dimethoxybenzyl, 2,4-dimethoxybenzyl, 2,4,6-trimethoxybenzyl, 2,4,6-trimethylbenzyl, pentamethylbenzyl, 3,4-methylenedioxybenzyl, benzhydryl, 4,4′-dimethoxybenzhydryl, 2,2′,4,4′-tetramethoxybenzhydryl, t-butyl, t-amyl, trityl, 4-methoxytrityl, 4,4′-dimethoxytrityl, 4,4′,4″-trimethoxytrityl, 2-phenylprop-2-yl, trimethylsilyl, t-butyldimethylsilyl, phenacyl, 2,2,2-trichloroethyl, .beta.-(di(n-butyl)methylsilyl)ethyl, p-toluenesulfonylethyl, 4-nitrobenzylsulfonylethyl, allyl, cinnamyl, 1-(trimethylsilylmethyl)prop-1-en-3-yl and like moieties. The species of protecting group employed is not critical so long as the derivatized protecting group can be selectively removed at the appropriate point without disrupting the remainder of the molecule. Further examples of protecting groups are found in E. Haslam, Protecting Groups in Organic Chemistry, (J. G. W. McOmie, ed., 1973), at Chapter 2; and T. W. Greene and P. G. M. Wuts, Protective Groups in Organic Synthesis, (1991), at Chapter 7, the disclosures of each of which are incorporated herein by reference in their entireties.

“N-Substituted glycine” refers to a residue of the formula —(NR—CH₂—CO)— where each R is a non-hydrogen moiety such as those independently selected from (C₂-C₆)alkyl, halo(C₁-C₆)alkyl, (C₂-C₆)alkenyl, (C₂-C₆)alkynyl, (C₆-C₁₀)cycloalkyl-aryl, amino(C₁-C₆)alkyl, ammonium(C₁-C₆)alkyl, hydroxy(C₁-C₆)alkyl, (C₁-C₆)alkoxy(C₁-C₆)alkyl, carboxy, carboxy(C₂-C₆)alkyl, carbamyl, carbamyl(C₂-C₆)alkyl, guanidino, guanidino(C₁-C₆)alkyl, amidino, amidino(C₁-C₆)alkyl, thiol, (C₁-C₆)alkylthiol, alkylthioalkyl of 2-10 carbon atoms, N-containing heterocyclyl, N-containing heterocyclyl(C₁-C₆)alkyl, imidazolyl, imidazolylalkyl of 4-10 carbon atoms, piperidyl, piperidylalkyl of 5-10 carbon atoms, indolyl, indolylalkyl of 9-15 carbon atoms, naphthyl, naphthylalkyl of 11-16 carbon atoms, and aryl(C₁-C₆)alkyl; where each R moiety is optionally substituted with 1-3 substituents independently selected from halogen, hydroxy and (C₁-C₆)alkoxy.

In some embodiments of —(NR—CH₂—CO)—, R is (C₂-C₆)alkyl, halo(C₁-C₆)alkyl, (C₂-C₆)alkenyl, (C₂-C₆)alkynyl, (C₆-C₁₀)cycloalkyl-aryl, amino(C₁-C₆)alkyl, hydroxy(C₁-C₆)alkyl, (C₁-C₆)alkoxy(C₁-C₆)alkyl, carboxy, carboxy(C₂-C₆)alkyl, carbamyl, carbamyl(C₂-C₆)alkyl, guanidino, guanidino(C₁-C₆)alkyl, thiol, (C₁-C₆)alkylthiol, alkylthioalkyl of 2-10 carbon atoms, imidazolyl, imidazolylalkyl of 4-10 carbon atoms, piperidyl, piperidylalkyl of 5-10 carbon atoms, indolyl, indolylalkyl of 9-15 carbon atoms, naphthyl, naphthylalkyl of 11-16 carbon atoms, diphenyl(C₁-C₆)alkyl or aryl(C₁-C₆)alkyl; where each R moiety is optionally substituted with 1-3 substituents independently selected from halogen, hydroxy and (C₁-C₆)alkoxy.

In some embodiments of —(NR—CH₂—CO)—, R is (C₂-C₆)alkyl, amino(C₁-C₆)alkyl, hydroxy(C₁-C₆)alkyl, (C₁-C₆)alkoxy(C₁-C₆)alkyl, guanidino(C₁-C₆)alkyl, indolylalkyl of 9-15 carbon atoms, naphthylalkyl of 11-16 carbon atoms, diphenyl(C₁-C₆)alkyl or aryl(C₁-C₆)alkyl, substituted with 1-3 substituents independently selected from halogen, hydroxy or (C₁-C₆)alkoxy.

In some embodiments of —(NR—CH₂—CO)—, R is a moiety that is charged at physiologically relevant pH. Examples of positively charged R at physiologically relevant pH include, for example, amino(C₁-C₆)alkyl, ammonium(C₁-C₆)alkyl, guanidino, guanidino(C₁-C₆)alkyl, amidino, amidino(C₁-C₆)alkyl, N-containing heterocyclyl, and N-containing heterocyclyl(C₁-C₆)alkyl, wherein each R moiety is optionally substituted with 1-3 substituents independently selected from halogen, C1-C3 methoxy, and C1-C3 alkyl.

In some embodiments of —(NR—CH₂—CO)—, R is a moiety that is neutral at physiologically relevant pH. Examples of neutral R at physiologically relevant pH include, for example, (C₂-C₆)alkyl, halo(C₁-C₆)alkyl, (C₂-C₆)alkenyl, (C₂-C₆)alkynyl, (C₆-C₁₀)cycloalkyl-aryl, (C₁-C₆)alkoxy(C₁-C₆)alkyl, alkylthioalkyl of 2-10 carbon atoms, diphenyl(C₁-C₆)alkyl, and aryl(C₁-C₆)alkyl. Further examples include ethyl, prop-1-yl, prop-2-yl, 1-methylprop-1-yl, 2-methylprop-1-yl, 3-phenylpropy-1-yl, 3-methylbutyl, benzyl, 4-chloro-benzyl, 4-methoxy-benzyl, 4-methyl-benzyl, 2-methylthioeth-1-yl, and 2,2-diphenylethyl.

In some embodiments of —(NR—CH₂—CO)—, R is amino(C₁-C₆)alkyl (e.g., aminobutyl).

Further example N-substituted glycines include those where R is ethyl, prop-1-yl, prop-2-yl, 1-methylprop-1-yl, 2-methylprop-1-yl, 3-phenylpropy-1-yl, 3-methylbutyl, benzyl, 4-hydroxybenzyl, 4-chloro-benzyl, 4-methoxy-benzyl, 4-methyl-benzyl, 2-hydroxyethyl, mercaptoethyl, 2-aminoethyl, 3-propionic acid, 3-aminopropyl, 4-aminobutyl, 2-methylthioeth-1-yl, carboxymethyl, 2-carboxyethyl, carbamylmethyl, 2-carbamylethyl, 3-guanidinoprop-1-yl, imidazolylmethyl, 2,2-diphenylethyl or indol-3-yl-ethyl.

Also included are salts, esters, and protected forms (e.g., N-protected with Fmoc or Boc, etc.) of the N-substituted glycines.

Methods for making amino acid substitutes, including N-substituted glycines, are disclosed, inter alia, in U.S. Pat. No. 5,811,387, which is incorporated herein by reference in its entirety.

“Monomer” or “subunit” refers to a molecule that can be linked to other monomers to form a chain, e.g., a peptide. Amino acids and N-substituted glycines are example monomers. When linked with other monomers, a monomer can be referred to as a “residue.”

### II. CONFORMATIONAL DISEASES

This invention relates to methods to detect pathogenic conformers of non-prion conformational disease proteins and methods to diagnose the diseases associated with such proteins. Conformational disease proteins and their corresponding diseases include those listed in the below table.

TABLE 2

| Conformational Disease | |
|---|---|
| Protein | Disease/Symptoms/Cause |
| Inflammation/Immunity | |
| Immunoglobulin light | Systemic amyloidosis - myeloma-associated |
| chain | |
| Immunoglobulin heavy | Systemic amyloidosis - myeloma-associated |
| chain | |
| Serum Amyloid A | Systemic amyloidosis, rheumatoid arthritis, |
| | chronic inflammation |
| Cystatin C | Systemic amyloidosis - familial |
| Lysozyme | Systemic amyloidosis - familial |
| Fibrinogen | Systemic amyloidosis - familial |
| Nervous System | |
| Amyloid β | Alzheimer's disease |
| Prion protein | Transmissible spongiform encephalopathies |
| Endocrine Hormones | |
| Prolactin | Pituitary - age-related |
| Islet amyloid polypeptide | Local amyloidosis - Type 2 diabetes-related |
| Atrial natriuretic factor | Localized atrial amyloidosis |
| Transport Proteins | |
| Transthyretin | Systemic amyloidosis - familial, senile |
| | systemic amyloidosis |
| β2-microglobulin | Chronic haemodialysis |
| Apolipoprotein AI | Systemic amyloidosis - familial |
| Ocular Proteins | |
| Gelsolin | Systemic amyloidosis - familial |
| Lactoferrrin | Familial corneal amyloidosis |
| Keratoepethelin | Familial corneal dystrophies |

Conformational diseases of this invention include any disease associated with proteins which form two or more different conformations other than those diseases associated with prions. Those of particular interest herein include amyloid diseases, all which display a cross-beta sheet signature, such as Alzheimer's disease, systemic amyloidoses, tauopathies, and synucleinopathies. Other diseases of interest are diabetes and poly-glutamine diseases.

In certain embodiments of the methods of the invention, a conformational disease protein-specific binding reagent (“CDPSB reagent”) is used to either capture or detect both non-pathogenic and pathogenic conformers. The particular CDPSB reagent used will depend on the pathogenic conformer being detected. For example, if the conformational disease to be diagnosed is Alzheimer's disease, then the CDPSB reagent may be an antibody which recognizes both the non-pathogenic and pathogenic conformers of the Alzheimer's disease protein Aβ.

### III. REAGENTS TO BE USED IN METHODS OF THIS INVENTION

Pathogenic conformer-specific binding reagents (“PCSB reagents”) to be used in this invention are those reagents which interact preferentially with pathogenic prion proteins.

Typically, PCSB reagents are derived from prion protein fragments. Preferably such PCSB reagents are either peptides or modified peptides, including those commonly known as peptoids.

In certain embodiments, such PCSB reagents are polycationic. Most preferably, the PCSB reagents have a net charge of at least positive three or positive four at physiological pH. While not wishing to be bound by theory, Applicants believe that the PCSB reagents described herein bind to non-prion pathogenic conformers via a mechanism similar to that by which PCSB reagents derived from prion protein fragments bind to PrP^{Sc} and therefore exhibit similar binding properties. Lau et al. (previously cited herein) demonstrate that core peptide sequences required for binding to PrP^{Sc} in both plasma and buffer have four positively charged amino acids. Peptides containing only three positively charged amino acid residues can only bind PrP^{Sc} in buffer. Furthermore, alanine scanning of a peptide which binds PrP^{Sc} in both buffer and plasma is reduced to background levels by removal of any single positively charged amino acid.

### A. Preferred Prion Protein Fragments

PCSB reagents are preferably derived from the amino acid sequences of certain prion protein fragments. These preferred regions are exemplified with respect to both the mouse prion sequence (SEQ ID NO:2) and the human prion sequence (SEQ ID NO:1). The PCSB reagents used in methods of the invention are preferably derived from those prion protein fragments which serve as the basis for other PCSB reagents known to interact preferentially with pathogenic prion proteins but can also be derived from any prion protein fragment as long as the resulting PCSB reagent can interact preferentially with pathogenic prion proteins. Specific preferred sequences are described below.

The PCSB reagents used in the invention can be derived from fragments of the amino acid sequences of any species or variant. The polynucleotide and amino acid sequence for prion proteins produced by many different species are known, including human, mouse, sheep and cattle. Variants to these sequences also exist within each species. For example, in certain embodiments, the peptide PCSB reagents described herein are derived from any of the sequences set forth in SEQ ID NOs:1-11. The sequences of the PCSB reagents that are specifically disclosed herein are based on either the mouse or human prion sequence, however, one skilled in the art can readily substitute corresponding sequences from other species when appropriate.

Preferred lengths of prion protein fragments from which the PCSB reagents can be derived include 3 to 5 residues in length, 6 to 10 residues in length (or any integer therebetween), 11 to 20 residues in length (or any integer therebetween), 21 to 75 residues in length (or any integer therebetween), 75 to 100 (or any integer therebetween), or polypeptides of greater than 100 residues in length. Preferably, the peptide is between about 3 and 100 residues in length. Generally, one skilled in art can easily select the maximum length in view of the teachings herein. Further, reagents as described herein, for example synthetic peptides, may include additional molecules such as labels, linkers, or other chemical moieties (e.g., biotin, amyloid-specific dyes such as Control Red or Thioflavin). Such moieties may further enhance interaction of the PCSB reagents with the pathogenic conformers and/or further detection of pathogenic conformers.

In preferred embodiments, the PCSB reagent is derived from prion protein fragments known to interact preferentially with the pathogenic prion protein, such as those having sequences corresponding to the following human prion protein fragments: PrP₁₉₋₃₀ (SEQ ID NO: 242), PrP₂₃₋₃₀ (SEQ ID NO: 243), PrP₁₀₀₋₁₁₁ (SEQ ID NO: 244), PrP₁₀₁₋₁₁₀ (SEQ ID NO: 245), PrP₁₅₄₋₁₆₅ (SEQ ID NO: 246), PrP₂₂₆₋₂₃₇ (SEQ ID NO: 247), SEQ ID NO:14, SEQ ID NO:50 and SEQ ID NO:68.

### B. PCSB Peptide Reagents

PCSB reagents derived from prion protein fragments may have the exact amino acid sequence of a prion protein fragment or may be variations or modified forms of a prion protein fragment. The PCSB reagents used in methods of the invention are preferably derived from those prion protein fragments which serve as the basis for other PCSB reagents known to interact preferentially with pathogenic prion proteins but can also be derived from any prion protein fragment as long as the resulting reagent can interact preferentially with pathogenic prion proteins.

PCSB reagents include derivatives of the amino acid sequences of prion protein fragments which have one or more substitution, addition and/or deletion, including one or more non-naturally occurring amino acid. Preferably, derivatives exhibit at least about 50% identity to any wild type or reference sequence, preferably at least about 70% identity, more preferably at least about 75%, 80%, 85%, 90%, 91%, 92%, 93%, 94%, 95%, 96%, 97%, 98%, 99% or 100% sequence identity to any wild type or reference sequence described herein. Sequence (or percent) identity can be determined using any method known to those of skill in the art, such as those described below. Such derivatives can include postexpression modifications of the polypeptide, for example, glycosylation, acetylation, phosphorylation, and the like.

Techniques for determining amino acid sequence similarity or percent identity are well known in the art. In general, “similarity” means the amino acid to amino acid comparison of two or more polypeptides at the appropriate place, where amino acids are identical or possess similar chemical and/or physical properties such as charge or hydrophobicity. A so-termed “percent identity” then can be determined between the compared polypeptide sequences. Techniques for determining nucleic acid and amino acid sequence identity also are well known in the art and include determining the nucleotide sequence of the mRNA for that gene (usually via a cDNA intermediate) and determining the amino acid sequence encoded thereby, and comparing this to a second amino acid sequence. In general, “identity” refers to an exact nucleotide to nucleotide or amino acid to amino acid correspondence of two polynucleotides or polypeptide sequences, respectively.

Two or more amino acid or polynucleotide sequences can be compared by determining their “percent identity.” Percent identity can be determined by a direct comparison of the sequence information between two molecules (the reference sequence and a sequence with unknown % identity to the reference sequence) by aligning the sequences, counting the exact number of matches between the two aligned sequences, dividing by the length of the reference sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353-358, National biomedical Research Foundation, Washington, D.C., which adapts the local homology algorithm of Smith and Waterman Advances in Appl. Math. 2:482-489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present invention is to use the MPSRCH™ package of programs copyrighted by the University of Edinburgh, developed by John F. Collins and Shane S. Sturrok, and available from numerous sources, for example on the internet. From this suite of packages the Smith—Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs are readily available

PCSB reagents can also include PCSB reagents with modifications to the native prion protein sequence, such as deletions, additions and substitutions (generally conservative in nature), so long as the polypeptide maintains the desired activity. In certain embodiments, conservative amino acid replacements are preferred. Conservative amino acid replacements are those that take place within a family of amino acids that are related in their side chains. Genetically encoded amino acids are generally divided into four families: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar=glycine, asparagine, glutamine, cysteine, serine, threonine, tyrosine. Phenylalanine, tryptophan, and tyrosine are sometimes classified jointly as aromatic amino acids. For example, it is reasonably predictable that an isolated replacement of a leucine with an isoleucine or valine, an aspartate with a glutamate, a threonine with a serine, or a similar conservative replacement of an amino acid with a structurally related amino acid will not have a major effect on the biological activity. These modifications may be deliberate, as through site-directed mutagenesis, or may be accidental, such as through mutations of hosts that produce the proteins or errors due to PCR amplification. Furthermore, modifications may be made that have one or more of the following effects: reducing toxicity; increasing affinity and/or specificity for pathogenic conformers; facilitating cell processing (e.g., secretion, antigen presentation, etc.); and facilitating presentation to B-cells and/or T-cells.

PCSB reagents may contain one or more analogs of an amino acid (including, for example, unnatural amino acids, etc.), peptides with substituted linkages, as well as other modifications known in the art, both naturally occurring and non-naturally occurring (e.g., synthetic). Thus, synthetic peptides, dimers, multimers (e.g., tandem repeats, multiple antigenic peptide (MAP) forms, linearly-linked peptides), cyclized, branched molecules and the like are considered to be peptides. This also include molecules comprising one or more N-substituted glycine residues (a “peptoid”) and other synthetic amino acids or peptides. (See, e.g., U.S. Pat. Nos. 5,831,005; 5,877,278; and 5,977,301; Nguyen et al. (2000) Chem. Biol. 7(7):463-473; and Simon et al. (1992) Proc. Natl. Acad. Sci. USA 89(20):9367-9371 for descriptions of peptoids).

For a general review of these and other amino acid analogs and peptidomimetics see, Nguyen et al. (2000) Chem. Biol. 7(7):463-473; Spatola, A. F., in Chemistry and Biochemistry of Amino Acids, Peptides and Proteins, B. Weinstein, eds., Marcel Dekker, New York, p. 267 (1983). See also, Spatola, A. F., Peptide Backbone Modifications (general review), Vega Data, Vol. 1, Issue 3, (March 1983); Morley, Trends Pharm Sci (general review), pp. 463-468 (1980); Hudson, D. et al., Int J Pept Prot Res, 14:177-185 (1979) (—CH₂NH—, CH₂CH₂—); Spatola et al., Life Sci, 38:1243-1249 (1986) (—CH₂—S); Hann J. Chem. Soc. Perkin Trans. I, 307-314 (1982) (—CH—CH—, cis and trans); Almquist et al., J Med Chem, 23:1392-1398 (1980) (—COCH₂—); Jennings-White et al., Tetrahedron Lett, 23:2533 (1982) (—COCH₂—); Szelke et al., European Appln. EP 45665 CA: 97:39405 (1982) (—CH(OH)CH₂—); Holladay et al., Tetrahedron Lett, 24:4401-4404 (1983) (—C(OH)CH₂—); and Hruby, Life Sci, 31:189-199 (1982) (—CH₂—S—); each of which is incorporated herein by reference.

It will also be apparent that any combination of the natural amino acids and non-natural amino acid analogs can be used to make the PCSB reagents described herein. Commonly encountered amino acid analogs that are not gene-encoded include, but are not limited to, ornithine (Orn); aminoisobutyric acid (Aib); benzothiophenylalanine (BtPhe); albizziin (Abz); t-butylglycine (Tle); phenylglycine (PhG); cyclohexylalanine (Cha); norleucine (Nle); 2-naphthylalanine (2-Nal); 1-naphthylalanine (1-Nal); 2-thienylalanine (2-Thi); 1,2,3,4-tetrahydroisoquinoline-3-carboxylic acid (Tic); N-methylisoleucine (N-MeIle); homoarginine (Har); Nα-methylarginine (N-MeArg); phosphotyrosine (pTyr or pY); pipecolinic acid (Pip); 4-chlorophenylalanine (4-ClPhe); 4-fluorophenylalanine (4-FPhe); 1-aminocyclopropanecarboxylic acid (1-NCPC); and sarcosine (Sar). Any of the amino acids used in the PCSB reagents may be either the D- or, more typically, L-isomer.

Other non-naturally occurring analogs of amino acids that may be used to form the PCSB reagents described herein include peptoids and/or peptidomimetic compounds such as the sulfonic and boronic acid analogs of amino acids that are biologically functional equivalents are also useful in the compounds of the present invention and include compounds having one or more amide linkages optionally replaced by an isostere. In the context of the present invention, for example, —CONH— may be replaced by —CH₂NH—, —NHCO—, —SO₂NH—, —CH₂O—, —CH₂CH₂—, —CH₂S—, —CH₂SO—, —CH—CH— (cis or trans), —COCH₂—, —CH(OH)CH₂— and 1,5-disubstituted tetrazole such that the radicals linked by these isosteres would be held in similar orientations to radicals linked by —CONH—. One or more residues in the PCSB reagents described herein may include peptoids.

Thus, the reagents also may include one or more N-substituted glycine residues (peptides having one or more N-substituted glycine residues may be referred to as “peptoids”). For example, in certain embodiments, one or more proline residues of any of the PCSB reagents described herein are replaced with N-substituted glycine residues. Particular N-substituted glycines that are suitable in this regard include, but are not limited to, N—(S)-(1-phenylethyl)glycine; N-(4-hydroxyphenyl)glycine; N-(cyclopropylmethyl)glycine; N-(isopropyl)glycine; N-(3,5-dimethoxybenzyl)glycine; and N-butylglycine. Other N-substituted glycines may also be suitable to replace one or more amino acid residues in the PCSB reagent sequences described herein.

The PCSB reagents described herein may be monomers, multimers, cyclized molecules, branched molecules, linkers and the like. Multimers (i.e., dimers, trimers and the like) of any of the sequences described herein or biologically functional equivalents thereof are also contemplated. The multimer can be a homomultimer, i.e., composed of identical monomers, e.g., each monomer is the same peptide sequence. Alternatively, the multimer can be a heteromultimer, by which is meant that not all the monomers making up the multimer are identical.

Multimers can be formed by the direct attachment of the monomers to each other or to substrate, including, for example, multiple antigenic peptides (MAPS) (e.g., symmetric MAPS), peptides attached to polymer scaffolds, e.g., a PEG scaffold and/or peptides linked in tandem with or without spacer units.

Alternatively, linking groups can be added to the monomeric sequences to join the monomers together and form a multimer. Non-limiting examples of multimers using linking groups include tandem repeats using glycine linkers; MAPS attached via a linker to a substrate and/or linearly linked peptides attached via linkers to a scaffold. Linking groups may involve using bifunctional spacer units (either homobifunctional or heterobifunctional) as are known to one of skill in the art. By way of example and not limitation, many methods for incorporating such spacer units in linking peptides together using reagents such as succinimidyl-4-(p-maleimidomethyl)cyclohexane-1-carboxylate (SMCC), succinimidyl-4-(p-maleimidophenyl)butyrate and the like are described in the Pierce Immunotechnology Handbook (Pierce Chemical Co., Rockville, Ill.) and are also available from Sigma Chemical Co. (St. Louis, Mo.) and Aldrich Chemical Co. (Milwaukee, Wis.) and described in “Comprehensive Organic Transformations”, VCK-Verlagsgesellschaft, Weinheim/Germany (1989). One example of a linking group which may be used to link the monomeric sequences together is —Y₁—F—Y₂ where Y₁ and Y₂ are identical or different and are alkylene groups of 0-20, preferably 0-8, more preferably 0-3 carbon atoms, and F is one or more functional groups such as —O—, —S—, —S—S—, —C(O)—O—, —NR—, —C(O)—NR—, —NR—C(O)—O—, —NR—C(O)—NR—, —NR—C(S)—NR—, —NR—C(S)—O—. Y₁ and Y₂ may be optionally substituted with hydroxy, alkoxy, hydroxyalkyl, alkoxyalkyl, amino, carboxyl, carboxyalkyl and the like. It will be understood that any appropriate atom of the monomer can be attached to the linking group.

Further, the PCSB reagents described herein may be linear, branched or cyclized. Monomer units can be cyclized or may be linked together to provide the multimers in a linear or branched fashion, in the form of a ring (for example, a macrocycle), in the form of a star (dendrimers) or in the form of a ball (e.g., fullerenes). Skilled artisans will readily recognize a multitude of polymers that can be formed from the monomeric sequences disclosed herein. In certain embodiments, the multimer is a cyclic dimer. Using the same terminology as above, the dimer can be a homodimer or a heterodimer.

Cyclic forms, whether monomer or multimer, can be made by any of the linkages described above, such as but not limited to, for example: (1) cyclizing the N-terminal amine with the C-terminal carboxylic acid either via direct amide bond formation between the nitrogen and the C-terminal carbonyl, or via the intermediacy of spacer group such as for example by condensation with an epsilon-amino carboxylic acid; (2) cyclizing via the formation of a bond between the side chains of two residues, e.g., by forming a amide bond between an aspartate or glutamate side chain and a lysine side chain, or by disulfide bond formation between two cysteine side chains or between a penicillamine and cysteine side chain or between two penicillamine side chains; (3) cyclizing via formation of an amide bond between a side chain (e.g., aspartate or lysine) and either the N-terminal amine or the C-terminal carboxyl respectively; and/or (4) linking two side chains via the intermediacy of a short carbon spacer group.

Furthermore, the PCSB reagents described herein may also include additional peptide or non-peptide components. Non-limiting examples of additional peptide components include spacer residues, for example two or more glycine (natural or derivatized) residues or aminohexanoic acid linkers on one or both ends or residues that may aid in solubilizing the peptide reagents, for example acidic residues such as aspartic acid (Asp or D). In certain embodiments, for example, the peptide reagents are synthesized as multiple antigenic peptides (MAPs). Typically, multiple copies of the peptide reagents (e.g., 2-10 copies) are synthesized directly onto a MAP carrier such as a branched lysine or other MAP carrier core. See, e.g., Wu et al. (2001) J Am Chem. Soc. 2001 123(28):6778-84; Spetzler et al. (1995) Int J Pept Protein Res. 45(1):78-85.

Non-limiting examples of non-peptide components (e.g., chemical moieties) that may be included in the PCSB reagents described herein include, one or more detectable labels, tags (e.g., biotin, His-Tags, oligonucleotides), dyes, members of a binding pair, and the like, at either terminus or internal to the peptide reagent. The non-peptide components may also be attached (e.g., via covalent attachment of one or more labels), directly or through a spacer (e.g., an amide group), to position(s) on the compound that are predicted by quantitative structure-activity data and/or molecular modeling to be non-interfering. PCSB Reagents as described herein may also include prion-specific chemical moieties such as amyloid-specific dyes (e.g., Congo Red, Thioflavin, etc.). Derivatization (e.g., labeling, cyclizing, attachment of chemical moieties, etc.) of compounds should not substantially interfere with (and may even enhance) the binding properties, biological function and/or pharmacological activity of the reagent.

The above described peptides can be prepared using standard methods known to those of skill in the art, including but not limited to expression from recombinant constructs and peptide synthesis.

### C. Examples of Preferred Peptides to be Used as Basis for PCSB Reagents

Non-limiting examples of peptides useful in making the pathogenic conformer-specific binding reagents of the invention are preferably derived from sequences shown in Table 3. The peptides in the table are represented by conventional one letter amino acid codes and are depicted with their amino-terminus at the left and carboxy-terminus at the right. X indicates that any amino acid can be located at that position.

Any of the sequences in the table may optionally include Gly linkers (Gn where n=1, 2, 3, or 4) at the amino- and/or carboxy-terminus. Amino acids in square brackets indicate alternative residues that can be used at that position in different peptides. Round brackets indicate the residue(s) may be present or absent from the peptide reagent. Double round brackets (e.g., SEQ ID NO: 111) followed by a “2” indicate that the sequence includes two copies of the peptide between the double brackets. The residue following the copy number designation (e.g., “K” in SEQ ID NO: 111) indicates the residue from which each copy of the peptide between the double brackets extends. Thus, SEQ ID NO: 111 is a dimer of QWNKPSKPKTN peptide sequences (i.e., SEQ ID NO: 14), each linked by their carboxy-terminus to a lysine (K) residue via the a- and e-amino functional groups of lysine. Sequences including “MAPS” indicate peptides with multiple antigenic sites. The number preceding the term “branch” indicates the number of copies. Thus, SEQ ID NO: 112 contains 4 copies of GGGKKRPKPGGWNTGGG, which is SEQ ID NO: 67 with Gly linkers at each terminus, while SEQ ID NO: 113 contains 8 copies of GGGKKRPKPGGWNTGGG, which again is SEQ ID NO: 67 with Gly linkers at each terminus

TABLE 3
Peptide sequences for making PCSB reagents

| | SEQ ID |
|---|---|
| Peptide sequence | NO |
| KKRPK | 12 |
| MANLGCWMLVLFVATWSDLGLC | 13 |
| (GGG)QWNKPSKPKTN | 14 |
| QWNKPSKPKTNMKHV | 15 |
| NQNN[N/T]FVHDCVNIT[I/V]K[Q/E]HTVTTTTKGEN | 16 |
| TTKGENFTETD | 17 |
| GENFTETD | 18 |
| GENFTETD[V/I]K[M/I]MERVVEQMC[I/V]TQY[E/Q] | 19 |
| ESQAYY[Q/D](G)(R)R[G/S][S/A]S | |
| NQNN[N/T]FVHDCVNIT[I/V]K[Q/E]HTVTTTTKGENF | 20 |
| TETD[V/I]K[M/I]MERVVEQMC[I/V]TQY[E/Q]ESQA | |
| YY[Q/D](G)(R)R[G/S][S/A]S | |
| [A/V/T/M][V/I]LFSSPPVILLISFLIFL[I/M]VG | 21 |
| G[N/S]D[W/Y]EDRYYRENM[H/Y]RYPNQVYYRP[M/V] | 22 |
| D[Q/E/R]Y[S/N]NQN[N/T]FVH | |
| N[N/T]FVHDCVNIT[I/V]K[Q/E]HTVTTTTK | 23 |
| VYYR | 24 |
| RYPNQVYYRP[M/V]D[Q/E/R] | 25 |
| KKRPKPGG(G)WNTGGSRYPGQGSPGGNRYPPQGG | 26 |
| WNTGGSRYPGQGSPGGNRYPPQGG(G) | 27 |
| WNTGGSRYPGQGSPGGNRYPPQGG(G)[G/T]GQPHGG | 28 |
| GGWGQGGGTHSQWNKPSKPKTN | 29 |
| GGTHSQWNKPSKPKTN | 30 |
| WNTGGSRYPGQGSPGGNRYPPQGG(G)[G/T]WGQPHGGGW | 31 |
| GQPHGGGWGQPHGG | |
| GQPHGGGW | 32 |
| RPIIHFGSDYEDRYYRENMHR | 33 |
| RPMIHFGNDWEDRYYRENMYR | 34 |
| (GGGG)C(GG)GGWGQGGGTHNQWNKPSKPKTNLKHV(GGG | 35 |
| G)C | |
| (GGGG)GGWGQGGGTHNQWNKPSKPKTNLKHV | 36 |
| GGWGQGGGTHNQWNKPSKPKTNLKHV(GGGG) | 37 |
| [M/L]KH[M/V] | 38 |
| KPKTN[M/L]KH[M/V] | 39 |
| C(GG)GGWGQGGGTHNQWNKPSKPKTNLKHV(GGGG)C | 40 |
| SRPIIHFGSDYEDRYYRENMHRYPN | 41 |
| PMIHFGNDWEDRYYRENMYRPVD | 42 |
| AGAAAAGAVVGGLGGYMLGSAM | 43 |
| RPMIHFGNDWEDRYYRENMYR(GGG) | 44 |
| GGGRPMIHFGNDWEDRYYRENMYRGG | 45 |
| (GG)C(GGG)RPMIHFGNDWEDRYYRENMYR(GGG)C | 46 |
| AGAAAAGAVVGGLGG | 47 |
| GGLGG | 48 |
| LGS | 49 |
| QWNKPSKPKTN(GGG) | 50 |
| QWNKPSKPKTN(GGG)QWNKPSKPKTN | 51 |
| QWNKPSKPKTNLKHV(GGG) | 52 |
| GGWGQGGGTHNQWNKPSKPKTN | 53 |
| GGTHNQWNKPSKPKTN | 54 |
| (GGG)AGAAAAGAVVGGLGGYMLGSAM | 55 |
| (GGG)AGAAAAGAVVGGLGG | 56 |
| (KKK)AGAAAAGAVVGGLGGYMLGSAM | 57 |
| YMLGSAM[S/N]R | 58 |
| [S/N]RPPM/I/L][I/L]H | 59 |
| YMLGSAM[S/N]RP[M/I/L][I/L]H | 60 |
| YMLGSAM[S/N]RP[M/I/L][I/L]HFG[N/S]D | 61 |
| [W/Y]EDRYYRENM[H/Y]RYPNQVYYRP[MN]D[Q/E/ | 62 |
| R]Y | |
| [W/Y]EDRYYRENM[H/Y]RYPNQVYYRP[M/V]D[Q/E/ | 63 |
| R]Y[S/N]NQN[N/T] | |
| D[Q/E/R]Y[S/N]NQN[N/T] | 64 |
| (KKK)AGAAAAGAVVGGLGG | 65 |
| (GGG)KKRPKPGGWNTGGSRYPGQGS | 66 |
| (GGG)KKRPKPGGWNTGG | 67 |
| (GGG)KKRPKPGG | 68 |
| PHGGGWGQHGGSWGQPHGGSWGQ | 69 |
| PHGGGWGQPHGGSWGQ | 70 |
| PHGGGWGQ | 71 |
| (GGG)KKRPKPGGGKKRPKPGG | 72 |
| (GGG)GPKRKGPK | 73 |
| (GGG)WNTGGSRYPGQGS | 74 |
| (GGG)WNKPSKPKT | 75 |
| (GGG)RPMIHFGNDWEDRYYRENMYR(GG)C | 76 |
| QWNKPSKPKTNLKHV(GGG) | 77 |
| (GGG)AGAAAAGAVVGGLGGYMLGSAM | 78 |
| (GGG)NKPSKPK | 79 |
| (GGG)KPSKPK | 80 |
| (GGG)KKRPKPGGGQWNKPSKPKTN | 81 |
| KKKAGAAAAGAVVGGLGGYMLGSAMDDD | 82 |
| DDDAGAAAAGAVVGGLGGYMLGSAM | 83 |
| KKKAGAAAAGAVVGGLGGYMLGSAMKKK | 84 |
| (GGG)KKKKKKKK | 85 |
| DDDAGAAAAGAVVGGLGGYMLGSAMDDD | 86 |
| (GGG)NNKQSPWPTKK | 87 |
| DKDKGGVGALAGAAVAAGGDKDK | 88 |
| (GGG)QANKPSKPKTN | 89 |
| (GGG)QWNKASKPKTN | 90 |
| (GGG)QWNKPSKAKTN | 91 |
| (GGG)QWNAPSKPKTN | 92 |
| (GGG)QWNKPSAPKTN | 93 |
| (GGG)QWNKPSKPATN | 94 |
| (GGG)QWNKASKAKTN | 95 |
| (GGG)KKRAKPGG | 96 |
| (GGG)KKRPKAGG | 97 |
| (GGG)KKRAKAGG | 98 |
| (GGG)QWNKASKPKTN | 99 |
| (GGG)QWAKPSKPKTN | 100 |
| (GGG)QWNKPAKPKTN | 101 |
| (GGG)QWNKPSKPKAN | 102 |
| (GGG)QWNKPSKPKTA | 103 |
| (GGG)AKRPKPGG | 104 |
| (GGG)KARPKPGG | 105 |
| (GGG)KKAPKPGG | 106 |
| (GGG)KKRPAPGG | 107 |
| (GGG)KKAPKAGG | 108 |
| (GGG)KKRPKPGGGWNTGG | 109 |
| QWNKPSKPKTNGGGQWNKPSKPKTNGGGQWNKPSKPKTN | 110 |
| ((QWNKPSKPKTN))2K | 111 |
| 4-branchMAPS-GGGKKRPKPGGWNTGGG | 112 |
| 8-branchMAPS-GGGKKRPKPGGWNTGGG | 113 |
| KKKAGAAAAGAVVGGLGG-CONH2 | 114 |
| DLGLCKKRPKPGGXWNTGG | 115 |
| DLGLCKKRPKPGGXWNTG | 116 |
| DLGLCKKRPKPGGXWNT | 117 |
| DLGLCKKRPKPGGXWN | 118 |
| DLGLCKKRPKPGGXW | 119 |
| DLGLCKKRPKPGGX | 120 |
| LGLCKKRPKPGGXWNTG | 121 |
| LGLCKKRPKPGGXWNT | 122 |
| LGLCKKRPKPGGXWN | 123 |
| LGLCKKRPKPGGXW | 124 |
| LGLCKKRPKPGGX | 125 |
| GLCKKRPKPGGXWNTGG | 126 |
| GLCKKRPKPGGXWNTG | 127 |
| GLCKKRPKPGGXWNT | 128 |
| GLCKKRPKPGGXWN | 129 |
| GLCKKRPKPGGXW | 130 |
| GLCKKRPKPGGX | 131 |
| LCKKRPKPGGXWNTGG | 132 |
| LCKKRPKPGGXWNTG | 133 |
| LCKKRPKPGGXWNT | 134 |
| LCKKRPKPGGXWN | 135 |
| LCKKRPKPGGXW | 136 |
| LCKKRPKPGGX | 137 |
| CKKRPKPGGXWNTGG | 138 |
| CKKRPKPGGXWNTG | 139 |
| CKKRPKPGGXWNT | 140 |
| CKKRPKPGGXWN | 141 |
| CKKRPKPGGXW | 142 |
| CKKRPKPGGX | 143 |
| KKRPKPGGXWNTGG | 144 |
| KKRPKPGGXWNTG | 145 |
| KKRPKPGGXWNT | 146 |
| KKRPKPGGXWN | 147 |
| KKRPKPGGXW | 148 |
| KKRPKPGGX | 149 |
| DVGLCKKRPKPGGXWNTGG | 150 |
| DVGLCKKRPKPGGXWNTG | 151 |
| DVGLCKKRPKPGGXWNT | 152 |
| DVGLCKKRPKPGGXWN | 153 |
| DVGLCKKRPKPGGXW | 154 |
| DVGLCKKRPKPGGX | 155 |
| VGLCKKRPKPGGXWNTG | 156 |
| VGLCKKRPKPGGXWNT | 157 |
| VGLCKKRPKPGGXWN | 158 |
| VGLCKKRPKPGGXW | 159 |
| VGLCKKRPKPGGX | 160 |
| THSQWNKPSKPKTNMKHM | 161 |
| THSQWNKPSKPKTNMKH | 162 |
| THSQWNKPSKPKTNMK | 163 |
| THSQWNKPSKPKTNM | 164 |
| THSQWNKPSKPKTN | 165 |
| HSQWNKPSKPKTNMKHM | 166 |
| HSQWNKPSKPKTNMKH | 167 |
| HSQWNKPSKPKTNMK | 168 |
| HSQWNKPSKPKTNM | 169 |
| HSQWNKPSKPKTN | 170 |
| SQWNKPSKPKTNMKHM | 171 |
| SQWNKPSKPKTNMKH | 172 |
| SQWNKPSKPKTNMK | 173 |
| SQWNKPSKPKTNM | 174 |
| SQWNKPSKPKTN | 175 |
| QWNKPSKPKTNMKHM | 176 |
| QWNKPSKPKTNMKH | 177 |
| QWNKPSKPKTNMK | 178 |
| QWNKPSKPKTNM | 179 |
| THSQWNKPSKPKTNMKHV | 180 |
| HSQWNKPSKPKTNMKHV | 181 |
| SQWNKPSKPKTNMKHV | 182 |
| QWNKPSKPKTNMKHV | 183 |
| THGQWNKPSKPKTNMKHM | 184 |
| THGQWNKPSKPKTNMKH | 185 |
| THGQWNKPSKPKTNMK | 186 |
| THGQWNKPSKPKTNM | 187 |
| THGQWNKPSKPKTN | 188 |
| HGQWNKPSKPKTNMKHM | 189 |
| HGQWNKPSKPKTNMKH | 190 |
| HGQWNKPSKPKTNMK | 191 |
| HGQWNKPSKPKTNM | 192 |
| HGQWNKPSKPKTN | 193 |
| GQWNKPSKPKTNMKHM | 194 |
| GQWNKPSKPKTNMKH | 195 |
| GQWNKPSKPKTNMK | 196 |
| GQWNKPSKPKTNM | 197 |
| GQWNKPSKPKTN | 198 |
| THGQWNKPSKPKTNMKHV | 199 |
| HGQWNKPSKPKTNMKHV | 200 |
| GQWNKPSKPKTNMKHV | 201 |
| THNQWNKPSKPKTNMKHM | 202 |
| THNQWNKPSKPKTNMKH | 203 |
| THNQWNKPSKPKTNMK | 204 |
| THNQWNKPSKPKTNM | 205 |
| THNQWNKPSKPKTN | 206 |
| HNQWNKPSKPKTNMKHM | 207 |
| HNQWNKPSKPKTNMKH | 208 |
| HNQWNKPSKPKTNMK | 209 |
| HNQWNKPSKPKTNM | 210 |
| HNQWNKPSKPKTN | 211 |
| NQWNKPSKPKTNMKHM | 212 |
| NQWNKPSKPKTNMKH | 213 |
| NQWNKPSKPKTNMK | 214 |
| NQWNKPSKPKTNM | 215 |
| NQWNKPSKPKTN | 216 |
| THNQWNKPSKPKTNMKHV | 217 |
| HNQWNKPSKPKTNMKHV | 218 |
| NQWNKPSKPKTNMKHV | 219 |
| PHGGGWGQPHGGGWGQPHGGGWGQ | 220 |
| GGWGQGGGTHSQWNKPSKPKTNMKHM | 221 |
| QWNKPSKPKTNMKHMGGGQWNKPSKPKTNMKHM | 222 |
| GGWGQGGGTH[N/S]QWNKPSKPKTN[L/M]KH[V/M](GG | 223 |
| GG) | |
| PHGGGWGQHG[G/S]SWGQPHGG[G/S]WGQ | 224 |
| QWNKPSKPKTN[L/M]KH[V/M](GGG) | 225 |
| GGGAWNKPSKPKTN | 226 |
| 4-branchMAPS-(GGG)QWNKPSKPKTN(GGG) | 227 |
| 8-branchMAPS-(GGG)KKRPKPGGWNT(GGG) | 228 |

In certain embodiments, the peptide fragment can be derived from any of those regions corresponding to residues 23-43 or 85-156 (e.g., 23-30, 86-111, 89-112, 97-107, 113-135, and 136-156) numbered according to the mouse prion sequence shown in SEQ ID NO: 2 of co-owned patent applications U.S. Ser. No. 10/917,646, filed Aug. 13, 2004, U.S. Ser. No. 11/056,950, filed Feb. 11, 2005, and International Application PCT/US2004/026363, filed Aug. 13, 2004, all entitled “Prion-Specific Peptide Reagents”, each of which are incorporated herein in its entirety.

In some embodiments, the peptide fragment is selected from any one of SEQ ID Nos. 14, 50, 51, 52, 12, 72, 68 or 115 through 219. In some embodiments, the peptide fragment is selected from any one of SEQ ID Nos. 14, 50, 51, 52, or 161 through 219. In some embodiments, the peptide fragment is selected from any one of SEQ ID Nos. 12, 72, 68 or 115 through 160. In some embodiments, the peptide fragment is selected from any one of SEQ ID Nos. 14, 50, or 68.

### D. Peptoid PCSB Reagents

In particularly preferred embodiments, the PCSB reagents are peptoids. Typically, the PCSB reagents are derived from prion protein fragments. Preferred peptoids are described below.

### Design of Peptoids

As a starting point, the peptoid PCSB reagent can be designed based on the sequences of prion protein fragments or any of the variants of such fragment described above by making replacements of amino acid residues in the sequence of the peptide fragment with N-substituted glycines, synthesis of the modified peptide using methods described in U.S. Pat. Nos. 5,811,387; 5,831,005; 5,877,278; 5,977,301; 6,075,121; 6,251,433; and 6,033,631, as well as Simon et al. (1992) Proc. Natl. Acad. Sci. USA 89: 9367, which publications are incorporated herein by reference in their entirety and testing of the modified peptide for binding to pathogenic conformers by the methods described herein. Additional replacements can be made according to the replacement scheme below until a suitable reagent is achieved.

In some embodiments, a PCSB reagent is designed by

a) providing a peptide fragment of a prion protein and replacing a first amino acid of a peptide fragment with an N-substituted glycine by the following replacement scheme:
i) Ala, Gly, Ile, Leu, Pro, and Val are replaced by N-(alkyl)glycine, N-(aralkyl)glycine, or N-(heteroarylalkyl)glycine;
ii) Asp, Asn, Cys, Gln, Glu, Met, Ser, and Thr are replaced by N-(hydroxyalkyl)glycine, N-(alkoxy)glycine, N-(aminoalkyl)glycine, or N-(guanidinoalkyl)glycine;
iii) Phe, Trp, and Tyr are replaced by N-(aralkyl)glycine, N-(heteroarylalkyl)glycine, N-(hydroxyaralkyl)glycine, or N-(alkoxyaralkyl)glycine; and
iv) Arg, His, and Lys are replaced by N-(aminoalkyl)glycine or N-(guanidinoalkyl)glycine;

b) replacing a second amino acid of the peptide fragment with an N-substituted glycine according to Step a);

c) replacing a third amino acid of the peptide fragment with an N-substituted glycine according to Step a); and

d) optionally, repeating step c) 1-27 times,

thereby, providing a designed peptoid PCSB reagent comprising 3 to 30 N-substituted glycines; and,

synthesizing the designed peptoid PCSB reagent.

The modified peptide can be tested for binding to the pathogenic conformers according to methods described herein. Additional replacements, according to the above scheme, of amino acid monomers with N-substituted glycines can be made and retested until suitable binding is obtained (i.e., PCSB reagents that interact preferentially with the pathogenic form of the prion).

Methods for making peptoids are disclosed in U.S. Pat. Nos. 5,811,387 and 5,831,005, each of which is incorporated herein by reference in its entirety, as well as methods disclosed herein.

The pathogenic conformer-specific binding reagents used in methods of the invention may have a formula of:

X^{a}-(Q)ₙ-X^{b}

wherein:

each Q is independently an amino acid or an N-substituted glycine, and -(Q)ₙ-defines a peptoid region;

X^{a} is H, (C₁-C₆)alkyl, cycloalkyl, aryl, aralkyl, heteroaryl, heteroarylalkyl, heterocycloalkyl, (C₁-C₆)acyl, amino(C₁₋₆)acyl, an amino acid, an amino protecting group, or a polypeptide of 2 to about 100 amino acids, wherein X^{a} is optionally substituted by a conjugate moiety that is optionally attached through a linker moiety;

X^{b} is H, (C₁-C₆)alkyl, aryl, aralkyl, heteroaryl, heteroarylalkyl, heterocycloalkyl, amino, alkylamino, dialkylamino, hydroxyl, (C₁-C₆)alkoxy, aryloxy, aralkoxy, a carboxy protecting group, an amino acid, or a polypeptide of 2 to about 100 amino acids, wherein X^{b} is optionally substituted by a conjugate moiety that is optionally attached through a linker moiety; and
n is 3 to about 30 (that is n is 3, 4, 5, 6, 7, 8, 9, 10, 11, 12, 13, 14, 15, 16, 17, 18, 19, 20, 21, 22, 23, 24, 25, 26, 27, 28, 29, or 30, or more);

wherein at least about 50% of the peptoid region -(Q)ₙ- includes, but is not limited to, N-substituted glycines.

In some embodiments, each Q is independently an N-substituted glycine.

In some embodiments, the PCSB reagent has a formula of X^{a}-(Q)ₙ-X^{b}, where n is about 4 to about 30, preferably about 5 to about 30, and where at least about 50% of the peptoid region -(Q)ₙ- includes, but is not limited to N-substituted glycines, provided that the peptoid region -(Q)ₙ- includes, but is not limited to at least one subregion independently selected from:

(a) -AABA-;

(b) -AABAB-

(c) -ABACC-;

(d) -AAAAA-;

(e) -ABCBA-;

(f) -AABCA-; or

(g) -ABABA-;

where A, B, and C are each different N-substituted glycines.

In some embodiments, X^{a} is (C₁-C₆)acyl or amino(C₁₋₆)acyl, each optionally substituted by a conjugate moiety that is optionally attached through a linker moiety.

In some embodiments, X^{a} is (C₁-C₆)acyl or amino(C₁₋₆)acyl, each optionally substituted by a conjugate moiety selected from a cross-linking or binding reagent each optionally attached through a linker moiety.

In some embodiments, X^{a} is (C₁-C₆)acyl or amino(C₁₋₆)acyl, each optionally substituted by a conjugate moiety selected from biotin or mercapto, where the conjugate moiety is optionally attached through a linker moiety.

In some embodiments, X^{b} is an amino acid optionally substituted by a conjugate moiety that is optionally attached through a linker moiety.

In some embodiments, X^{b} is amino, alkylamino, dialkylamino. In some embodiments, X^{b} is amino.

In some embodiments, n is about 5 to about 15; 5 to about 10; or 6.

In some embodiments, n is 4 to 10, 4 to 8, 5 to 7 or 6.

In some embodiments, X^{b} is an amino acid optionally substituted by a conjugate moiety and n is 6.

In some embodiments, the linker moiety contains a region having the formula

—{NH(CH₂)ₘC(O)}ₚ—.

In some embodiments, m is 1 to 10.

In some embodiments, m is 1 to 8.

In some embodiments, m is 5.

In some embodiments, p is 1 to 5.

In some embodiments, p is 1 to 3.

In some embodiments, p is 1 or 2.

In some embodiments, X^{b} is an amino acid optionally substituted by a conjugate moiety that is optionally attached through a linker moiety, and n is 6.

In some embodiments, X^{b} is amino, alkylamino, or dialkylamino; X^{a} is H, (C₁-C₆)alkyl, (C₁-C₆)acyl, amino(C₁₋₆)acyl, an amino acid, or an amino protecting group, wherein X^{a} is optionally substituted by a conjugate moiety that is optionally attached through a linker moiety; and n is 6.

In some embodiments, X^{b} is amino, alkylamino, or dialkylamino; X^{a} is H, (C₁-C₆)alkyl, (C₁-C₆)acyl, amino(C₁₋₆)acyl, an amino acid, or an amino protecting group, wherein X^{a} is substituted by a conjugate moiety selected from a crosslinking agent or binding agent, wherein the conjugate moiety is optionally attached through a linker moiety; and n is 6.

In some embodiments, X^{b} is amino, alkylamino, or dialkylamino; X^{a} is H, (C₁-C₆)alkyl, (C₁-C₆)acyl, amino(C₁₋₆)acyl, an amino acid, or an amino protecting group, wherein X^{a} is substituted by a conjugate moiety comprising biotin or mercapto, wherein the conjugate moiety is optionally attached through a linker moiety wherein at least a portion of the linker moiety has the formula —{NH(CH₂)ₘC(O)}ₚ—; n is 6; m is 1 to 10; and p is 1 to 5.

In some embodiments, each Q is independently an amino acid or an N-substituted glycine having the formula —(NR—CH₂—CO)— wherein each R is independently selected from (C₂-C₆)alkyl, halo(C₁-C₆)alkyl, (C₂-C₆)alkenyl, (C₂-C₆)alkynyl, (C₆-C₁₀)cycloalkyl-aryl, amino(C₁-C₆)alkyl, ammonium(C₁-C₆)alkyl, hydroxy(C₁-C₆)alkyl, (C₁-C₆)alkoxy(C₁-C₆)alkyl, carboxy, carboxy(C₂-C₆)alkyl, carbamyl, carbamyl(C₂-C₆)alkyl, guanidino, guanidino(C₁-C₆)alkyl, amidino, amidino(C₁-C₆)alkyl, thiol, (C₁-C₆)alkylthiol, alkylthioalkyl of 2-10 carbon atoms, N-containing heterocyclyl, N-containing heterocyclyl(C₁-C₆)alkyl, imidazolyl, imidazolylalkyl of 4-10 carbon atoms, piperidyl, piperidylalkyl of 5-10 carbon atoms, indolyl, indolylalkyl of 9-15 carbon atoms, naphthyl, naphthylalkyl of 11-16 carbon atoms, and aryl(C₁-C₆)alkyl; where each R moiety is optionally substituted with 1-3 substituents independently selected from halogen, hydroxy and (C₁-C₆)alkoxy.

In some embodiments, each Q is independently an amino acid or an N-substituted glycine having the formula —(NR—CH₂—CO)— wherein each R is independently selected from (C₂-C₆)alkyl, halo(C₁-C₆)alkyl, (C₂-C₆)alkenyl, (C₂-C₆)alkynyl, (C₆-C₁₀)cycloalkyl-aryl, amino(C₁-C₆)alkyl, hydroxy(C₁-C₆)alkyl, (C₁-C₆)alkoxy(C₁-C₆)alkyl, carboxy, carboxy(C₂-C₆)alkyl, carbamyl, carbamyl(C₂-C₆)alkyl, guanidino, guanidino(C₁-C₆)alkyl, thiol, (C₁-C₆)alkylthiol, alkylthioalkyl of 2-10 carbon atoms, imidazolyl, imidazolylalkyl of 4-10 carbon atoms, piperidyl, piperidylalkyl of 5-10 carbon atoms, indolyl, indolylalkyl of 9-15 carbon atoms, naphthyl, naphthylalkyl of 11-16 carbon atoms, diphenyl(C₁-C₆)alkyl or aryl(C₁-C₆)alkyl; where each R moiety is optionally substituted with 1-3 substituents independently selected from halogen, hydroxy and (C₁-C₆)alkoxy.

In some embodiments, each Q is independently an amino acid or an N-substituted glycine of the formula —(NR—CH₂—CO)— wherein each R is independently selected from (C₂-C₆)alkyl, amino(C₁-C₆)alkyl, hydroxy(C₁-C₆)alkyl, (C₁-C₆)alkoxy(C₁-C₆)alkyl, guanidino(C₁-C₆)alkyl, indolylalkyl of 9-15 carbon atoms, naphthylalkyl of 11-16 carbon atoms, diphenyl(Cᵣ C₆)alkyl or aryl(C₁-C₆)alkyl, substituted with 1-3 substituents independently selected from halogen, hydroxy or (C₁-C₆)alkoxy.

In some embodiments, each Q is independently an amino acid or is an N-substituted glycine selected from N-(4-aminobutyl)glycine, N-(1-phenylethyl)glycine, N-(2-aminoethyl)glycine, N-(2-[4-methoxyphenyl]ethyl)glycine, N-(2-methoxyethyl)glycine, N-(2-hydroxyethyl)glycine, N-((1H-indol-3-yl)methyl)glycine, or N-benzylglycine.

In some embodiments, each Q is independently an amino acid or is an N-substituted glycine selected from N-(4-aminobutyl)glycine or N-benzylglycine.

In some embodiments, each Q is independently an N-substituted glycine.

In some embodiments, the peptoid region -(Q)ₙ- includes, but is not limited to at least 3 or at least 4 N-substituted glycines which are charged at physiologically relevant pH. In some embodiments, the charge is positive. In some embodiments, the remaining N-substituted glycines of the peptoid region are neutral at physiologically relevant pH.

In some embodiments, the peptoid region -(Q)ₙ- includes, but is not limited to 2 to 6, 3 to 5, or 4 N-substituted glycines which are charged at physiologically relevant pH. In some embodiments, the charge is positive. In some embodiments, the remaining N-substituted glycines of the peptoid region are neutral at physiologically relevant pH.

In some embodiments, two N-substituted glycine residues of the peptoid region -(Q)ₙ- are positively charged at physiologically relevant pH and the remaining N-substituted glycine residues of the peptoid region are neutral at physiologically relevant pH.

In some embodiments, three N-substituted glycine residues of the peptoid region -(Q)ₙ- are positively charged at physiologically relevant pH and the remaining N-substituted glycine residues of the peptoid region are neutral at physiologically relevant pH.

In some embodiments, four N-substituted glycine residues of the peptoid region -(Q)ₙ- are positively charged at physiologically relevant pH and the remaining N-substituted glycine residues of the peptoid region are neutral at physiologically relevant pH.

In some embodiments, five N-substituted glycine residues of the peptoid region -(Q)ₙ- are positively charged at physiologically relevant pH and the remaining N-substituted glycine residues of the peptoid region are neutral at physiologically relevant pH.

In some embodiments, the peptoid region -(Q)ₙ- is polyionic at physiologically relevant pH.

In some embodiments, the peptoid region -(Q)ₙ- is polycationic at physiologically relevant pH.

In some embodiments, the peptoid region -(Q)ₙ- is polyanionic at physiologically relevant pH.

In some embodiments, the peptoid region -(Q)ₙ- has a net charge of at least 3+ at physiologically relevant pH.

In some embodiments, the peptoid region -(Q)ₙ- has a net charge of at least 4+ at physiologically relevant pH.

In some embodiments, the peptoid region -(Q)ₙ- has a net charge of 2+ to 6+ at physiologically relevant pH.

In some embodiments, the peptoid region -(Q)ₙ- has a net charge of 3+ to 5+ at physiologically relevant pH.

In some embodiments, the peptoid region -(Q)ₙ- has a net charge of 4+ at physiologically relevant pH.

In some embodiments, the peptoid region -(Q)ₙ- includes, but is not limited to at least 3 N-substituted glycines that are positively charged at physiologically relevant pH.

In some embodiments, wherein the peptoid region -(Q)ₙ- includes, but is not limited to at least 4 N-substituted glycines that are positively charged at physiologically relevant pH.

In some embodiments, the peptoid region -(Q)ₙ- includes, but is not limited to from 2 to 6 N-substituted glycines that are positively charged at physiologically relevant pH.

In some embodiments, the peptoid region -(Q)ₙ- includes, but is not limited to from 3 to 5 N-substituted glycines that are positively charged at physiologically relevant pH.

In some embodiments, the peptoid region -(Q)ₙ- includes, but is not limited to 4 N-substituted glycines that are positively charged at physiologically relevant pH.

In some embodiments, the N-substituted glycines of peptoid region -(Q)ₙ- have the formula —(NR—CH₂—CO)—, wherein R is independently selected from (C₂-C₆)alkyl, halo(C₁-C₆)alkyl, (C₂-C₆)alkenyl, (C₂-C₆)alkynyl, (C₆-C₁₀)cycloalkyl-aryl, amino(C₁-C₆)alkyl, ammonium(C₁-C₆)alkyl, hydroxy(C₁-C₆)alkyl, (C₁-C₆)alkoxy(C₁-C₆)alkyl, carboxy, carboxy(C₂-C₆)alkyl, carbamyl, carbamyl(C₂-C₆)alkyl, guanidino, guanidino(C₁-C₆)alkyl, amidino, amidino(C₁-C₆)alkyl, thiol, (C₁-C₆)alkylthiol, alkylthioalkyl of 2-10 carbon atoms, N-containing heterocyclyl, N-containing heterocyclyl(C₁-C₆)alkyl, imidazolyl, imidazolylalkyl of 4-10 carbon atoms, piperidyl, piperidylalkyl of 5-10 carbon atoms, indolyl, indolylalkyl of 9-15 carbon atoms, naphthyl, naphthylalkyl of 11-16 carbon atoms, and aryl(C₁-C₆)alkyl; where each R moiety is optionally substituted with 1-3 substituents independently selected from halogen, hydroxy and (C₁-C₆)alkoxy, and the peptoid region -(Q)ₙ- includes, but is not limited to at least 3, at least 4, 2 to 6, 3 to 5, or 4 N-substituted glycines wherein R is a moiety that is charged at physiologically relevant pH.

In some embodiments, all the N-substituted glycines of the peptoid region are contiguous.

In some embodiments, the peptoid PCSB reagent includes, but is not limited to at least one conjugate moiety.

In some embodiments, the peptoid PCSB reagent includes, but is not limited to at least one conjugate moiety attached through a linker moiety.

In preferred embodiments, the peptoid PCSB reagent includes, but is not limited to an amino-terminal region, a carboxy-terminal region, and at least one peptoid region between the amino-terminal region and the carboxy-terminal region where the peptoid region includes, but is not limited to about 3 to about 30 N-substituted glycines and optionally one or more amino acids. In some embodiments, the peptoid region includes, but is not limited to about 4 to about 30 or about 5 to about 30 N-substituted glycines. In some such embodiments, the peptoid region includes, but is not limited to about 4 to about 30, or about 5 to about 30 N-substituted glycines and a peptoid subregion selected from:

(a) -AABA-;

(b) -AABAB-

(c) -ABACC-;

(d) -AAAAA-;

(e) -ABCBA-;

(f) AABCA-; or

(g) -ABABA-;

wherein A, B, and C are each different N-substituted glycines, and each subregion sequence is read from left to right in the amino-terminal to carboxy-terminal direction.

In some embodiments, the peptoid region includes, but is not limited to about 50 to about 100%, about 75 to about 100%, or 100% N-substituted glycines.

In some embodiments, the peptoid region is about 5 to about 50, about 5 to about 30, about 5 to about 15, about 5 to about 7, or 6 subunits in length.

In some embodiments, the peptoid reagent has a total length of about 5 to about 50, about 5 to about 30, about 5 to about 15, or about 6 to about 9 subunits.

In some embodiments, at least one peptoid region is greater than about 50%, greater than about 75%, or greater than about 90% of the total length of the peptoid reagent.

In some embodiments, all the N-substituted glycines are contiguous in the peptoid region.

In some embodiments, the N-substituted glycines of the peptoid region have the formula —(NR—CH₂—CO)— wherein R is as defined herein throughout.

In some embodiments, the peptoid region is polyionic at physiologically relevant pH and has characteristics according to any of the embodiments described herein throughout for charged peptoid regions.

In other embodiments, the PCSB reagent includes a peptoid region having 3 to 15 contiguous N-substituted glycines, and wherein the peptoid region has a net charge at physiologically relevant pH. In some embodiments, the net charge is a net positive charge such as a net charge of at least 3+ or at least 4+ at physiologically relevant pH. In some embodiments, the reagent itself has a net charge of 2+ to 6+, 3+ to 5+, or 4+ at physiologically relevant pH.

In some embodiments, at least two, at least 3, or at least 4 of the contiguous N-substituted glycines of the peptoid region are charged at physiologically relevant pH. In further embodiments, at least two of the contiguous N-substituted glycines of the peptoid region comprise at least one moiety selected from primary amino, secondary amino, tertiary amino, ammonium (quaternary amino), guanidino, amidino, or N-containing heterocyclyl.

In yet further embodiments, at least two of the contiguous N-substituted glycines of the peptoid region includes, but is not limited to at least one N-substituent selected from primary amino, secondary amino, ammonium, guanidino, amidino, or N-containing heterocyclyl.

In yet further embodiments, at least two of the contiguous N-substituted glycines comprise an N-substituent which is an R group according to the definitions provided herein.

In yet further embodiments, the peptoid PCSB reagent includes, but is not limited to a peptoid region of 6 contiguous N-substituted glycines and the peptoid PCSB reagent itself has a net charge of 3+ or 4+ at physiologically relevant pH.

A “peptoid reagent” is a molecule having an amino-terminal region, a carboxy-terminal region, and at least one “peptoid region” between the amino-terminal region and the carboxy-terminal region. The amino-terminal region refers to a region on the amino-terminal side of the reagent that typically does not contain any N-substituted glycines. The amino-terminal region can be H, alkyl, substituted alkyl, acyl, an amino protecting group, an amino acid, a peptide, or the like. In some embodiments, the amino-terminal region corresponds to X^{a}. The carboxy-terminal region refers to a region on the carboxy-terminal end of the peptoid that does not contain any N-substituted glycines. The carboxy-terminal region can include H, alkyl, alkoxy, amino, alkylamino, dialkylamino, a carboxy protecting group, an amino acid, a peptide, or the like. In some embodiments, the carboxy-terminal region corresponds to X^{b}. In some embodiments, the peptoid PCSB reagent has a total length of about 5 to about 50 subunits; about 5 to about 30 subunits; about 5 to about 15 subunits; or about 6 to about 9 subunits. In some embodiments, a peptoid is a carboxy-terminal amide. The peptoid region generally refers to a portion of a PCSB reagent in which at least three of the amino acids therein are replaced by N-substituted glycines.

The “peptoid region” (also designated “-(Q)ₙ-” herein) can be identified as the region starting with and including the N-substituted glycine closest to the amino-terminus and ending with and including the N-substituted glycine closest to the carboxy-terminus. In some embodiments, the peptoid region includes, but is not limited to at least about 50%, at least about 60%, at least about 70%, at least about 80%, at least about 90%, at least about 95%, at least about 99%, or 100% N-substituted glycines. In some embodiments, the peptoid region includes, but is not limited to about 25 to about 100%; about 50 to about 100%; about 75 to about 100% N-substituted glycines. In some embodiments, the peptoid region includes, but is not limited to 100% N-substituted glycines. In some embodiments, the peptoid region is greater than about 50% (e.g., about 50-100%) of the total length of the peptoid PCSB reagent. In some embodiments, the peptoid region is greater than about 60% (e.g., about 60-100%) of the total length of the peptoid reagent. In some embodiments, the peptoid region is greater than about 75% (e.g., about 75-100%) of the total length of the peptoid reagent. In some embodiments, the peptoid region is greater than about 90% (e.g., about 90-100%) of the total length of a reagent. In some embodiments, the peptoid region is 100% of the total length of the reagent.

In some embodiments, the peptoid region contains at least 3 N-substituted glycines. In some embodiments, the peptoid region contains at least 4 N-substituted glycines. In some embodiments, the peptoid region contains at least 5 N-substituted glycines. In some embodiments, the peptoid region contains at least 6 N-substituted glycines. In some embodiments, the peptoid region contains 3 to about 30; about 5 to about 30 N-substituted glycines; and optionally one or more amino acids. In some embodiments, the peptoid region is about 5 to about 50, 5 to about 30, 5 to about 15, 5 to about 10, 5 to about 9, 5 to about 8, or 5 to about 7 subunits in length. In some embodiments, the peptoid region is about 3, 4, 5, 6, 7, 8, 9, or 10 subunits in length. In some embodiments, the peptoid region is 6 subunits in length. In some embodiments, all of the N-substituted glycines in the peptoid region are contiguous. In some embodiments, all of the subunits of the peptoid region are N-substituted glycines.

In further embodiments, the PCSB reagent includes, but is not limited to a peptoid region of 4 to 12, 4 to 10, 4 to 9, 4, to 8, 5 to 7, or 6 contiguous N-substituted glycines.

According to some embodiments, the peptoid region can be polyionic at physiologically relevant pH. By the term “polyionic” is meant that the peptoid region contains two or more residues that are charged at physiologically relevant pH. In some embodiments, the peptoid region is polycationic or polyanionic at physiologically relevant pH. In further embodiments, the peptoid region has a net charge of at least 3+ or at least 4+ at physiologically relevant pH. In yet further embodiments, the peptoid region has a net charge of 2+ to 6+, 3+ to 5+, or 4+ at physiologically relevant pH.

Non-limiting examples of N-substituted glycine residues that are charged include N-(5-aminopentyl)glycine, N-(4-aminobutyl)glycine, N-(3-aminopropyl)glycine, N-(2-aminoethyl)glycine, N-(5-guanidinopentyl)glycine, N-(4-guanidinobutyl)glycine, N-(3-guanidinopropyl)glycine, and N-(2-guanidinoethyl)glycine.

In some embodiments, the peptoid region contains at least 3 or at least 4 N-substituted glycines that are positively charged at physiologically relevant pH.

In some embodiments, the peptoid region contains from 2 to 6, 3 to 5, or 4 amino N-substituted glycines that are positively charged at physiologically relevant pH.

In some embodiments, the peptoid region contains residues having the formula —(NR—CH₂—CO)— where at least 3, at least 4, 2 to 6, 3 to 5, or 4 of the residues are charged at physiologically relevant pH.

In some embodiments, the charged residues of the peptoid region have the formula —(NR—CH₂—CO)— wherein R is independently selected from amino(C₁-C₆)alkyl, ammonium(C₁-C₆)alkyl, guanidino, guanidino(C₁-C₆)alkyl, amidino, amidino(C₁-C₆)alkyl, N-containing heterocyclyl, and N-containing heterocyclyl(C₁-C₆)alkyl, wherein each R moiety is optionally substituted with 1-3 substituents independently selected from halogen, C₁-C₃ methoxy, and C₁-C₃ alkyl. In some embodiments, R is amino(C₁-C₆)alkyl such as aminobutyl.

In some embodiments, the PCSB reagent has a net charge of at least 3+ or at least 4+ at physiologically relevant pH. In yet further embodiments, the reagent has a net charge of 2+ to 6+, 3+ to 5+, or 4+ at physiologically relevant pH.

The peptoid region of the PCSB reagent used in methods of the invention can contain at least one peptoid subregion, which refers to a sequence of contiguous N-substituted glycines of 2, 3, 4, 5, 6, 7, 8, 9, 10, 11 or 12 or more residues. In some embodiments, the peptoid region contains at least one peptoid subregion independently selected from:

(a) -AABA-;

(b) -AABAB-

(c) -ABACC-;

(d) -AAAAA-;

(e) -ABCBA-;

(f) -AABCA-; or

(g) -ABABA-.

A, B, and C each represent different N-substituted glycines. For example, each A occurring in the subregion refers to a particular N-substituted glycine, and each B occurring in the subregion refers to another particular N-substituted glycine, but A and B are different from each other. Accordingly, C is an N-substituted glycine that is different from either A or B. The subregion sequence is meant to be read from left to right in the amino to carboxy direction. In some embodiments, when A is a hydrophobic residue, then B is a hydrophilic residue, and vice versa. In some embodiments, the peptoid subregion is homogenous, i.e., contains only one type of N-substituted glycine. In some embodiments, when A is an aliphatic residue, B is a cyclic residue. In some embodiments, when B is an aliphatic residue, A is a cyclic residue. In some embodiments, both A and B are aliphatic. In some embodiments, A and B are aliphatic and C is cyclic. In some embodiments, all the N-substituted glycines are aliphatic such as for subregion -AABA-, e.g., —(N-(2-methoxyethyl)glycine)₂-N-(4-aminobutyl)glycine-(N-(2-methoxyethyl)glycine)-, where A is N-(2-methoxyethyl)glycine and B is N-(4-aminobutyl)glycine.

In some embodiments, the peptoid region contains a tripeptoid, i.e., three contiguous N-substituted glycines. Example tripeptoid peptoid subregions include —(N-(2-(4-hydroxyphenyl)ethyl)glycine)₂-N-(4-guanidinobutyl)glycine-, —N-(4-aminobutyl)glycine-(V)₂—, where V is N-benzylglycine or N-(2-methoxyethyl)glycine, —N-benzylglycine-W—N-benzylglycine-, where W is N-(4-aminobutyl)glycine or N-(2-methoxyethyl)glycine, and —N-(4-aminoethyl)glycine-(N-(2-(4-methoxyphenyl)ethyl)glycine)₂-. In some embodiments, the tripeptoid subregion contains at least one aliphatic and one cyclic residue, e.g., (A)₂-B, B₂-A, or B-A-B where A is an aliphatic residue and B is a cyclic residue.

In some embodiments, the peptoid subregion is a dipeptoid such as a N-(4-aminobutyl)glycine-(S)—N-(1-phenylethyl)glycine dipeptoid.

A PCSB reagent useful in methods of the invention includes monomers, multimers, cyclized molecules, branched molecules, linkers and the like. Multimers (i.e., dimers, trimers and the like) of any of the sequences described herein or biologically functional equivalents thereof are also contemplated. The multimer can be a homomultimer, i.e., composed of identical monomers. Alternatively, the multimer can be a heteromultimer, i.e., all the monomers comprising the multimer are not identical.

Multimers can be formed by the direct attachment of the monomers to each other or to substrate, including, for example, multiple antigenic peptides (MAPS) (e.g., symmetric MAPS), peptides attached to polymer scaffolds, e.g., a PEG scaffold and/or peptides linked in tandem with or without spacer units. Alternatively, a linker can be added to the monomers to join them to form a multimer. Non-limiting examples of multimers using linkers include, for example, tandem repeats using glycine linkers, MAPS attached via a linker to a substrate and/or linearly linked peptides attached via linkers to a scaffold. Linker moieties may involve using bifunctional spacer units (either homobifunctional or heterobifunctional) as are known to one of skill in the art.

In some embodiments, the PCSB reagent interacts with pathogenic conformers with an affinity of at least about 2 fold; 5 fold; 10 fold; 20 fold; 50 fold; 100 fold; 200 fold; 500 fold; or 1000 fold greater than that for the non-pathogenic form of the conformational disease protein. In some embodiments, the affinity is at least about 10 fold greater than that for the non-pathogenic form of the conformational disease protein. In some embodiments, the affinity is at least 100 fold greater.

The detection methods of the invention can utilize any of the PCSB reagents described herein. In some embodiments, the detection method of the present invention utilizes a PCSB reagent having a formula of:

X^{a}-(Q)ₙ-X^{b}

wherein:

each Q is independently an amino acid or an N-substituted glycine, and -(Q)ₙ-defines a peptoid region;

X^{a} is H, (C₁-C₆)alkyl, cycloalkyl, aryl, aralkyl, heteroaryl, heteroarylalkyl, heterocycloalkyl, (C₁-C₆)acyl, amino(C₁₋₆)acyl, an amino acid, an amino protecting group, or a polypeptide of 2 to about 100 amino acids, wherein X^{a} is optionally substituted by a conjugate moiety that is optionally attached through a linker moiety;

X^{b} is H, (C₁-C₆)alkyl, aryl, aralkyl, heteroaryl, heteroarylalkyl, heterocycloalkyl, amino, alkylamino, dialkylamino, hydroxyl, (C₁-C₆)alkoxy, aryloxy, aralkoxy, a carboxy protecting group, an amino acid, or a polypeptide of 2 to about 100 amino acids, wherein X^{b} is optionally substituted by a conjugate moiety that is optionally attached through a linker moiety; and n is 3 to about 30; where at least about 50% of the peptoid region -(Q)ₙ- includes, but is not limited to N-substituted glycines.

In some such embodiments, n is about 4 to about 30, preferably about 5 to about 30, and the peptoid region -(Q)ₙ- has at least one subregion independently selected from:

(a) -AABA-;

(b) -AABAB-

(c) -ABACC-;

(d) -AAAAA-;

(e) -ABCBA-;

(f) -AABCA-; or

(g) -ABABA-;

where A, B, and C are each different N-substituted glycines.

In some embodiments of the method of detection, the PCSB reagent contains an amino-terminal region, a carboxy-terminal region, and at least one peptoid region between the amino-terminal region and the carboxy-terminal region, where the peptoid region contains about 3 to about 30 N-substituted glycines and optionally one or more amino acids. In some such embodiments, the peptoid region has a peptoid subregion selected from:

(a) -AABA-;

(b) -AABAB-

(c) -ABACC-;

(d) -AAAAA-;

(e) -ABCBA-;

(f) -AABCA-; and

(g) -ABABA-;

where A, B, and C are each different N-substituted glycines.

In some embodiments of the methods of the present invention, the PCSB reagent includes, but is not limited to peptoid analog of a 3 to 30 amino acid peptide fragment of the prion protein, where the peptide fragment is SEQ ID Nos. 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 135, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, or 228 where:

(a) at least one non-proline residue of the peptide fragment is replaced by an N-substituted glycine to form the peptoid analog; or

(b) at least five amino acid residues of the peptide fragment are each replaced by an N-substituted glycine to form the peptoid analog.

In some embodiments of the above method, the replacement of any one or more amino acid residue of the peptide fragment with an N-substituted glycine corresponds to the following replacement scheme:

i) Ala, Gly, Ile, Leu, Pro, and Val are replaced by N-(alkyl)glycine, N-(aralkyl)glycine, or N-(heteroarylalkyl)glycine;

ii) Asp, Asn, Cys, Gln, Glu, Met, Ser, and Thr are replaced by N-(hydroxyalkyl)glycine, N-(alkoxy)glycine, N-(aminoalkyl)glycine, or N-(guanidinoalkyl)glycine;

iii) Phe, Trp, and Tyr are replaced by N-(aralkyl)glycine, N-(heteroarylalkyl)glycine, N-(hydroxyaralkyl)glycine, or N-(alkoxyaralkyl)glycine; and

iv) Arg, His, and Lys are replaced by N-(aminoalkyl)glycine or N-(guanidinoalkyl)glycine.

In some such embodiments, the PCSB reagent is a peptoid analog of a 5 to 30 amino acid peptide fragment of the prion protein as described above.

### Preferred Peptoid Region Sequences

Table 4 lists example peptoid regions (amino to carboxy directed) suitable for preparing PCSB reagents to be used in this invention. Table 5 provides a key to the abbreviations used in Table 1. Table 6 provides the relevant structures of each of the sequences. Preparations of the specific PCSB reagents are described hereinbelow.

TABLE 4
Representative peptoid regions for PCSB reagents

| | SEQ ID |
|---|---|
| Peptoid Region Sequence | NO: |
| Nab-Nab-Nab-Nab-Nab | 229 |
| Nab-Nab-Ngb-Nspe-Nab-Nspe | 230 |
| Nae-Nmpe-Nmpe-Nae-Nmpe-Nmpe-Nae-Nmpe-Nmpe | 231 |
| Nme-Ntrp-Nme-Nab-Nspe-Nhye-Nab-Nspe-Nhye- | 232 |
| Nme | |
| Nspe-Nab-Nspe-Nab-Nspe-Nspe-Nab-Nspe-Nab- | 233 |
| Nspe-Nspe | |
| Nbn-Nab-Nbn-Nab-Nbn-Nbn-Nab-Nbn-Nab-Nbn- | 234 |
| Nbn | |
| Nme-Nab-Nme-Nab-Nnm-Nme-Nab-Nnm-Nab-Nme- | 235 |
| Nme | |
| Nme-Nab-Nme-Nab-Nme-Nme-Nab-Nme-Nab-Nme- | 236 |
| Nme | |
| Nab-Nab-Nab-Nspe-Nab-Nspe | 237 |
| Nab-Nspe-Nab-Nab-Nspe-Nab | 238 |
| Nab-Nab-Nab-Nspe-Nab-Nspe | 239 |
| Nab-Nab-Nab-Nbn-Nab-Nbn | 240 |
| Nme-Nbn-Nme-Nbn-Nme-Nbn | 241 |

TABLE 5
Abbreviations key to Table 1.

| Peptoid Residue | |
|---|---|
| Abbreviation | Amino Acid Substitute |
| Ntyr | N-(2-(4-hydroxyphenyl)ethyl)glycine |
| Nhph | N-(4-hydroxyphenyl)glycine |
| Nspe | (S)-N-(1-phenylethyl)glycine |
| Nme | N-(2-methoxyethyl)glycine |
| Ncpm | N-(cyclopropylmethyl)glycine |
| Ntrp | N-(2-3′-indolylethyl)glycine |
| Nab | N-(4-aminobutyl)glycine |
| Nmpe | N-(2-(4-methoxyphenyl)ethyl)glycine |
| Ndmb | N-(3,5-dimethoxybenzyl)glycine |
| Nbn | N-benzylglycine |
| Nhye | N-(2-hydroxyethyl)glycine |
| Nip | N-isopropylglycine |
| Nnm | N-((8′-naphthyl)methyl)glycine |
| Ngb | N-(4-guanidinobutyl)glycine |
| Nae | N-(4-aminoethyl)glycine |

TABLE 6
Relevant structures of peptoid regions of Table 4.

| SEQ ID NO: | Structure |
|---|---|
| 229 | 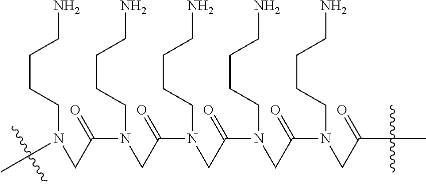 |
| 230 | 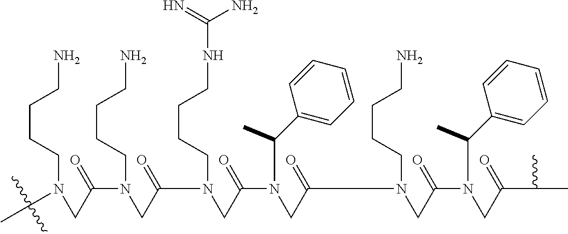 |
| 231 | 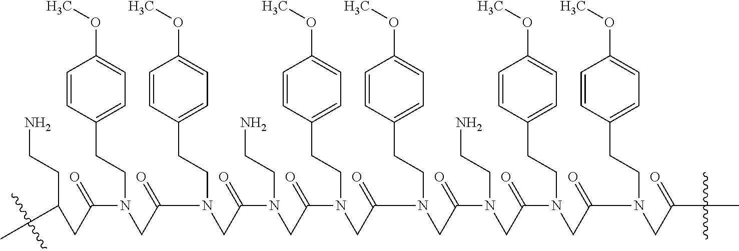 |
| 232 | 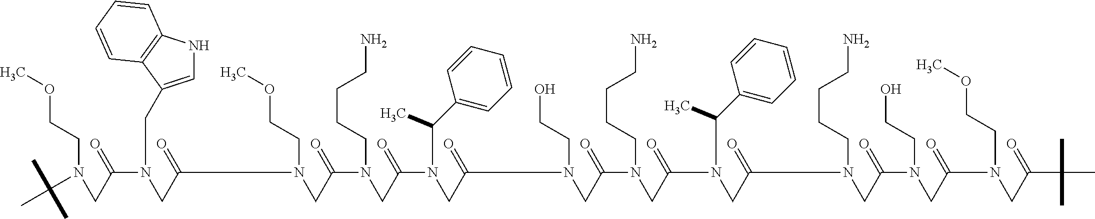 |
| 233 | 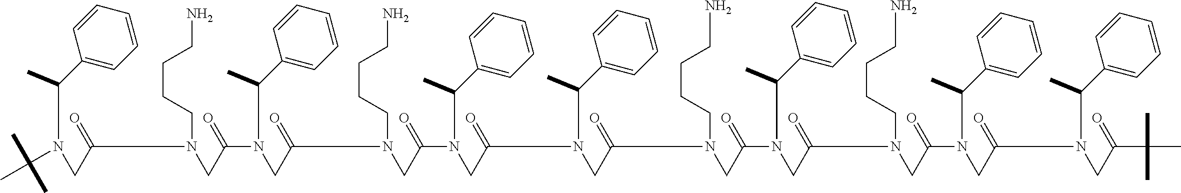 |
| 234 | 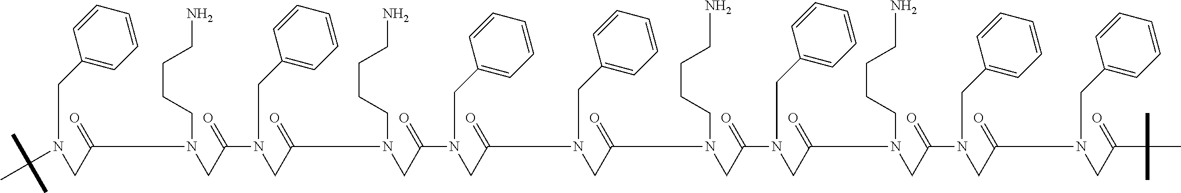 |
| 235 | 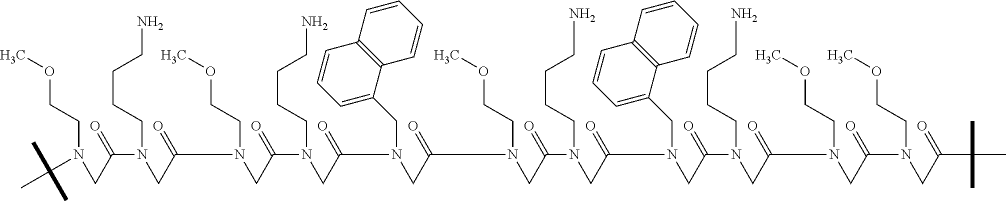 |
| 236 | 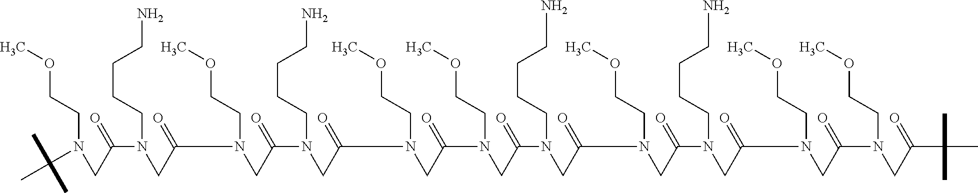 |
| 237 | 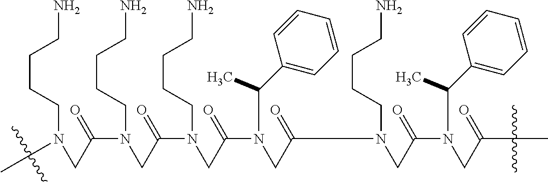 |
| 238 | 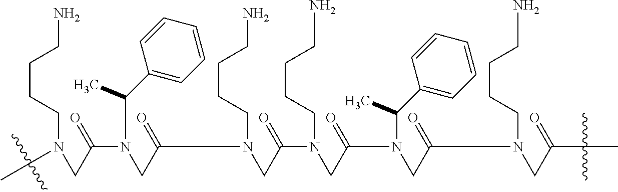 |
| 239 | 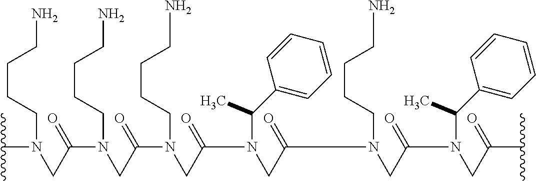 |
| 240 | 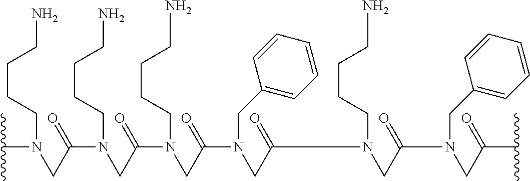 |
| 241 | 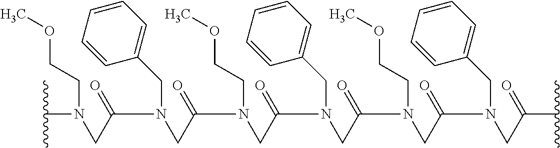 |

In some embodiments of the methods of this invention, the PCSB reagent includes, but is not limited to a sequence as described herein, for example, sequence of SEQ ID NOs: 229, 230, 231, 232, 233, 234, 235, 236, 237, 238, 239, 240, or 241. In some embodiments, the PCSB reagent includes a sequence selected from SEQ ID NO: 229, 230, 232, 233, 234, 235, 237, 238, 239, or 240. In some embodiments, the PCSB reagent includes, but is not limited to a sequence selected from SEQ ID NO: 229, 230, 235, 237, 238, 239, or 240. In some embodiments, the PCSB reagent includes, but is not limited to a sequence selected from SEQ ID NO: 230, 237, 238, 239, or 240. In some embodiments, the PCSB reagent used in the method includes, but is not limited to a sequence selected from SEQ ID NOs: 229, 236, 231, 232, 233, 234 or 235. In some embodiments, the PCSB reagent includes, but is not limited to a sequence selected from SEQ ID NOs: 230, 237, 238, 239, or 240. In some such embodiments, the PCSB reagent includes, but is not limited to SEQ ID NO: 230, 237 or 240. In some such embodiments, the PCSB reagent includes, but is not limited to SEQ ID NO: 240.

### E. Examples of Preferred Peptoids

In preferred embodiments, the pathogenic-specific conformer binding reagents to be used in methods of this invention are those which interact preferentially with pathogenic conformers of the prion protein, such as one or more of reagents depicted in I, II, VII, IX, X, XIa, XIb, XIIa, or XIIb described in Example 4.

In preferred embodiments, the PCSB reagents are peptoids derived from the human prion protein fragments PrP₁₉₋₃₀ (SEQ ID NO: 242), PrP₂₃₋₃₀ (SEQ ID NO: 243), PrP₁₀₀₋₁₁₁ (SEQ ID NO: 244), PrP₁₀₁₋₁₁₀ (SEQ ID NO: 245), PrP₁₅₄₋₁₆₅ (SEQ ID NO: 246), PrP₂₂₆₋₂₃₇ (SEQ ID NO: 247) peptides, SEQ ID NO:14, SEQ ID NO:50, or SEQ ID NO: 68.

In a particularly preferred embodiment, the PCSB reagent comprises the structure of

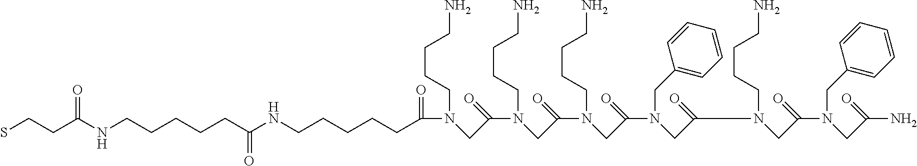

F. Identifying PCSB Reagents to be Used in Methods of this Invention

The PCSB reagents to be used in methods of this invention interact preferentially with pathogenic conformers of the prion protein. This property can be can be tested using any known binding assay, for example standard immunoassays such as ELISAs, Western blots and the like; labeled peptides; ELISA-like assays; and/or cell-based assays, in particular those assays described in the below section regarding “Detection of Pathogenic Conformers by Binding of Pathogenic Conformer to PCSB Reagent”.

One convenient method of testing the specificity of the PCSB reagents used in methods of the present invention is to select a sample containing both pathogenic and non-pathogenic conformers. Typically such samples include tissue from diseased animals. PCSB reagents as described herein that are known to bind specifically to pathogenic forms are attached to a solid support (by methods well-known in the art and as further described below) and used to separate (“pull down”) pathogenic conformer from the other sample components and obtain a quantitative value directly related to the number of reagent-protein binding interactions on the solid support. This result can be compared to that of a PCSB reagent with unknown binding specificity to determine whether such reagent can interact preferentially with pathogenic conformers.

These assays may utilize the fact that pathogenic conformers are generally resistant to certain proteases, such as proteinase K. The same proteases are able to degrade non-pathogenic conformers of conformational disease proteins. Therefore, when using a protease, the sample can be separated into two equal volumes. Protease can be added to the second sample and the same test performed. Because the protease in the second sample will degrade any non-pathogenic conformers, any reagent-protein binding interactions in the second sample can be attributed to pathogenic conformers.

### IV. DETECTION OF PATHOGENIC CONFORMERS BY BINDING OF PATHOGENIC CONFORMER TO PCSB REAGENT

The described PCSB reagents can be used in a variety of assays to screen samples (e.g., biological samples such as blood, brain, spinal cord, CSF or organ samples), for example, to detect the presence or absence of pathogenic forms of conformational disease proteins in these samples. Unlike many current diagnostic reagents, the PCSB reagents described herein will allow for detection in virtually any type of biological or non-biological sample, including blood sample, blood products, CSF, or biopsy samples. The detection methods can be used, for example, in methods for diagnosing a conformational protein disease and any other situation where knowledge of the presence or absence of the pathogenic conformer is important.

### Use of Pathogenic Conformer-Specific Binding Reagents as Either Capture or Detection Reagents

The PCSB reagents to be used in methods of the invention are typically derived from prion protein fragments and also interact preferentially with pathogenic conformers of the prion protein. It is expected that at least some of these PCSB reagents will interact preferentially to the same degree with both pathogenic prions and other pathogenic conformers. For samples expected to contain pathogenic conformers of more than one conformational disease protein or where it is critical for purposes of the method to determine which type of pathogenic conformer is present, pathogenic conformer-specific binding reagents should be used for detection in combination with CDPSB reagents which have different binding specificities and/or affinities for different types of conformational disease proteins. For example, if the pathogenic conformer-specific binding reagent is used as a capture reagent, a conformational disease protein-specific binding reagent should be used as a detection reagent or vice versa. If, however, the particular sample to be assayed is expected to only contain a single type of pathogenic conformer or if it is not critical for the purposes of the method to determine which pathogenic conformer is present, then the PCSB reagent can be used as both a capture and detection reagent.

Some PCSB reagents used in methods of the invention will interact preferentially to different degrees with pathogenic prions as compared to other pathogenic conformers such that the detection assay can be conducted in a manner that permits detection of only the non-prion pathogenic conformer. In this case, the PCSB reagent can be used as both a capture and detection reagent.

### Methods Using Pathogenic Conformer-Specific Binding Reagents as Capture Agents

In preferred embodiments, the invention provides methods for detecting the presence of a non-prion pathogenic conformer in a sample by contacting a sample suspected of containing a non-prion pathogenic conformer with a pathogenic conformer-specific binding reagent under conditions that allow binding of the reagent to the pathogenic conformer, if present; and detecting the presence the pathogenic conformer, if any, in the sample by its binding to the reagent; where the pathogenic conformer-specific binding reagent is derived from a prion protein fragment and interacts preferentially with pathogenic conformers of the prion protein.

For use in methods of the invention, the sample can be anything known to, or suspected of, containing a non-prion pathogenic conformer. The sample can be a biological sample (that is, a sample prepared from a living or once-living organism) or a non-biological sample. Suitable biological samples include, but are not limited to organs, whole blood, blood fractions, blood components, plasma, platelets, serum, cerebrospinal fluid (CSF), brain tissue, nervous system tissue, muscle tissue, bone marrow, urine, tears, non-nervous system tissue, organs, and/or biopsies or necropsies. Preferred biological samples include plasma and CSF.

The sample is contacted with one or more PCSB reagents described herein under conditions that allow the binding of the PCSB reagent(s) to the pathogenic conformer if it is present in the sample. It is well within the competence of one of ordinary skill in the art to determine the particular conditions based on the disclosure herein. Typically, the sample and the PCSB reagent(s) are incubated together in a suitable buffer at about neutral pH (e.g., a TBS buffer at pH 7.5) at a suitable temperature (e.g., about 4° C.), for a suitable time period (e.g., about 1 hour to overnight) to allow the binding to occur.

In these embodiments of the method, the pathogenic conformer-specific binding reagent is a capture reagent and the presence of pathogenic conformer in the sample is detected by its binding to the pathogenic conformer-specific binding reagent. After capture, the presence of the pathogenic conformer may be detected by the very same pathogenic conformer-specific binding reagent serving simultaneously as a capture and detection reagent. Alternatively, there can be a distinct detection reagent, which can be either a different pathogenic conformer-specific binding reagent or, preferably, one or more conformational disease protein-specific binding reagents. In preferred embodiments, after the capture step, the unbound sample is removed, the pathogenic conformer is dissociated from the complex it forms with the capture reagent and denatured for detection. The capture reagent is preferably coupled to a solid support.

### Methods Using Pathogenic Conformer-Specific Binding Reagents as Detection Agents

In other embodiments, the invention provides methods for detecting the presence of a non-prion pathogenic conformer in a sample by contacting a sample suspected of containing a non-prion pathogenic conformer with a conformational disease protein-specific binding reagent which binds to both the pathogenic and non-pathogenic forms of the conformational disease protein under conditions that allow the binding of the CDPSB reagent to the conformational disease protein, if present, to form a first complex; contacting the first complex with a PCSB reagent under conditions that allow binding, and detecting the presence the pathogenic conformer, if any, in the sample by its binding to the PCSB reagent, where the PCSB reagent is derived from a prion protein fragment and interacts preferentially with pathogenic prion protein.

### A. Reagents to Capture Pathogenic Conformers

In preferred embodiments, the capture reagent is a pathogenic conformer-specific binding reagent which is derived from a prion protein fragment and preferentially interacts with a pathogenic conformer of a prion protein. In other embodiments, the capture reagent is a conformational disease protein-specific binding reagent which binds to both the pathogenic and non-pathogenic forms of the conformational disease protein.

Capture reagents are contacted with samples under conditions that allow any non-prion pathogenic conformers in the sample to bind to the reagent and form a complex. Such binding conditions are readily determined by one of ordinary skill in the art and are further described herein. Typically, the method is carried out in the wells of a microtiter plate or in small volume plastic tubes, but any convenient container will be suitable. The sample is generally a liquid sample or suspension and may be added to the reaction container before or after the capture reagent.

The capture reagent is preferably coupled to a solid support, which is described in further detail in the following section. In some embodiments, the solid support is attached prior to application of the sample. A solid support (e.g., magnetic beads) is first reacted with a capture reagent as described herein such that the capture reagent is sufficiently immobilized to the support. The solid support with attached capture reagent is then contacted with a sample suspected of containing pathogenic conformers under conditions that allow the capture reagent to bind to pathogenic conformers.

Alternatively, the capture reagent may be first contacted with the sample suspected of containing non-prion pathogenic conformers before being attached to the solid support, followed by attachment of the capture reagent to the solid support (for example, the reagent can be biotinylated and the solid support comprise avidin or streptavidin).

In certain embodiments, after a complex between the capture reagent and pathogenic conformer is established, unbound sample material (that is, any components of the sample that have not bound to the capture reagent, including any unbound pathogenic conformers) can be removed. For example, if the capture reagent is coupled to a solid support, unbound materials can be reduced by separating the solid support from the reaction solution (containing the unbound sample materials) for example, by centrifugation, precipitation, filtration, magnetic force, etc. The solid support with the complex may optionally be subjected to one or more washing steps to remove any residual sample materials before carrying out the next steps of the method.

In some embodiments, following the removal of unbound sample materials and any optional washes, the bound pathogenic conformers are dissociated from the complex and detected using any known detection method. Alternatively, the bound pathogenic conformers in the complex are detected without dissociation from the capture reagent.

### B. Dissociation and Denaturation of Pathogenic Conformer

After being bound to the capture reagent to form a complex, the pathogenic conformer may be treated to facilitate detection of the pathogenic conformer.

In some embodiments, the unbound material is removed and the pathogenic conformer is then dissociated from the complex. “Dissociation” refers to the physical separation of the pathogenic conformer from the capture reagent such that the pathogenic conformer can be detected separately from capture reagent. Dissociation of the pathogenic conformer from the complex can be accomplished, for example using low concentration (e.g., 0.4 to 1.0 M) of guanidinium hydrochloride or guanidinium isothiocyanate.

When the CDPSB reagent used in the method is only capable of detecting denatured protein, the dissociated pathogenic conformer is also denatured. “Denaturation” refers to disrupting the native conformation of a polypeptide. Denaturation without dissociation from the reagent can be accomplished, for example, if the reagent contains an activatable reactive group (e.g., a photoreactive group) that covalently links the reagent and the pathogenic conformer.

In preferred embodiments, the pathogenic conformer is simultaneously dissociated and denatured.

Pathogenic conformers may be simultaneously dissociated and denatured using high concentrations of salt or chaotropic agent, e.g., between about 3M to about 6M of a guanidinium salt such as guanidinium thiocyanate (GdnSCN), or guanidinium HCl (GdnHCl). Preferably, the chaotropic agent is removed or diluted before detection is carried out because they may interfere with binding of the detection reagent.

In other embodiments, the pathogenic conformer is simultaneously dissociated from the complex with the capture reagent and denatured by altering pH, e.g., by either raising the pH to 12 or above (“high pH”) or lowering the pH to 2 or below (“low pH”). Exposure of the complex to high pH is preferred. A pH of between 12.0 and 13.0 is generally sufficient; preferably, a pH of between 12.5 and 13.0 is used; more preferably, a pH of 12.7 to 12.9; most preferably a pH of 12.9. Alternatively, exposure of the complex to a low pH can be used to dissociate and denature the pathogenic prion protein from the reagent. For this alternative, a pH of between 1.0 and 2.0 is sufficient. In some embodiments, the pathogenic conformer is treated with pH 12.5-13.2 for a suitable amount of time, e.g., 90 C for 10 minutes.

Exposure of the first complex to either a high pH or a low pH is generally carried out for only a short time e.g. 60 minutes, preferably for no more than 15 minutes, more preferably for no more than 10 minutes. In some embodiments, the exposure is carried out above room temperature, for example, at about 60° C., 70° C., 80° C., or 90° C. After exposure for sufficient time to dissociate the pathogenic conformer, the pH can be readily readjusted to neutral (that is, pH of between about 7.0 and 7.5) by addition of either an acidic reagent (if high pH dissociation conditions are used) or a basic reagent (if low pH dissociation conditions are used). One of ordinary skill in the art can readily determine appropriate protocols and examples are described herein.

In general, to effect a high pH dissociation condition, addition of NaOH to a concentration of about 0.05 N to about 0.2 N is sufficient. Preferably, NaOH is added to a concentration of between about 0.05 N to about 0.15 N; more preferably, about 0.1 N NaOH is used. Once the dissociation is accomplished, the pH can be readjusted to neutral (that is, between about 7.0 and 7.5) by addition of suitable amounts of an acidic solution, e.g., phosphoric acid, sodium phosphate monobasic.

In general, to effect a low pH dissociation condition, addition of H₃PO₄ to a concentration of about 0.2 M to about 0.7 M is sufficient. Preferably, H₃PO₄ is added to a concentration of between 0.3 M and 0.6 M; more preferably, 0.5 M H₃PO₄ is used. Once the dissociation is accomplished, the pH can be readjusted to neutral (that is, between about 7.0 and 7.5) by addition of suitable amounts of a basic solution, e.g., NaOH or KOH.

If desirable, dissociation of the pathogenic conformer from the complex can also be accomplished without denaturing the protein, for example using low concentration (e.g., 0.4 to 1.0 M) of guanidinium hydrochloride or guanidinium isothiocyanate. See, WO2006076497 (International Application PCT/US2006/001090) for additional conditions for dissociating the pathogenic conformer from the complex without denaturing the protein. Alternatively, the captured pathogenic conformers can be also denatured without dissociation from the reagent if, for example, the reagent is modified to contain an activatable reactive group (e.g., a photoreactive group) that can be used to covalently link the reagent and the pathogenic conformer.

After dissociation, the pathogenic conformer is then separated from the capture reagent. This separation can be accomplished in similar fashion to the removal of the unbound sample materials described above except that the portion containing the unbound materials (now the dissociated pathogenic conformer) is retained and the portion containing the capture reagent is discarded.

### C. Detection of Captured Pathogenic Conformer

Detection of pathogenic conformers may be accomplished using a conformational disease protein-specific binding reagent. In preferred embodiments, the CDPSB reagent is an antibody (monoclonal or polyclonal) that recognizes an epitope on the conformational disease protein.

Detection of the captured pathogenic conformers in the sample may also be accomplished by using a PCSB reagent. Such a reagent may be used in embodiments where the capture reagent is either a same or different pathogenic conformer-specific binding reagent or a conformational disease protein-specific binding agent.

When the method utilizes a first pathogenic conformer-specific binding reagent and a second pathogenic conformer-specific binding reagent, the first and second reagents can be the same or different. By “the same” is meant that the first and second reagents differ only in the inclusion of a detectable label in the second reagent. The first and second reagents are “different,” for example, if they have a different structure or are derived from fragments from a different region of a prion protein.

### General Detection Methods

Any suitable means of detection can then be used to identify binding between the capture reagent and pathogenic conformers.

Analytical methods suitable for use to detect binding include methods such as UV/Visible spectroscopy, FTIR, nuclear magnetic resonance spectroscopy, Raman spectroscopy, mass spectrometry, HPLC, capillary electrophoresis, surface plasmon resonance spectroscopy, Micro-Electro-Mechanical Systems (MEMS), or any other method known in the art.

Binding may also be detected through the use of labeled reagents or antibodies, often in the form of an ELISA. Detectable labels suitable for use in the invention include any molecule capable of detection, including, but not limited to, radioactive isotopes, fluorescers, chemiluminescers, chromophores, fluorescent semiconductor nanocrystals, enzymes, enzyme substrates, enzyme cofactors, enzyme inhibitors, chromophores, dyes, metal ions, metal sols, ligands (e.g., biotin, strepavidin or haptens) and the like. Additional labels include, but are not limited to, those that use fluorescence, including those substances or portions thereof that are capable of exhibiting fluorescence in the detectable range. Particular examples of labels that may be used in the invention include, but are not limited to, horseradish peroxidase (HRP), fluorescein, FITC, rhodamine, dansyl, umbelliferone, dimethyl acridinium ester (DMAE), Texas red, luminol, NADPH and β-galactosidase. Additionally, the detectable label may include an oligonucleotide tag, which can be detected by a method of nucleic acid detection including, e.g., polymerase chain reaction (PCR), transcription-mediated amplification (TMA), branched DNA (b-DNA), nucleic acid sequence-based amplification (NASBA), and the like. Preferred detectable labels include enzymes, especially alkaline phosphatase (AP), horseradish peroxidase (HRP), and fluorescent compounds. As is well known in the art, the enzymes are utilized in combination with a detectable substrate, e.g., a chromogenic substrate or a fluorogenic substrate, to generate a detectable signal.

In addition to the use of labeled detection reagents (described above), immunoprecipitation may be used to separate out reagents that are bound to the pathogenic conformer. Preferably, the immunoprecipitation is facilitated by the addition of a precipitating enhancing agent. A precipitation-enhancing agent includes moieties that can enhance or increase the precipitation of the reagents that are bound to proteins. Such precipitation enhancing agents include polyethylene glycol (PEG), protein G, protein A and the like. Where protein G or protein A are used as precipitation enhancing agents, the protein can optionally be attached to a bead, preferably a magnetic bead. Precipitation can be further enhanced by use of centrifugation or with the use of magnetic force. Use of such precipitating enhancing agents is known in the art.

Western blots, for example, typically employ a tagged primary antibody that detects denatured protein from an SDS-PAGE gel, on samples obtained from a “pull-down” assay (as described herein), that has been electroblotted onto nitrocellulose or PVDF. The primary antibody is then detected (and/or amplified) with a probe for the tag (e.g., streptavidin-conjugated alkaline phosphatase, horseradish peroxidase, ECL reagent, and/or amplifiable oligonucleotides). Binding can also be evaluated using detection reagents such as a peptide with an affinity tag (e.g., biotin) that is labeled and amplified with a probe for the affinity tag (e.g., streptavidin-conjugated alkaline phosphatase, horseradish peroxidase, ECL reagent, or amplifiable oligonucleotides).

Cell based assays can also be employed, for example, where the pathogenic conformer is detected directly on individual cells (e.g., using a fluorescently labeled reagent that enables fluorescence based cell sorting, counting, or detection of the specifically labeled cells).

Assays that amplify the signals from the detection reagent are also known. Examples of which are assays that utilize biotin and avidin, and enzyme-labeled and mediated immunoassays, such as ELISA assays. Further examples include the use of branched DNA for signal amplification (see, e.g., U.S. Pat. Nos. 5,681,697; 5,424,413; 5,451,503; 5,4547,025; and 6,235,483); applying target amplification techniques like PCR, rolling circle amplification, Third Wave's invader (Arruda et al. 2002 Expert. Rev. Mol. Diagn. 2:487; U.S. Pat. Nos. 6,090,606, 5,843,669, 5,985,557, 6,090,543, 5,846,717), NASBA, TMA etc. (U.S. Pat. No. 6,511,809; EP 0544212A1); and/or immuno-PCR techniques (see, e.g., U.S. Pat. No. 5,665,539; International Publications WO 98/23962; WO 00/75663; and WO 01/31056).

In addition, microtitre plate procedures similar to sandwich ELISA may be used, for example, a pathogenic conformer-specific binding reagent or a conformational disease protein-specific binding reagent as described herein is used to immobilize protein(s) on a solid support (e.g., well of a microtiter plate, bead, etc.) and an additional detection reagent which could include, but is not limited to, another pathogenic conformer-specific binding reagent or a conformational disease protein-specific binding reagent with an affinity and/or detection label such as a conjugated alkaline phosphatase, horseradish peroxidase, ECL reagent, or amplifiable oligonucleotides is used to detect the pathogenic conformer.

### Preferred Methods for Detecting Dissociated Captured Pathogenic Conformer

If the capture reagent and bound pathogenic conformer are dissociated prior to detection, the dissociated pathogenic conformers can be detected in an ELISA type assay, either as a direct ELISA or an antibody Sandwich ELISA type assay, which are described more fully below. Although the term “ELISA” is used to describe the detection with antibodies, the assay is not limited to ones in which the antibodies are “enzyme-linked.” The detection antibodies can be labeled with any of the detectable labels described herein and well-known in the immunoassay art. ELISAs as described in Lau et al. PNAS USA 104(28): 11551-11556 (2007) can be performed to quantify the amount of pathogenic conformer dissociated from the capture reagent.

The dissociated pathogenic conformer can be prepared for a standard ELISA by passively coating it onto the surface of a solid support. Methods for such passive coating are well known and typically are carried out in 100 mM NaHCO₃ at pH 8 for several hours at about 37° C. or overnight at 4° C. Other coating buffers are well-known (e.g, 50 mM carbonate pH 9.6, 10 mM Tris pH 8, or 10 mM PBS pH 7.2) The solid support can be any of the solid supports described herein or well-known in the art but preferably the solid support is a microtiter plate, e.g., a 96-well polystyrene plate. Where the dissociation has been carried out using a high concentration of chaotropic agent, the concentration of the chaotropic agent will be reduced by dilution by at least about 2-fold prior to coating on the solid support. Where the dissociation has been carried out using a high or low pH, followed by neutralization, the dissociated pathogenic conformer can be used for coating without any further dilution. The plate(s) can be washed to remove unbound material.

If a standard ELISA is to be performed, then a detectably labeled binding molecule, such as a conformational disease protein-specific binding reagent or a pathogenic conformer-specific binding reagent (either the same one used for capture or a different one) is added. This detectably labeled binding molecule is allowed to react with any captured pathogenic conformer, the plate washed and the presence of the labeled molecule detected using methods well known in the art. The detection molecule need not be specific for the pathogenic form but can bind to both forms, as long as the capture reagent is specific for the pathogenic form. In preferred embodiments, the detectably labeled binding molecule is an antibody. Such antibodies include ones that are well known as well as antibodies that are generated by well known methods which are either specific for the pathogenic conformer or both the pathogenic and non-pathogenic forms of a conformational disease protein.

In an alternative embodiment, the dissociated pathogenic conformers are detected using an antibody sandwich type ELISA. In this embodiment, the dissociated pathogenic conformer is “recaptured” on a solid support having a first antibody specific for the pathogenic conformer or the conformational disease protein. The solid support with the recaptured pathogenic conformer is optionally washed to remove any unbound materials, and then contacted with a second antibody specific for the conformational disease protein or pathogenic conformer under conditions that allow the second antibody to bind to the recaptured pathogenic conformer.

The first and second antibodies will typically be different antibodies and will preferably recognize different epitopes on the conformational disease protein. For example, the first antibody will recognize an epitope at the N-terminal end of the conformational disease protein and the second antibody will recognize an epitope at other than the N-terminal, or vice versa. Other combinations of first and second antibody can be readily selected. In this embodiment, the second antibody, but not the first antibody, will be detectably labeled.

When the dissociation of the pathogenic conformer from the reagent is carried out using a chaotropic agent, the chaotropic agent should be removed or diluted by at least 15-fold prior to carrying out the detection assay. When the dissociation is effected using a high or low pH and neutralization, the dissociated pathogenic conformer can be used without further dilution. When the dissociated pathogenic conformer is denatured prior to carrying out the detection, the first and second antibodies will both bind to the denatured conformer.

### Preferred Methods for Detecting Undissociated Captured Pathogenic Conformer

In other exemplary assays, the capture reagent and bound pathogenic conformer are not dissociated prior to detection. For example, a solid support (e.g., the wells of a microtiter plate) is linked to a pathogenic conformer-specific binding reagent. A sample containing or suspected of containing non-prion pathogenic conformer is then added to the solid support. After a period of incubation sufficient to allow any pathogenic conformers to bind to the reagent, the solid support can be washed to remove unbound moieties and a detectably labeled secondary binding molecule as described above, such as a conformational disease protein-specific binding reagent or a second same or different pathogenic conformer-specific binding reagent, is added. Alternatively, a conformational disease protein-specific binding is coupled to a solid support (e.g., coated onto the wells of a microtiter plate) and detection can be accomplished using a pathogenic conformer-specific detection reagent.

### Preferred Methods for Detection of Pathogenic Alzheimer's Disease Conformer

In preferred embodiments of the methods of the invention when an pathogenic Alzheimer's disease conformer is being detected, a sandwich ELISA is used. Following capture of the pathogenic Alzheimer's disease conformer from a sample using a PCSB reagent and removal of unbound sample, the captured pathogenic Alzheimer's disease conformer is typically dissociated and denatured for example, by incubation with guanidine thiocyanate or a high pH dissociation condition. In preferred embodiments where the pathogenic Alzheimer's disease conformer is Aβ, Aβ is dissociated from the complex with the PCSB reagent and denatured with about 0.05N NaOH or about 0.1N NaOH, at about 90° C. or about 80° C. In particularly preferred embodiments, Aβ is dissociated and denatured at about 0.1 N NaOH at about 80° C. for about 30 minutes.

To recapture the pathogenic Alzheimer's disease conformer, a solid support can be coated with an antibody specific for Alzheimer's disease protein. This recaptured pathogenic Alzheimer's disease conformer can be then be detected using another antibody specific for Alzheimer's disease proteins which is detectably labeled. Particularly preferred antibodies include 11A50-B10 (Covance), a antibody specific for C-terminus of Aβ40; 12F4 (Covance), a antibody specific for C-terminus of Aβ42; 4G8, specific for Aβ amino acids 17-24; 20.1, specific for Aβ amino acids 1-10; and 6E10, specific for Aβ amino acids 3-8. In a preferred embodiment, 20.1 is the capture antibody and 12F4 or 11A50-B10 are used as detection antibodies.

### D. Solid Supports Used in Assays

In certain embodiments, the PCSB reagent or CDPSB reagent are provided on a solid support. The PCSB reagent or CDPSB reagent can be provided on a solid support prior to contacting the sample or the reagent can be adapted for binding to the solid support after contacting the sample and binding to any pathogenic conformer therein (e.g., by using a biotinylated reagent and a solid support comprising an avidin or streptavidin).

A solid support, for purposes of the invention, can be any material that is an insoluble matrix and can have a rigid or semi-rigid surface to which a molecule of interest (e.g., reagents of the invention, conformational disease proteins, antibodies, etc) can be linked or attached. Exemplary solid supports include, but are not limited to, substrates such as nitrocellulose, polyvinylchloride; polypropylene, polystyrene, latex, polycarbonate, nylon, dextran, chitin, sand, silica, pumice, agarose, cellulose, glass, metal, polyacrylamide, silicon, rubber, polysaccharides, polyvinyl fluoride, diazotized paper, activated beads, magnetically responsive beads, and any materials commonly used for solid phase synthesis, affinity separations, purifications, hybridization reactions, immunoassays and other such applications. The support can be particulate or can be in the form of a continuous surface and includes membranes, mesh, plates, pellets, slides, disks, capillaries, hollow fibers, needles, pins, chips, solid fibers, gels (e.g. silica gels) and beads, (e.g., pore-glass beads, silica gels, polystyrene beads optionally cross-linked with divinylbenzene, grafted co-poly beads, polyacrylamide beads, latex beads, dimethylacrylamide beads optionally crosslinked with N—N′-bis-acryloylethylenediamine, iron oxide magnetic beads, and glass particles coated with a hydrophobic polymer.

PCSB reagents or CDPSB reagents as described herein can be readily coupled to the solid support using standard techniques which attach the PCSB reagent or CDPSB reagent, for example covalently, by absorption, coupling or through the use of binding pairs.

Immobilization to the support may be enhanced by first coupling the PCSB reagent or CDPSB reagent to a protein (e.g., when the protein has better solid phase-binding properties). Suitable coupling proteins include, but are not limited to, macromolecules such as serum albumins including bovine serum albumin (BSA), keyhole limpet hemocyanin, immunoglobulin molecules, thyroglobuline, ovalbumin, and other proteins well known to those skilled in the art. Other reagents that can be used to bind molecules to the support include polysaccharides, polylactic acids, polyglycolic acids, polymeric amino acids, amino acid copolymers, and the like. Such molecules and methods of coupling these molecules to proteins, are well known to those of ordinary skill in the art. See, e.g., Brinkley, M. A., (1992) Bioconjugate Chem., 3:2-13; Hashida et al. (1984) J. Appl. Biochem., 6:56-63; and Anjaneyulu and Staros (1987) International J. of Peptide and Protein Res. 30:117-124.

If desired, the PCSB reagents or CDPSB reagents to be added to the solid support can readily be functionalized to create styrene or acrylate moieties, thus enabling the incorporation of the molecules into polystyrene, polyacrylate or other polymers such as polyimide, polyacrylamide, polyethylene, polyvinyl, polydiacetylene, polyphenylene-vinylene, polypeptide, polysaccharide, polysulfone, polypyrrole, polyimidazole, polythiophene, polyether, epoxies, silica glass, silica gel, siloxane, polyphosphate, hydrogel, agarose, cellulose and the like. In preferred embodiments, the solid support is a magnetic bead, more preferably a polystyrene/iron oxide bead.

The PCSB reagents or CDPSB reagents can be attached to the solid support through the interaction of a binding pair of molecules. Such binding pairs are well known and examples are described elsewhere herein. One member of the binding pair is coupled by techniques described above to the solid support and the other member of the binding pair is attached to the reagent (before, during, or after synthesis). The PCSB reagent or CDPSB reagent thus modified can be contacted with the sample and interaction with the pathogenic conformer, if present, can occur in solution, after which the solid support can be contacted with the reagent (or reagent-proteincomplex). Preferred binding pairs for this embodiment include biotin and avidin, and biotin and streptavidin. In addition to biotin-avidin and biotin-streptavidin, other suitable binding pairs for this embodiment include, for example, antigen-antibody, hapten-antibody, mimetope-antibody, receptor-hormone, receptor-ligand, agonist-antagonist, lectin-carbohydrate, Protein A-antibody Fc. Such binding pairs are well known (see, e.g., U.S. Pat. Nos. 6,551,843 and 6,586,193) and one of ordinary skill in the art would be competent to select suitable binding pairs and adapt them for use with the present invention. When the capture reagent is adapted for attachment to the support as described above, the sample can be contacted with the capture reagent before or after the capture reagent is attached to the support.

Alternatively, the PCSB reagents or CDPSB reagents can be covalently attached to the solid support using conjugation chemistries that are well known in the art. For example, thiol containing PCSB or CDPSB reagents can be directly attached to solid supports, e.g., carboxylated magnetic beads, using standard methods known in the art (See, e.g., Chrisey, L. A., Lee, G. U. and O'Ferrall, C. E. (1996). Covalent attachment of synthetic DNA to self-assembled monolayer films. Nucleic Acids Research 24(15), 3031-3039; Kitagawa, T., Shimozono, T., Aikawa, T., Yoshida, T. and Nishimura, H. (1980). Preparation and characterization of hetero-bifunctional cross-linking reagents for protein modifications. Chem. Pharm. Bull. 29(4), 1130-1135). Carboxylated magnetic beads are first coupled to a heterobifunctional cross-linker that contains a maleimide functionality (BMPH from Pierce Biotechnology Inc.) using carbodiimide chemistry. The thiolated PCSB or CDPSB reagent is then covalently coupled to the maleimide functionality of the BMPH coated beads. When used in the embodiments of the detection methods of the invention, the solid support aids in the separation of the complex comprising the reagent and the pathogenic conformer from the unbound sample. Particularly convenient magnetic beads for thiol coupling are Dynabeads™ M-270 Carboxylic Acid from Dynal. The PCSB or CDPSB reagent may also comprise a linker, for example, one or more aminohexanoic acid moieties.

### E. Preferred Detection Methods

Preferred embodiments are described below.

In preferred embodiments, the methods of the invention capture and detect the non-prion pathogenic conformer using a PCSB reagent derived from a prion protein fragment, including a peptoid reagent as described herein, which interacts preferentially with a pathogenic prion protein, said method comprising contacting a sample suspected of containing the non-prion pathogenic conformer with a PSCB reagent under conditions that allow binding of the PCSB reagent to the non-prion pathogenic conformer, if present, to form a complex; and detecting the non-prion pathogenic conformer, if any, in the sample by its binding to the PCSB reagent. Binding of the non-prion pathogenic conformer can be detected, for example, by dissociating the complex and detecting non-prion pathogenic conformer with a CDPSB reagent.

In one embodiment, the non-prion pathogenic conformer to be captured is a pathogenic conformer associated with Alzheimer's disease, such as Aβ40, Aβ42, or tau. In such a case, the sample is preferably plasma or cerebrospinal fluid. The PCSB reagent is preferably derived from PrP19-30 (SEQ ID NO: 242), PrP23-30 (SEQ ID NO: 243), PrP100-111 (SEQ ID NO: 244), PrP101-110 (SEQ ID NO: 245), PrP154-165 (SEQ ID NO: 246), PrP226-237 (SEQ ID NO: 247), SEQ ID NO:14, SEQ ID NO: 50, SEQ ID NO: 68, and includes peptoid reagents such as

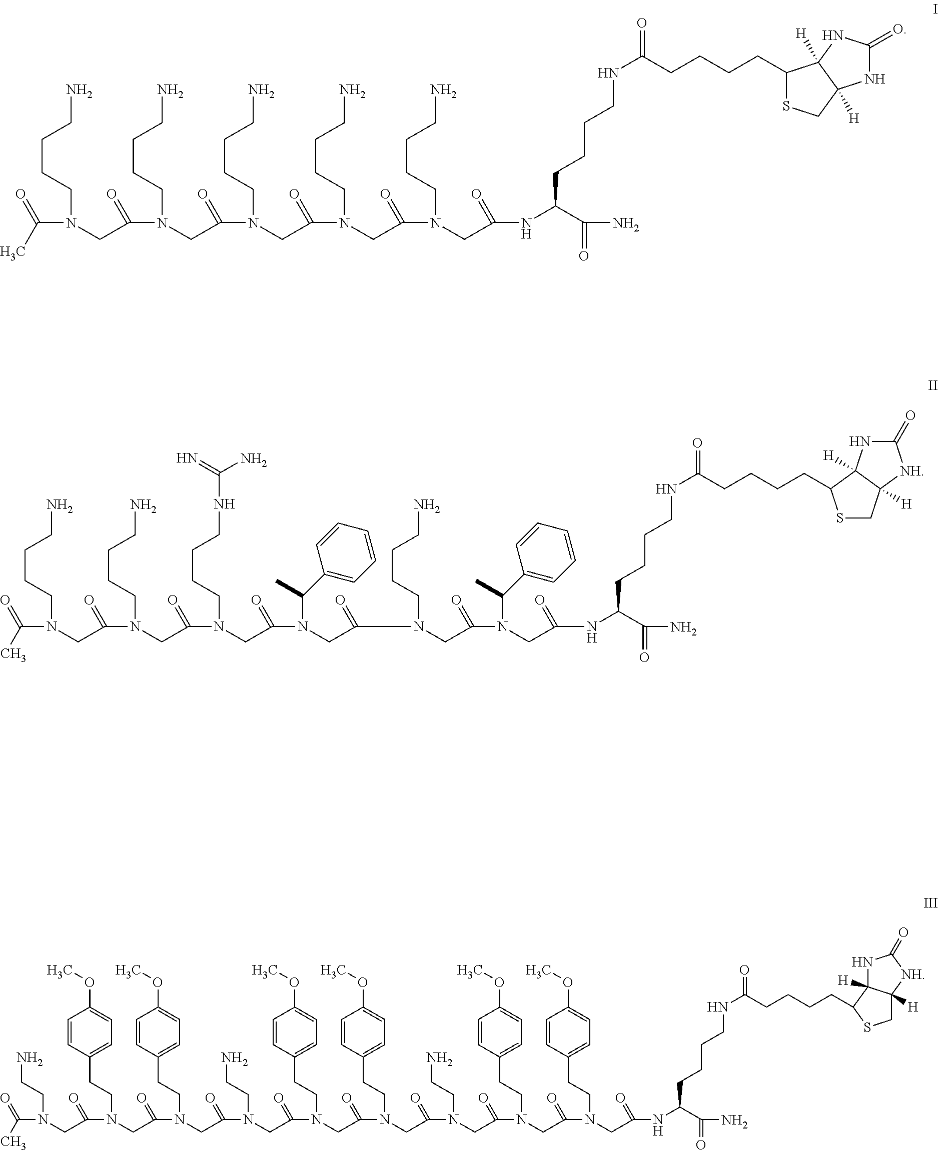

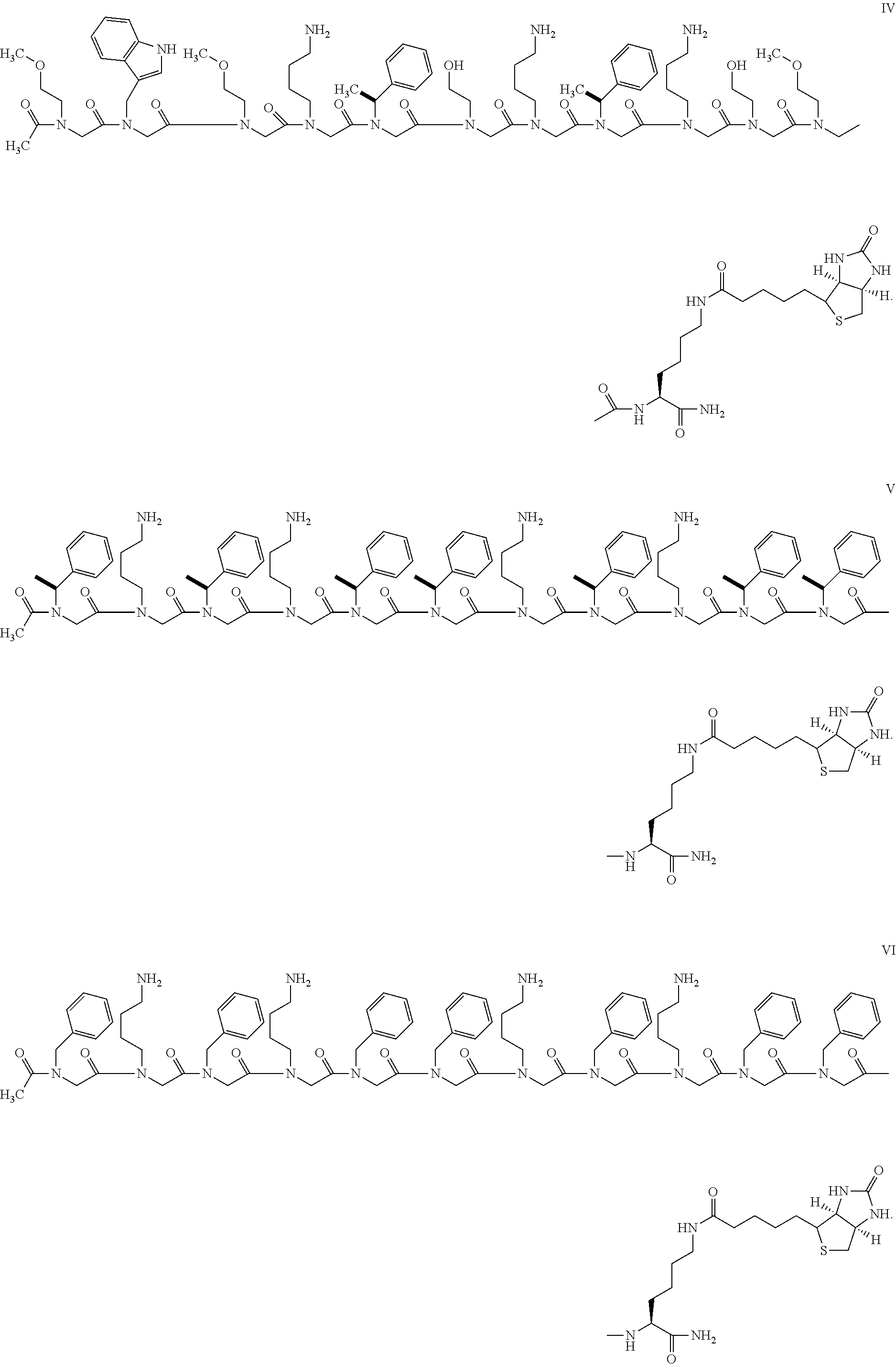

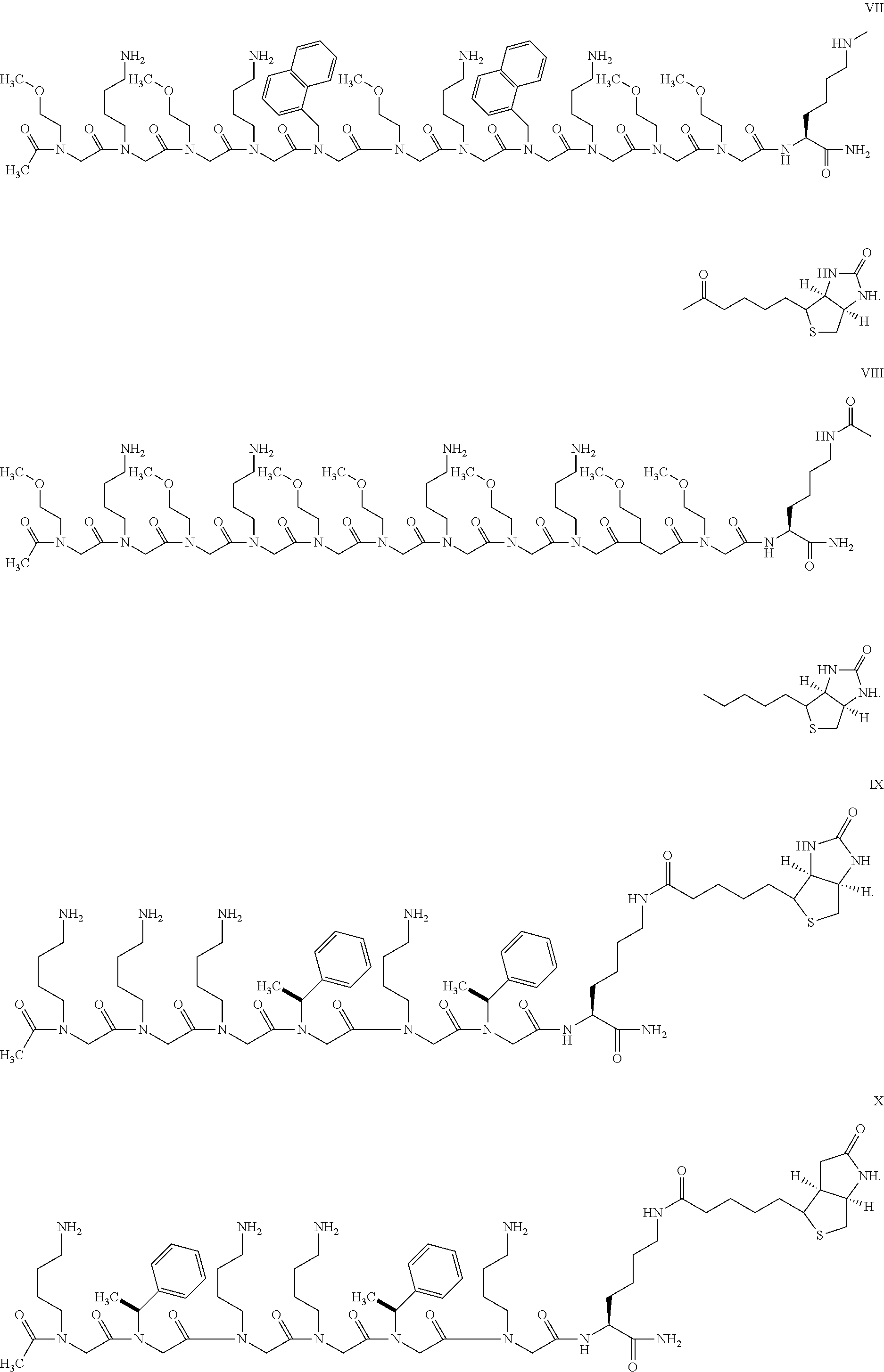

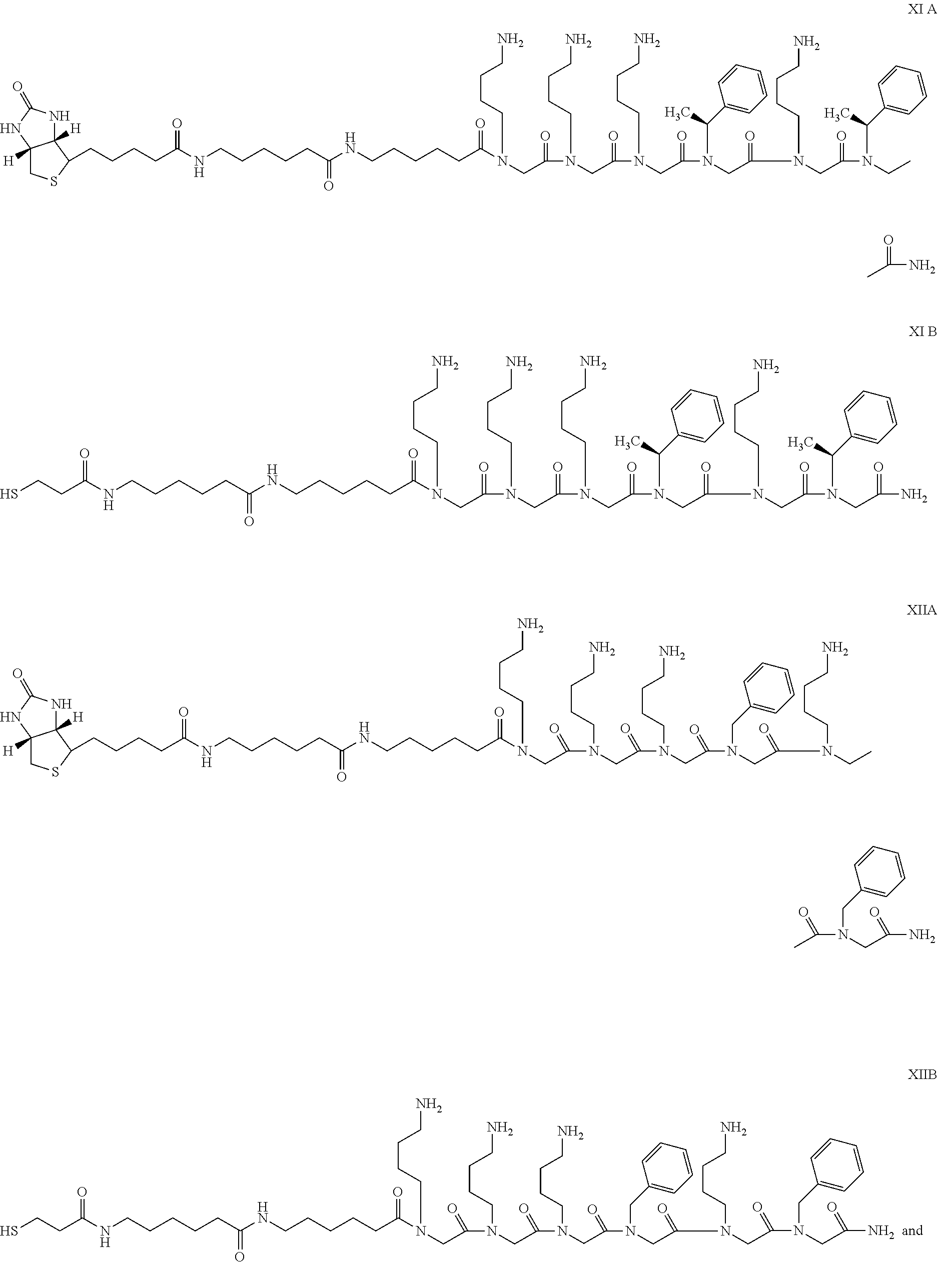

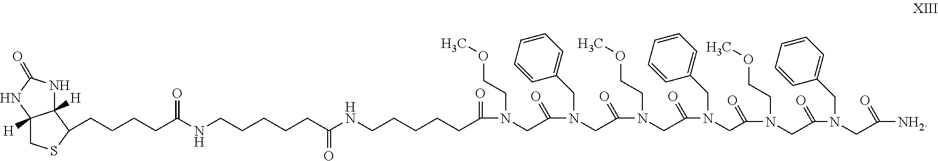

In other preferred embodiments, the methods of the invention capture the non-prion conformer using a PCSB reagent derived from a prion protein fragment, including a peptoid reagent as described herein, which interacts preferentially with a pathogenic prion protein, and detect the non-prion conformer using a CDPSB reagent. The method comprises contacting a sample suspected of containing the non-prion pathogenic conformer with a PCSB reagent under conditions that allow the binding of the reagent to the non-prion pathogenic conformer, if present, to form a first complex; contacting the first complex with a CDPSB reagent under conditions that allow binding; and detecting the presence of the non-prion pathogenic conformer, if any, in the sample by its binding to the CDPSB binding reagent. Typically, unbound sample is removed after forming the first complex and before contacting the first complex with the CDPSB reagent. The CDPSB binding reagent can be a labeled anti-conformational disease protein antibody.

In still yet another preferred embodiment, the methods of the invention capture and detect the presence of a non-prion pathogenic conformer using a PCSB reagent derived from a prion protein fragment, including a peptoid as described herein, which interacts preferentially with a pathogenic prion protein and a CDPSB reagent. The method comprises contacting a sample suspected of containing the non-prion pathogenic conformer with a PCSB reagent under conditions that allow the binding of the PCSB reagent to the non-prion pathogenic conformer, if present, to form a first complex; removing unbound sample materials; dissociating the non-prion pathogenic conformer from the first complex thereby providing dissociated non-prion pathogenic conformer; contacting the dissociated non-prion pathogenic conformer with a CDPSB reagent under conditions that allow binding to form a second complex; and detecting the presence of the non-prion pathogenic conformer, if any, in the sample by detecting the formation of the second complex. The formation of the second complex is preferably detected using a detectably labeled second CDPSB reagent and the PCSB reagent is preferably coupled to a magnetic bead.

In an alternative, the invention provides a method for capturing the non-prion pathogenic conformer using a first PCSB reagent derived from a prion protein fragment, including a peptoid reagent as described herein, which interacts preferentially with a pathogenic prion protein and detecting the non-prion pathogenic conformer using a second PCSB reagent as described herein. The method involves contacting a sample suspected of containing the non-prion pathogenic conformer with the first PCSB reagent under conditions that allow binding of the first reagent to the non-prion pathogenic conformer, if present, to form a first complex; contacting the sample suspected of containing the non-prion pathogenic conformer with a second PCSB reagent under conditions that allow binding of the second reagent to the non-prion pathogenic conformer in the first complex, wherein the second reagent has a detectable label; and detecting the non-prion pathogenic conformer, if any, in a sample by its binding to the second reagent.

In yet another alternative, the invention provides a method for capturing the non-prion pathogenic conformer using a CDPSB reagent and detecting the non-prion pathogenic conformer using a PCSB reagent derived from a prion protein fragment, including a peptoid reagent as described herein, which interacts preferentially with a pathogenic prion protein. The method involves (a) contacting a sample suspected of containing the non-prion pathogenic conformer with a CDPSB reagent under conditions that allow binding of the reagent to the non-prion pathogenic conformer, if present, to form a complex; (b) removing unbound sample materials; (c) contacting the complex with a PCSB reagent under conditions that allow the binding of the PCSB reagent to the non-prion pathogenic conformer, wherein the PCSB reagent comprises a detectable label; and detecting the non-prion pathogenic conformer, if any, in the sample by its binding to the PCSB reagent; wherein the PCSB reagent is derived from a prion protein fragment and interacts preferentially with a pathogenic prion protein.

In all of the above methods “unbound sample” refers to those components within the sample that are not captured in the contacting steps. The unbound sample may be removed by methods that are well known in the art, for example, by washing, centrifugation, filtration, magnetic separation and combinations of these techniques. Preferably, in the methods of the invention, unbound samples are removed by washing the complexes with buffer and/or magnetic separation.

In preferred embodiments, methods of the invention are used for detection of amyloid diseases, including systemic amyloidoses, tauopathies, and synucleinopathies.

### F. Detection Methods for Alzheimer's Disease

Methods for detection of pathogenic Alzheimer's disease conformers such as Aβ40, Aβ42, or tau are provided.

In particularly preferred embodiments, these methods capture the pathogenic Alzheimer's disease conformer with a PCSB reagent derived from a prion protein fragment, including peptoid reagents as described herein, which interacts preferentially with a pathogenic prion protein and detect the captured conformer with a CDPSB reagent.

In particular, the methods comprise contacting a sample suspected of containing the pathogenic Alzheimer's disease conformer with a PCSB reagent under conditions that allow the binding of the PCSB reagent to the pathogenic Alzheimer's disease conformer, if present, to form a first complex; removing unbound sample materials; dissociating the pathogenic Alzheimer's disease conformer from the first complex thereby providing dissociated pathogenic Alzheimer's disease conformer; contacting the dissociated pathogenic Alzheimer's disease conformer with a CDPSB reagent under conditions that allow binding to form a second complex; and detecting the presence of the pathogenic Alzheimer's disease conformer, if any, in the sample by detecting the formation of the second complex. The pathogenic Alzheimer's disease conformer in the first complex is preferably dissociated and denatured with about 0.05N NaOH or about 0.1N NaOH, at about 90° C. or about 80° C. before contacting the CDPSB reagent. When the pathogenic Alzheimer's disease conformer is Aβ40 or Aβ42, it is preferably dissociated and denatured at about 0.1 N NaOH at about 80° C. for about 30 minutes.

Dissociation and/or denaturation can be accomplished using the methods described in Section IV(B). Typically, the pathogenic Alzheimer's disease conformer is simultaneously dissociated and denatured by altering the pH from low to high or high to low pH.

In preferred embodiments, the PCSB reagent is derived from PrP19-30 (SEQ ID NO: 242), PrP23-30 (SEQ ID NO: 243), PrP100-111 (SEQ ID NO: 244), PrP101-110 (SEQ ID NO: 245), PrP154-165 (SEQ ID NO: 246), PrP226-237 (SEQ ID NO: 247), SEQ ID NO:14, SEQ ID NO: 50, SEQ ID NO: 68, or

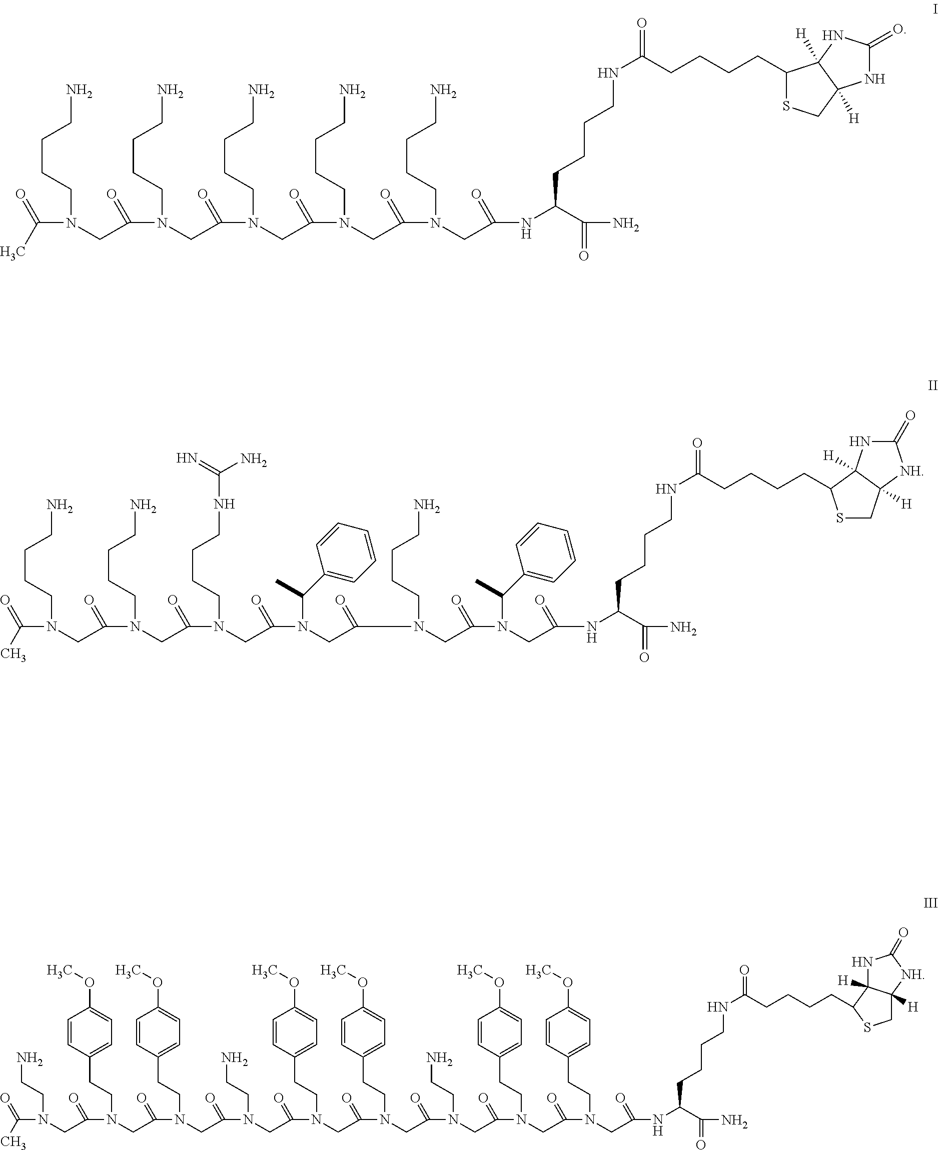

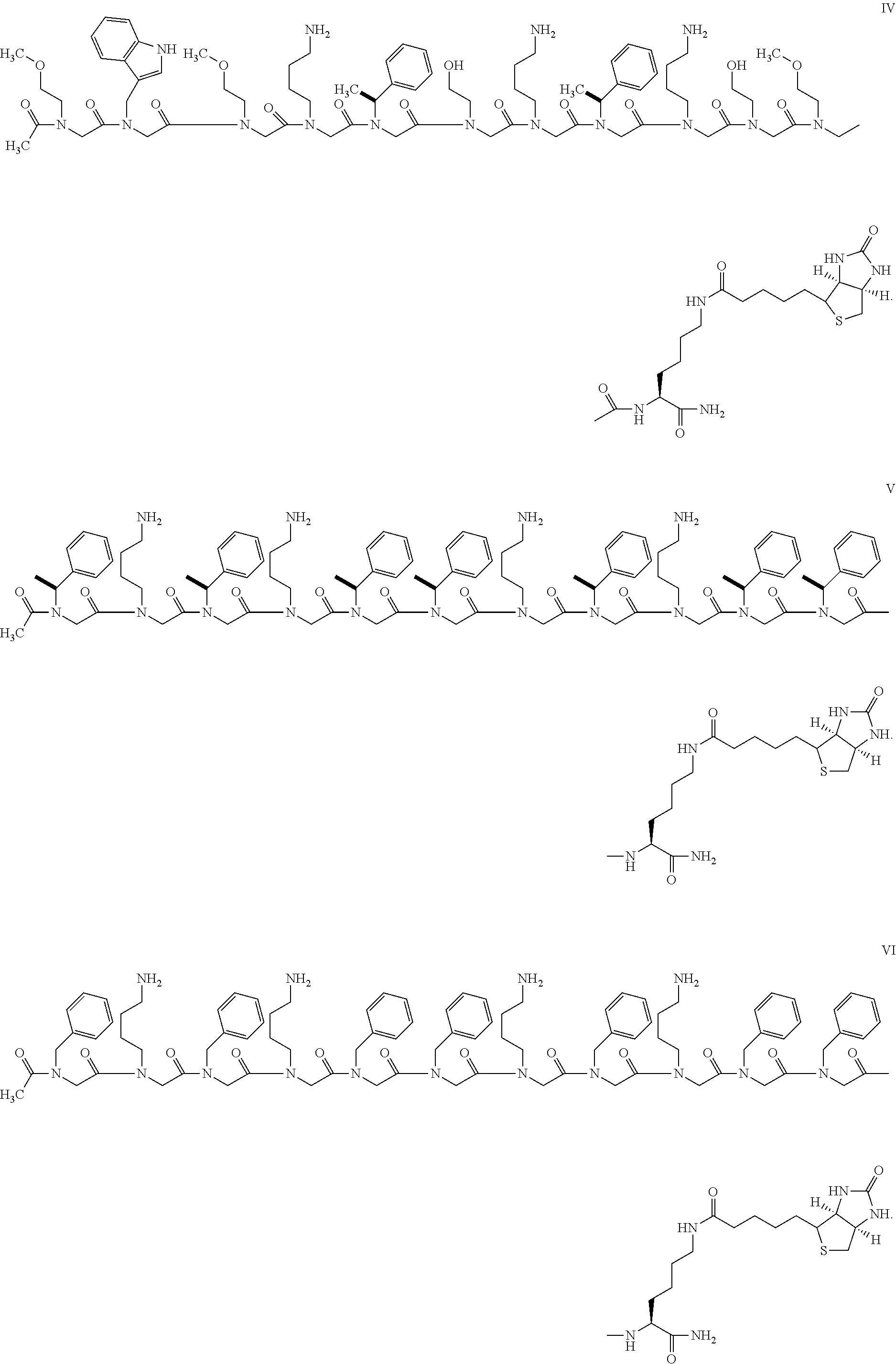

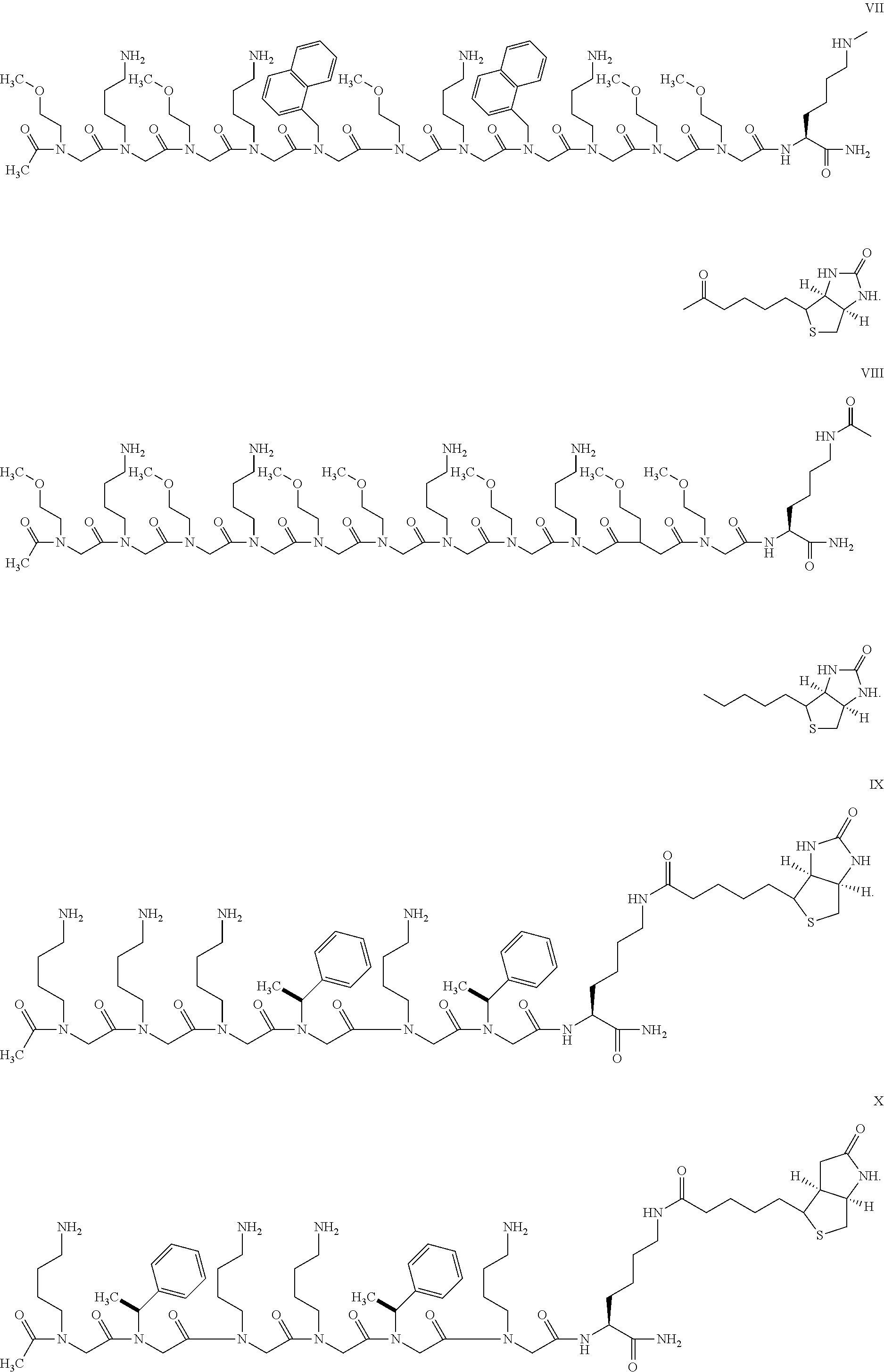

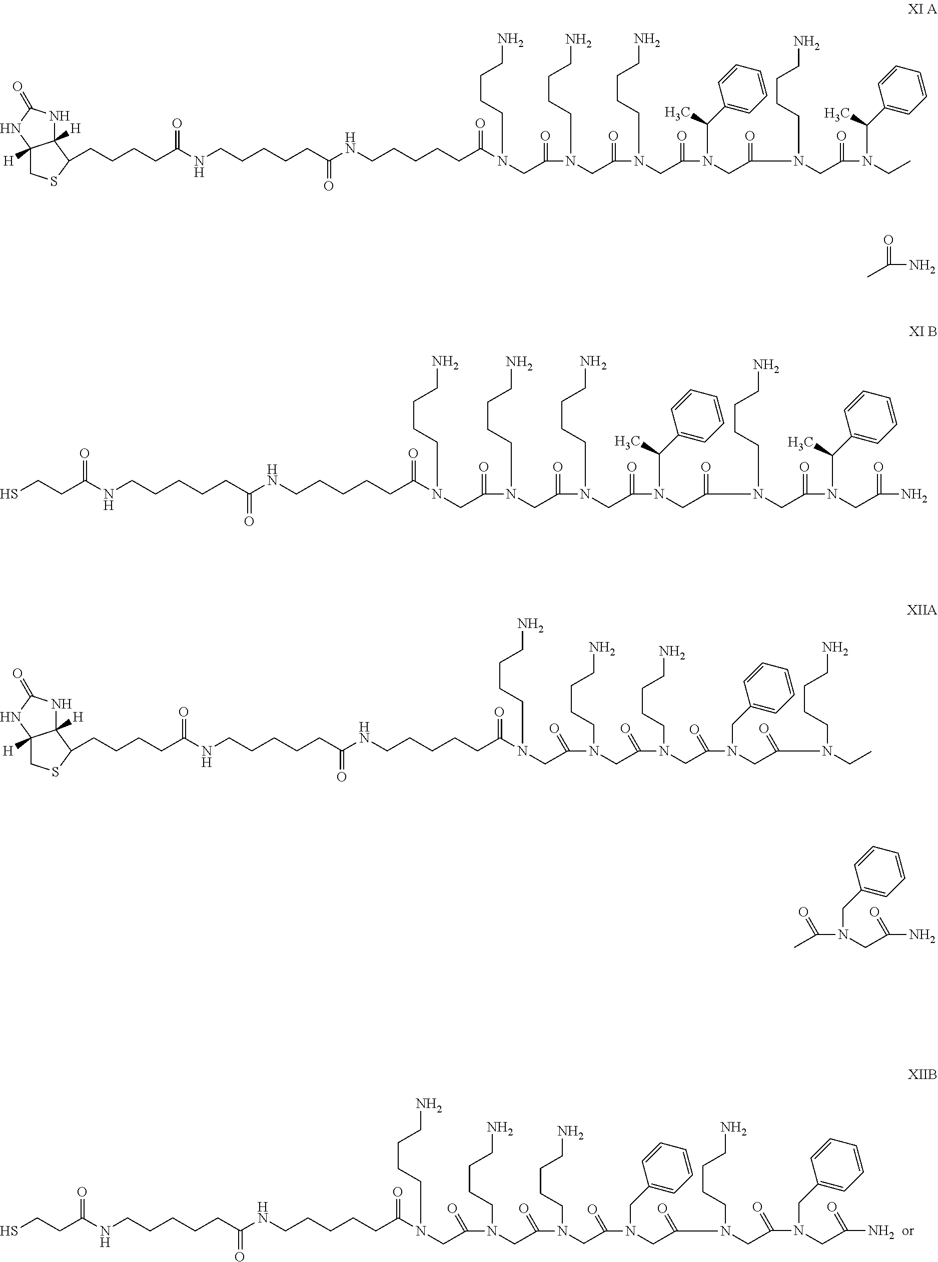

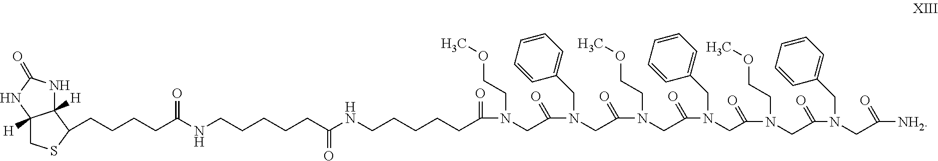

coupled to a solid support, such as a magnetic bead.

The CDPSB reagent is preferably an anti-Alzheimer's disease protein antibody coupled to a solid support such as a microtiter plate and formation of the second complex is preferably detected using a second detectably labeled CDPSB reagent. When the pathogenic Alzheimer's disease conformer is Aβ40 or Aβ42, preferred anti-Alzheimer's disease protein antibodies include 11A50-B10 (Covance), a antibody specific for C-terminus of Aβ40; 12F4 (Covance), a antibody specific for C-terminus of Aβ42; 4G8, specific for Aβ amino acids 18-22; 20.1, specific for Aβ amino acids 1-10; and 6E10, specific for Aβ amino acids 3-8. In particularly preferred embodiments, 20.1 is the capture antibody on an ELISA plate and 12F4 or 11A50-B10 are used as the second detectably labeled CDPSB reagent. The sample is preferably plasma or cerebrospinal fluid (CSF).

Thus, in particularly preferred embodiments, methods for detecting the presence of a pathogenic Alzheimer's disease conformer include, but are not limited to, the steps of: contacting a sample of plasma or CSF suspected of containing pathogenic Alzheimer's disease conformer with peptoid XIIb coupled to a magnetic bead under conditions that allow the binding of the peptoid XIIb to a pathogenic Alzheimer's disease conformer, if present, to form a first complex; removing unbound sample materials; dissociating the pathogenic Alzheimer's disease conformer from the first complex by altering pH, thereby providing a dissociated pathogenic Alzheimer's disease conformer; contacting the dissociated pathogenic Alzheimer's disease conformer with an anti-Alzheimer's disease protein antibody bound to a solid support under conditions that allow binding to form a second complex; and detecting the formation of the second complex by incubating with a second labeled anti-Alzheimer's disease protein antibody.

### G. Competition Assays

In some aspects, the methods of this invention detect pathogenic conformers via competitive binding. Means of detection can be used to determine when a ligand which weakly binds to the PCSB binding reagent is displaced by pathogenic conformer. For instance, PCSB reagent may be adsorbed onto a solid support. Subsequently, the solid support is combined with a detectably labeled ligand that binds to the PCSB reagent with a binding affinity weaker than that with which the pathogenic conformer binds to the PCSB reagent. The ligand-PCSB reagent complexes are detected. Sample is then added. Since the binding affinity of the detectably labeled ligand is weaker than the binding affinity of the pathogenic conformer for the PCSB reagent, the pathogenic conformer will replace the labeled ligand and the decrease in detected amounts of the labeled ligand bound to the PCSB reagent indicate complexes formed between the PCSB reagent and pathogenic conformers in the sample.

Thus, in certain embodiments, the presence of a non-prion pathogenic conformer is detected by providing a solid support comprising a PCSB reagent; combining the solid support with a detectably labeled ligand, wherein the PCSB reagent's binding affinity to the detectably labeled ligand is weaker than the PCSB reagent's binding affinity to the non-prion pathogenic conformer; combining a sample suspected of containing a non-prion pathogenic conformer with the solid support under conditions which allow the non-prion pathogenic conformer, when present in the sample, to bind to the PCSB reagent and replace the ligand; and detecting complexes formed between the PCSB reagent and the non-prion pathogenic conformer from the sample; wherein the PCSB reagent is derived from a prion protein fragment and interacts preferentially with a pathogenic prion protein.

### V. OTHER METHODS

In general, the PCSB reagents described herein are able to interact preferentially with pathogenic conformers of conformational disease proteins Thus, these reagents allow for ready detection of the presence of pathogenic conformers in virtually any sample, biological or non-biological, including living or dead brain, spinal cord, or other nervous system tissue as well as blood. The reagents are thus useful in a wide range of isolation, purification, detection, diagnostic and therapeutic applications.

For example, the reagents described herein may be used to isolate pathogenic conformers using affinity supports. The reagents can be affixed to a solid support by, for example, adsorption, covalent linkage, etc., so that the reagents retain their pathogenic conformer-selective binding activity. Optionally, spacer groups may be included, for example so that the binding site of the reagent remains accessible. The immobilized molecules can then be used to bind the pathogenic conformer from a biological sample, such as blood, plasma, brain, spinal cord, and other tissues. The bound reagents or complexes are recovered from the support by, for example, a change in pH or the pathogenic conformer may be dissociated from the complex.

Thus, in certain embodiments, the invention provides a method for discriminating between a non-prion pathogenic conformer and a non-prion non-pathogenic conformer by contacting a sample suspected of containing the non-prion pathogenic conformer with a PCSB reagent under conditions that allow binding of the reagent to the non-prion pathogenic conformer, if present, to form a complex; and discriminating between the non-prion pathogenic conformer and the non-prion non-pathogenic conformer by binding of the pathogenic conformer to the reagent; wherein the PCSB reagent is derived from a prion protein fragment and interacts preferentially with a pathogenic prion protein.

In other embodiments, the invention provides a method for diagnosing a non-prion conformational disease by contacting a sample suspected of containing a non-prion pathogenic conformer with a PCSB reagent under conditions that allow binding of the reagent to the non-prion pathogenic conformer, if present, to form a complex; detecting the non-prion pathogenic conformer, if any, in the sample by its binding to the reagent; and diagnosing a conformational disease if the non-prion pathogenic conformer is detected; wherein the PCSB reagent is derived from a prion protein fragment and interacts preferentially with a pathogenic prion protein.

Several variations and combinations using the reagents described herein may be applied in the methods of the invention. The following non-limiting examples are described for illustration.

### EXAMPLES

### Example 1

### PSR1 Binds Preferentially to PrP^{Sc}

This Example shows that PSR1 (peptoid reagent XIIb attached to magnetic beads (Streptavidin M-280 Dynabeads)—selectively pulls down PrP^{Sc}.

vCJD or normal brain homogenate (BH) was spiked into 50% pooled normal human plasma in TBS (Tris-buffered saline) with 1% Tween20 and 1% Triton-X 100. No BH was added to Control samples. 100 μL of each sample (containing 10 mL or none of 10% BH) were mixed with 3 μL of XIIb-beads (30 mg/mL) and the resulting mixture was incubated at 37° C. for 1 hr with constant shaking at 750 rpm. Unbound sample materials were removed by washing the beads four times with TBST containing 0.05% Tween20. For each wash, TBST was added and the beads were collected using magnetic force, and the wash buffer was removed. PrP^{Sc} bound to beads was dissociated by addition of 0.1N NaOH. The denatured prion protein was later neutralized by 0.3 M NaH₂PO₄ and transferred to ELISA plate.

Pull-down efficiency was calculated by comparing the signals from the pulldown samples to those from identical samples that were denatured by guanidinium thiocyanate (GdnSCN) without any pulldown. Prion protein from vCJD or normal brain was denatured by mixing equal volume of 5% BH and 6 M GdnSCN, and incubated at room temperature for 10 min. The sample was then diluted in TBST to the same concentration of pulldown samples, with TBST only as control. 100 μL of each directly denatured sample was later transferred to the same ELISA plate for pulldown samples.

The ELISA plate was coated by anti-prion antibody 3F4 at 2.5 ug/mL in 0.1M NaHCO₃. The coating procedure was performed at 4° C. overnight, and then washed three times by TBST. The plate was next blocked by 1% casein in TBS at 37° C. for 1 hr. Prion protein from both pulldown and directly denatured samples were incubated in ELISA plate with 3F4 for 1 hr at 37° C., with constant shaking at 300 rpm, and the plate was washed six times with TBST. Alkaline phosphatase (AP) conjugated detection antibody was diluted to 0.1 μg/mL in 0.1% casein in TBST, and then added to ELISA plate. The plate was later incubated at 37° C. for 1 hr, and washed six times by TBST. The signal was developed using enhanced Lumi-Phos Plus chemiluminescent substrate, and read by a luminometer in relative light units (RLU).

Results are shown below. Prion protein from brain tissue can be completely denatured by 3 M GdnSCN and detected by an anti-prion antibody. In this experiment, we compared signal generated by prion protein pulldown using XIIb-beads to signal obtained from directly denatured protein by GdnSCN. Data showed that the background (no BH) for pulldown and directly denatured samples was 9.0 and 7.7 RLU respectively. Directly denatured 10 mL of 10% normal BH had signal of 14.6 RLU, reflecting PrP^{c} level in normal brain. Meanwhile, 10 mL of 10% normal BH detected by pulldown method showed reading of 9.9 RLU, which is similar to its background. This demonstrated the specificity of peptoid XIIb. When 10 mL of 10% vCJD sample was tested by pulldown and direct denature methods, data showed 53.0 and 56.3 RLU, which means the pulldown efficiency of XIIb-beads reached almost 100%.

TABLE 7

| | vCJD BH | | | Nomal BH | | | No BH | | |
|---|---|---|---|---|---|---|---|---|---|
| | (RLU) | | | (RLU) | | | (RLU) | | |
| | ave | Sd | % cv | ave | sd | % cv | ave | sd | % cv |
| Pulldown | 53.0 | 6.5 | 12.3 | 9.9 | 0.8 | 8.0 | 9.0 | 1.1 | 12.3 |
| No pulldown | 56.3 | 2.6 | 4.6 | 14.6 | 0.7 | 4.5 | 7.7 | 2.3 | 29.4 |

### Example 2

### PSR1Also Preferentially Interacts with Aggregated Amyloid-Beta Protein (Aβ)

This Example shows that PSR1 also preferentially interacts with aggregated Aβ.

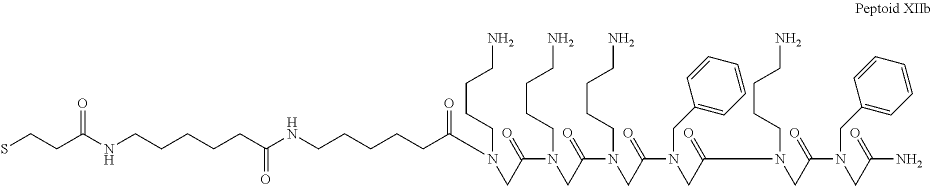

This Example demonstrates the presence of insoluble aggregated Aβ40/42 in several Alzheimer's disease brains and that detection of these aggregates could only be achieved after their denaturation to expose antibody epitopes masked within the aggregate. PSR1 capture of aggregated Aβ from these brains was specific and mediated by peptoid XIIb rather than the pulldown bead. The Example demonstrates that PSR1 selectively captures aggregated Aβ40/42 spiked into plasma, a sample matrix containing soluble Aβ40/42 that is not recognized by PSR1. Finally, the Example confirms that PSR1 captures aggregated Aβ40/42 by demonstrating that capture was disrupted by denaturation of the samples with a chemical denaturant prior to pulldown.

### Quantitation of Total Aβ40/42 in Alzheimer's Disease and Normal Brains

Total Aβ40/42 in brains was quantitated using a sandwich ELISA employing commercially available antibodies from Covance. Individual wells of the 96-well capture plate were coated with antibodies, either mAb 11A50-B10 (detects only soluble Aβ40) or mAb 12F4 (detects only soluble Aβ42). Aβ40 or Aβ42 that was captured on the ELISA plate was detected by mAb 4G8 (which recognizes all forms of Aβ that contain the sequence VFFAE) conjugated to alkaline phosphatase (AP). Lumiphos Plus (Lumigen) was used as the chemiluminescent substrate. Chemiluminescence was read on a Luminoskan luminometer (Thermo).

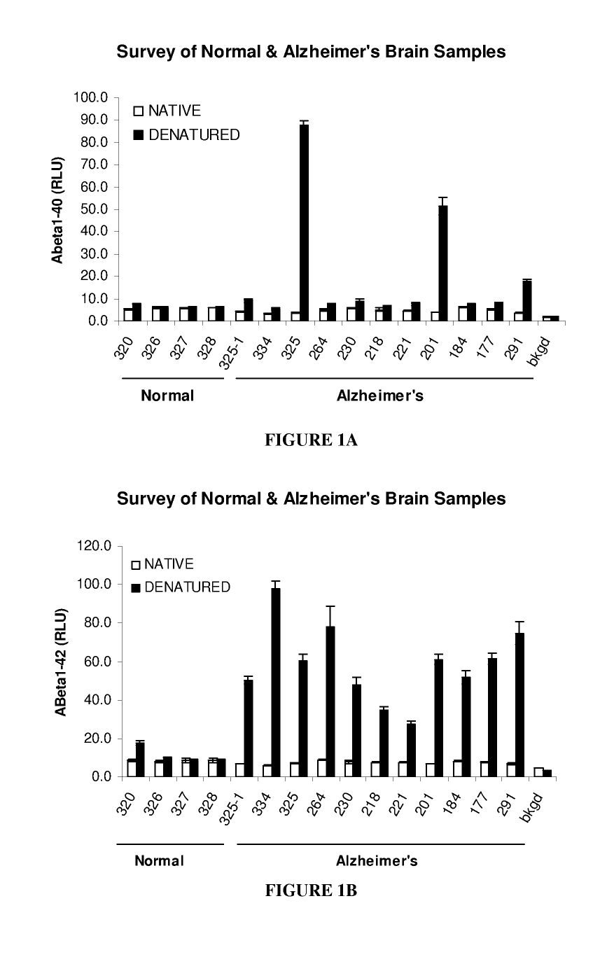

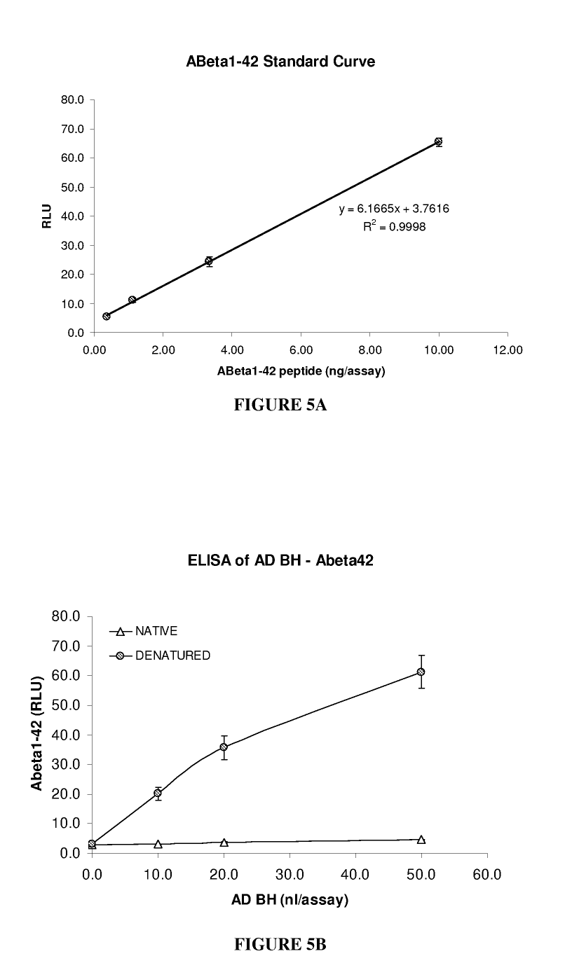

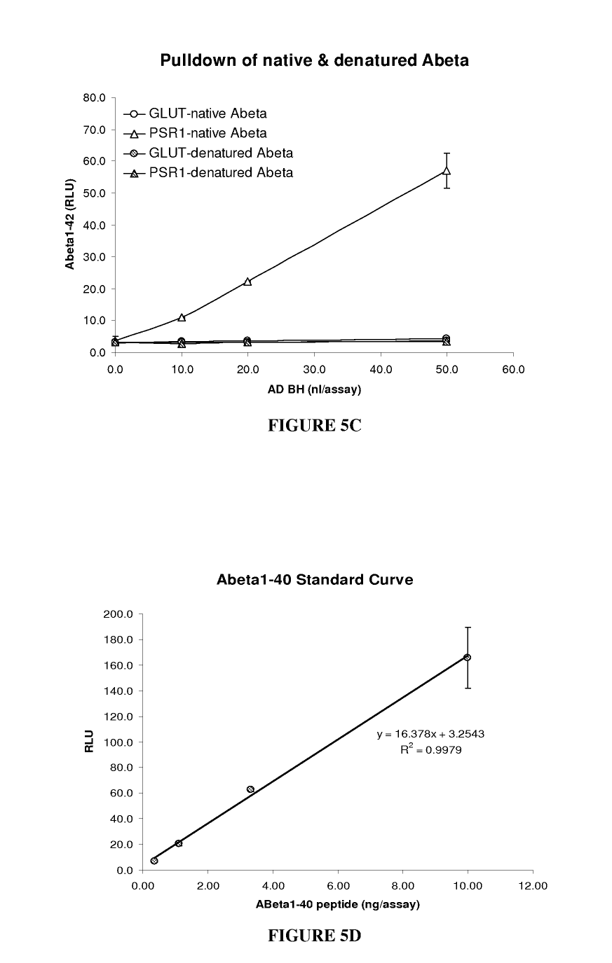

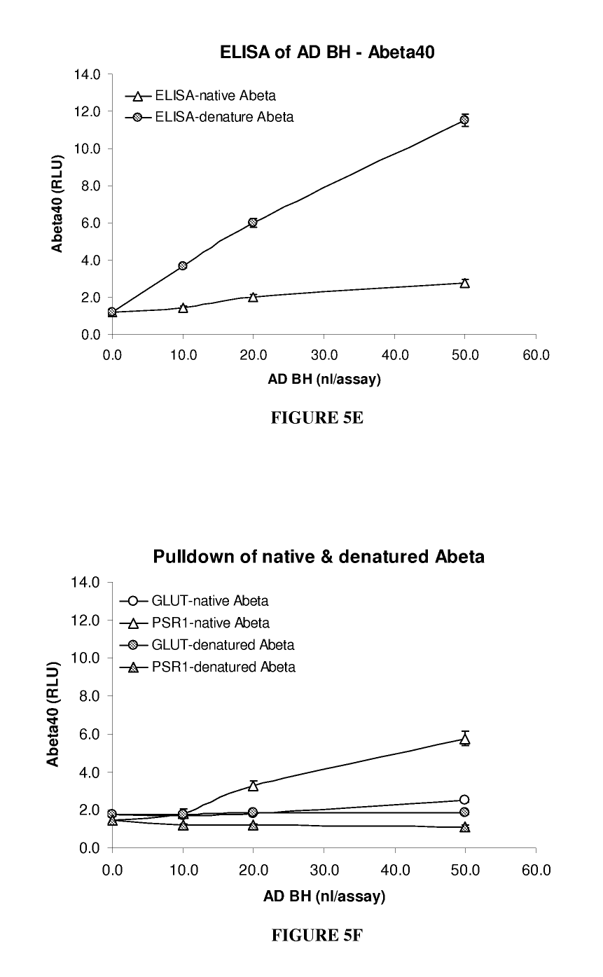

Aβ40 and Aβ42 levels in AD (patient identification #325-1, 334, 325, 264, 230, 218, 221, 201, 184, 177, 291) and control (patients 320, 326, 327, 328) brain homogenates were determined using this sandwich ELISA detection system. Denaturation of the brain homogenates with 5.4 M GdnSCN was required to detect most of the Aβ peptide (FIGS. 1, 5B, and 5E). It is well accepted that antibodies may not bind to epitopes that are conformationally altered or masked within aggregated material. This property has been previously observed for some antibodies recognizing prion and other amyloid proteins. The ELISA data demonstrates accumulation of pathogenic aggregated Aβ in AD brains but not in age matched non-AD control brains. Control brains did not contain very high levels of any Aβ forms (soluble or aggregated), but both Aβ40 and Aβ42 were easily detected from AD patient samples. (FIG. 1).

A titration of one AD brain homogenate (patient # 291) detected by ELISA in parallel with a standard curve (denatured synthetic Aβ peptide) allowed the determination of the concentration of Aβ40 and Aβ42 in this brain homogenate (FIG. 5A, B and 5D, E) which were calculated to be 10 and 240 μg/mL, respectively. Aβ40/42 in AD Brains is Aggregated

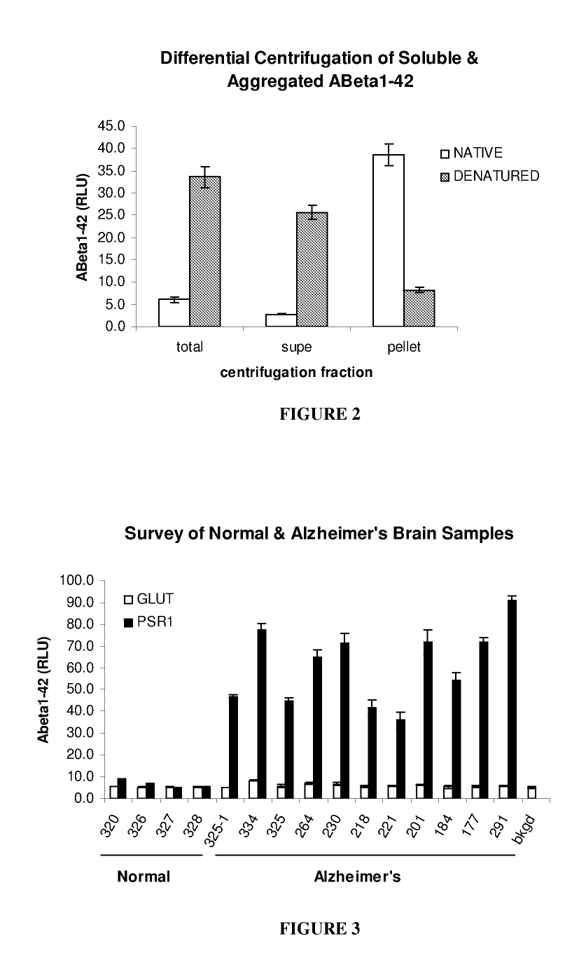

Aβ from AD BHs was considered to be aggregated because treatment with denaturant prior to the ELISA increased detection by sandwich ELISA (FIG. 1). To test if this Aβ exhibited insolubility, another characteristic of aggregates, AD BHs were centrifuged and the supernatant and pellet fractions applied to the ELISA. (FIG. 2). The majority of Aβ signal was in the pellet fraction for un-denatured AD BH. However, pretreatment of the BH with GdnSCN shifted the Aβ signal to the supernatant fraction. This experiment demonstrates that the Aβ aggregates found in AD BH are insoluble by our centrifugation conditions and that pretreatment with chemical denaturant renders Aβ soluble. Therefore, by directly examining the physical properties of Aβ in AD BH, we confirm that the majority of Aβ peptide from AD BHs is aggregated. This is a well known phenomenon that has been observed for a certain class of maladies described as amyloid diseases. These diseases, which include Prion diseases (e.g., CJD), AD, Parkinson, Diabetes type II, and systematic amyloidosis to name few, are associated with the presence of ordered aggregated proteins and the conversion of some protein from the normal conformation into β-sheet conformation.

PSR1 binding to Aβ is mediated by the peptoid reagent XIIb

PSR1 captured Aβ from AD brain homogenate spiked into 80% plasma in TBSTT buffer (FIG. 3). Brain homogenate from AD or control brains was incubated with PSR1 beads or control glutathione beads (no peptoid XIIb). The beads were washed and captured material was dissociated from the beads and denatured with 6 M GdnSCN. The beads were removed using a magnet and the denatured samples were diluted, and applied onto an ELISA plate pre-coated with anti-Aβ42 antibody 12F4. Detection was carried out with AP labeled 4G8 antibody. PSR1 captured Aβ from AD samples, while the control bead (M270-Carboxylic acid beads reacted with maleimide and glutathione (M270-Glutathione, produced in-house)) was unable to capture detectable amounts of Aβ from any brain sample. This experiment establishes that the ability to capture Aβ is due to the peptoid XIIb covalently linked to the bead and not the bead itself.

### PSR1 Selectively Binds to Aggregated Aβ in the Presence of Soluble Aβ

Plasma contains significant levels of soluble Aβ40 and Aβ42. Therefore, to assess whether PSR1 was capable of selectively binding to aggregated Aβ in the presence of soluble Aβ, brain homogenate from AD patient #291 was spiked into plasma and subjected to PSR1 pulldown.

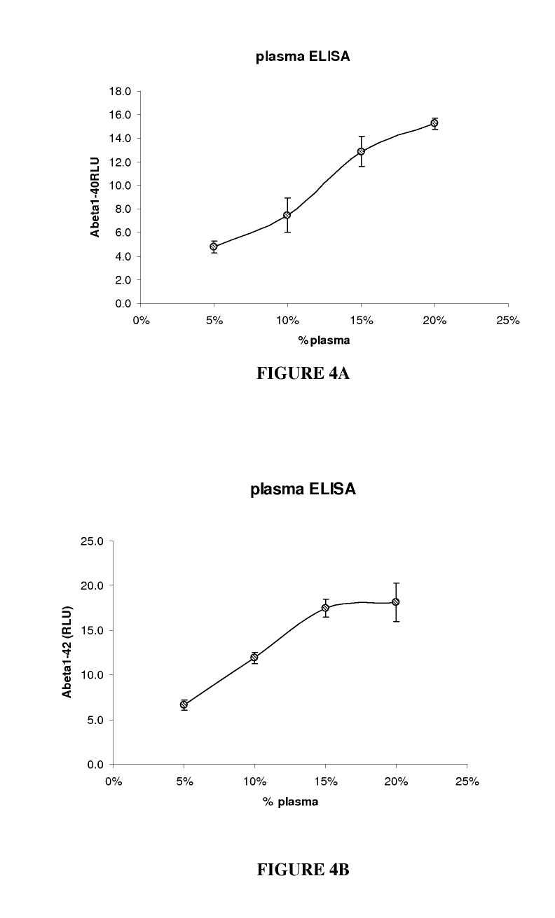

FIGS. 4A and 4B show the quantitation of the endogenous Aβ levels using our sandwich ELISA in increasing concentrations of plasma (normal human plasma from commercial sources) diluted in TBST. Soluble Aβ40 and Aβ42 levels were found to be in the 10-100 ng/ml range.

First, an ELISA was performed on native and denatured AD BH patient #291 samples to assess the amount of aggregated Aβ40/42 present in the samples (FIG. 5B-Aβ42; FIG. 5D-Aβ40).

Next, samples having increasing amounts of AD brain homogenate spiked into plasma (80% plasma in TBSTT buffer) were subjected to PSR1 pulldown. PSR1 was able to selectively capture Aβ42 and Aβ40 from the spiked AD brain homogenate (10, 20, 50 mL spike), but not any soluble Aβ42 or Aβ40 from plasma alone (0 mL spike) (FIGS. 5C&F, see white triangles: PSR1-native Abeta). This suggests that PSR1 captures only aggregated Aβ peptide found in Alzheimer's Disease brains and not the soluble Aβ found in plasma. The results also indicate that plasma components, which include a high concentration of various proteins, lipids, and ions, do not interfere with PSR1 binding.

### PSR1 Capture is Disrupted by Solubilization of Sample Prior to Pulldown

When the same AD brain homogenates were denatured with 5.4 GdnSCN to solubilize aggregates prior to incubation with plasma and PSR1, no Aβ was detected. (FIGS. 5C&F—see gray triangles: PSR1-denatured Abeta). This supports the idea that PSR1 recognizes misfolded aggregated properties of Aβ found in AD samples which can be solubilized by denaturation and not another determinant specific to the AD-derived Aβ peptide.

### Example 3

### Peptides Derived from Prion Protein Fragments have Similar Capture Profiles For Pathogenic Prion Proteins and Aggregated Aβ from Diseased Brain Homogenates Spiked into Buffer or Plasma

WO05/016137, WO07/030,804 describe various peptides and peptoids derived from prion protein fragments which preferentially interact with the pathogenic conformer of the prion protein. This experiment suggests that these reagents capture Aβ by a mechanism similar to that by which they capture pathogenic prions.

Six different peptides derived from a prion fragment having three different capture profiles were tested. The amino acid sequence of each peptide corresponds to a fragment of the human prion protein sequence and is described below. The subscripted numbers indicate the amino acid position of the first and last amino acids of the fragment.

1) Group 1: PrP₁₉₋₃₀ and PrP₁₀₀₋₁₁₁, which capture PrP^{Sc} in both plasma and buffer
2) Group 2: PrP₁₅₄₋₁₆₅ and PrP₂₂₆₋₂₃₇, which can capture PrP^{Sc} only in buffer
3) Group 3: PrP₃₇₋₄₈ and PrP₁₈₁₋₁₉₂, peptides which are not capable of capturing PrP^{Sc}.
PrP₃₇₋₄₈ and PrP₁₈₁₋₁₉₂ were chosen as negative controls since they are peptides with similar physicochemical properties to PrP₁₅₄₋₁₆₅ and PrP₂₂₆₋₂₃₇, but with different amino acid sequences.

| Fragment | |
|---|---|
| Name | Sequence |
| Modified | Biotin-Ahx-LGLCKKRPKPGG-CONH2 |
| PrP19-30 | (SEQ ID NO: 256) |
| | (Ahx = aminohexanoic acid) |
| Modified | Biotin-Ahx-RYPGQGSPGGNR-CONH2 |
| PrP37-48 | (SEQ ID NO: 257) |
| Modified | Biotin-Ahx-NKPSKPKTNMKH-CONH2 |
| PrP100-111 | (SEQ ID NO: 258) |
| Modified | Biotin-Ahx-MHRYPNQVYYRP-CONH2 |
| PrP154-165 | (SEQ ID NO: 259) |
| Modified | Biotin-Ahx-NITIKQHTVTTT-CONH2 |
| PrP181-192 | (SEQ ID NO: 260) |
| Modified | Biotin-Ahx-YQRGSSMVLFSS-CONH2 |
| PrP226-237 | (SEQ ID NO: 261) |

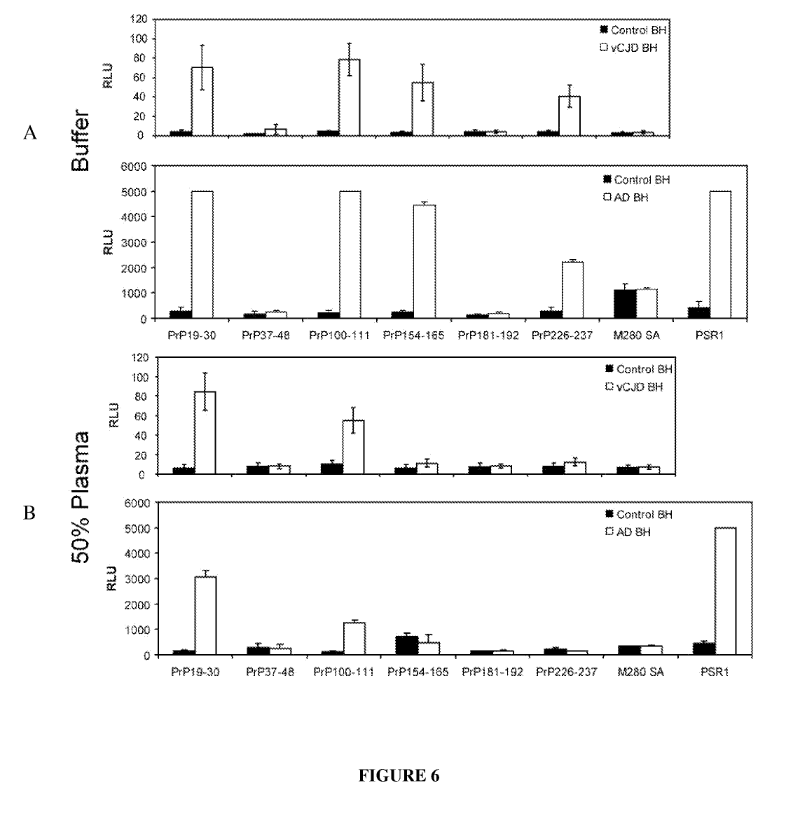

The results were very surprising. The activity profile of aggregated Aβ42 captured by the peptides was nearly identical to the profile of captured PrP^{Sc} from patients afflicted with Creutzfeldt-Jakob Disease, a prion-based disease (FIG. 6): group 1 peptides captured aggregated Aβ42 spiked into either plasma or buffer; group 2 peptides captured aggregated Aβ42 spiked into buffer but not plasma; group 3 peptides did not capture any Aβ42. This result is very strong evidence that the binding mechanism between tested PCSB reagents with PrP^{Sc} and Aβ aggregates is similar. The most probable interacting domain is a motif that is common and part of the general amyloid structure regardless of the protein amino acid sequence.

### SUMMARY

This Example and Example 2 together demonstrate that reagents which are capable of preferentially interacting with pathogenic prions are also capable of preferentially interacting with aggregated Aβ The ability of these reagents to bind both pathogenic prions and aggregated Aβ with such similar binding characteristics was completely unexpected.

The ability of these reagents to capture aggregated proteins allows direct detection of Alzheimer's disease-associated Aβ protein aggregates. This is advantageous compared to tests of more indirect markers of AD disease. This is also advantageous compared to reagents such as anti-Aβ antibodies which bind specifically to non-aggregated Aβ. Such antibodies can only associate with soluble Aβ which is present in normal and AD patients and whose concentrations in certain biological fluids are only indirect markers of AD disease. Use of such reagents to detect aggregated Aβ would require comparing native samples with samples treated to solubilize aggregates. Unfortunately, this analysis is ineffective in samples containing only low levels of aggregates and requires sample manipulation that is likely to dilute Aβ levels below detectable limits.

### Example 4

### Effect of Sample Matrix on Pulldown of Aggregated Aβ42

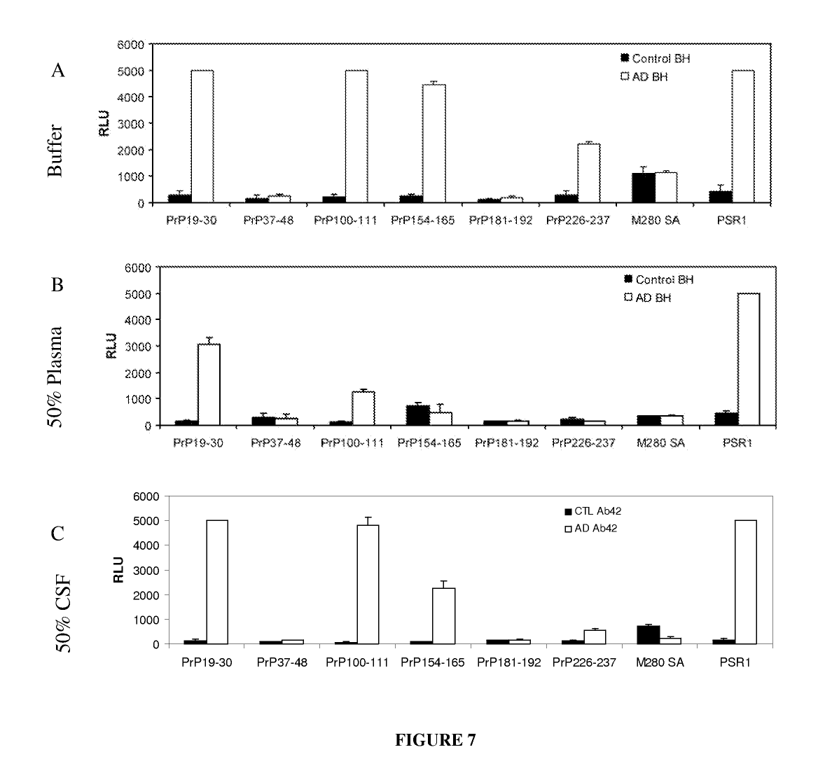

The effect of sample matrix on pulldown of aggregated Aβ42 was evaluated by testing PSR1 and the peptides described above with three different Alzheimer's disease brain samples: 1) brain homogenate spiked into buffer; 2) brain homogenate spiked into plasma; and 3) brain homogenate spiked into CSF (See FIG. 7).

The samples were prepared and processed as described for FIG. 6. The brain homogenate spiked into CSF sample was prepared by spiking 50 mL of BH into 100 microliters of 50% plasma in TBSTT buffer.

This experiment confirms other studies showing that PSR1 preferentially interacts with aggregated Aβ42 in the presence of plasma and demonstrates that PSR1 also preferentially interacts with aggregated Aβ42 in the presence of CSF.

The tested peptides exhibited different capture profiles. The differences in capture behavior in buffer, plasma, and CSF suggest that there are interfering components in plasma and CSF which disrupt the mechanism of binding. A broader range of reagents can capture aggregated Aβ42 in CSF as compared to in plasma. PrP₁₉₋₃₀ and PrP₁₀₀₋₁₁₁, which can capture Aβ42 in the presence of plasma, can also do so in the presence of CSF. However, PrP₁₅₄₋₁₆₅ and PrP₂₂₆₋₂₃₇, which are not capable of capturing Aβ in the presence of plasma, can do so in the presence of CSF. This finding is consistent with the fact that CSF has a lower protein concentration than plasma and is a less complex sample matrix. It is therefore less likely to contain components which may interfere with the interaction between the reagent and aggregated Aβ.

To summarize, these experiments show that positively charged reagents PSR1, PrP₁₉₋₃₀, and PrP₁₀₀₋₁₁₁ were able to interact with aggregated Aβ in both simple buffer (TBSTT) as well as body fluids more applicable for ante-mortem diagnosis of Alzheimer's disease, such as CSF and plasma.

### Example 5

### Dissociation and Denaturation of Aβ for ELISA—Optimization of NaOH Concentration and Incubation Temperature

Brain homogenate (BH) from normal or Alzheimer's disease (AD) patient were spiked into normal human plasma (NHP) and captured by PSR1 beads. After washing, the beads were re-suspended with NaOH (0.01N-0.3N) in a PCR thin-wall tube and incubated at 60° C., 70° C., 80° C., or 90° C. for 10 min on a Perkin Elmer MasterCycler. The denatured Aβ samples were neutralized to approximately pH 7.5, and then proceeded to sandwich ELISA detection.

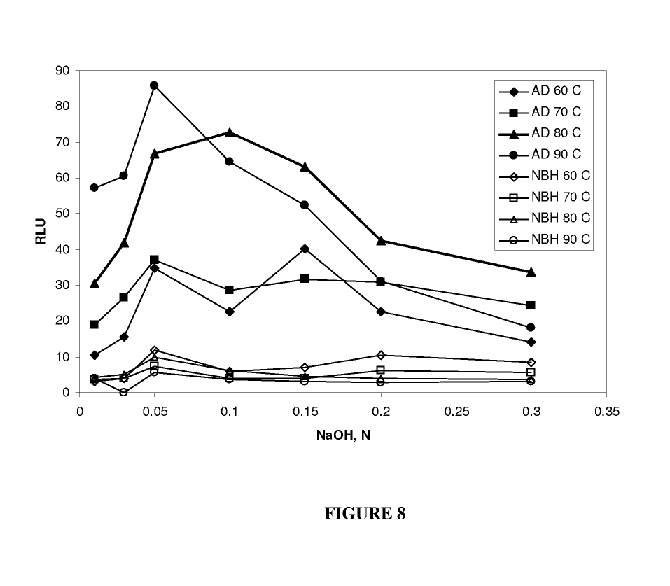

The results showed the highest signal when Aβ was denatured at 0.05N NaOH at 90° C. but with a small dynamic range (see FIG. 8). When denatured at 80° C., the Aβ signal reached a plateau from 0.05N to 0.15N of NaOH, and 0.1N NaOH showed the second highest signal. Therefore, further study focused on denaturing Aβ at 80° C. with 0.1N NaOH.

Aβ requires NaOH denaturation at a higher temperature and for a longer incubation than the prion protein. Denaturation of Aβ at 0.1N NaOH at 80° C. for 30 minutes works well for Aβ, whereas 0.1N NaOH at room temperature for 10 minutes works well for prions.

### Example 6

### PSR1 Capture/Sandwich ELISA for Detection of Amyloid Beta

This Example describes a protocol for detection of Aβ using PSR1 as a pathogenic conformer-specific binding (PCSB) reagent. The assay has three basic steps: 1) capture of Aβ aggregates from the sample using PCSB reagent, 2) denaturation and dissociation of the bound material from the PCSB reagent using a chaotropic agent, and 3) a sandwich ELISA using commercially available antibodies. Use of 4G8-AP as a detection reagent in the ELISA provides a linear response which is superior to 4G8-HRP.

### Sample Preparation

100 mL of 10% brain homogenate (BH) is spiked into 100 μL/assay of the following: a) 50% normal human plasma (NHP) and 1×TBSTT (50 mM Tris, pH 7.5; 150 mM NaCl; 1% Tween-20; 1% Triton X-100); b) 50% normal human cerebrospinal fluid (CSF) and 1×TBSTT; or c) 0.1% bovine serum albumin (BSA) and 1×TBSTT.

### Bead Capture

These samples are added to 3 μL/assay of PSR1 peptoid covalently coupled to Dynal M270-carboxylic acid beads at 30 mg/mL and incubated at 37° C. for 1 hour with shaking. Alternatively, the sample is added to 10 μL/assay of M280-streptavidin beads (10 mg/mL) coated with biotinylated PSR1 peptoid.

The beads are washed 4 times with 275 μL of TBST (50 mM Tris, pH 7.5, 150 mM NaCl, 0.05% Tween-20). For each wash, the washed beads are collected with a magnet and the wash buffer is removed.

2 μL/assay 6M guanidine thiocyanate is then added and the sample is incubated at room temperature for 30 minutes to elute and denature the captured material. 98 μL/assay of TBST is then added to dilute the guanidine thiocyanate.

To ensure that the correct amount of 6M guanidine thiocyanate is added, sample preparation and bead capture are done in bulk (multiple reactions in one tube or well). After diluting the GdnSCN, samples are transferred to 96-well microtiter plates for the rest of the protocol.

The beads are removed by magnetic separation and the supernatant is transferred onto the capture plate.

### Detection

A capture plate (Microlite 2+ from Thermo Scientific) is coated with either mouse monoclonal antibody (mAb)11A50-B10 (C-terminal antibody; specifically binds Aβ1-40) or mAb 12F4 (C-terminal antibody; specifically binds Aβ1-42) at 2.5 m/mL. Both antibodies are commercially available from Covance.

The supernatant is incubated on the capture plate for 1 hour at 37° C. The capture plate is washed 4 times with 275 μL/well of TBST.

0.2 μg/mL mAb 4G8, conjugated to alkaline phosphatase, in 0.1% BSA and TBST is added to the capture plate for detection. The purified antibody is available from Covance. The AP conjugate is made in-house with the starting material (antibody) from Covance.

The capture plate is then incubated with detection antibody for 1 hour at 37° C. and washed 4 times with 275 μL/well of TBST.

100 μL of enhanced Lumiphos Plus (0.55% SDS added at a ratio of 91 μL per mL of Lumiphos Plus, the chemiluminescent substrate for detection) is added to the capture plate. Then the capture plate is incubated for 30 min at 37° C. and the plate is read on an LuminoSkan

### Example 7

### Synthesis of the PCSB Reagents to be Used in Methods of the Invention

Peptide fragments of prion proteins were chemically synthesized using standard peptide synthesis techniques, essentially as described in Merrifield (1969) Advan. Enzymol. 32: 221 and Holm and Medal (1989), Multiple column peptide synthesis, p. 208E, Bayer and G. Jung (ed.), Peptides 1988, Walter de Gruyter & Co. Berlin-N.Y. Peptides were purified by HPLC and sequence verified by mass spectroscopy.

In certain cases, the peptides synthesized included additional residues at the N or C terminus, for example GGG residues and/or included one or more amino acid substitutions as compared to wild-type sequences.

### A. Peptoid Substitutions

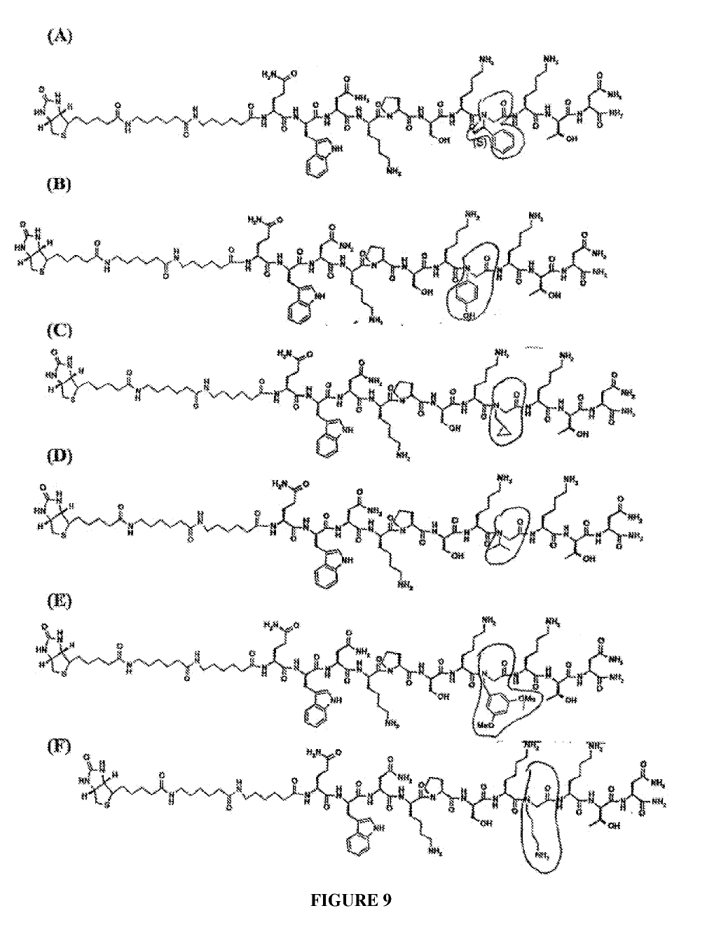

Peptoid substitutions were also made in the peptide presented in SEQ ID NO:14 (QWNKPSKPKTN, corresponding to residues 97 to 107 of SEQ ID NO:2), SEQ ID NO:67 (KKRPKPGGWNTGG, corresponding to residues 23-36 of SEQ ID NO:2) and SEQ ID NO:68 (KKRPKPGG, corresponding to residues 23-30 of SEQ ID NO:2). In particular, one or more proline residues of these peptides were substituted with various N-substituted peptoids. See, FIG. 9 or peptoids that can be substituted for any proline. Peptoids were prepared and synthesized as described in U.S. Pat. Nos. 5,877,278 and 6,033,631, both of which are incorporated by reference in their entireties herein; Simon et al. (1992) Proc. Natl. Acad. Sci. USA 89:9367.

### B. Multimerization

Certain peptide reagents were also prepared as multimers, for example by preparing tandem repeats (linking multiple copies of a peptide via linkers such as GGG), multiple antigenic peptides (MAPS) and/or linearly-linked peptides.

In particular, MAPS were prepared using standard techniques, essentially as described in Wu et al. (2001) J Am Chem. Soc. 2001 123(28):6778-84; Spetzler et al. (1995) Int Pept Protein Res. 45(1):78-85.

Linear and branched peptides (e.g., PEG linker multimerization) were also prepared using polyethylene glycol (PEG) linkers, using standard techniques. In particular, branched multipeptide PEG scaffolds were created with the following structures: Biotin-PEG-Lys-PEG-Lys-PEG-Lys-PEG-Lys-PEG-Lys (no peptide control) and Biotin-PEG-Lys(Peptide)-PEG-Lys(Peptide)-PEG-Lys(Peptide)-PEG-Lys(Peptide)-PEG-Lys(Peptide). In addition, peptide to Lys linkages were prepared: Lys-epsilon-NH—CO—(CH₂)³-Mal-S-Cys-peptide. See, FIG. 10

### C. Biotinylation

Peptides were biotinylated using standard techniques following synthesis and purification. Biotin was added to the N- or C-terminal of the peptide.

### Example 8

### PCSB Peptoid Reagents

The following PCSB peptoid reagents were prepared using synthetic methods for preparation of peptoid molecules containing N-substituted glycine residues such as the procedures disclosed in U.S. Pat. Nos. 5,811,387; 5,831,005; 5,877,278; 5,977,301; 6,075,121; 6,251,433; and 6,033,631, as well as Simon et al. (1992) Proc. Natl. Acad. Sci. USA 89: 9367, each of which is incorporated herein by reference in its entirety.

Peptoid Reagent I

The below peptoid reagent includes, but is not limited to SEQ ID NO: 229.

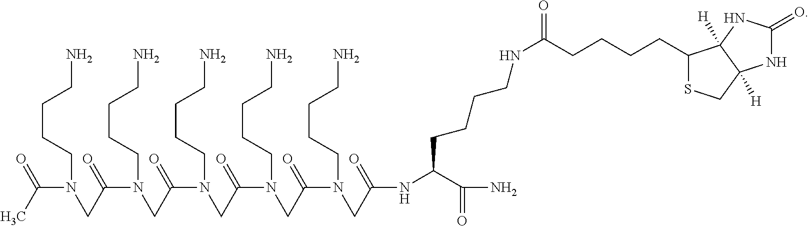

Calculated Mass: 1054.42; Observed Mass: 1054.2. All observed mass measurements were measured on a Waters (Milford, Mass.) Micromass ZQ LC/MS System.

### Peptoid Reagent II

The below peptoid reagent includes, but is not limited to SEQ ID NO: 230.

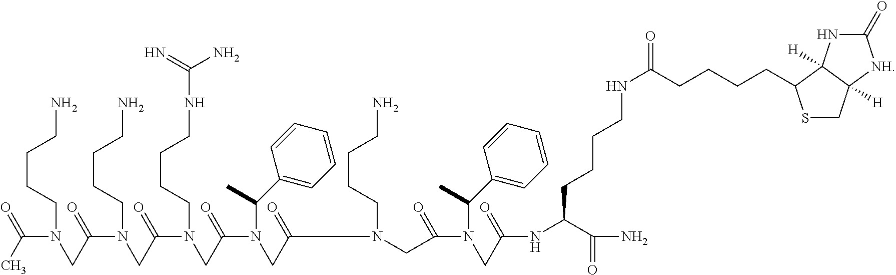

### Calculated Mass: 1290.70; Observed Mass: 1290.8.

### Peptoid Reagent III

The below peptoid reagent includes, but is not limited to SEQ ID NO: 231.

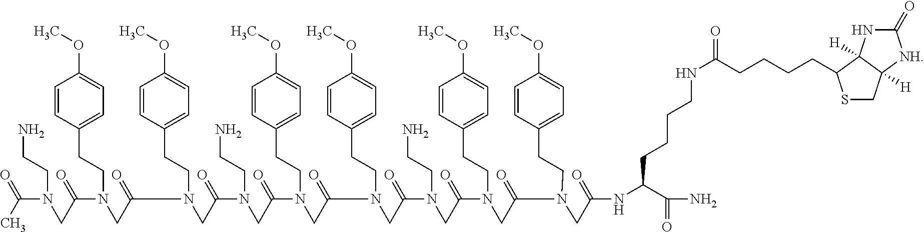

### Calculated Mass: 1861.30; Observed Mass: 1861.6.

### Peptoid Reagent IV

The below peptoid reagent includes, but is not limited to SEQ ID NO: 232.

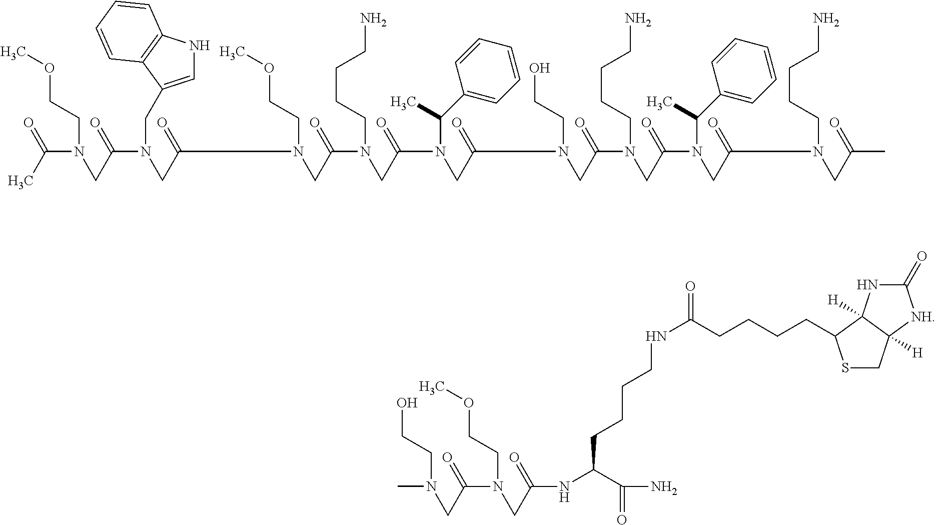

### Peptoid Reagent V

The below peptoid reagent includes, but is not limited to SEQ ID NO: 233.

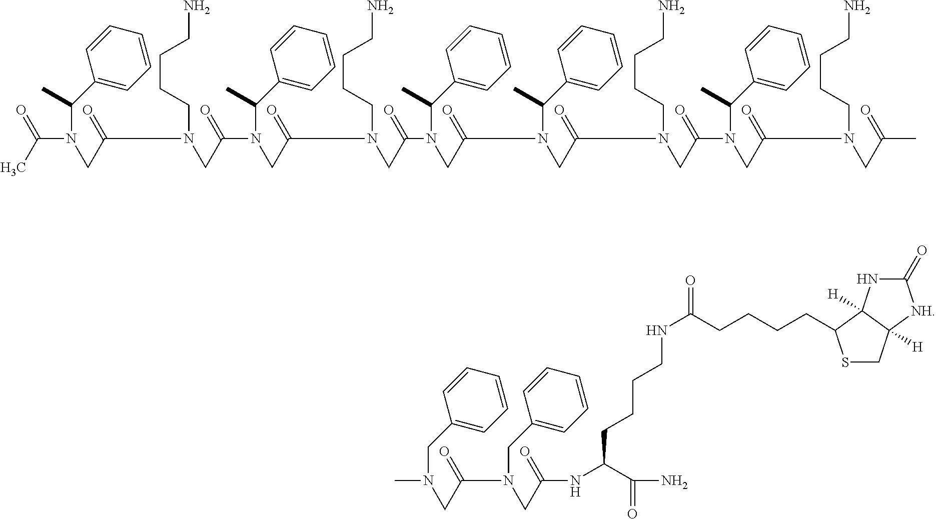

### Peptoid Reagent VI

The below peptoid reagent includes, but is not limited to SEQ ID NO: 234.

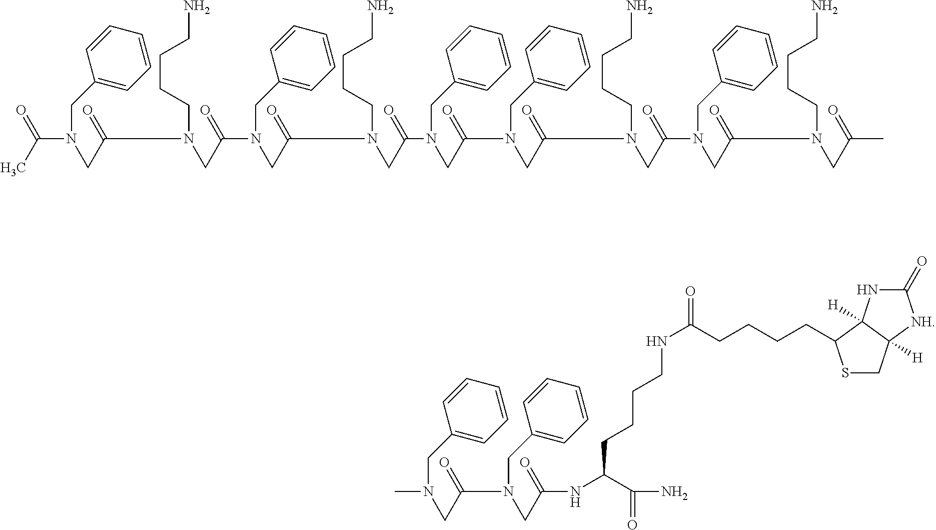

### Calculated Mass: 1956.49; Observed Mass: 1956.2.

### Peptoid Reagent VII

The below peptoid reagent includes, but is not limited to SEQ ID NO: 235.

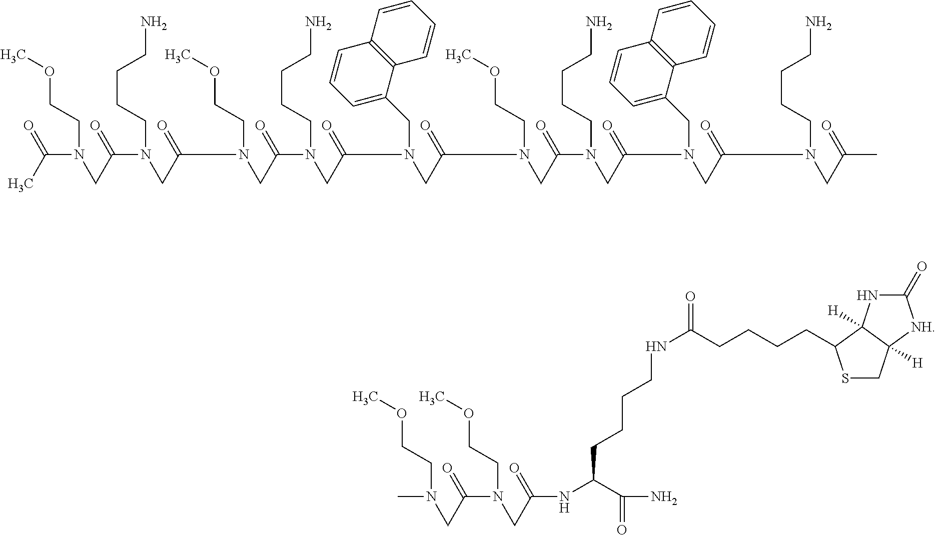

### Calculated Mass: 1896.39; Observed Mass: 1896.4.

### Peptoid Reagent VIII

The below peptoid reagent includes, but is not limited to SEQ ID NO: 236.

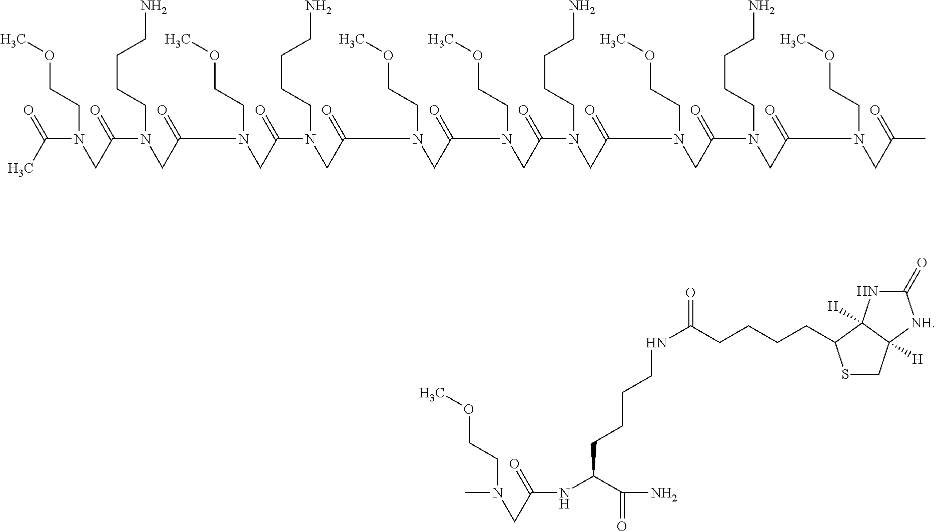

### Calculated Mass: 1732.18; Observed Mass: 1732.4.

### Peptoid Reagent IX

The below peptoid reagent includes, but is not limited to SEQ ID NO: 237.

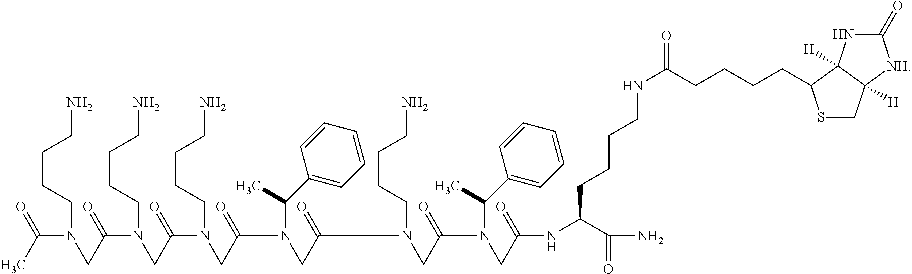

### Calculated Mass: 1248.65; Observed Mass: 1248.4.

### Peptoid Reagent X

The below peptoid reagent includes, but is not limited to SEQ ID NO: 238.

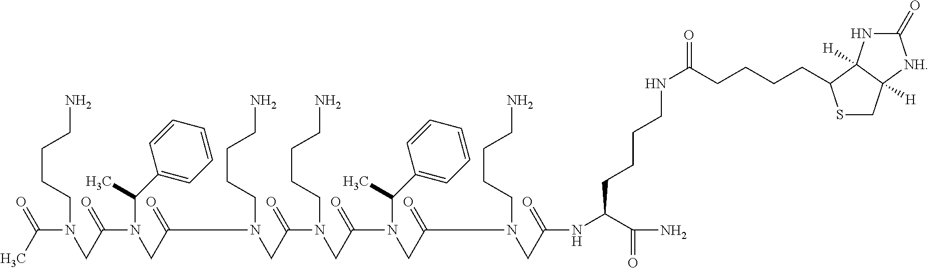

### Calculated Mass: 1248.65; Observed Mass: 1248.4.

### Peptoid Reagent XIa and XIb

The below peptoid reagents, XIa and XIb, comprise SEQ ID NO: 239.

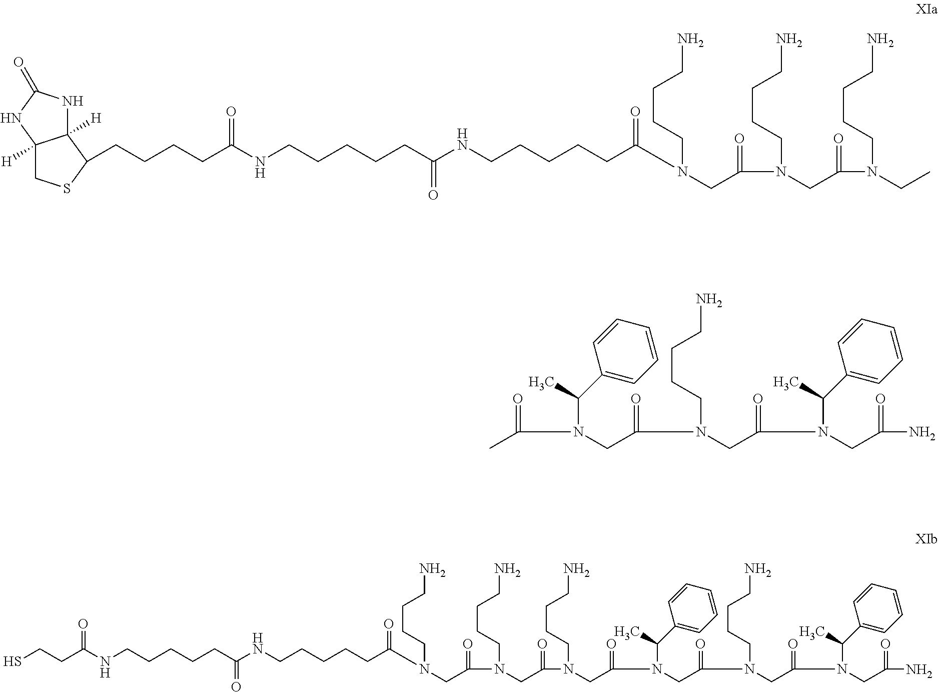

### XIa: Calculated Mass: 1304.76; Observed Mass: 1304.6.

### XIb: Calculated Mass: 1166.59; Observed Mass: 1166.2.

### Peptoid Reagent XIIa and XIIb

The below peptoid reagents of formula XIIa and XIIb comprise SEQ ID NO: 240.

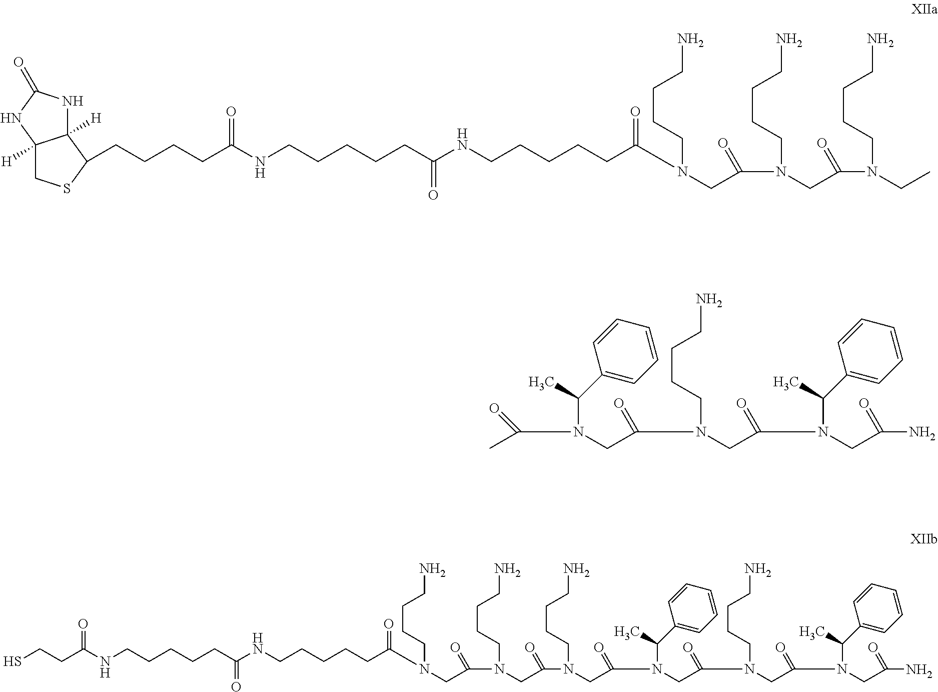

### XIIa: Calculated Mass: 1276.71; Observed Mass: 1276.6.

### Peptoid Reagent XIII

The below peptoid reagent includes, but is not limited to SEQ ID NO: 241.

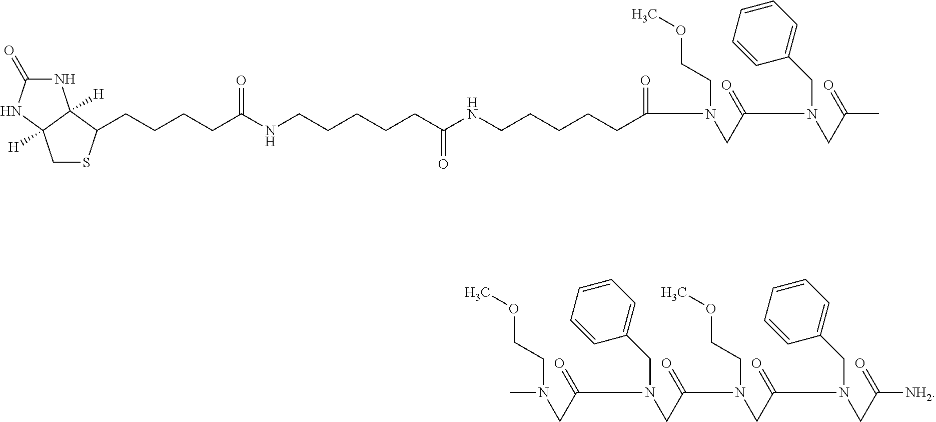

### Calculated Mass: 1256.58; Observed Mass: 1256.6.

### Example 9

### PSR1 and PrP23-30 Demonstrate Capture of Aβ Superior to β-sheet blockers

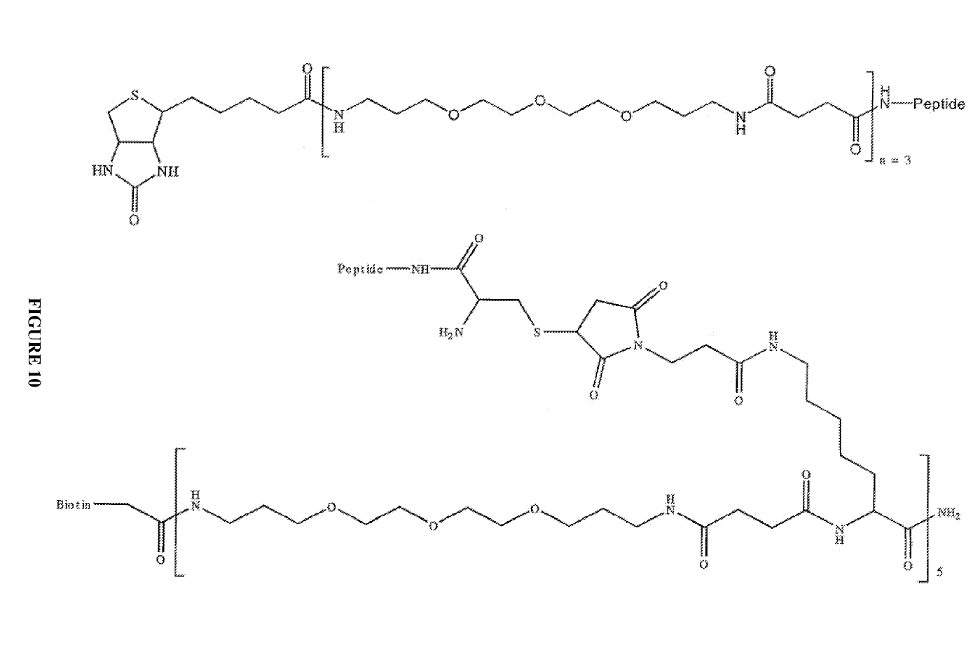

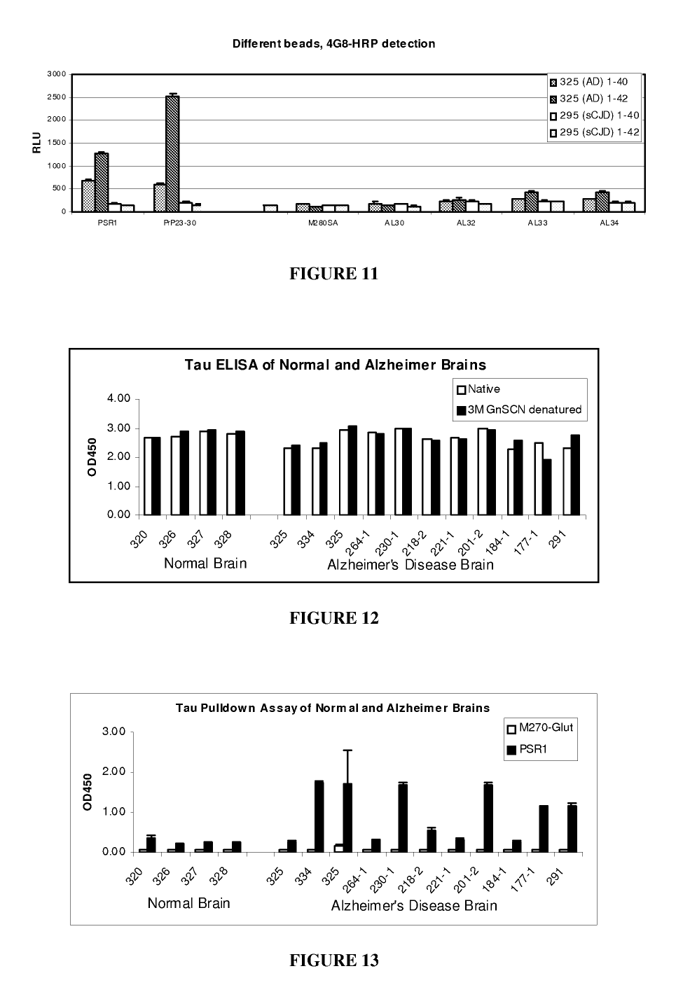

β-sheet breakers are small molecules which are thought to disrupt the process of Aβ fibrillization by binding to regions of Aβ which mediate aggregation. This example demonstrates that PSR1 capture of Aβ is superior to capture by β-sheet breakers (see FIG. 11).

Capture of Aβ using PSR1, PrP23-30, the M280 SA bead alone, and β-sheet breakers AL30, AL32, AL33, and AL34 (structures of which are detailed in the below table) and detection via ELISA with a 4G8-HRP reagent was conducted using the methods described in Example 2.

| | |
|---|---|
| AL30 | biotin-AHX-D(FFVLK)-CONH2 |
| (Aβ20-16 | (SEQ ID NO: 252) |
| reverse | [uses D-amino acids instead of L-amino |
| sequence) | acids] |
| AL32 | biotin-AHX-FFVLK-CONH2 |
| (Aβ20-16) | (SEQ ID NO: 253) |
| | [uses normal L-amino acids] |
| AL33 | biotin-AHX-KLVFF-NmeL-CONH2 |
| (Aβ16-20- | (SEQ ID NO: 254) [NmeL is N- |
| NmeL) | methylated lysine, a “standard” amino |
| | acid modification available from most |
| | custom peptide synthesis companies] |
| AL34 | biotin-AHX-KLVFF-NmeL-AHX- |
| (Aβ(16-20- | KLVFF-NmeL-CONH2 |
| NmeL)₂) | (SEQ ID NO: 255) |
| | [dimer of the above] |

### Example 10

### PSR1 Captures Aggregated Total Tau

Total levels of aggregated tau (phosphorylated, non-phosphorylated, or hyperphosphorylated) are associated with Alzheimer's Disease. Hyperphosphorylated aggregated tau seems to have a particularly strong association with Alzheimer's Disease.

Tau hyperphosphorylation, caused by Aβ aggregation and oxidation stress, is believed to be involved in AD development (Formichi, P., et al. J. of Cellular Physiology. 208-1: 39-46, 2006). Hyperphosphorylated tau protein has high self-aggregation activity and forms paired helical filaments and neurofibrillary tangles which are found in the brains of neurodegenerative disease (Goedert, M. et al. Trends Neurosci 16: 460-465, 1993; Iqbal et al., J Neural Transm 53: 169-180, 1998). Tau phosphorylation at Thr 181 and 231 has been tested as AD biomarkers in CSF. The ratio of p-tau to Aβ₄₂ has a high diagnostic value for discriminating patients with AD from healthy controls and other dementias (Buerger et al. Arch Neurol 59: 1267-1272, 2002; Maddalena et al. Arch Neurol 60: 1202-1206, 2003).

This Example demonstrates that PSR1 captures AD-associated aggregated tau.

### Measurement of Total Tau Levels in Normal and Alzheimer's Disease Brains:

Normal human and Alzheimer's disease brain homogenates were either untreated (native) or treated with 3M GnSCN (denatured) for 1 hour at room temperature. 100 mL of 10% brain homogenate was aliquoted into each ELISA well (BioSource Tau immunoassay kit KHB0042/KHB0041). Total tau standards were diluted by Dilution solution with 0.006M GnSCN.

Each ELISA plate was covered and incubated at room temperature overnight. On the second day, the ELISA plate was washed 4 times with 400 uL per well of Diluted Wash Buffer and 100 uL of rabbit anti-Tau antibody was added to each well. The plate was incubated at room temperature for 1 hour and washed 4 times with 400 uL per well of Diluted Wash Buffer. 100 uL of working anti-rabbit-HRP was added to each well. The plate was then incubated at room temperature for 30 minutes and washed 4 times with 400 uL per well of Diluted Wash Buffer. Next, 100 uL of Stabilized Chromagen was added. The plate was then incubated at room temperature for 30 minutes in the dark. The reaction was stopped by adding 100 uL of Stop Solution to all wells and read at 450 nm.

Significant levels of tau were detected in both normal and Alzheimer's disease brains (FIG. 12). Unlike prions and Aβ aggregates, denaturation of tau had no significant effect on detectable tau levels suggesting that tau antibody binding epitopes are not conformationally altered in pathological isoforms of tau.

Pulldown with PSR1

400 mL of 10% normal or Alzheimer's disease brain homogenates was diluted with 100 uL of TBSTT for each pulldown reaction. 3 uL per reaction of M270-glutathione or PSR1 beads was plated in a 500 uL round bottom Corning plate. Reactions were incubated for 1 hour at 37° C. with 750 rpm shaking. The beads were then washed with TBST on a BioTek BLx405. After taking out the residual solution, 5 uL of 6M GnSCN was added and incubated with the beads for 1 hour at room temperature with 750 rpm shaking to dissociate the protein. 120 uL of H₂O was added to each well.

Next, the amount of tau captured by each pulldown reagent was quantitated. 50 uL per well of Standard Dilution buffer was added to a tau ELISA plate. The pulldown plate was placed on a magnetic stand for 2 minutes and 50 uL/well of solution was transferred on the tau ELISA plate (equivalent to 160 nL 10% BH/well). Tau standards were diluted by Dilution solution with 0.12M GnSCN. The ELISA plate was covered and incubated at room temperature overnight. On the second day, the ELISA plate was washed 4 times with 400 uL per well of Diluted Wash Buffer. 100 uL of rabbit anti-Tau antibody was added to each well. The plate was incubated at room temperature for 1 hour and washed 4 times with 400 uL per well of Diluted Wash Buffer. 100 uL of working anti-rabbit-HRP was added to each well. The plate was incubated at room temperature for 30 minutes and washed 4 times with 400 uL per well of Diluted Wash Buffer. 100 uL of Stabilized Chromagen was added. The plate was incubated at room temperature for 30 minutes in the dark. The reaction was stopped by adding 100 uL of Stop Solution to all wells and read at 450 nm.

In most Alzheimer's disease brain samples, PSR1 bound significantly more tau than in normal brain samples, suggesting that PSR1 binds selectively to aggregated tau (FIG. 13). Very little tau was detected in control glutathione bead pulldown samples.

Detected tau levels are quantitated in the below Table 8.

| | | ELISA | | Pulldown | | |
|---|---|---|---|---|---|---|
| ug/mL | | Native | 3M GnSCN | M270-Glut | PSR1 | % binding |
| Normal | 320 | 1.398 | 1.389 | −0.001 | 0.080 | 5.739 |
| Normal | 326 | 1.409 | 1.512 | −0.003 | 0.041 | 2.733 |
| Normal | 327 | 1.515 | 1.534 | −0.002 | 0.048 | 3.121 |
| Normal | 328 | 1.487 | 1.519 | −0.002 | 0.045 | 2.959 |
| AD | 325 | 1.216 | 1.251 | −0.003 | 0.058 | 4.608 |
| AD | 334 | 1.190 | 1.300 | −0.002 | 0.476 | 36.620 |
| AD | 325 | 1.535 | 1.612 | 0.027 | 0.469 | 29.074 |
| AD | 264-1 | 1.498 | 1.477 | −0.002 | 0.066 | 4.442 |
| AD | 230-1 | 1.559 | 1.556 | 0.000 | 0.454 | 29.152 |
| AD | 218-2 | 1.387 | 1.358 | −0.003 | 0.132 | 9.717 |
| AD | 221-1 | 1.399 | 1.371 | −0.002 | 0.067 | 4.871 |
| AD | 201-2 | 1.556 | 1.535 | −0.003 | 0.459 | 29.928 |
| AD | 184-1 | 1.185 | 1.344 | −0.003 | 0.056 | 4.166 |
| AD | 177-1 | 1.295 | 1.004 | −0.001 | 0.295 | 29.370 |
| AD | 291 | 1.201 | 1.446 | −0.003 | 0.302 | 20.911 |

### Example 11

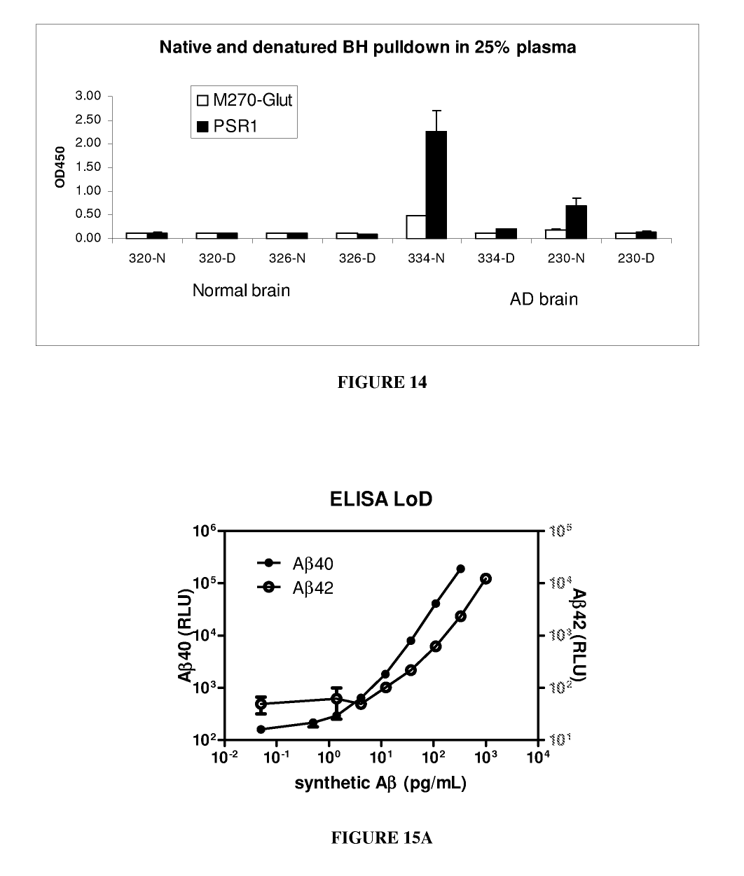

### Dissociated Aggregated Tau is No Longer Pulled Down by PSR1 Beads (FIG. 14)

Normal or Alzheimer's brain homogenates were incubated with or without 5M GnSCN for 1 hr at room temperature. 400 mL of 10% normal or Alzheimer's disease brain homogenates was diluted in 200 uL of 25% human plasma—TBSTT for each pulldown reaction.

3 uL per reaction of M270-glutathione or PSR1 beads was plated in a 500 uL round bottom Corning plate. Reactions were incubated for 1 hour at 37° C. with 750 rpm shaking. The beads were then washed with TBST on a BioTek BLx405. After taking out the residual solution, 10 uL of 6M GnSCN was added and incubated with the beads for 1 hour at room temperature with 750 rpm shaking to dissociate the protein. 120 uL of H₂O was added to each well.

The pulldown plate was placed on a magnetic stand for 2 minutes and 50 uL/well of solution was transferred on the tau ELISA plate (equivalent to 160 nL 10% BH/well). The ELISA plate was covered and incubated at room temperature overnight. On the second day, the ELISA plate was washed 4 times with 400 uL per well of Diluted Wash Buffer. 100 uL of rabbit anti-Tau antibody was added to each well. The plate was incubated at room temperature for 1 hour and washed 4 times with 400 uL per well of Diluted Wash Buffer. 100 uL of working anti-rabbit-HRP was added to each well. The plate was incubated at room temperature for 30 minutes and washed 4 times with 400 uL per well of Diluted Wash Buffer. 100 uL of Stabilized Chromagen was added. The plate was incubated at room temperature for 30 minutes in the dark. The reaction was stopped by adding 100 uL of Stop Solution to all wells and read at 450 nm.

Dissociated aggregated tau was no longer bound by PSR1 (see FIG. 14). To date we have shown that insoluble ordered protein aggregates of three different proteins; Prion, Aβ, and Tau bind PSR1 and other PCSB reagents., Denaturation of these aggregates results their solubility and eliminates their binding to PSR1 and other PCSB reagents. This observation further supports the presence of an interacting domain that is characteristic of ordered amyloid structures independent of the protein amino acid sequence.

TABLE 9

| | | | | Glut | | | | | | PSR1 | | | | | |
|---|---|---|---|---|---|---|---|---|---|---|---|---|---|---|---|
| | | | | | | | M270-Glut | sd | % cv | | | | PSR1 | sd | % cv |
| NBH | 320 | Native | 320-N | 0.12 | 0.12 | 0.12 | 0.12 | 0.00 | 1.44 | 0.12 | 0.12 | 0.13 | 0.12 | 0.00 | 3.62 |
| | | Denatured | 320-D | 0.11 | 0.11 | 0.11 | 0.11 | 0.00 | 2.35 | 0.11 | 0.11 | 0.11 | 0.11 | 0.00 | 1.36 |
| | 326 | Native | 326-N | 0.12 | 0.12 | 0.12 | 0.12 | 0.00 | 1.49 | 0.11 | 0.11 | 0.11 | 0.11 | 0.00 | 3.22 |
| | | Denatured | 326-D | 0.11 | 0.10 | 0.10 | 0.10 | 0.01 | 5.49 | 0.10 | 0.10 | 0.10 | 0.10 | 0.00 | 2.54 |
| ADBH | 334 | Native | 334-N | 0.48 | 0.48 | 0.48 | 0.48 | 0.00 | 0.74 | 1.88 | 2.74 | 2.15 | 2.26 | 0.44 | 19.39 |
| | | Denatured | 334-D | 0.12 | 0.11 | 0.11 | 0.11 | 0.01 | 8.91 | 0.20 | 0.20 | 0.21 | 0.20 | 0.01 | 2.49 |
| | 230-1 | Native | 230-N | 0.18 | 0.17 | 0.20 | 0.18 | 0.02 | 9.13 | 0.65 | 0.87 | 0.54 | 0.69 | 0.17 | 24.09 |
| | | Denatured | 230-D | 0.12 | 0.11 | 0.11 | 0.12 | 0.01 | 4.59 | 0.12 | 0.16 | 0.13 | 0.13 | 0.02 | 14.65 |

### Example 12

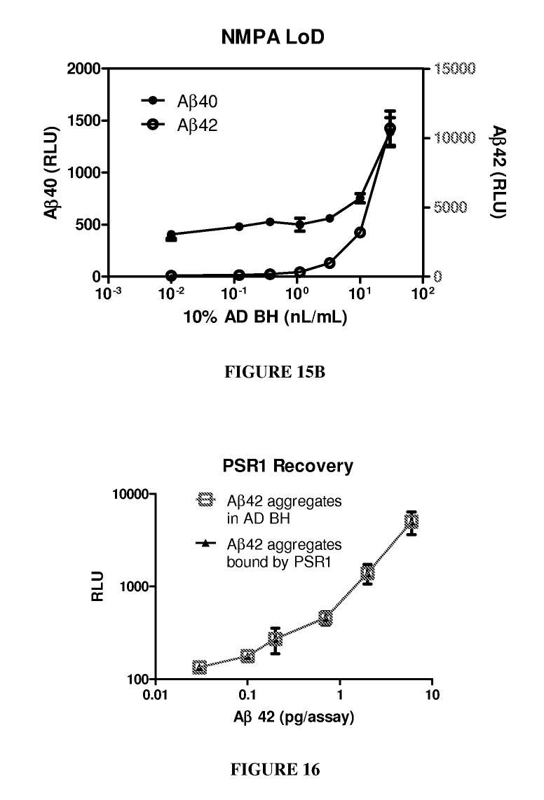

### PSR1 Bead Pulldown Assay Limit of Detection (LOD) for AD Aβ Aggregates in Human CSF (FIG. 15)

The Limit of Detection (LOD) of the PSR1 bead pulldown assay was evaluated by determining the lowest quantity of aggregated Aβ detectable over background.

The sensitivity of a sandwich ELISA for detecting monomeric soluble Aβ was evaluated using the MesoScale Discovery (MSD) technology (FIG. 15A). Standard synthetic Aβ40 or 42 was captured by mAb specific to the C-terminus of Aβ and detected by mAb 4G8. The limit of detection (LOD) at a signal/noise ratio of 2 was 1.6 pg/mL for Aβ40 and 12 pg/mL for Aβ42.

PSR1 sensitivity of detecting Aβ40 and 42 aggregates in Alzheimer's Disease (AD) brain homogenate (BH) was measured (FIG. 15B). 10% AD BH was spiked into 200 uL of pooled normal human CSF, and captured by 3 uL PSR1 beads. The captured Aβ was dissociated by 0.1N NaOH at 80° C. into monomeric Aβ and detected by sandwich MSD ELISA as described above. The limit of detection (LOD) at a signal/noise ratio of 2 was 1 pg/mL (11.4 mL of 10% AD BH spiked into 1 mL of CSF) for Aβ40, and 1 pg/mL (0.3 mL of 10% AD BH spiked into 1 mL of CSF) for Aβ42.

### Example 13

### PSR1 Recovery (Binding Efficiency) of Aβ42 Aggregates Spiked into Human Sera (FIG. 16)

The efficiency of recovery of Aβ aggregates by PSR1 was evaluating by comparing the amount of total aggregated Aβ42 in AD BH to that captured by PSR1 pulldown.

10% AD BH was spiked into 200 uL of diluted normal human sera (200-fold dilution in TBS), and captured by 3 uL PSR1 beads (FIG. 16, square). The captured Aβ42 aggregates were dissociated by 0.1N NaOH at 80° C. and detected by sandwich MSD ELISA as described in Example 12.

The same amount of AD BH not subject to PSR1 capture was denatured by 0.1 NaOH at 80° C. and total Aβ42 measured by sandwich ELISA as described in Example 12 (FIG. 16, triangle).

The concentration of Aβ was calculated from a standard curve using synthetic Aβ as described in Example 12.

The amount of Aβ42 aggregates captured by PSR1 was equal to the total Aβ42 aggregates in AD BH, indicating that PSR1 recovery is about 100% and that PSR1 is a highly efficient capture reagent.

### Example 14

### Detection of Aβ40 and 42 from Normal CSF by PSR1-bead pulldown assay

To monitor PSR1's ability to bind Aβ monomers in CSF from individuals without Alzheimer's disease, increasing concentrations of PSR1 (3, 9, 15 uL from 30 mg/mL stock) were added to 50 uL CSF Lot 410 or Lot 411 mixed with 50 uL of 2×TBSTT (Tris-buffer saline containing 1% Tween 20 and 1% Triton-X 100). The resulting mixture was incubated at 37° C. for 1 hr with constant shaking at 600 rpm. Unbound sample was removed by washing the beads six times with TBST (Tris-buffer saline containing 0.05% Tween 20). For each wash, after adding TBST the beads were collected using magnetic force, and TBST was removed. The bead-bound protein was dissociated by 0.1N NaOH at 80° C. for 30 min with 750 rpm constant shaking. 0.12M NaH2PO4/0.4% Tween 20 was used to neutralize the solution. The supernatant was transferred to ELISA plate to measure Aβ40 and 42. Readings were compared to ELISA of Aβ40 and 42 standard curves to quantification. ELISA was carried out according to MSD 96-Well MULTI_SPOT Human/Rodent 4G8 Aβ Triplex Ultra-Sensitive Assay (Meso Scale Discovery). The results in Table 10 show binding of Aβ40 and 42 to PSR1-bead with increasing bead amount in terms of pg/ml and percent of global. Minute binding of Aβ40 is apparent at all bead concentrations. The detected amount represents less than 1% of the total Aβ40 present in normal CSF. Binding of Aβ42 is apparent and represents 1-5% of all Aβ42 present in normal CSF.

The observed binding may indicate the presence of oligomeric Aβ in normal CSF. Alternatively these finding may suggest that PSR1 can bind monomeric Aβ at low affinity.

TABLE 10
Human CSF Lot 410

| | |
|---|---|
| Aβ 40 2020 pg/ml | Aβ 42 212.6 pg/ml |

| Binding to PSR1 | |
|---|---|
| Aβ 40 | Aβ 42 |

| PSR1 | pg/ml | % of global | pg/ml | % of global |
|---|---|---|---|---|
| 3 uL | 10.28 | 0.51 | 3.28 | 1.54 |
| 9 uL | 15.4 | 0.76 | 7.1 | 3.34 |
| 15 uL | 17.3 | 0.86 | 11.8 | 5.55 |

Human CSF Lot 411

| | |
|---|---|
| Aβ 40 1950 pg/ml | Aβ 42 195 pg/ml |

| Binding to PSR1 | |
|---|---|
| Aβ 40 | Aβ 42 |

| PSR1 | pg/ml | % of global | pg/ml | % of global |
|---|---|---|---|---|
| 3 uL | 5.25 | 0.27 | — | — |
| 9 uL | 13.57 | 0.70 | 4.9 | 2.52 |
| 15 uL | 15.3 | 0.78 | 7.5 | 3.86 |

### Example 15

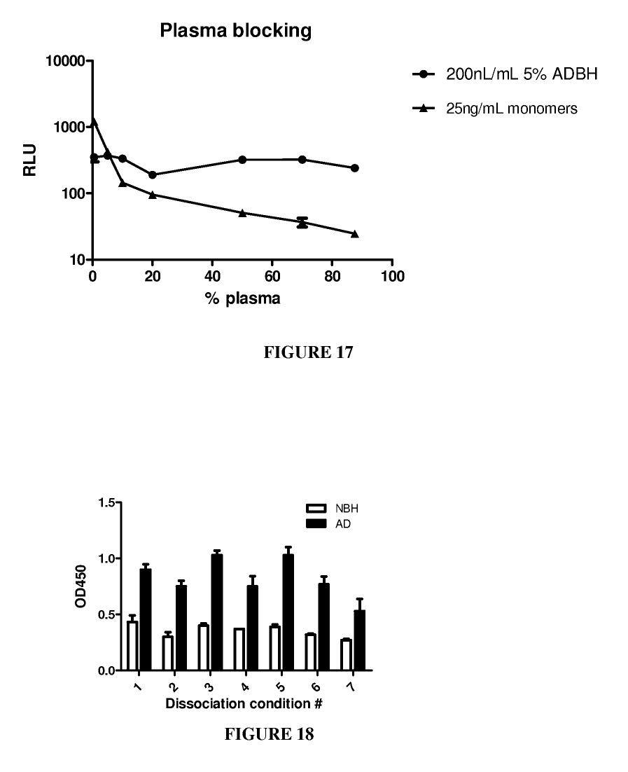

### Binding of Aβ42 Monomers to PSR1 can be Blocked by Low Concentrations of Plasma (FIG. 17)

The affinity of PSR1 binding to monomeric Aβ and aggregated Aβ was evaluated by examining the effect of increasing concentrations of plasma.

To test blocking of monomeric Aβ binding to PSR1, beads were incubated with a large excess of monomeric Aβ42 (25 ng/ml) in the presence of increasing concentration of plasma (FIG. 17, triangles). As the concentration of plasma increased, the amount of monomer binding decreased. Twenty percent plasma inhibited more than 90% of PSR1 binding to monomers.

To test blocking of aggregated Aβ binding to PSR1, beads were incubated with 200 mL/mL 5% AD brain homogenate in the presence of increasing concentrations of plasma (FIG. 17, circles). In contrast to monomeric Aβ, binding of aggregated Aβ was not affected by up to 85% plasma.

These findings suggest that monomeric Aβ binds PSR1 with low affinity which can be blocked by other proteins while aggregated Aβ binds PSR1 with higher affinity.

TABLE 11

| | |
|---|---|
| 200 nL/mL 5% | 25 ng/mL Abeta |
| ADBH | monomers 1-42 |

| % plasma | Ave | SD | Ave | SD |
|---|---|---|---|---|
| 0.5 | 349.2 | 48.3 | 1202.3 | 63.6 |
| 5 | 369.7 | 23.5 | 423.7 | 13.9 |
| 10 | 334.6 | 27.9 | 145.3 | 14.9 |
| 20 | 190.2 | 21.8 | 95.5 | 8.7 |
| 50 | 321.4 | 35.4 | 50.9 | 3.8 |
| 70 | 323.3 | 13.9 | 36.8 | 5.6 |
| 87.5 | 241.1 | 21.4 | 24.6 | 1.9 |

### Example 16

### HCl Dissociates the Binding of Aggregated Tau Protein and PSR1-Bead (FIG. 18)

To conduct efficient immuno-detection of PSR1-bound aggregate, the aggregate should be eluted and dissociated into protein monomers compatible with ELISA. The chaotropic severities of dissociation will depend on the physicochemical properties of the aggregate. To optimize dissociation of aggregated tau from PSR1, the following acidic conditions were tested:

| # | Dissociation conditions |
|---|---|
| 1 | 6M GdnSCN/Room temperature/30 min |
| 2 | 0.1N HCl/glycine with 150 mM NaCl at pH 2.3/Room temperature/ |
| | 30 min |
| 3 | 0.25N HCl/Room temperature/30 min |
| 4 | 0.1N HCl/glycine with 150 mM NaCl at pH 2.3/50° C./30 min |
| 5 | 0.25N HCl/50° C./30 min |
| 6 | 0.1N HCl/glycine with 150 mM NaCl at pH 2.3/80° C./30 min |
| 7 | 0.25N HCl/80° C./30 min |

Brain homogenates (BH) from normal or Alzheimer's disease (AD) were spiked into TBSTT (Tris-buffer saline containing 1% Tween 20 and 1% Triton-X 100). 100 uL of each sample was mixed with 3 uL of PSR1-beads (30 mg/mL). The resulting mixture was incubated at 37° C. for 1 hr with constant shaking at 750 rpm. Unbound sample materials were removed by washing the beads six times with TBST (Tris-buffer saline containing 0.05% Tween 20). For each wash, after adding TBST the beads were collected using magnetic force and TBST was removed. The binding of aggregated Tau and beads was dissociated by different conditions (indicated in the table) with 750 rpm constant shaking. The dissociation reactions were on three separate plates for three different temperatures. 6M GdnSCN was diluted by H₂O and HCl was neutralized by NaOH to pH 7.5. The supernatants were transferred to same ELISA plate on magnetic force. Tau was quantified by Human Tau (Total) ELISA (Biosource).

The results are depicted in FIG. 18. The results show that 0.25N HCl at room temperature (RT) and 50° C. dissociates the binding of aggregated Tau from AD BH with PSR1-bead. These dissociation conditions have the same efficiency as 6M GdnSCN at RT, which is the standard since it dissociates the aggregated proteins without damage to the proteins. Dissociation by 0.25N HCl at 80° C. eliminated Tau signal on the ELISA, most likely due to the damage of Tau protein. 0.1N HCl-glycine with 150 mM NaCl at pH 2.3 failed to achieve the same dissociation efficiency as 6M GdnSCN. For normal BH, the signals were at the same levels for all dissociation conditions. Condition 3 (0.25N HCl at room temperature for 30 min with 750 rpm shaking) is a preferred dissociation condition for aggregated Tau from PSR1 beads.

### Example 17

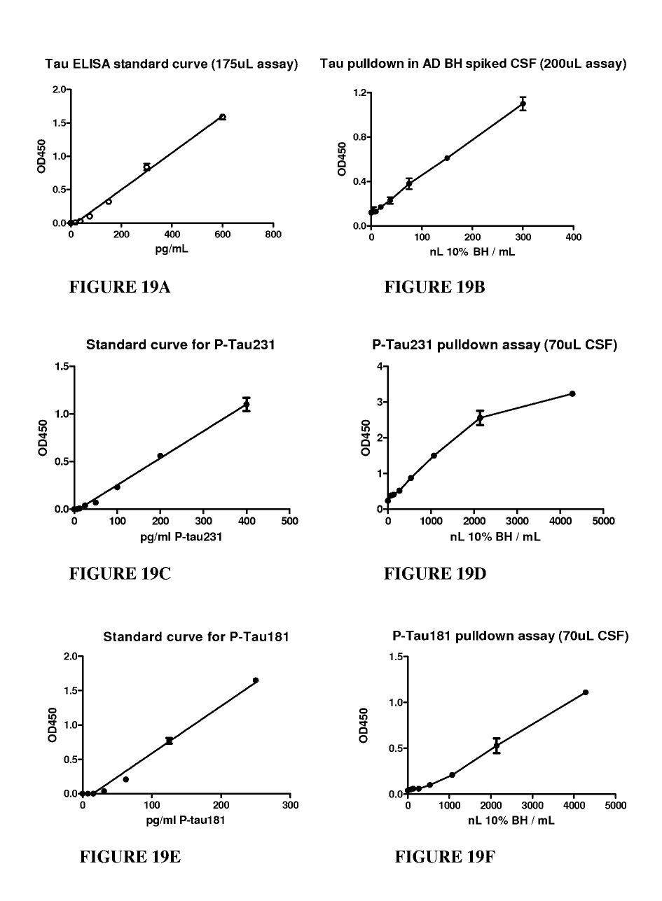

### PSR1 Bead Pulldown Assay Limits of Detection for Total Tau, P-Tau231, and P-Tau181 in Normal Human CSF Spiked with AD BH (FIG. 19A-F)

### Limit of Detection for Total Tau

Alzheimer's disease brain homogenate (AD BH) was spiked into normal human CSF. 200 uL of each sample was mixed with 50 uL of 5×TBSTT (Tris-buffer saline containing 1% Tween 20 and 1% Triton-X 100) and 12 uL of PSR1-beads (30 mg/mL). The resulting mixture was incubated at 37° C. for 1 hr with constant shaking at 550 rpm. Unbound sample materials were removed by washing the beads six times with TBST (Tris-buffer saline containing 0.05% Tween 20). For each wash, after adding TBST the beads were collected using magnetic force, and TBST was removed. The bead bound aggregated Tau was dissociated by 0.25N HCl at room temperature for 30 min with 750 rpm constant shaking. 0.25N NaOH was used to neutralize the solution. The supernatant was transferred onto INNOTEST hTAU Ag ELISA Plate on magnetic force. INNOTEST hTAU Ag ELISA was modified to 175 uL per reaction in order to composite the ending sample volume from PSR1-bead pulldown assay.

The assay limit of detection was determined using a ratio of S/N (signal vs. assay background) equal or greater than 2. ELISA assay background was considered to be the signal for buffer only. Pulldown assay background was considered to be the signal for CSF without AD BH spiking. The limit of detection for 175 uL INNOTEST hTAU Ag ELISA was 0.032 fmol per assay or 0.32 μM. The limit of detection for the Tau PSR1 bead pulldown assay was 0.038 fmol per assay or 0.19 μM.

### Limit of Detection for P-Tau231

Alzheimer's disease brain homogenate (AD BH) was spiked into normal human CSF. 70 uL of each sample was mixed with 30 uL of 3.3×TBSTT (Tris-buffer saline containing 1% Tween 20 and 1% Triton-X 100) and 3 uL of PSR1-beads (30 mg/mL). The resulting mixture was incubated at 37° C. for 1 hr with constant shaking at 750 rpm. Unbound sample materials were removed by washing the beads six times with TBST (Tris-buffer saline containing 0.05% Tween 20). For each wash, after adding TBST the beads were collected using magnetic force, and TBST was removed. The bead bound aggregated Tau was dissociated by 0.25N HCl at room temperature for 30 min with 750 rpm constant shaking. 0.25N NaOH was used to neutralize the solution. The supernatant was transferred to Human Tau [pT231] ELISA Plate (Biosource) on magnetic force.

The assay limit of detection was determined using a ratio of S/N (signal vs. assay background) equal or greater than 2. ELISA assay background was considered to be the signal for buffer only. Pulldown assay background was considered to be the signal for CSF without AD BH spiking. The limit of detection for Human Tau [pT231] ELISA was 0.09 fmol per assay or 1.72 pM. Limit of detection for Tau [pT231] pulldown assay was 0.20 fmol per assay or 2.71 pM.

### Limit of Detection for P-Tau181

Alzheimer's disease brain homogenate (AD BH) was spiked into normal human CSF. 70 uL of each sample was mixed with 30 uL of 3.3×TBSTT (Tris-buffer saline containing 1% Tween 20 and 1% Triton-X 100) and 3 uL of PSR1-beads (30 mg/mL). The resulting mixture was incubated at 37° C. for 1 hr with constant shaking at 750 rpm. Unbound sample materials were removed by washing the beads six times with TBST (Tris-buffer saline containing 0.05% Tween 20). For each wash, after adding TBST the beads were collected using magnetic force, and TBST was removed. The bead bound aggregated Tau was dissociated by 0.25N HCl at room temperature for 30 min with 750 rpm constant shaking. 0.25N NaOH was used to neutralize the solution. The supernatant was transferred to INNOTEST PHOSPHO-TAU_{(181P)} ELISA Plate on magnetic force.

Assay limit detection was determined by using ratio of S/N (signal vs. assay background) equal or greater than 2. ELISA assay background was considered to be the signal for buffer only. Pulldown assay background was considered to be the signal for CSF without AD BH spiking. The limit of detection for INNOTEST PHOSPHO-TAU_{(181P)} ELISA was 0.04 fmol per assay or 0.54 pM. The limit of detection for Tau [pT231] pulldown assay was 0.03 fmol per assay or 0.44 pM.

What is claimed is:

1. A method for detecting the presence of a non-prion pathogenic conformer comprising the steps of:
contacting a sample suspected of containing said non-prion pathogenic conformer with a pathogenic conformer-specific binding reagent under conditions that allow binding of said reagent to said non-prion pathogenic conformer, if present, to form a complex; and
detecting said non-prion pathogenic conformer, if any, in said sample by its binding to said pathogenic conformer-specific binding reagent;
wherein said pathogenic conformer-specific binding reagent is derived from a prion protein fragment and interacts preferentially with a pathogenic prion protein.

2. The method of claim 1, wherein said non-prion pathogenic conformer is a conformer associated with an amyloid disease.

3. The method of claim 2, wherein said amyloid disease is selected from the group consisting of a systemic amyloidosis, tauopathy, and synucleinopathy.

4. The method of claim 1, wherein said non-prion pathogenic conformer is a conformer associated with a disease selected from the group consisting of: Alzheimer's disease, ALS, immunoglobulin-related diseases, serum amyloid A-related diseases, and diabetes type II.

5. The method of claim 1, wherein said non-prion pathogenic conformer is an Alzheimer's disease conformer.

6. The method of claim 5, wherein said Alzheimer's disease conformer is an amyloid-beta (Aβ) protein.

7. The method of claim 5, wherein said Alzheimer's disease conformer is a tau protein.

8. The method of claim 6, wherein said pathogenic conformer-specific binding reagent is derived from compounds selected from the group consisting of: PrP₁₉₋₃₀ (SEQ ID NO: 242), PrP₂₃₋₃₀ (SEQ ID NO: 243), PrP₁₀₀₋₁₁₁ (SEQ ID NO: 244), PrP₁₀₁₋₁₁₀ (SEQ ID NO: 245), PrP₁₅₄₋₁₆₅ (SEQ ID NO: 246), PrP₂₂₆₋₂₃₇ (SEQ ID NO: 247), SEQ ID NO:14, SEQ ID NO: 50, SEQ ID NO: 68,

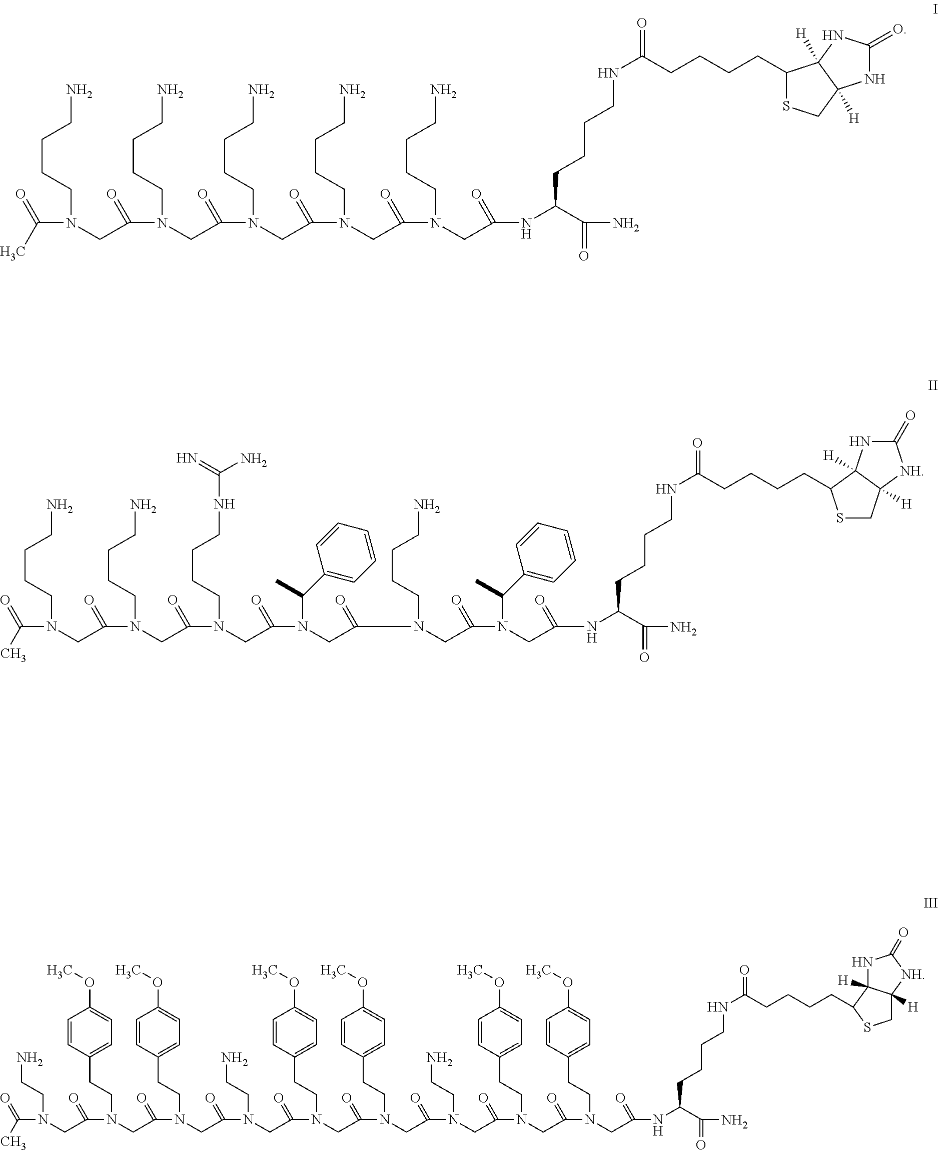

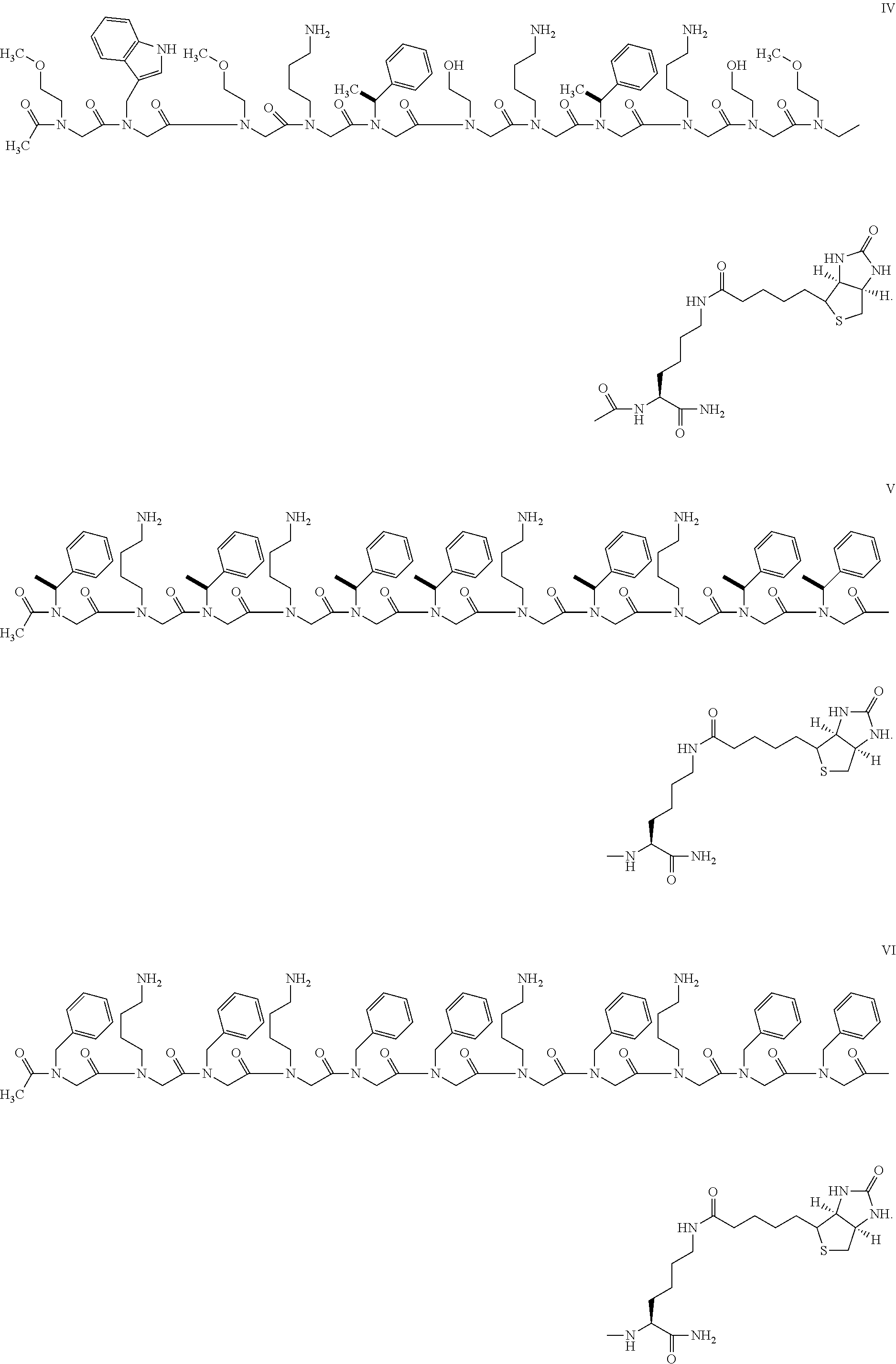

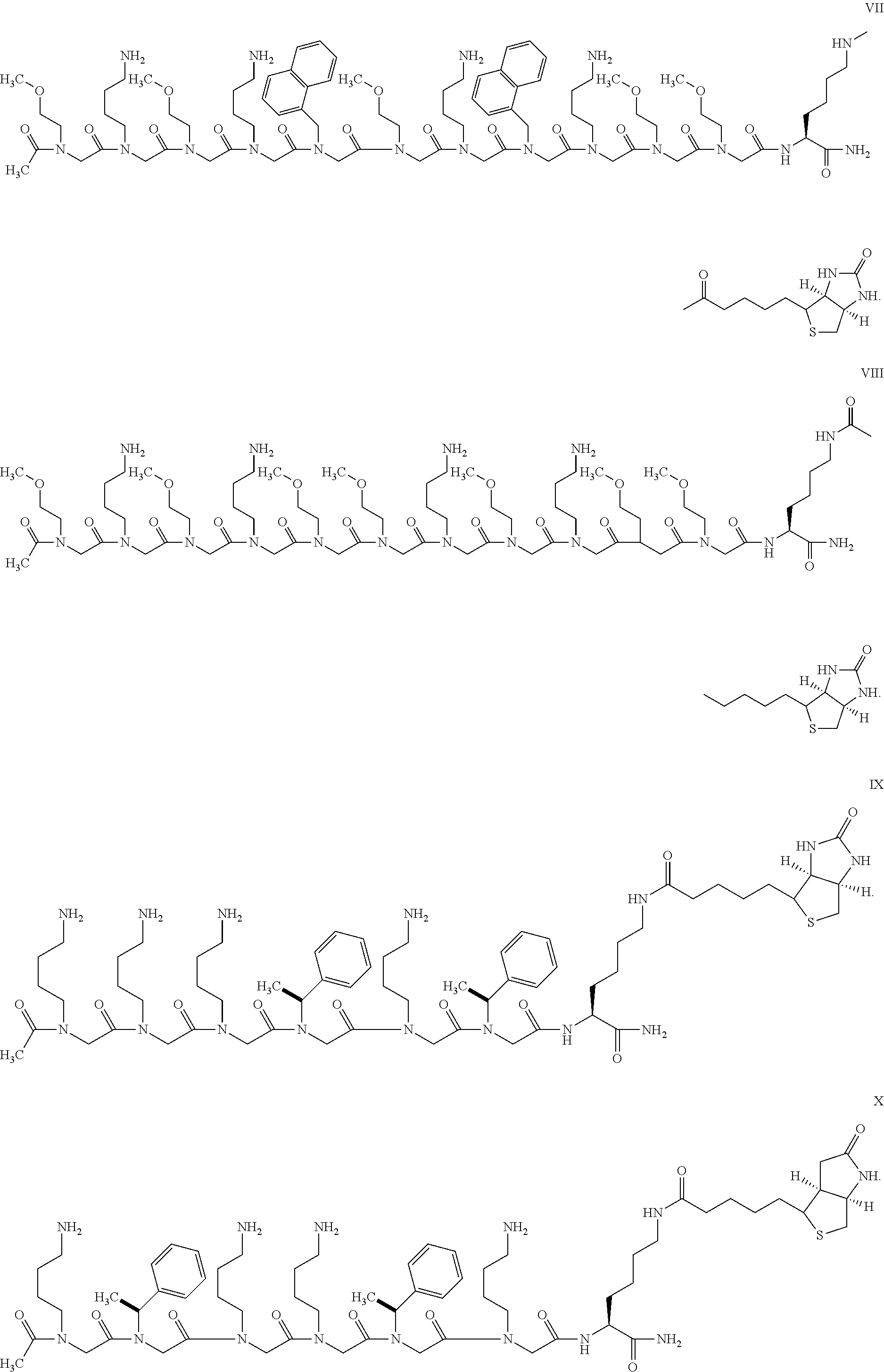

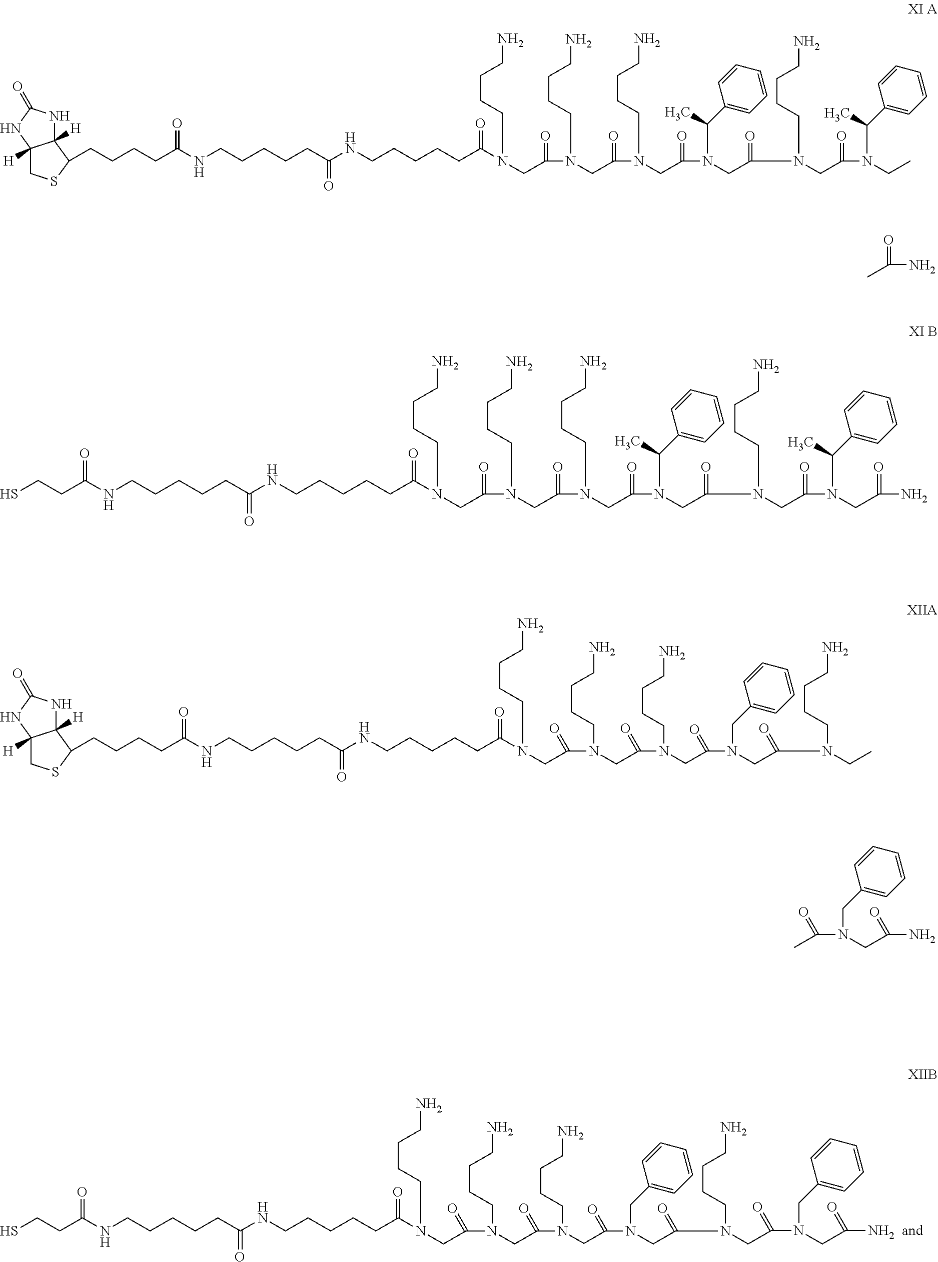

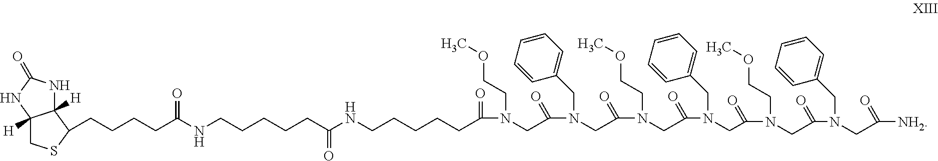

9. The method of claim 1, wherein said sample is selected from the group consisting of: organs, whole blood, blood fractions, blood components, plasma, platelets, serum, cerebrospinal fluid (CSF), brain tissue, nervous system tissue, muscle tissue, bone marrow, urine, tears, non-nervous system tissue, biopsies and necropsies.

10. The method of claim 1, wherein said sample comprises plasma or cerebrospinal fluid.

11. The method of claim 1, wherein said prion protein fragment is selected from the group of peptides consisting of PrP₁₉₋₃₀ (SEQ ID NO: 242), PrP₂₃₋₃₀ (SEQ ID NO: 243), PrP₁₀₀₋₁₁₁ (SEQ ID NO: 244), PrP₁₀₁₋₁₁₀ (SEQ ID NO: 245), PrP₁₅₄₋₁₆₅ (SEQ ID NO: 246), PrP₂₂₆₋₂₃₇ (SEQ ID NO: 247), SEQ ID NO:14, SEQ ID NO: 50 and SEQ ID NO: 68.

12. The method of claim 1, wherein said prion protein fragment is selected from the group consisting of: PrP₁₉₋₃₀ (SEQ ID NO: 242), PrP₂₃₋₃₀ (SEQ ID NO: 243), PrP₁₀₀₋₁₁₁ (SEQ ID NO:244), PrP₁₀₁₋₁₁₀ (SEQ ID NO: 245), SEQ ID NO:14, SEQ ID NO: 50 and SEQ ID NO: 68.

13. The method of claim 1, wherein said pathogenic conformer-specific binding reagent comprises an amino acid sequence selected from the group consisting of: SEQ ID NO: 242, SEQ ID NO: 243, SEQ ID NO: 244, SEQ ID NO: 245, SEQ ID NO: 246, SEQ ID NO: 247, SEQ ID NO:14, SEQ ID NO: 50 and SEQ ID NO: 68.

14. The method of claim 1, wherein said pathogenic conformer-specific binding reagent comprises an amino acid sequence selected from the group consisting of SEQ ID NO: 242, SEQ ID NO: 243, SEQ ID NO:244, SEQ ID NO: 245, SEQ ID NO:14, SEQ ID NO: 50 and SEQ ID NO: 68.

15. The method of claim 1, wherein the pathogenic conformer specific binding reagent comprises a peptoid reagent selected from the group consisting of:
(a) SEQ ID NO: 229, 230, 231, 232, 233, 234, 235, 236, 237, 238, 239, 240, or 241;
(b) SEQ ID NO: 229, 230, 232, 233, 237, 238, 239, or 240;
(c) SEQ ID NO: 230, 237, 238, 239, or 240;
(d) SEQ ID NO: 240;
(e) 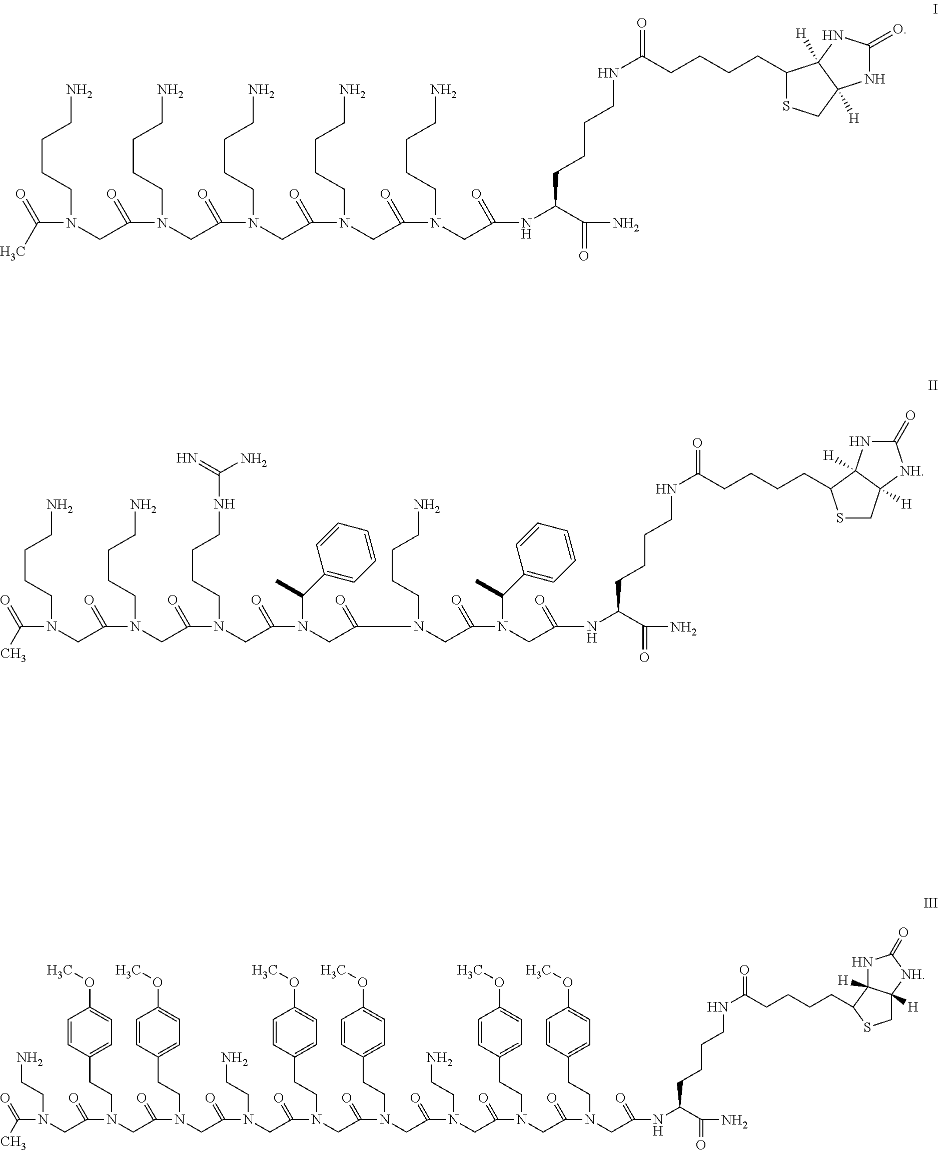 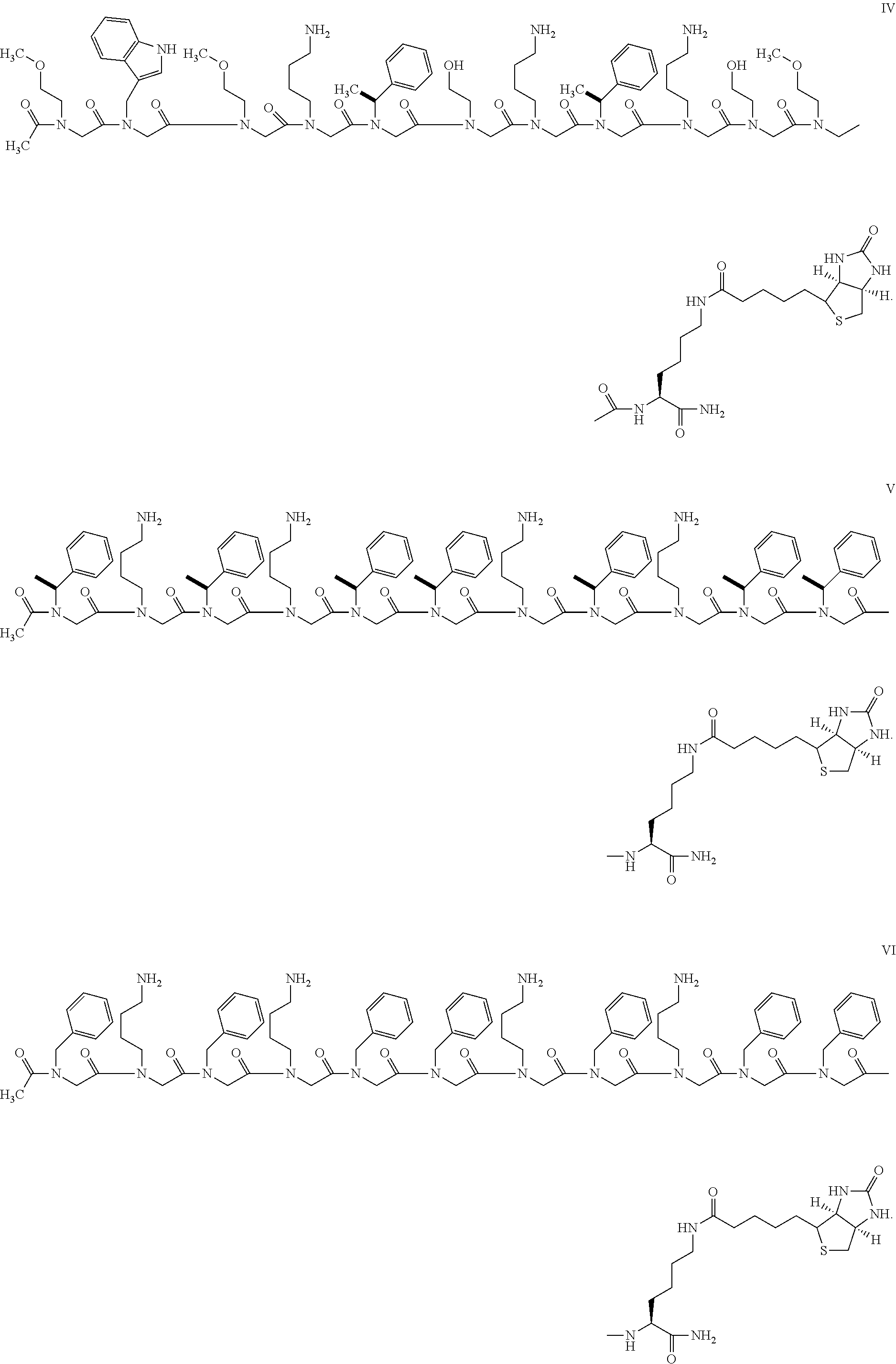 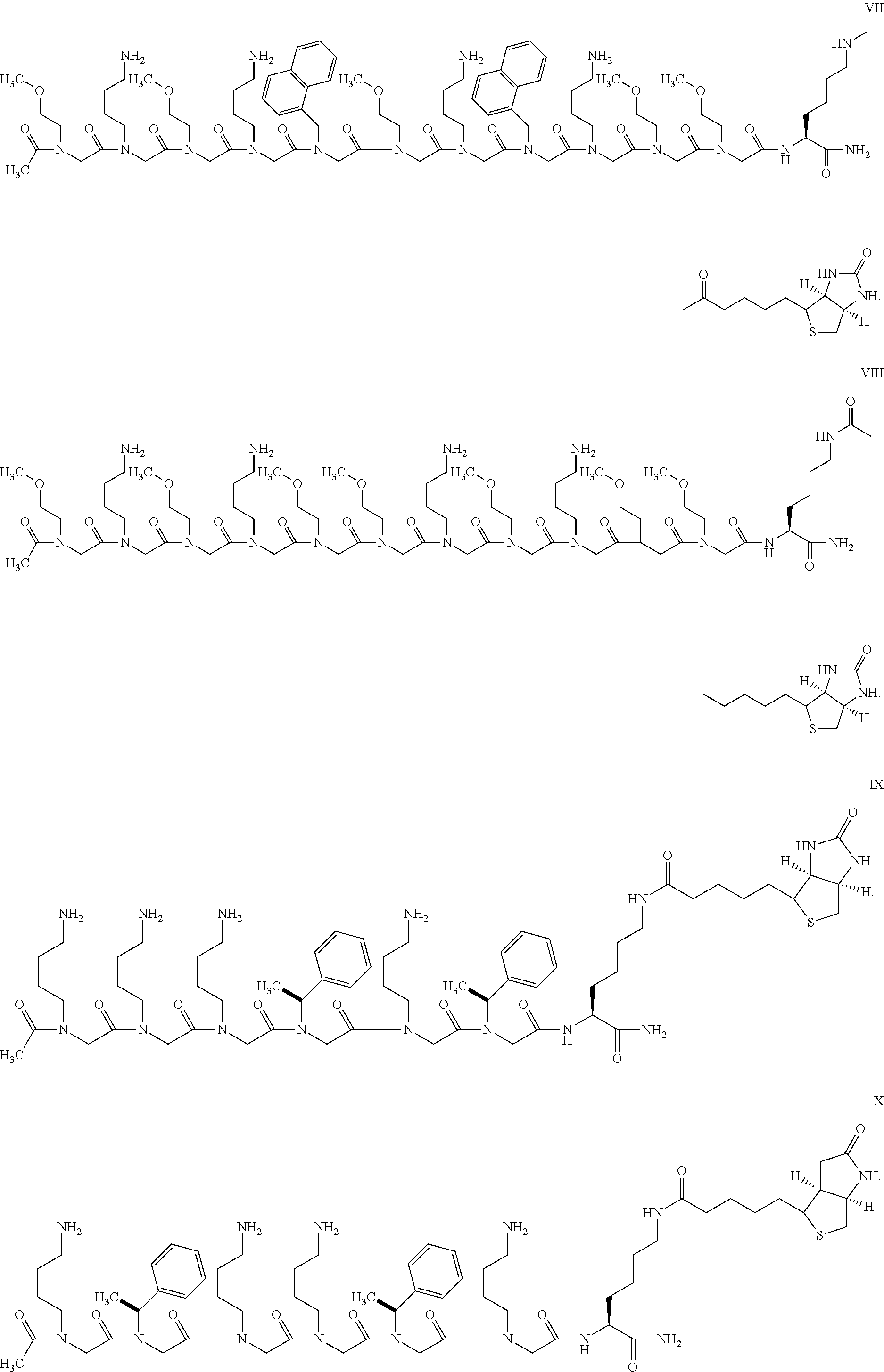 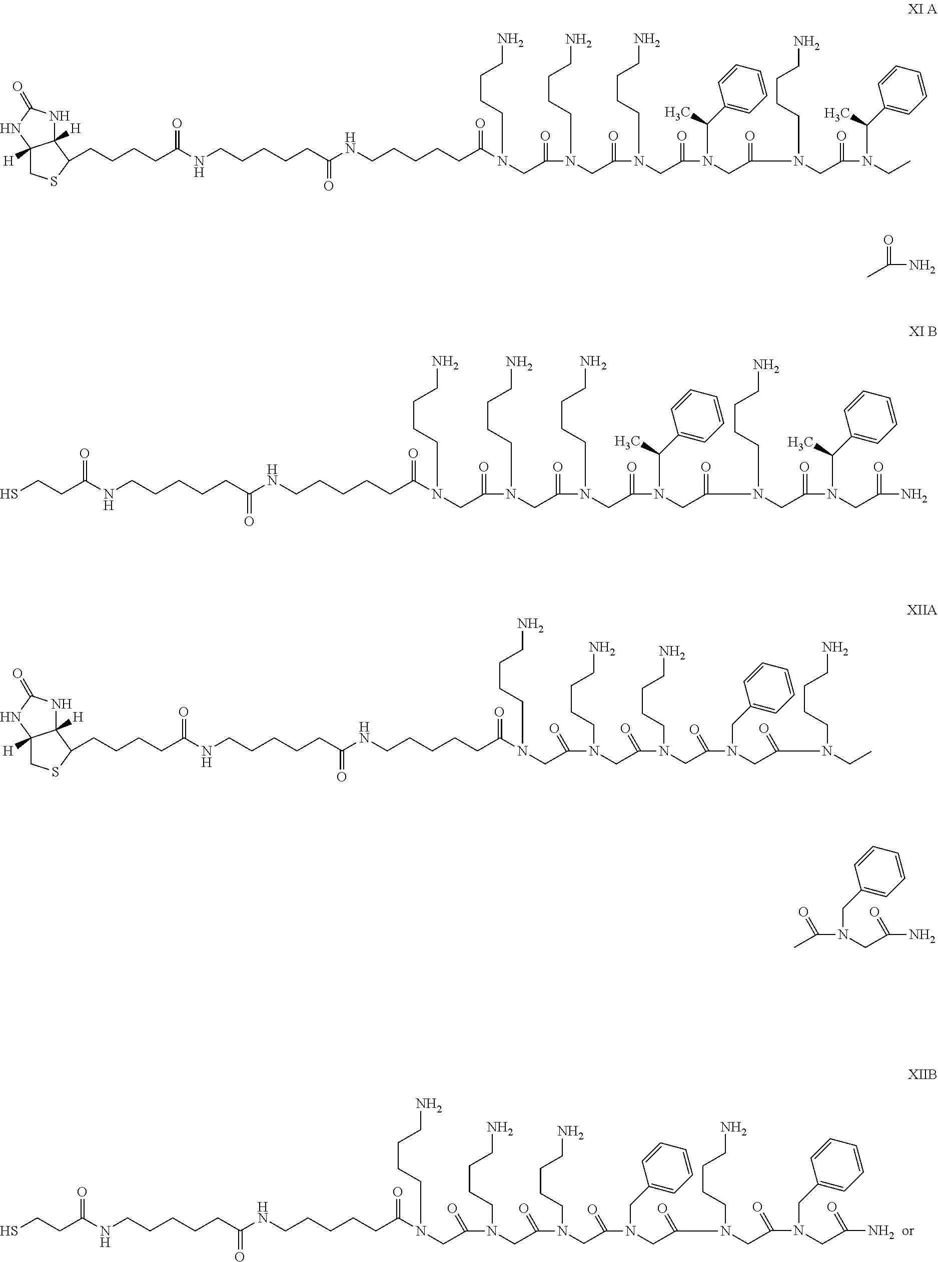 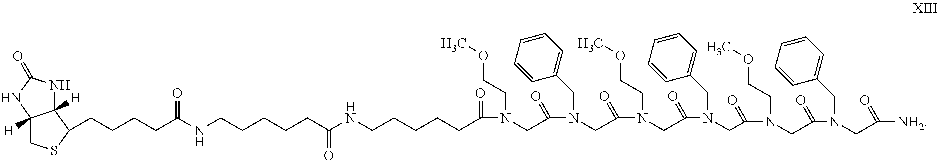
and
(f)

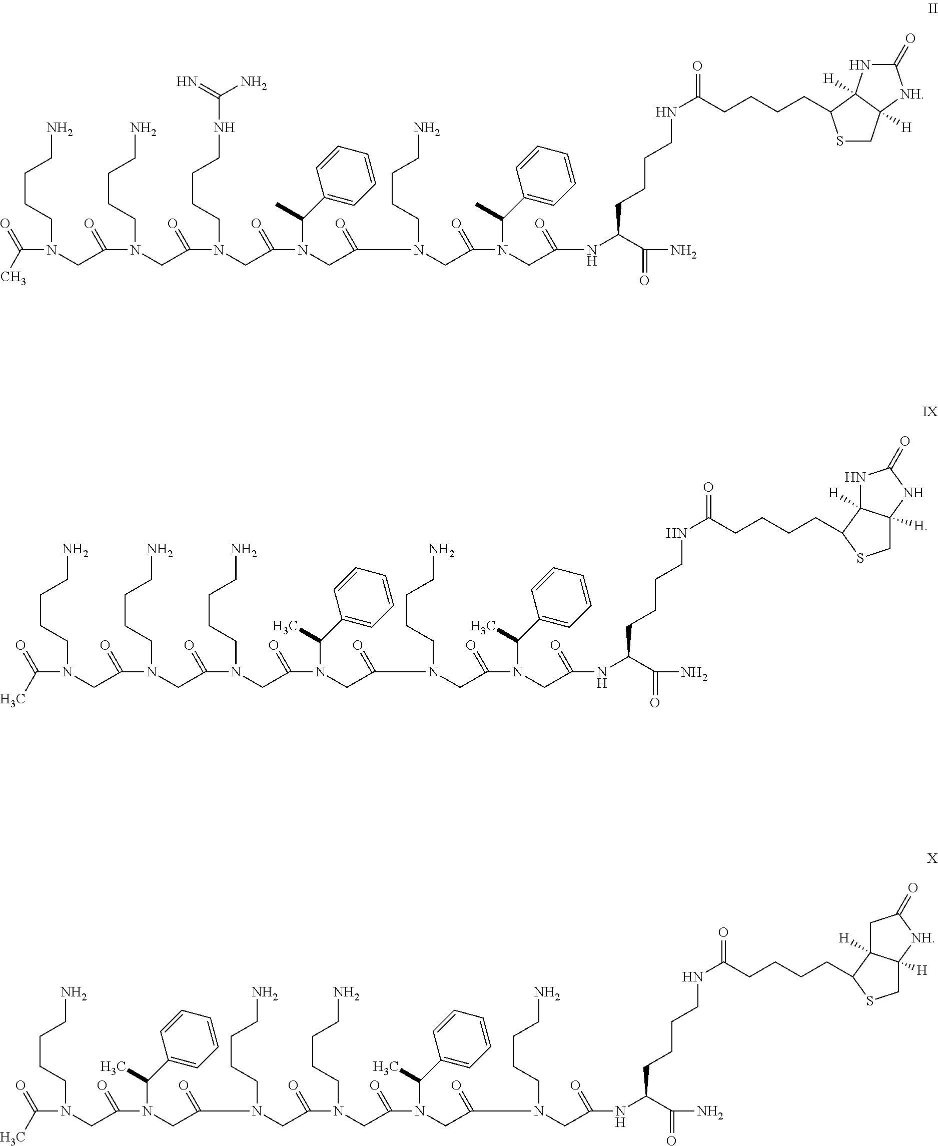

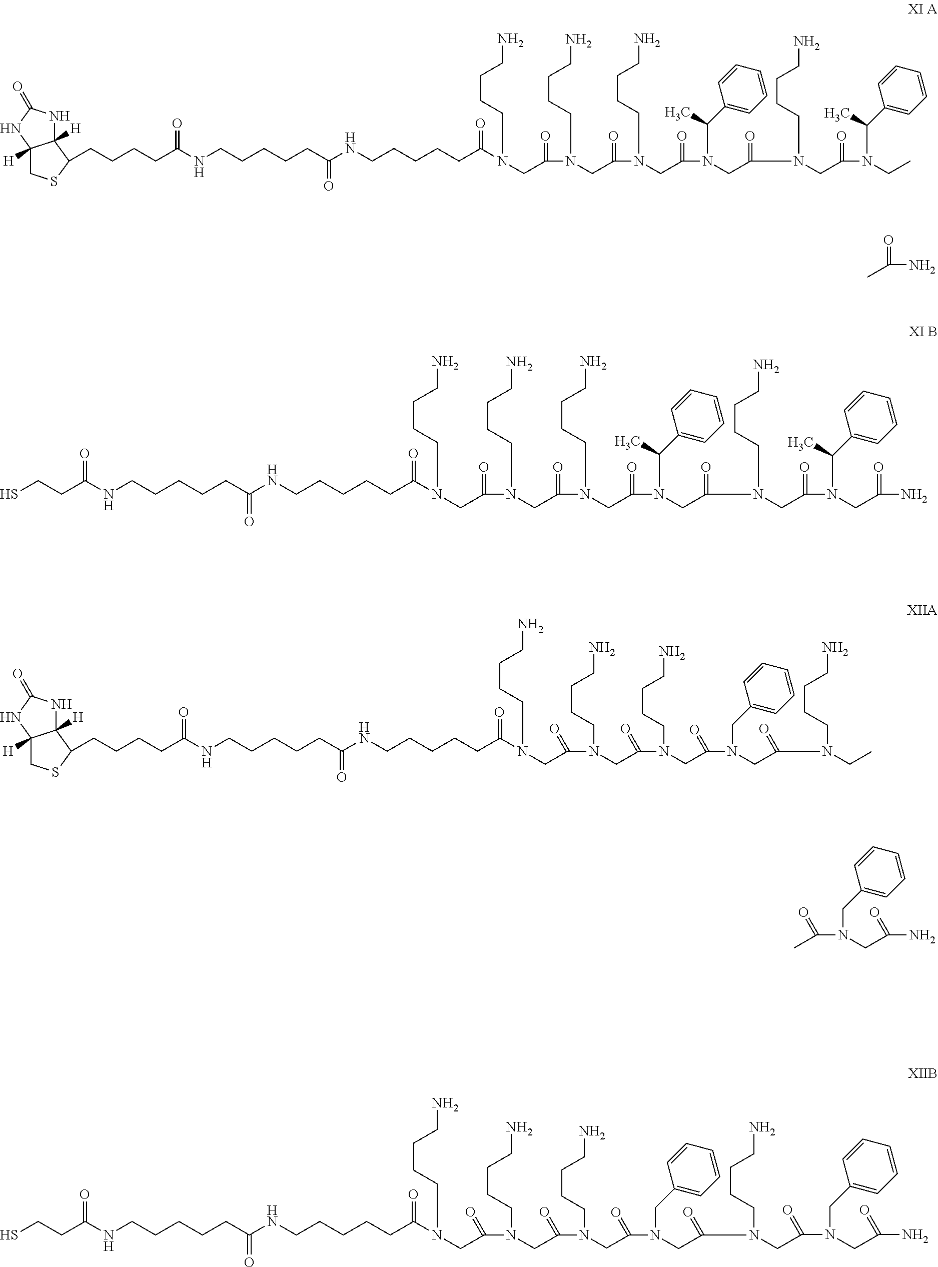

16. The method of claim 1, wherein said pathogenic conformer-specific binding reagent has a net charge of at least positive three at physiological pH.

17. The method of claim 16, wherein said reagent has a net charge of least positive four at physiological pH.

18. The method of claim 1, wherein said pathogenic conformer-specific binding reagent is detectably labeled.

19. The method of claim 18, wherein said reagent is detectably labeled with biotin.

20. The method of claim 1, wherein said reagent is attached to a solid support.

21. The method of claim 20, wherein said solid support is selected from the group consisting of: nitrocellulose, polystyrene latex, polyvinyl fluoride, diazotized paper, nylon membranes, activated beads, and magnetically responsive beads.

22. A method for detecting the presence of a non-prion pathogenic conformer comprising the steps of:
contacting a sample suspected of containing said non-prion pathogenic conformer with a pathogenic conformer-specific binding reagent under conditions that allow the binding of said reagent to said non-prion pathogenic conformer, if present, to form a complex;
contacting said complex with a conformational disease protein-specific binding reagent under conditions that allow binding; and
detecting the presence of said non-prion pathogenic conformer, if any, in said sample by its binding to said conformational disease protein-specific binding reagent;
wherein said pathogenic conformer-specific binding reagent is derived from a prion protein fragment and interacts preferentially with a pathogenic prion protein.

23. The method of claim 22, wherein said method further comprises removing unbound sample materials after forming said complex.

24. The method of claim 22, wherein said conformational disease protein-specific binding reagent is a labeled antibody.

25. The method of claim 22, wherein said non-prion pathogenic conformer is an Aβ protein and said conformational disease protein-specific binding reagent is an anti-Aβ antibody.

26. A method for detecting the presence of a non-prion pathogenic conformer comprising the steps of:
contacting a sample suspected of containing said non-prion pathogenic conformer with a pathogenic conformer-specific binding reagent under conditions that allow the binding of said reagent to said non-prion pathogenic conformer, if present, to form a first complex;
removing unbound sample materials;
dissociating said non-prion pathogenic conformer from said first complex thereby providing dissociated non-prion pathogenic conformer;
contacting said dissociated non-prion pathogenic conformer with a first conformational disease protein-specific binding reagent under conditions that allow binding to form a second complex; and
detecting the presence of said non-prion pathogenic conformer, if any, in the sample by detecting the formation of said second complex;
wherein said pathogenic conformer-specific binding reagent is derived from a prion protein fragment and interacts preferentially with a pathogenic prion protein.

27. The method of claim 26, wherein the formation of said second complex is detected using a detectably labeled second conformational disease protein-specific binding reagent.

28. The method of claim 26, wherein said pathogenic conformer-specific reagent is coupled to a solid support.

29. The method of claim 26, wherein said first conformational disease protein-specific binding reagent is coupled to a solid support.

30. The method of claim 26, wherein said non-prion pathogenic conformer is dissociated from said first complex by exposing said first complex to guanidine thiocyanate.

31. The method of claim 26, wherein said non-prion pathogenic conformer is dissociated from said first complex by exposing said complex to high pH or low pH.

32. The method of claim 31 further comprising the step of neutralizing the high pH or the low pH after the dissociating.

33. The method of claim 26, wherein said non-prion pathogenic conformer is an Aβ protein and said conformational disease protein-specific binding reagent is an anti-Aβ antibody.

34. The method of claim 33, wherein said Aβ protein is dissociated from said first complex by exposing said complex to a high pH condition.

35. The method of claim 34, wherein said high pH condition is about 0.1 N NaOH at about 80° C.

36. A method for detecting the presence of a non-prion pathogenic conformer comprising the steps of:
contacting a sample suspected of containing said non-prion pathogenic conformer with a first pathogenic conformer-specific binding reagent under conditions that allow binding of said first reagent to said non-prion pathogenic conformer, if present, to form a first complex;
contacting said sample suspected of containing said non-prion pathogenic conformer with a second pathogenic conformer-specific binding reagent under conditions that allow binding of said second reagent to said non-prion pathogenic conformer in said first complex, wherein said second reagent comprises a detectable label; and
detecting said non-prion pathogenic conformer, if any, in a sample by its binding to said second reagent;
wherein said first and second pathogenic conformer-specific binding reagents are derived from a prion protein fragment and interact preferentially with a pathogenic prion protein.

37. A method for detecting the presence of a non-prion pathogenic conformer comprising the steps of:
(a) contacting a sample suspected of containing said non-prion pathogenic conformer with a conformational disease protein-specific binding reagent under conditions that allow binding of said reagent to said non-prion pathogenic conformer, if present, to form a complex;
(b) removing unbound sample materials;
(c) contacting said complex with a pathogenic conformer-specific binding reagent under conditions that allow the binding of said pathogenic conformer-specific binding reagent to said non-prion pathogenic conformer, wherein said pathogenic conformer-specific binding reagent comprises a detectable label; and
detecting said non-prion pathogenic conformer, if any, in said sample by its binding to said pathogenic conformer-specific binding reagent;
wherein said pathogenic conformer-specific binding reagent is derived from a prion protein fragment and interacts preferentially with a pathogenic prion protein.

38. A method for detecting the presence of a non-prion pathogenic conformer comprising the steps of:
providing a solid support comprising a pathogenic conformer-specific binding reagent;
combining said solid support with a detectably labeled ligand, wherein said pathogenic conformer-specific binding reagent's binding affinity to said detectably labeled ligand is weaker than said reagent's binding affinity to said non-prion pathogenic conformer;
combining a sample with said solid support under conditions which allow said non-prion pathogenic conformer, when present in said sample, to bind to said reagent and replace said ligand; and
detecting complexes formed between said reagent and said non-prion pathogenic conformer from said sample;
wherein said pathogenic conformer-specific binding reagent is derived from a prion protein fragment and with preferentially with a pathogenic prion protein.

39. A method for discriminating between a non-prion pathogenic conformer and a non-prion non-pathogenic conformer comprising the steps of:
contacting a sample suspected of containing said non-prion pathogenic conformer with a pathogenic conformer-specific binding reagent under conditions that allow binding of said reagent to said non-prion pathogenic conformer, if present, to form a complex; and
discriminating between said non-prion pathogenic conformer and said non-prion non-pathogenic conformer by binding of said pathogenic conformer to said reagent;
wherein said pathogenic conformer-specific binding reagent is derived from a prion protein fragment and interacts preferentially with a pathogenic prion protein.

40. A method for diagnosing a non-prion conformational disease comprising the steps of:
contacting a sample suspected of containing a non-prion pathogenic conformer with a pathogenic conformer-specific binding reagent under conditions that allow binding of said reagent to said non-prion pathogenic conformer, if present, to form a complex;
detecting said non-prion pathogenic conformer, if any, in said sample by its binding to said reagent; and
diagnosing a conformational disease if said non-prion pathogenic conformer is detected;
wherein said pathogenic conformer-specific binding reagent is derived from a prion protein fragment and interacts preferentially with a pathogenic prion protein.

41. A method for detecting the presence of a non-prion pathogenic conformer comprising the steps of:
contacting a sample suspected of containing said non-prion pathogenic conformer with a pathogenic conformer-specific binding reagent under conditions that allow binding of said reagent to said non-prion pathogenic conformer, if present, to form a complex; and
detecting said non-prion pathogenic conformer, if any, in said sample by its binding to said pathogenic conformer-specific binding reagent;
wherein said pathogenic conformer-specific binding reagent comprises a peptoid region comprising SEQ ID NO: 229, 230, 231, 232, 233, 234, 235, 236, 237, 238, 239, 240, or 241.

42. A method for detecting the presence of a non-prion pathogenic conformer comprising the steps of:
contacting a sample suspected of containing said non-prion pathogenic conformer with a pathogenic conformer-specific binding reagent under conditions that allow binding of said reagent to said non-prion pathogenic conformer, if present, to form a complex; and
detecting said non-prion pathogenic conformer, if any, in said sample by its binding to said pathogenic conformer-specific binding reagent;
wherein said pathogenic conformer-specific binding reagent is selected from:

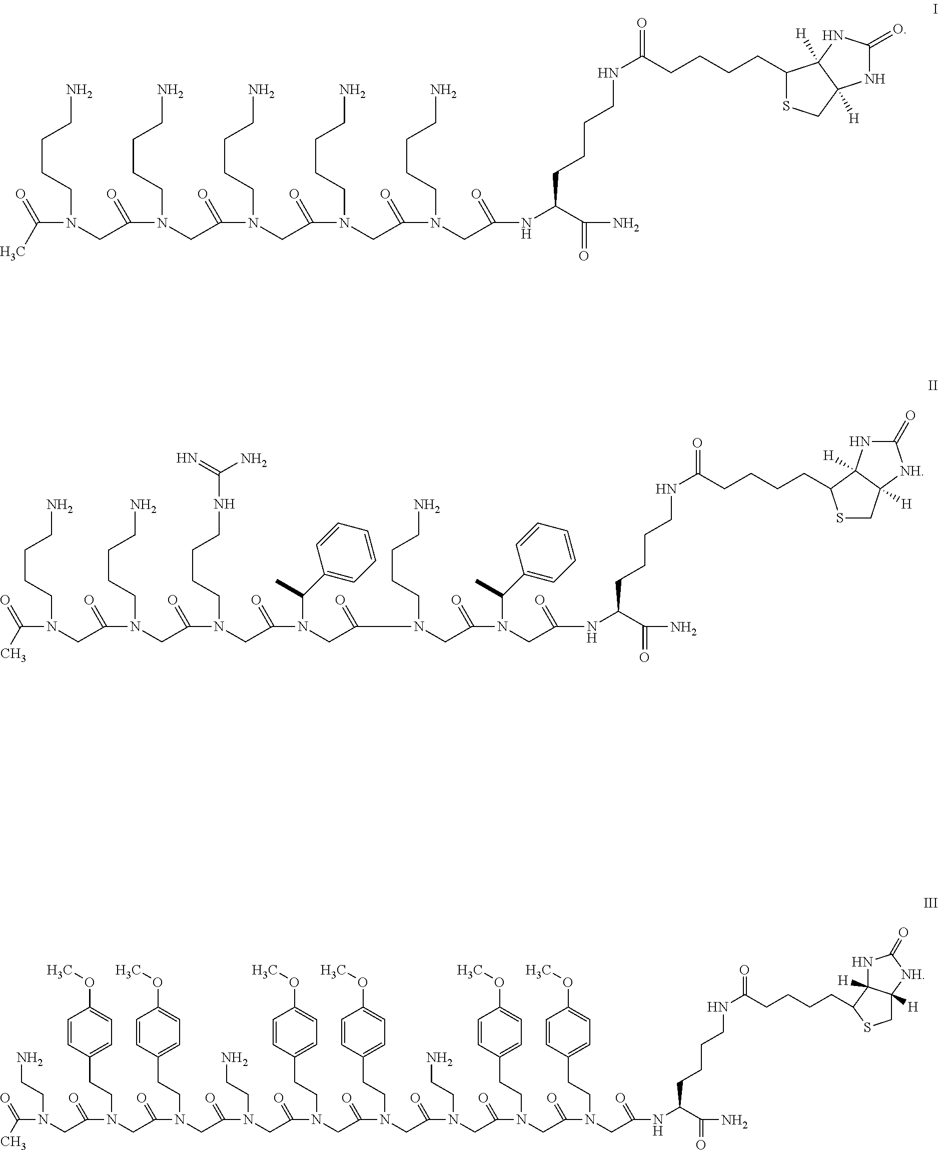

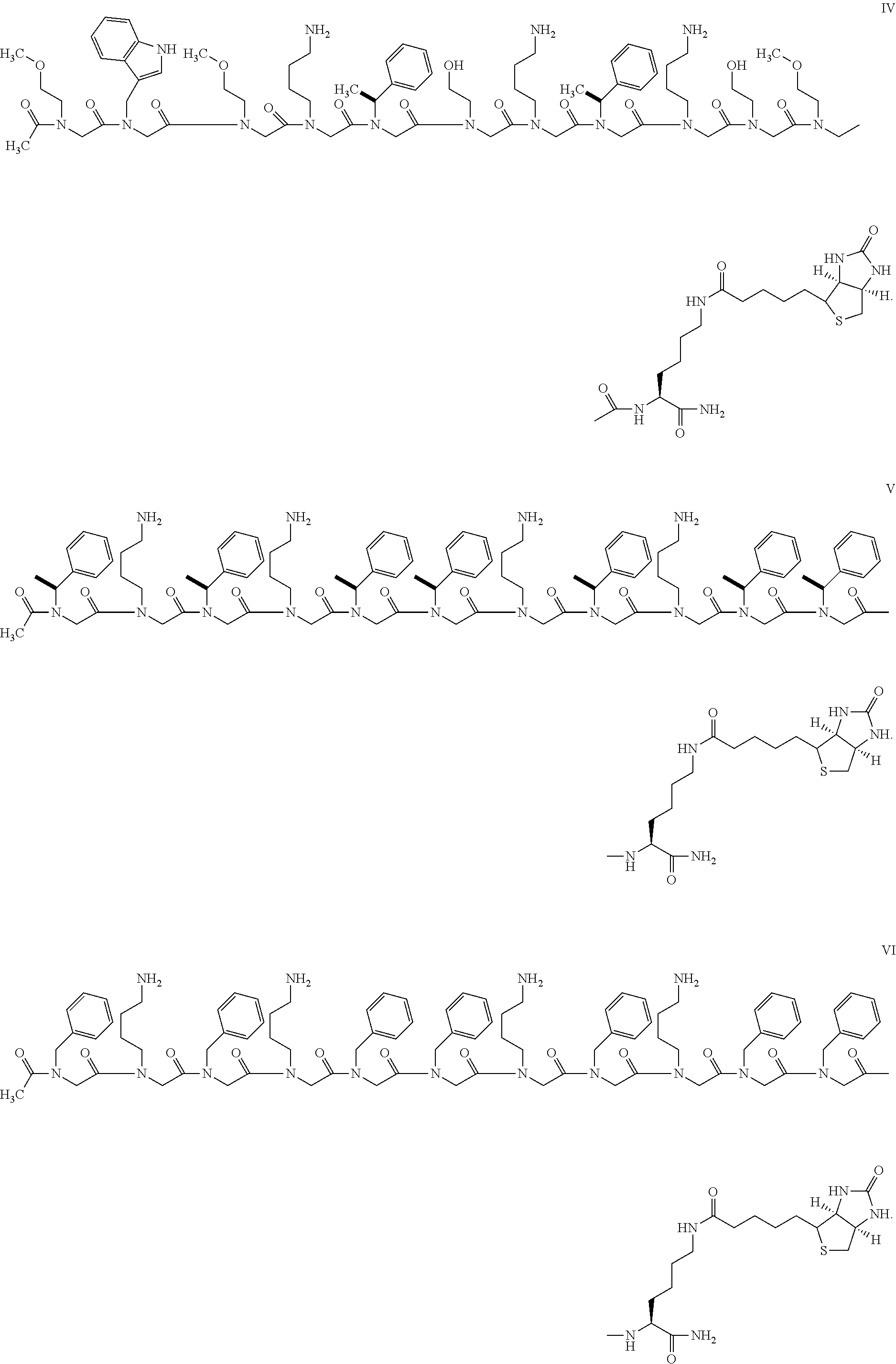

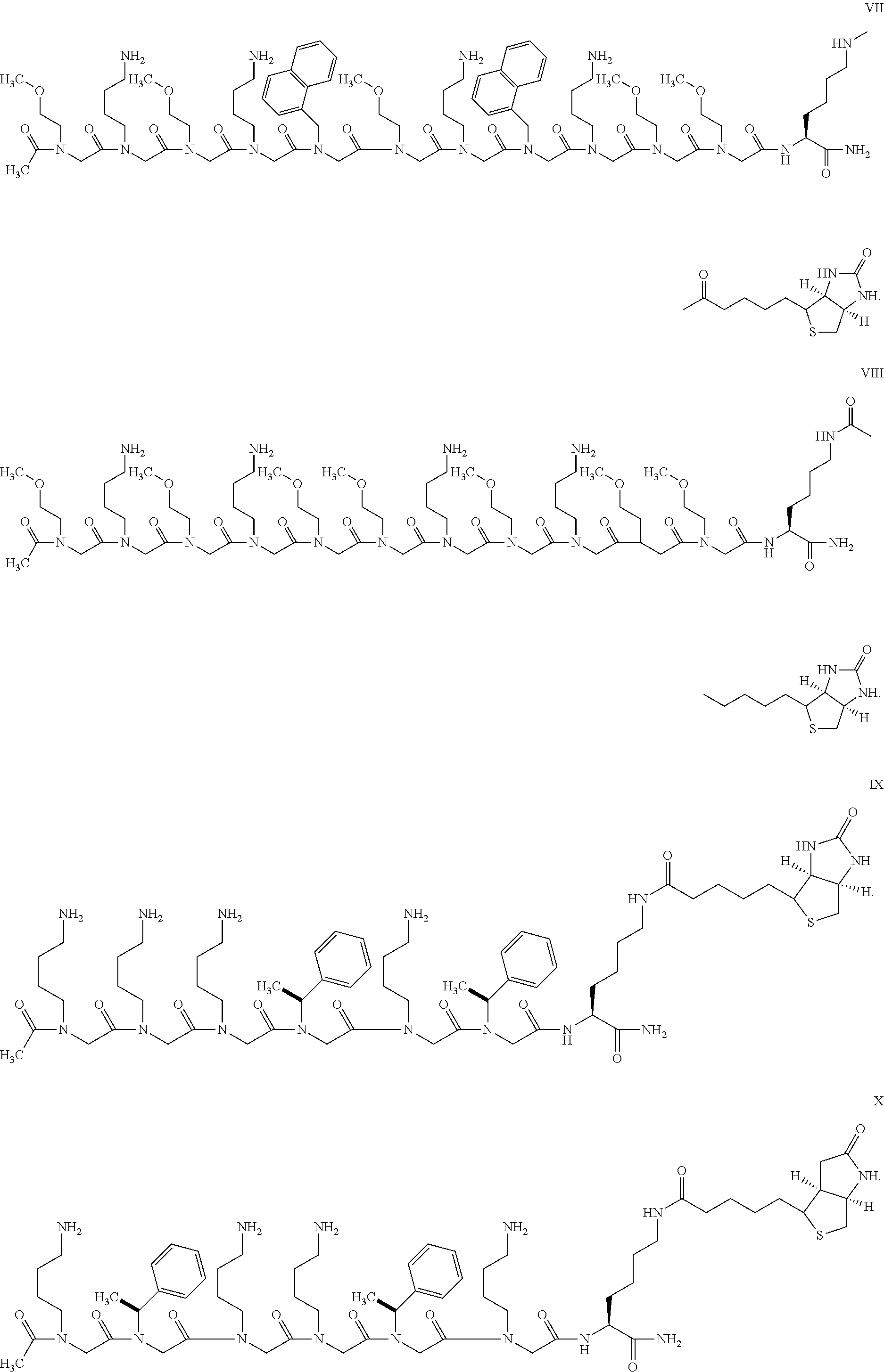

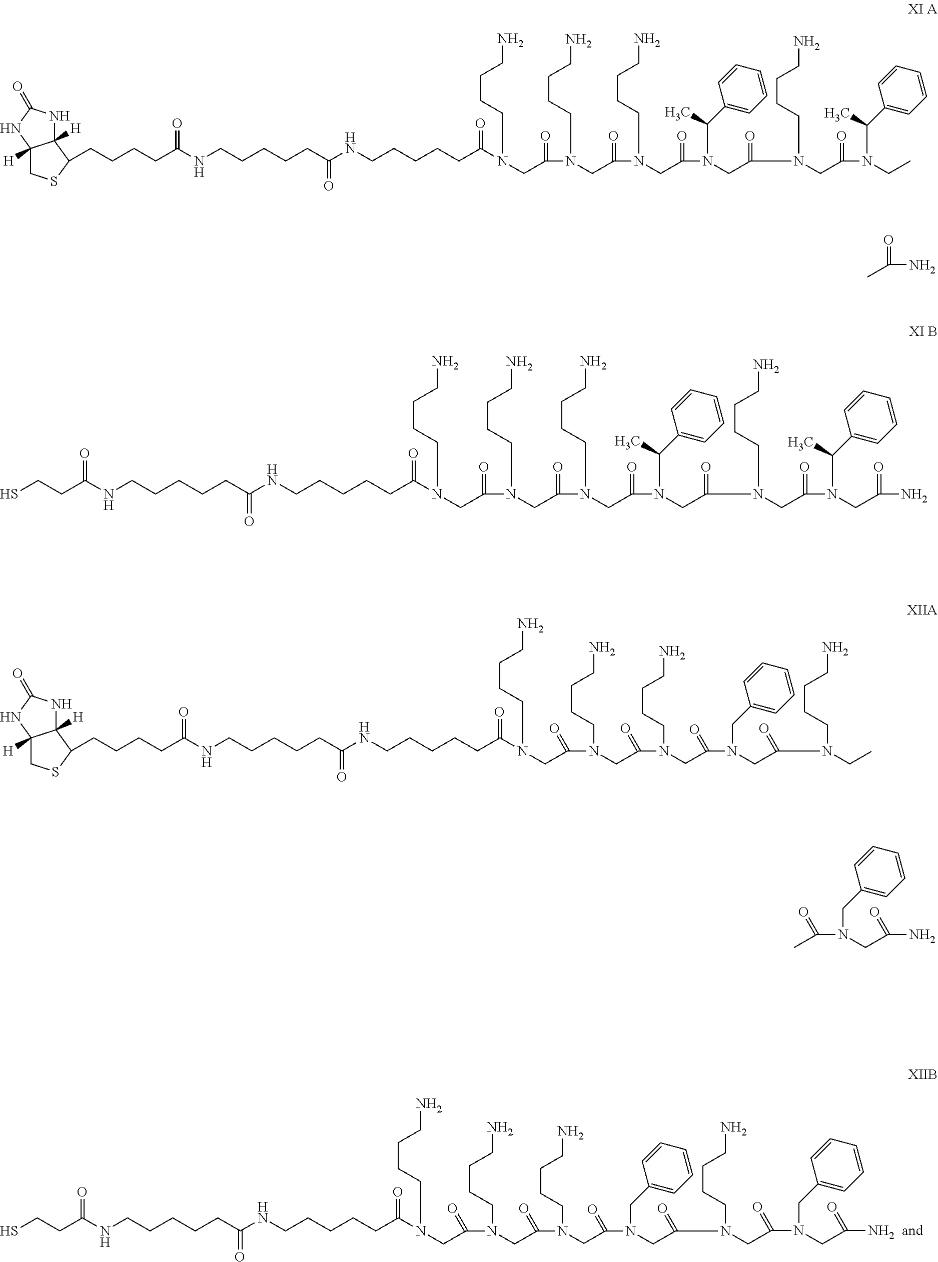

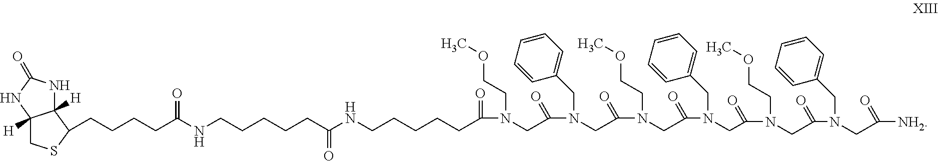

43. A method for detecting the presence of a pathogenic Alzheimer's disease conformer comprising the steps of:
contacting a sample suspected of containing said pathogenic Alzheimer's disease conformer with a pathogenic conformer-specific binding reagent under conditions that allow binding of said reagent to said pathogenic Alzheimer's disease conformer, if present, to form a complex;
contacting said complex with a conformational disease protein-specific binding reagent under conditions that allow binding; and
detecting the presence of said pathogenic Alzheimer's disease conformer, if any, in said sample by its binding to said conformational disease protein-specific binding reagent;
wherein said pathogenic conformer-specific binding reagent is

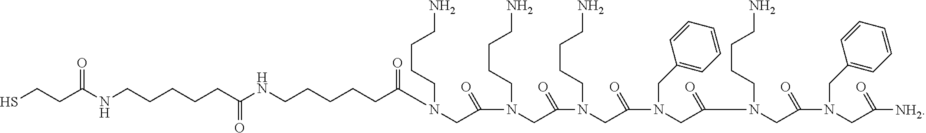

44. The method of claim 43, wherein said pathogenic Alzheimer's disease conformer is an Aβ protein and said conformational disease protein-specific binding reagent is an anti-Aβ antibody.

45. The method of claim 43, wherein said pathogenic Alzheimer's disease conformer is a tau protein and said conformational disease protein-specific binding reagent is an anti-tau antibody.

46. The method of claim 43, wherein said pathogenic conformer-specific binding reagent is coupled to a magnetic bead.


SEQUENCE LISTING (261 sequences)

SEQ ID NO: 1 - protein, 253 aa, Homo sapiens
MANLGCWMLVLFVATWSDLGLCKKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGGYMLGSAMSRPIIHFGSDYEDRYYRENMHRYPNQVYYRPMDEYSNQNNFVHDCVNITIKQHTVTTTTKGENFTETDVKMMERVVEQMCITQYERESQAYYQRGSSMVLFSSPPVILLISFLIFLIVG

SEQ ID NO: 2 - protein, 254 aa, Mus musculus
MANLGYWLLALFVTMWTDVGLCKKRPKPGGWNTGGSRYPGQGSPGGNRYPPQGGTWGQPHGGGWGQPHGGSWGQPHGGSWGQPHGGGWGQGGGTHNQWNKPSKPKTNLKHVAGAAAAGAVVGGLGGYMLGSAMSRPMIHFGNDWEDRYYRENMYRYPNQVYYRPVDQYSNQNNFVHDCVNITIKQHTVTTTTKGENFTETDVKMMERVVEQMCVTQYQKESQAYYDGRRSSSTVLFSSPPVILLISFLIFLIVG

SEQ ID NO: 3 - protein, 253 aa, Homo sapiens; identical to SEQ ID NO: 1

SEQ ID NO: 4 - protein, 254 aa, Mesocricetus auratus
MANLSYWLLALFVAMWTDVGLCKKRPKPGGWNTGGSRYPGQGSPGGNRYPPQGGGTWGQPHGGGWGQPHGGGWGQPHGGGWGQPHGGGWGQGGGTHNQWNKPSKPKTNMKHMAGAAAAGAVVGGLGGYMLGSAMSRPMMHFGNDWEDRYYRENMNRYPNQVYYRPVDQYNNQNNFVHDCVNITIKQHTVTTTTKGENFTETDIKIMERVVEQMCTTQYQKESQAYYDGRRSSAVLFSSPPVILLISFLIFLMVG

SEQ ID NO: 5 - protein, 265 aa, Bos taurus
  VARIANT 46, 155: Xaa = Any Amino Acid
MVKSHIGSWILVLFVAMWSDVGLCKKRPKPGGGWNTGGSRYPGQGXPGGNTRYPPQGGGGWGQPHGGGWGQPHGGGWGQPHGGGWGQPHGGGWGQPHGGGGWGQGGTHGQWNKPSKPKTNMKHVAGAAAAGAVVGGLGGYMLGSAMSRPLIHFGXDYEDRYYRENMHRYPNQVYYRPVDQYSNQNNFVHDCVNITVKEHTVTTTTKGENFTETDIKMMERVVEQMCITQYQRESQAYYQRGASVILFSSPPVILLISFLIFLIVG

SEQ ID NO: 6 - protein, 256 aa, Ovis aries
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGSHSQWNKPSKPKTNMKHVAGAAAAGAVVGGLGGYMLGSAMSRPLIHFGNDYEDRYYRENMYRYPNQVYYRPVDRYSNQNNFVHDCVNITVKQHTVTTTTKGENFTETDIKIMERVVEQMCITQYQRESQAYYQRGASVILFSSPPVILLISFLIFLIVG

SEQ ID NO: 7 - protein, 254 aa, Mus musculus; identical to SEQ ID NO: 2

SEQ ID NO: 8 - protein, 256 aa, Cervus elaphus
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGTHSQWNKPSKPKTNMKHVAGAAAAGAVVGGLGGYMLGSAMSRPLIHFGNDYEDRYYRENMYRYPNQVYYRPVDQYNNQNTFVHDCVNITVKQHTVTTTTKGENFTETDIKMMERVVEQMCITQYQRESEAYYQRGASVILFSSPPVILLISFLIFLIVG

SEQ ID NO: 9 - protein, 256 aa, Dama dama
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGTHSQWNKPSKPKTNMKHVAGAAAAGAVVGGLGGYMLGSAMNRPLIHFGNDYEDRYYRENMYRYPNQVYYRPVDQYNNQNTFVHDCVNITVKQHTVTTTTKGENFTETDIKMMERVVEQMCITQYQRESEAYYQRGASVILFSSPPVILLISFLIFLIVG

SEQ ID NO: 10 - protein, 256 aa, Odocoileus hemionus
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGTHSQWNKPSKPKTNMKHVAGAAAAGAVVGGLGGYMLGSAMSRPLIHFGNDYEDRYYRENMYRYPNQVYYRPVDQYNNQNTFVHDCVNITVKQHTVTTTTKGENFTETDIKMMERVVEQMCITQYQRESQAYYQRGASVILFSSPPVILLISFLIFLIVG

SEQ ID NO: 11 - protein, 256 aa, Odocoileus virginianus; identical to SEQ ID NO: 10

SEQ ID NO: 12 - protein, 5 aa, Artificial Sequence
  Synthetically generated peptide
KKRPK

SEQ ID NO: 13 - protein, 22 aa, Artificial Sequence
  Synthetically generated peptide
MANLGCWMLVLFVATWSDLGLC

SEQ ID NO: 14 - protein, 14 aa, Artificial Sequence
  Synthetically generated peptide
GGGQWNKPSKPKTN

SEQ ID NO: 15 - protein, 15 aa, Artificial Sequence
  Synthetically generated peptide
QWNKPSKPKTNMKHV

SEQ ID NO: 16 - protein, 28 aa, Artificial Sequence
  Synthetically generated peptide
NQNNXFVHDCVNITXKXHTVTTTTKGEN

SEQ ID NO: 17 - protein, 11 aa, Artificial Sequence
  Synthetically generated peptide
TTKGENFTETD

SEQ ID NO: 18 - protein, 8 aa, Artificial Sequence
  Synthetically generated peptide
GENFTETD

SEQ ID NO: 19 - protein, 38 aa, Artificial Sequence
  Synthetically generated peptide
GENFTETDXKXMERVVEQMCXTQYXESQAYYXGRRXXS

SEQ ID NO: 20 - protein, 63 aa, Artificial Sequence
  Synthetically generated peptide
NQNNXFVHDCVNITXKXHTVTTTTKGENFTETDXKXMERVVEQMCXTQYXESQAYYXGRRXXS

SEQ ID NO: 21 - protein, 22 aa, Artificial Sequence
  Synthetically generated peptide
XXLFSSPPVILLISFLIFLXVG

SEQ ID NO: 22 - protein, 36 aa, Artificial Sequence
  Synthetically generated peptide
GXDXEDRYYRENMXRYPNQVYYRPXDXYXNQNXFVH

SEQ ID NO: 23 - protein, 22 aa, Artificial Sequence
  Synthetically generated peptide
NXFVHDCVNITXKXHTVTTTTK

SEQ ID NO: 24 - protein, 4 aa, Artificial Sequence
  Synthetically generated peptide
VYYR

SEQ ID NO: 25 - protein, 13 aa, Artificial Sequence
  Synthetically generated peptide
RYPNQVYYRPXDX

SEQ ID NO: 26 - protein, 33 aa, Artificial Sequence
  Synthetically generated peptide
KKRPKPGGGWNTGGSRYPGQGSPGGNRYPPQGG

SEQ ID NO: 27 - protein, 25 aa, Artificial Sequence
  Synthetically generated peptide
WNTGGSRYPGQGSPGGNRYPPQGGG

SEQ ID NO: 28 - protein, 33 aa, Artificial Sequence
  Synthetically generated peptide
WNTGGSRYPGQGSPGGNRYPPQGGGXWGQPHGG

SEQ ID NO: 29 - protein, 22 aa, Artificial Sequence
  Synthetically generated peptide
GGWGQGGGTHSQWNKPSKPKTN

SEQ ID NO: 30 - protein, 16 aa, Artificial Sequence
  Synthetically generated peptide
GGTHSQWNKPSKPKTN

SEQ ID NO: 31 - protein, 49 aa, Artificial Sequence
  Synthetically generated peptide
WNTGGSRYPGQGSPGGNRYPPQGGGXWGQPHGGGWGQPHGGGWGQPHGG

SEQ ID NO: 32 - protein, 8 aa, Artificial Sequence
  Synthetically generated peptide
GQPHGGGW

SEQ ID NO: 33 - protein, 21 aa, Artificial Sequence
  Synthetically generated peptide
RPIIHFGSDYEDRYYRENMHR

SEQ ID NO: 34 - protein, 21 aa, Artificial Sequence
  Synthetically generated peptide
RPMIHFGNDWEDRYYRENMYR

SEQ ID NO: 35 - protein, 38 aa, Artificial Sequence
  Synthetically generated peptide
GGGGCGGGGWGQGGGTHNQWNKPSKPKTNLKHVGGGGC

SEQ ID NO: 36 - protein, 30 aa, Artificial Sequence
  Synthetically generated peptide
GGGGGGWGQGGGTHNQWNKPSKPKTNLKHV

SEQ ID NO: 37 - protein, 30 aa, Artificial Sequence
  Synthetically generated peptide
GGWGQGGGTHNQWNKPSKPKTNLKHVGGGG

SEQ ID NO: 38 - protein, 4 aa, Artificial Sequence
  Synthetically generated peptide
XKHX

SEQ ID NO: 39 - protein, 9 aa, Artificial Sequence
  Synthetically generated peptide
KPKTNXKHX

SEQ ID NO: 40 - protein, 34 aa, Artificial Sequence
  Synthetically generated peptide
CGGGGWGQGGGTHNQWNKPSKPKTNLKHVGGGGC

SEQ ID NO: 41 - protein, 25 aa, Artificial Sequence
  Synthetically generated peptide
SRPIIHFGSDYEDRYYRENMHRYPN

SEQ ID NO: 42 - protein, 23 aa, Artificial Sequence
  Synthetically generated peptide
PMIHFGNDWEDRYYRENMYRPVD

SEQ ID NO: 43 - protein, 22 aa, Artificial Sequence
  Synthetically generated peptide
AGAAAAGAVVGGLGGYMLGSAM

SEQ ID NO: 44 - protein, 24 aa, Artificial Sequence
  Synthetically generated peptide
RPMIHFGNDWEDRYYRENMYRGGG

SEQ ID NO: 45 - protein, 26 aa, Artificial Sequence
  Synthetically generated peptide
GGGRPMIHFGNDWEDRYYRENMYRGG

SEQ ID NO: 46 - protein, 31 aa, Artificial Sequence
  Synthetically generated peptide
GGCGGGRPMIHFGNDWEDRYYRENMYRGGGC

SEQ ID NO: 47 - protein, 15 aa, Artificial Sequence
  Synthetically generated peptide
AGAAAAGAVVGGLGG

SEQ ID NO: 48 - protein, 5 aa, Artificial Sequence
  Synthetically generated peptide
GGLGG

SEQ ID NO: 49 - protein, 3 aa, Artificial Sequence
  Synthetically generated peptide
LGS

SEQ ID NO: 50 - protein, 14 aa, Artificial Sequence
  Synthetically generated peptide
QWNKPSKPKTNGGG

SEQ ID NO: 51 - protein, 25 aa, Artificial Sequence
  Synthetically generated peptide
QWNKPSKPKTNGGGQWNKPSKPKTN

SEQ ID NO: 52 - protein, 18 aa, Artificial Sequence
  Synthetically generated peptide
QWNKPSKPKTNLKHVGGG

SEQ ID NO: 53 - protein, 22 aa, Artificial Sequence
  Synthetically generated peptide
GGWGQGGGTHNQWNKPSKPKTN

SEQ ID NO: 54 - protein, 16 aa, Artificial Sequence
  Synthetically generated peptide
GGTHNQWNKPSKPKTN

SEQ ID NO: 55 - protein, 25 aa, Artificial Sequence
  Synthetically generated peptide
GGGAGAAAAGAVVGGLGGYMLGSAM

SEQ ID NO: 56 - protein, 18 aa, Artificial Sequence
  Synthetically generated peptide
GGGAGAAAAGAVVGGLGG

SEQ ID NO: 57 - protein, 25 aa, Artificial Sequence
  Synthetically generated peptide
KKKAGAAAAGAVVGGLGGYMLGSAM

SEQ ID NO: 58 - protein, 9 aa, Artificial Sequence
  Synthetically generated peptide
YMLGSAMXR

SEQ ID NO: 59 - protein, 6 aa, Artificial Sequence
  Synthetically generated peptide
XRPXXH

SEQ ID NO: 60 - protein, 13 aa, Artificial Sequence
  Synthetically generated peptide
YMLGSAMXRPXXH

SEQ ID NO: 61 - protein, 17 aa, Artificial Sequence
  Synthetically generated peptide
YMLGSAMXRPXXHFGXD

SEQ ID NO: 62 - protein, 25 aa, Artificial Sequence
  Synthetically generated peptide
XEDRYYRENMXRYPNQVYYRPXDXY

SEQ ID NO: 63 - protein, 30 aa, Artificial Sequence
  Synthetically generated peptide
XEDRYYRENMXRYPNQVYYRPXDXYXNQNX

SEQ ID NO: 64 - protein, 8 aa, Artificial Sequence
  Synthetically generated peptide
DXYXNQNX

SEQ ID NO: 65 - protein, 18 aa, Artificial Sequence
  Synthetically generated peptide
KKKAGAAAAGAVVGGLGG

SEQ ID NO: 66 - protein, 24 aa, Artificial Sequence
  Synthetically generated peptide
GGGKKRPKPGGWNTGGSRYPGQGS

SEQ ID NO: 67 - protein, 16 aa, Artificial Sequence
  Synthetically generated peptide
GGGKKRPKPGGWNTGG

SEQ ID NO: 68 - protein, 11 aa, Artificial Sequence
  Synthetically generated peptide
GGGKKRPKPGG

SEQ ID NO: 69 - protein, 23 aa, Artificial Sequence
  Synthetically generated peptide
PHGGGWGQHGGSWGQPHGGSWGQ

SEQ ID NO: 70 - protein, 16 aa, Artificial Sequence
  Synthetically generated peptide
PHGGGWGQPHGGSWGQ

SEQ ID NO: 71 - protein, 8 aa, Artificial Sequence
  Synthetically generated peptide
PHGGGWGQ

SEQ ID NO: 72 - protein, 20 aa, Artificial Sequence
  Synthetically generated peptide
GGGKKRPKPGGGKKRPKPGG

SEQ ID NO: 73 - protein, 11 aa, Artificial Sequence
  Synthetically generated peptide
GGGGPKRKGPK

SEQ ID NO: 74 - protein, 16 aa, Artificial Sequence
  Synthetically generated peptide
GGGWNTGGSRYPGQGS

SEQ ID NO: 75 - protein, 12 aa, Artificial Sequence
  Synthetically generated peptide
GGGWNKPSKPKT

SEQ ID NO: 76 - protein, 27 aa, Artificial Sequence
  Synthetically generated peptide
GGGRPMIHFGNDWEDRYYRENMYRGGC

SEQ ID NO: 77 - protein, 18 aa, Artificial Sequence
  Synthetically generated peptide
QWNKPSKPKTNLKHVGGG

SEQ ID NO: 78 - protein, 25 aa, Artificial Sequence
  Synthetically generated peptide
GGGAGAAAAGAVVGGLGGYMLGSAM

SEQ ID NO: 79 - protein, 10 aa, Artificial Sequence
  Synthetically generated peptide
GGGNKPSKPK

SEQ ID NO: 80 - protein, 9 aa, Artificial Sequence
  Synthetically generated peptide
GGGKPSKPK

SEQ ID NO: 81 - protein, 23 aa, Artificial Sequence
  Synthetically generated peptide
GGGKKRPKPGGGQWNKPSKPKTN

SEQ ID NO: 82 - protein, 28 aa, Artificial Sequence
  Synthetically generated peptide
KKKAGAAAAGAVVGGLGGYMLGSAMDDD

SEQ ID NO: 83 - protein, 25 aa, Artificial Sequence
  Synthetically generated peptide
DDDAGAAAAGAVVGGLGGYMLGSAM

SEQ ID NO: 84 - protein, 28 aa, Artificial Sequence
  Synthetically generated peptide
KKKAGAAAAGAVVGGLGGYMLGSAMKKK

SEQ ID NO: 85 - protein, 11 aa, Artificial Sequence
  Synthetically generated peptide
GGGKKKKKKKK

SEQ ID NO: 86 - protein, 28 aa, Artificial Sequence
  Synthetically generated peptide
DDDAGAAAAGAVVGGLGGYMLGSAMDDD

SEQ ID NO: 87 - protein, 14 aa, Artificial Sequence
  Synthetically generated peptide
GGGNNKQSPWPTKK

SEQ ID NO: 88 - protein, 21 aa, Artificial Sequence
  Synthetically generated peptide
DKDKGGVGAGAAVAAGGDKDK

SEQ ID NO: 89 - protein, 14 aa, Artificial Sequence
  Synthetically generated peptide
GGGQANKPSKPKTN

SEQ ID NO: 90 - protein, 14 aa, Artificial Sequence
  Synthetically generated peptide
GGGQWNKASKPKTN

SEQ ID NO: 91 - protein, 14 aa, Artificial Sequence
  Synthetically generated peptide
GGGQWNKPSKAKTN

SEQ ID NO: 92 - protein, 14 aa, Artificial Sequence
  Synthetically generated peptide
GGGQWNAPSKPKTN

SEQ ID NO: 93 - protein, 14 aa, Artificial Sequence
  Synthetically generated peptide
GGGQWNKPSAPKTN

SEQ ID NO: 94 - protein, 14 aa, Artificial Sequence
  Synthetically generated peptide
GGGQWNKPSKPATN

SEQ ID NO: 95 - protein, 14 aa, Artificial Sequence
  Synthetically generated peptide
GGGQWNKASKAKTN

SEQ ID NO: 96 - protein, 11 aa, Artificial Sequence
  Synthetically generated peptide
GGGKKRAKPGG

SEQ ID NO: 97 - protein, 11 aa, Artificial Sequence
  Synthetically generated peptide
GGGKKRPKAGG

SEQ ID NO: 98 - protein, 11 aa, Artificial Sequence
  Synthetically generated peptide
GGGKKRAKAGG

SEQ ID NO: 99 - protein, 14 aa, Artificial Sequence
  Synthetically generated peptide
GGGQWNKASKPKTN

SEQ ID NO: 100 - protein, 14 aa, Artificial Sequence
  Synthetically generated peptide
GGGQWAKPSKPKTN

SEQ ID NO: 101 - protein, 14 aa, Artificial Sequence
  Synthetically generated peptide
GGGQWNKPAKPKTN

SEQ ID NO: 102 - protein, 14 aa, Artificial Sequence
  Synthetically generated peptide
GGGQWNKPSKPKAN

SEQ ID NO: 103 - protein, 14 aa, Artificial Sequence
  Synthetically generated peptide
GGGQWNKPSKPKTA

SEQ ID NO: 104 - protein, 11 aa, Artificial Sequence
  Synthetically generated peptide
GGGAKRPKPGG

SEQ ID NO: 105 - protein, 11 aa, Artificial Sequence
  Synthetically generated peptide
GGGKARPKPGG

SEQ ID NO: 106 - protein, 11 aa, Artificial Sequence
  Synthetically generated peptide
GGGKKAPKPGG

SEQ ID NO: 107 - protein, 11 aa, Artificial Sequence
  Synthetically generated peptide
GGGKKRPAPGG

SEQ ID NO: 108 - protein, 11 aa, Artificial Sequence
  Synthetically generated peptide
GGGKKAPKAGG

SEQ ID NO: 109 - protein, 17 aa, Artificial Sequence
  Synthetically generated peptide
GGGKKRPKPGGGWNTGG

SEQ ID NO: 110 - protein, 39 aa, Artificial Sequence
  Synthetically generated peptide
QWNKPSKPKTNGGGQWNKPSKPKTNGGGQWNKPSKPKTN

SEQ ID NO: 111 - protein, 23 aa, Artificial Sequence
  Synthetically generated peptide
QWNKPSKPKTNQWNKPSKPKTNK

SEQ ID NO: 112 - protein, 17 aa, Artificial Sequence
  Synthetically generated peptide
GGGKKRPKPGGWNTGGG

SEQ ID NO: 113 - protein, 17 aa, Artificial Sequence
  Synthetically generated peptide
GGGKKRPKPGGWNTGGG

SEQ ID NO: 114 - protein, 18 aa, Artificial Sequence
  Synthetically generated peptide
KKKAGAAAAGAVVGGLGX

SEQ ID NO: 115 - protein, 19 aa, Artificial Sequence
  Synthetically generated peptide
DLGLCKKRPKPGGXWNTGG

SEQ ID NO: 116 - protein, 18 aa, Artificial Sequence
  Synthetically generated peptide
DLGLCKKRPKPGGXWNTG

SEQ ID NO: 117 - protein, 17 aa, Artificial Sequence
  Synthetically generated peptide
DLGLCKKRPKPGGXWNT

SEQ ID NO: 118 - protein, 16 aa, Artificial Sequence
  Synthetically generated peptide
DLGLCKKRPKPGGXWN

SEQ ID NO: 119 - protein, 15 aa, Artificial Sequence
  Synthetically generated peptide
DLGLCKKRPKPGGXW

SEQ ID NO: 120 - protein, 14 aa, Artificial Sequence
  Synthetically generated peptide
DLGLCKKRPKPGGX

SEQ ID NO: 121 - protein, 17 aa, Artificial Sequence
  Synthetically generated peptide
LGLCKKRPKPGGXWNTG

SEQ ID NO: 122 - protein, 16 aa, Artificial Sequence
  Synthetically generated peptide
LGLCKKRPKPGGXWNT

SEQ ID NO: 123 - protein, 15 aa, Artificial Sequence
  Synthetically generated peptide
LGLCKKRPKPGGXWN

SEQ ID NO: 124 - protein, 14 aa, Artificial Sequence
  Synthetically generated peptide
LGLCKKRPKPGGXW

SEQ ID NO: 125 - protein, 13 aa, Artificial Sequence
  Synthetically generated peptide
LGLCKKRPKPGGX

SEQ ID NO: 126 - protein, 17 aa, Artificial Sequence
  Synthetically generated peptide
GLCKKRPKPGGXWNTGG

SEQ ID NO: 127 - protein, 16 aa, Artificial Sequence
  Synthetically generated peptide
GLCKKRPKPGGXWNTG

SEQ ID NO: 128 - protein, 15 aa, Artificial Sequence
  Synthetically generated peptide
GLCKKRPKPGGXWNT

SEQ ID NO: 129 - protein, 14 aa, Artificial Sequence
  Synthetically generated peptide
GLCKKRPKPGGXWN

SEQ ID NO: 130 - protein, 13 aa, Artificial Sequence
  Synthetically generated peptide
GLCKKRPKPGGXW

SEQ ID NO: 131 - protein, 12 aa, Artificial Sequence
  Synthetically generated peptide
GLCKKRPKPGGX

SEQ ID NO: 132 - protein, 16 aa, Artificial Sequence
  Synthetically generated peptide
LCKKRPKPGGXWNTGG

SEQ ID NO: 133 - protein, 15 aa, Artificial Sequence
  Synthetically generated peptide
LCKKRPKPGGXWNTG

SEQ ID NO: 134 - protein, 14 aa, Artificial Sequence
  Synthetically generated peptide
LCKKRPKPGGXWNT

SEQ ID NO: 135 - protein, 13 aa, Artificial Sequence
  Synthetically generated peptide
LCKKRPKPGGXWN

SEQ ID NO: 136 - protein, 12 aa, Artificial Sequence
  Synthetically generated peptide
LCKKRPKPGGXW

SEQ ID NO: 137 - protein, 11 aa, Artificial Sequence
  Synthetically generated peptide
LCKKRPKPGGX

SEQ ID NO: 138 - protein, 15 aa, Artificial Sequence
  Synthetically generated peptide
CKKRPKPGGXWNTGG

SEQ ID NO: 139 - protein, 14 aa, Artificial Sequence
  Synthetically generated peptide
CKKRPKPGGXWNTG

SEQ ID NO: 140 - protein, 13 aa, Artificial Sequence
  Synthetically generated peptide
CKKRPKPGGXWNT

SEQ ID NO: 141 - protein, 12 aa, Artificial Sequence
  Synthetically generated peptide
CKKRPKPGGXWN

SEQ ID NO: 142 - protein, 11 aa, Artificial Sequence
  Synthetically generated peptide
CKKRPKPGGXW

SEQ ID NO: 143 - protein, 10 aa, Artificial Sequence
  Synthetically generated peptide
CKKRPKPGGX

SEQ ID NO: 144 - protein, 14 aa, Artificial Sequence
  Synthetically generated peptide
KKRPKPGGXWNTGG

SEQ ID NO: 145 - protein, 13 aa, Artificial Sequence
  Synthetically generated peptide
KKRPKPGGXWNTG

SEQ ID NO: 146 - protein, 12 aa, Artificial Sequence
  Synthetically generated peptide
KKRPKPGGXWNT

SEQ ID NO: 147 - protein, 11 aa, Artificial Sequence
  Synthetically generated peptide
KKRPKPGGXWN

SEQ ID NO: 148 - protein, 10 aa, Artificial Sequence
  Synthetically generated peptide
KKRPKPGGXW

SEQ ID NO: 149 - protein, 9 aa, Artificial Sequence
  Synthetically generated peptide
KKRPKPGGX

SEQ ID NO: 150 - protein, 19 aa, Artificial Sequence
  Synthetically generated peptide
DVGLCKKRPKPGGXWNTGG

SEQ ID NO: 151 - protein, 18 aa, Artificial Sequence
  Synthetically generated peptide
DVGLCKKRPKPGGXWNTG

SEQ ID NO: 152 - protein, 17 aa, Artificial Sequence
  Synthetically generated peptide
DVGLCKKRPKPGGXWNT

SEQ ID NO: 153 - protein, 16 aa, Artificial Sequence
  Synthetically generated peptide
DVGLCKKRPKPGGXWN

SEQ ID NO: 154 - protein, 15 aa, Artificial Sequence
  Synthetically generated peptide
DVGLCKKRPKPGGXW

SEQ ID NO: 155 - protein, 14 aa, Artificial Sequence
  Synthetically generated peptide
DVGLCKKRPKPGGX

SEQ ID NO: 156 - protein, 17 aa, Artificial Sequence
  Synthetically generated peptide
VGLCKKRPKPGGXWNTG

SEQ ID NO: 157 - protein, 16 aa, Artificial Sequence
  Synthetically generated peptide
VGLCKKRPKPGGXWNT

SEQ ID NO: 158 - protein, 15 aa, Artificial Sequence
  Synthetically generated peptide
VGLCKKRPKPGGXWN

SEQ ID NO: 159 - protein, 14 aa, Artificial Sequence
  Synthetically generated peptide
VGLCKKRPKPGGXW

SEQ ID NO: 160 - protein, 13 aa, Artificial Sequence
  Synthetically generated peptide
VGLCKKRPKPGGX

SEQ ID NO: 161 - protein, 18 aa, Artificial Sequence
  Synthetically generated peptide
THSQWNKPSKPKTNMKHM

SEQ ID NO: 162 - protein, 17 aa, Artificial Sequence
  Synthetically generated peptide
THSQWNKPSKPKTNMKH

SEQ ID NO: 163 - protein, 16 aa, Artificial Sequence
  Synthetically generated peptide
THSQWNKPSKPKTNMK

SEQ ID NO: 164 - protein, 15 aa, Artificial Sequence
  Synthetically generated peptide
THSQWNKPSKPKTNM

SEQ ID NO: 165 - protein, 14 aa, Artificial Sequence
  Synthetically generated peptide
THSQWNKPSKPKTN

SEQ ID NO: 166 - protein, 17 aa, Artificial Sequence
  Synthetically generated peptide
HSQWNKPSKPKTNMKHM

SEQ ID NO: 167 - protein, 16 aa, Artificial Sequence
  Synthetically generated peptide
HSQWNKPSKPKTNMKH

SEQ ID NO: 168 - protein, 15 aa, Artificial Sequence
  Synthetically generated peptide
HSQWNKPSKPKTNMK

SEQ ID NO: 169 - protein, 14 aa, Artificial Sequence
  Synthetically generated peptide
HSQWNKPSKPKTNM

SEQ ID NO: 170 - protein, 13 aa, Artificial Sequence
  Synthetically generated peptide
HSQWNKPSKPKTN

SEQ ID NO: 171 - protein, 16 aa, Artificial Sequence
  Synthetically generated peptide
SQWNKPSKPKTNMKHM

SEQ ID NO: 172 - protein, 15 aa, Artificial Sequence
  Synthetically generated peptide
SQWNKPSKPKTNMKH

SEQ ID NO: 173 - protein, 14 aa, Artificial Sequence
  Synthetically generated peptide
SQWNKPSKPKTNMK

SEQ ID NO: 174 - protein, 13 aa, Artificial Sequence
  Synthetically generated peptide
SQWNKPSKPKTNM

SEQ ID NO: 175 - protein, 12 aa, Artificial Sequence
  Synthetically generated peptide
SQWNKPSKPKTN

SEQ ID NO: 176 - protein, 15 aa, Artificial Sequence
  Synthetically generated peptide
QWNKPSKPKTNMKHM

SEQ ID NO: 177 - protein, 14 aa, Artificial Sequence
  Synthetically generated peptide
QWNKPSKPKTNMKH

SEQ ID NO: 178 - protein, 13 aa, Artificial Sequence
  Synthetically generated peptide
QWNKPSKPKTNMK

SEQ ID NO: 179 - protein, 12 aa, Artificial Sequence
  Synthetically generated peptide
QWNKPSKPKTNM

SEQ ID NO: 180 - protein, 18 aa, Artificial Sequence
  Synthetically generated peptide
THSQWNKPSKPKTNMKHV

SEQ ID NO: 181 - protein, 17 aa, Artificial Sequence
  Synthetically generated peptide
HSQWNKPSKPKTNMKHV

SEQ ID NO: 182 - protein, 16 aa, Artificial Sequence
  Synthetically generated peptide
SQWNKPSKPKTNMKHV

SEQ ID NO: 183 - protein, 15 aa, Artificial Sequence
  Synthetically generated peptide
QWNKPSKPKTNMKHV

SEQ ID NO: 184 - protein, 18 aa, Artificial Sequence
  Synthetically generated peptide
THGQWNKPSKPKTNMKHM

SEQ ID NO: 185 - protein, 17 aa, Artificial Sequence
  Synthetically generated peptide
THGQWNKPSKPKTNMKH

SEQ ID NO: 186 - protein, 16 aa, Artificial Sequence
  Synthetically generated peptide
THGQWNKPSKPKTNMK

SEQ ID NO: 187 - protein, 15 aa, Artificial Sequence
  Synthetically generated peptide
THGQWNKPSKPKTNM

SEQ ID NO: 188 - protein, 14 aa, Artificial Sequence
  Synthetically generated peptide
THGQWNKPSKPKTN

SEQ ID NO: 189 - protein, 17 aa, Artificial Sequence
  Synthetically generated peptide
HGQWNKPSKPKTNMKHM

SEQ ID NO: 190 - protein, 16 aa, Artificial Sequence
  Synthetically generated peptide
HGQWNKPSKPKTNMKH

SEQ ID NO: 191 - protein, 15 aa, Artificial Sequence
  Synthetically generated peptide
HGQWNKPSKPKTNMK

SEQ ID NO: 192 - protein, 14 aa, Artificial Sequence
  Synthetically generated peptide
HGQWNKPSKPKTNM

SEQ ID NO: 193 - protein, 13 aa, Artificial Sequence
  Synthetically generated peptide
HGQWNKPSKPKTN

SEQ ID NO: 194 - protein, 16 aa, Artificial Sequence
  Synthetically generated peptide
GQWNKPSKPKTNMKHM

SEQ ID NO: 195 - protein, 15 aa, Artificial Sequence
  Synthetically generated peptide
GQWNKPSKPKTNMKH

SEQ ID NO: 196 - protein, 14 aa, Artificial Sequence
  Synthetically generated peptide
GQWNKPSKPKTNMK

SEQ ID NO: 197 - protein, 13 aa, Artificial Sequence
  Synthetically generated peptide
GQWNKPSKPKTNM

SEQ ID NO: 198 - protein, 12 aa, Artificial Sequence
  Synthetically generated peptide
GQWNKPSKPKTN

SEQ ID NO: 199 - protein, 18 aa, Artificial Sequence
  Synthetically generated peptide
THGQWNKPSKPKTNMKHV

SEQ ID NO: 200 - protein, 17 aa, Artificial Sequence
  Synthetically generated peptide
HGQWNKPSKPKTNMKHV

SEQ ID NO: 201 - protein, 16 aa, Artificial Sequence
  Synthetically generated peptide
GQWNKPSKPKTNMKHV

SEQ ID NO: 202 - protein, 18 aa, Artificial Sequence
  Synthetically generated peptide
THNQWNKPSKPKTNMKHM

SEQ ID NO: 203 - protein, 17 aa, Artificial Sequence
  Synthetically generated peptide
THNQWNKPSKPKTNMKH

SEQ ID NO: 204 - protein, 16 aa, Artificial Sequence
  Synthetically generated peptide
THNQWNKPSKPKTNMK

SEQ ID NO: 205 - protein, 15 aa, Artificial Sequence
  Synthetically generated peptide
THNQWNKPSKPKTNM

SEQ ID NO: 206 - protein, 14 aa, Artificial Sequence
  Synthetically generated peptide
THNQWNKPSKPKTN

SEQ ID NO: 207 - protein, 17 aa, Artificial Sequence
  Synthetically generated peptide
HNQWNKPSKPKTNMKHM

SEQ ID NO: 208 - protein, 16 aa, Artificial Sequence
  Synthetically generated peptide
HNQWNKPSKPKTNMKH

SEQ ID NO: 209 - protein, 15 aa, Artificial Sequence
  Synthetically generated peptide
HNQWNKPSKPKTNMK

SEQ ID NO: 210 - protein, 14 aa, Artificial Sequence
  Synthetically generated peptide
HNQWNKPSKPKTNM

SEQ ID NO: 211 - protein, 13 aa, Artificial Sequence
  Synthetically generated peptide
HNQWNKPSKPKTN

SEQ ID NO: 212 - protein, 16 aa, Artificial Sequence
  Synthetically generated peptide
NQWNKPSKPKTNMKHM

SEQ ID NO: 213 - protein, 15 aa, Artificial Sequence
  Synthetically generated peptide
NQWNKPSKPKTNMKH

SEQ ID NO: 214 - protein, 14 aa, Artificial Sequence
  Synthetically generated peptide
NQWNKPSKPKTNMK

SEQ ID NO: 215 - protein, 13 aa, Artificial Sequence
  Synthetically generated peptide
NQWNKPSKPKTNM

SEQ ID NO: 216 - protein, 12 aa, Artificial Sequence
  Synthetically generated peptide
NQWNKPSKPKTN

SEQ ID NO: 217 - protein, 18 aa, Artificial Sequence
  Synthetically generated peptide
THNQWNKPSKPKTNMKHV

SEQ ID NO: 218 - protein, 17 aa, Artificial Sequence
  Synthetically generated peptide
HNQWNKPSKPKTNMKHV

SEQ ID NO: 219 - protein, 16 aa, Artificial Sequence
  Synthetically generated peptide
NQWNKPSKPKTNMKHV

SEQ ID NO: 220 - protein, 24 aa, Artificial Sequence
  Synthetically generated peptide
PHGGGWGQPHGGGWGQPHGGGWGQ

SEQ ID NO: 221 - protein, 26 aa, Artificial Sequence
  Synthetically generated peptide
GGWGQGGGTHSQWNKPSKPKTNMKHM

SEQ ID NO: 222 - protein, 33 aa, Artificial Sequence
  Synthetically generated peptide
QWNKPSKPKTNMKHMGGGQWNKPSKPKTNMKHM

SEQ ID NO: 223 - protein, 30 aa, Artificial Sequence
  Synthetically generated peptide
GGWGQGGGTHXQWNKPSKPKTNXKHXGGGG

SEQ ID NO: 224 - protein, 23 aa, Artificial Sequence
  Synthetically generated peptide
PHGGGWGQHGXSWGQPHGGXWGQ

SEQ ID NO: 225 - protein, 18 aa, Artificial Sequence
  Synthetically generated peptide
QWNKPSKPKTNXKHXGGG

SEQ ID NO: 226 - protein, 14 aa, Artificial Sequence
  Synthetically generated peptide
GGGAWNKPSKPKTN

SEQ ID NO: 227 - protein, 17 aa, Artificial Sequence
  Synthetically generated peptide
GGGQWNKPSKPKTNGGG

SEQ ID NO: 228 - protein, 17 aa, Artificial Sequence
  Synthetically generated peptide
GGGKKRPKPGGWNTGGG

SEQ ID NO: 229 - protein, 5 aa, Artificial Sequence
  Synthetically generated peptide
XXXXX

SEQ ID NO: 230 - protein, 6 aa, Artificial Sequence
  Synthetically generated peptide
XXXXXX

SEQ ID NO: 231 - protein, 9 aa, Artificial Sequence
  Synthetically generated peptide
XXXXXXXXX

SEQ ID NO: 232 - protein, 10 aa, Artificial Sequence
  Synthetically generated peptide
XXXXXXXXXX

SEQ ID NO: 233 - protein, 11 aa, Artificial Sequence
  Synthetically generated peptide
XXXXXXXXXXX

SEQ ID NO: 234 - protein, 11 aa, Artificial Sequence
  Synthetically generated peptide
XXXXXXXXXXX

SEQ ID NO: 235 - protein, 11 aa, Artificial Sequence
  Synthetically generated peptide
XXXXXXXXXXX

SEQ ID NO: 236 - protein, 11 aa, Artificial Sequence
  Synthetically generated peptide
XXXXXXXXXXX

SEQ ID NO: 237 - protein, 6 aa, Artificial Sequence
  Synthetically generated peptide
XXXXXX

SEQ ID NO: 238 - protein, 6 aa, Artificial Sequence
  Synthetically generated peptide
XXXXXX

SEQ ID NO: 239 - protein, 6 aa, Artificial Sequence
  Synthetically generated peptide
XXXXXX

SEQ ID NO: 240 - protein, 6 aa, Artificial Sequence
  Synthetically generated peptide
XXXXXX

SEQ ID NO: 241 - protein, 6 aa, Artificial Sequence
  Synthetically generated peptide
XXXXXX

SEQ ID NO: 242 - protein, 12 aa, Artificial Sequence
  Synthetically generated peptide
LGLCKKRPKPGG

SEQ ID NO: 243 - protein, 8 aa, Artificial Sequence
  Synthetically generated peptide
KKRPKPGG

SEQ ID NO: 244 - protein, 12 aa, Artificial Sequence
  Synthetically generated peptide
NKPSKPKTNMKH

SEQ ID NO: 245 - protein, 10 aa, Artificial Sequence
  Synthetically generated peptide
KPSKPKTNMK

SEQ ID NO: 246 - protein, 12 aa, Artificial Sequence
  Synthetically generated peptide
MHRYPNQVYYRP

SEQ ID NO: 247 - protein, 12 aa, Artificial Sequence
  Synthetically generated peptide
YQRGSSMVLFSS

SEQ ID NO: 248 - protein, 12 aa, Artificial Sequence
  Synthetically generated peptide
RYPGQGSPGGNR

SEQ ID NO: 249 - protein, 12 aa, Artificial Sequence
  Synthetically generated peptide
NITIKQHTVTTT

SEQ ID NO: 250 - protein, 12 aa, Artificial Sequence
  Synthetically generated peptide
QWNKPSKPKTNG

SEQ ID NO: 251 - protein, 12 aa, Artificial Sequence
  Synthetically generated peptide
LGLCKKRPKPGG

SEQ ID NO: 252 - protein, 7 aa, Artificial Sequence
  Synthetically generated peptide
XDFFVLX

SEQ ID NO: 253 - protein, 6 aa, Artificial Sequence
  Synthetically generated peptide
XFFVLX

SEQ ID NO: 254 - protein, 7 aa, Artificial Sequence
  Synthetically generated peptide
XKLVFFX

SEQ ID NO: 255 - protein, 14 aa, Artificial Sequence
  Synthetically generated peptide
XKLVFFXXKLVFFX

SEQ ID NO: 256 - protein, 13 aa, Artificial Sequence
  Synthetically generated peptide
XLGLCKKRPKPGX

SEQ ID NO: 257 - protein, 13 aa, Artificial Sequence
  Synthetically generated peptide
XRYPGQGSPGGNX

SEQ ID NO: 258 - protein, 13 aa, Artificial Sequence
  Synthetically generated peptide
XNKPSKPKTNMKX

SEQ ID NO: 259 - protein, 13 aa, Artificial Sequence
  Synthetically generated peptide
XMHRYPNQVYYRX

SEQ ID NO: 260 - protein, 13 aa, Artificial Sequence
  Synthetically generated peptide
XNITIKQHTVTTX

SEQ ID NO: 261 - protein, 13 aa, Artificial Sequence
  Synthetically generated peptide
XYQRGSSMVLFSX